SEQUENCE LISTING (80 sequences)

SEQ ID NO: 1 - DNA, 870 bp, Brasssica napus
  Napus_1A
atgtcaactacaacaatcgtccctctccgtcgcacttctaactctctcaatgaataccacactaacgcagtcgcctttgacggaatcgtcgggtcagcaagtactagccaaatggaggagattgttacgcaaaccgacgactgctacgccaaccccaacggagatggagggagaagcaagacgtcgttaatgacgtggaggatgtgcaatcctgtccacgtggtgagagtccattggataccgtgtttgttagcggtaggagttctgttcttcacgtgcgtagaggagtacatgctccagatgattccggcgagttctgagccgttcgatattggttttgtggcgacgggctctctgtatcgcctcttggcttcttcaccggatcttaataccgttttagctgctctcaacacggtgtttgtagggatgcaaacgacgtatattttatggacatggttggtggaaggacgaccacgagcgaccatctcggcttgcttcatgtttacttgccgtggcattctgggttactctactcagctccctcttcctcaggattttctaggatcaggggtagattttccggtaggaaacgtctcgttcttcctcttctactcaggccatgtcgcagggtcgacgatagcatccttggatatgaggagaatgaagaggttgagacttgccttgctttttgacatcctcaatgtattacaatcgatcaggcttctcgggacgagaggacaatacacgatcgatctcgctgtcggagttggcgctggggttctctttgactcactggctggaaaatacgaagagatgatgagcaagagacacaatgtaggcaatggttttagtttgatttcgtctcgctag

SEQ ID NO: 2 - protein, 289 aa, Brasssica napus
  Napus_1A
MSTTTIVPLRRTSNSLNEYHTNAVAFDGIVGSASTSQMEEIVTQTDDCYANPNGDGGRSKTSLMTWRMCNPVHVVRVHWIPCLLAVGVLFFTCVEEYMLQMIPASSEPFDIGFVATGSLYRLLASSPDLNTVLAALNTVFVGMQTTYILWTWLVEGRPRATISACFMFTCRGILGYSTQLPLPQDFLGSGVDFPVGNVSFFLFYSGHVAGSTIASLDMRRMKRLRLALLFDILNVLQSIRLLGTRGQYTIDLAVGVGAGVLFDSLAGKYEEMMSKRHNVGNGFSLISSR

SEQ ID NO: 3 - DNA, 708 bp, Brasssica napus
  Napus_2A
atgtctcaaatggacatttctacgagaactgaggaaggaggatggagaagcaagccttcgttcatgacgtggagagcgcgcgacgttgtctacgtgatgagacaccattggataccgtgtctgttcgcggccggattcttgttcgtcgtaagcgtggagtcctcgatcaagatggtttccgagagttctccaccgttcgatattgggtttgtggccacggagtctctgcatcatatcttggcttcttcaccggatctgaacaccggtttggccgctctaaactcggtgttaggagtgatgcaagtatcgtatattgcatggacatggttaatagaaggacggccacgagccaccatcacggctttattcctcttcacttgtcgcggtgttctcggttactgtactcagcttcctctttcaaaggagtatctaggttcagcaatcgatttcccgctaggaaacctgtcgttcttctattttttctcgggtcacgtggcaggcgcgaccatcgcatctttggacatgaggaggatgcagaggttgagacttgcgatggtttttgacatcctcaatgtactacagtcgatcaggctgctggcgacgagaggacactacacgatcgatctcgcaggtggagttgccgccgcgattctctttgactcattggccggcaagtacgaagctaatacaaggaagaggcaattgtag

SEQ ID NO: 4 - protein, 235 aa, Brasssica napus
  Napus_2A
MSQMDISTRTEEGGWRSKPSFMTWRARDVVYVMRHHWIPCLFAAGFLFVVSVESSIKMVSESSPPFDIGFVATESLHHILASSPDLNTGLAALNSVLGVMQVSYIAWTWLIEGRPRATITALFLFTCRGVLGYCTQLPLSKEYLGSAIDFPLGNLSFFYFFSGHVAGATIASLDMRRMQRLRLAMVFDILNVLQSIRLLATRGHYTIDLAGGVAAAILFDSLAGKYEANTRKRQL

SEQ ID NO: 5 - DNA, 870 bp, Brassica carinata
  Carinata_1B
atgtcaactacaacaattatccctctccgtcggagttctaactctctcaatgaataccacactaactcagtcgcctttgacggaatcgtcgggtcaacaagtactagccaaatggaggagattgttacgcaaacggacgaaggctacgccaaccccaacggagatggagggagaagcaaggtgtcgttcatgacgtggaggatgtgcagtgctgtccacgtggtgagagtccattggataccatgtttgttagcggtaggagttctgttcttcacgggggtggaggagtacatgctccagatgattcccccgagttctgagccgttcgatattggttttgtggcgacgcgctctctctatcgcctcttggcttcttcaccggatcttaacaccgttttagccgctctcaacacggtgttcgtagggatgcaaacgacgtatattgtatggacatggttgatggaaggacgaccaagagcgaccatttcggcttgcttcatgtttacctgtcgcggcattcttggttactctacgcagctccctcttcctcaggattttctaggatcaggggtagattttccggtaggaaacgtctcgttcttcctcttctactcaggccatgtggcagggtcgacgatagcatccttggacatgaggagaatgaagaggttgagactagccttgctttttgacatcctcaatgtattacaatcgatcaggcttctcgggacgagaggacaatacacgatcgatctcgctgtcggagttggcgctggggttctctttgactcactggctggaaaatacgaagagatgatgagcaagagacacaatgtaggcaatggttttagtttaatttcgactcgctag

SEQ ID NO: 6 - protein, 289 aa, Brassica carinata
  Carinata_1B
MSTTTIIPLRRSSNSLNEYHTNSVAFDGIVGSTSTSQMEEIVTQTDEGYANPNGDGGRSKVSFMTWRMCSAVHVVRVHWIPCLLAVGVLFFTGVEEYMLQMIPPSSEPFDIGFVATRSLYRLLASSPDLNTVLAALNTVFVGMQTTYIVWTWLMEGRPRATISACFMFTCRGILGYSTQLPLPQDFLGSGVDFPVGNVSFFLFYSGHVAGSTIASLDMRRMKRLRLALLFDILNVLQSIRLLGTRGQYTIDLAVGVGAGVLFDSLAGKYEEMMSKRHNVGNGFSLISTR

SEQ ID NO: 7 - DNA, 870 bp, Brassica carinata
  Carinata_1C
atgtcaactacaacaatcgtccctctccgtcgcagttctaactctctcaatgaataccacactaacgcagtcgcctttgacggaatcgtcgggtcaacaagtactagccaaatggaggagattgttacgcaaaccgacgactgctacgccaatcacaacggagatggagggagaagcaaggcatcgtttatgacgtggaggatgtgcaatcctgtccaggtggcgagagtccattggataccgtgtttgctagcggtaggagttctgttcttcacgggcgtagaggagtacatgctccagatgattccggcgagttctgagccgttcgatattggttttgtggcgacgcgctctctgtatcgactcttggcttcttcaccggatcttaataccgttttagctgctctcaacacggtgtttgtagggatgcaaacgacgtatattttatggacatggttggtggaaggacgaccacgagcgaccatctcggcttgcttcatgtttacttgtcgtggcattcttggttactctactcagctccctcttcctcaggattttctaggatcaggggtagattttccggtaggaaacgtctcgttcttcctcttctattctggccacgtagccggttcaatgatcacatccttggacatgaggagaatgcagaggttgagactagccttgctttttgacatcctcaatgtattacaatcgatcaggcttctcgggacgagaggacaatacacgatcgatctcgctgtcggagttggcgctggggttctctttgactcattggccgggaagtacgaagagatgatgagcaagagacgcaatgtaggcaatggttttagtttgatttcgtctcgctag

SEQ ID NO: 8 - protein, 289 aa, Brassica carinata
  Carinata_1C
MSTTTIVPLRRSSNSLNEYHTNAVAFDGIVGSTSTSQMEEIVTQTDDCYANHNGDGGRSKASFMTWRMCNPVQVARVHWIPCLLAVGVLFFTGVEEYMLQMIPASSEPFDIGFVATRSLYRLLASSPDLNTVLAALNTVFVGMQTTYILWTWLVEGRPRATISACFMFTCRGILGYSTQLPLPQDFLGSGVDFPVGNVSFFLFYSGHVAGSMITSLDMRRMQRLRLALLFDILNVLQSIRLLGTRGQYTIDLAVGVGAGVLFDSLAGKYEEMMSKRRNVGNGFSLISSR

SEQ ID NO: 9 - DNA, 708 bp, Brassica carinata
  Carinata_2B
atgtctcaaatggacatttctatgagaacggaggaaggaggatggagaagcaagccgtcgtttatgacgtggagagcgcgcgacgttgtctacgtgttgagacaccattggataccgtgtttgttcgcggccggattcttgttcgtcgtaagcgtggagtcctcgatcaagatggtttccgagagttctccgccgctcgatattgggtttgtggccactcgctctctccatcatctcttggcttcttcaccggatctgaacaccggtttggccgctctaaactcggtgttaggagtgatgcaagtatcgtatattgcatggacatggttaatagaaggacggccacgagccaccatcacggctttattcctcttcacttgtcgcggcgttctcggttactgtactcagctccctctttcaaaggagtatctaggatcagcaatcgatttcccgctaggaaacctctcgttcttctatttcttctcgggtcacgtggcaggcacgaccatcgcatctttggacatgaggagaatgcagaggttgagacttgcgatggtttttgacatcctcaatgtattacaatcgatcaggctgctagcgacgagaggacactacacgatcgatctcgcaggtggagttgccgccgcgattctctttgactcattggccggcaagtacgaagcaagtacaagaaagaggcaattgtag

SEQ ID NO: 10 - protein, 235 aa, Brassica carinata
  Carinata_2B
MSQMDISMRTEEGGWRSKPSFMTWRARDVVYVLRHHWIPCLFAAGFLFVVSVESSIKMVSESSPPLDIGFVATRSLHHLLASSPDLNTGLAALNSVLGVMQVSYIAWTWLIEGRPRATITALFLFTCRGVLGYCTQLPLSKEYLGSAIDFPLGNLSFFYFFSGHVAGTTIASLDMRRMQRLRLAMVFDILNVLQSIRLLATRGHYTIDLAGGVAAAILFDSLAGKYEASTRKRQL

SEQ ID NO: 11 - DNA, 708 bp, Brassica carinata
  Carinata_2C
atgtctcaaatggacatttctacgagaacggaggaaggaggatggagaagcaagccgtcgtttatgacgtggagagcgcgcgacgttgtctacgtgttgagacaccattggataccgtgtttgttcgcggccggattcttgttcgtcgtaagcgtggagtcctcgatcaagatggtttccgagagttctccgccgctcgatattgggtttgtggccactcgctctctccatcatctcttggcttcttcaccggatctgaacaccggtttggccgctctaaactcggtgttaggagtgatgcaagtatcgtatattgcatggacatggttaatagaaggacggccacgagccaccatcacggctttattcctcttcacttgtcgcggcgttctcggttactgtactcagctccctctttcaaaggagtatctaggatcagcaatcgatttcccgctaggaaacctctcgttcttctatttcttctcgggtcacgtggcaggcacgaccatcgcatctttggacatgaggagaatgcagaggttgagacttgcgatggtttttgacatcctcaatgtattacaatcgatcaggctgctagcgacgagaggacactacacgatcgatctcgcaggtggagttgccgccgcgattctctttgactcattggccggcaagtacgaagcaaatacaagaaagaggcaattgtag

SEQ ID NO: 12 - protein, 235 aa, Brassica carinata
  Carinata_2C
MSQMDISTRTEEGGWRSKPSFMTWRARDVVYVLRHHWIPCLFAAGFLFVVSVESSIKMVSESSPPLDIGFVATRSLHHLLASSPDLNTGLAALNSVLGVMQVSYIAWTWLIEGRPRATITALFLFTCRGVLGYCTQLPLSKEYLGSAIDFPLGNLSFFYFFSGHVAGTTIASLDMRRMQRLRLAMVFDILNVLQSIRLLATRGHYTIDLAGGVAAAILFDSLAGKYEANTRKRQL

SEQ ID NO: 13 - DNA, 708 bp, Brassica juncea
  BjROD1-B4
atgtctcaaatggacatttctacgagaactgaggaaggaggatggagaagcaagccttcgttcatgacgtggagagcgcgcgacgttgtctacgtgatgagacaccattggataccgtgtctgttcgcggccggattcttgttcgtcgtaagcgtggagtcctcgatcaagatggtttccgagagttctccaccgttcgatattgggtttgtggccacggagtctctgcatcatatcttggcttcttcaccggatctgaacaccggtttggccgctctaaactcggtgttaggagtgatgcaagtatcgtatattgcatggacatggttaatagaaggacggccacgagccaccatcacggctttattcctcttcacttgtcgcggcgttctcggttactgtacgcagctccctctttcaaaggagtatctaggatcagcaatcgatttcccgctaggaaacctctcgttcttctattttttctcgggtcacgtggcaggcacgaccatcgcatctttggacatgaggagaatgcagaggttgagacttgcgatggtttttgacatcctcaatgtattacagtcgatcaggctgcttgcgacgagaggacactacacgatcgatctcgcaggtggagttgccgccgcgattctctttgactcattggccggcaagtacgaagcaaatacaagaaagaggcaattgtag

SEQ ID NO: 14 - protein, 235 aa, Brassica juncea
  BjROD1-B4
MSQMDISTRTEEGGWRSKPSFMTWRARDVVYVMRHHWIPCLFAAGFLFVVSVESSIKMVSESSPPFDIGFVATESLHHILASSPDLNTGLAALNSVLGVMQVSYIAWTWLIEGRPRATITALFLFTCRGVLGYCTQLPLSKEYLGSAIDFPLGNLSFFYFFSGHVAGTTIASLDMRRMQRLRLAMVFDILNVLQSIRLLATRGHYTIDLAGGVAAAILFDSLAGKYEANTRKRQL

SEQ ID NO: 15 - DNA, 870 bp, Brassica juncea
  BjROD1-A3
atgtcaactacaacaatcgtccctctccgtcgcacttctaactctctcaatgaataccacactaacgcagtcgcctttgacggaatcgtcgggtcagcaagtactagccaaatggaggagattgttacgcaaaccgacgactgctacgccaaccccaacggagatggagggagaagcaaggtgtcgttaatgacgtggaggatgtgcaatcctgtccacgtggtgagagtccattggataccgtgtttgttagcggtaggagttctgttcttcacgtgcgtagaggagtacatgctccagatgattccggcgagttctgagccgttcgatattggttttgtggcgacgggctctctgtatcgcctcttggcttcttcaccggatcttaataccgttttagctgctctcaacacggtgtttgtagggatgcaaacgacgtatattgtatggacatggttgatggaaggacgaccacgagcgaccatctcggcttgctttatgtttacttgccgtggcattctgggttactctactcagctccctcttcctcaggattttctaggatcaggggtagattttccggtaggaaacgtctcgttcttcctcttctactcaggccatgtcgcagggtcgacgatagcatccttggatatgaggagaatgaagaggttgagacttgccttgctttttgacatcctcaatgtattacaatcgatcaggcttctcgggacgagaggacaatacacgatcgatctcgctgtcggagttggcgctggggttctctttgactcactggctggaaaatacgaagagatgatgagcaagagacacaatgtaggcaatggttttagtttaatttcgactcgctag

SEQ ID NO: 16 - protein, 289 aa, Brassica juncea
  BjROD1-A3
MSTTTIVPLRRTSNSLNEYHTNAVAFDGIVGSASTSQMEEIVTQTDDCYANPNGDGGRSKVSLMTWRMCNPVHVVRVHWIPCLLAVGVLFFTCVEEYMLQMIPASSEPFDIGFVATGSLYRLLASSPDLNTVLAALNTVFVGMQTTYIVWTWLMEGRPRATISACFMFTCRGILGYSTQLPLPQDFLGSGVDFPVGNVSFFLFYSGHVAGSTIASLDMRRMKRLRLALLFDILNVLQSIRLLGTRGQYTIDLAVGVGAGVLFDSLAGKYEEMMSKRHNVGNGFSLISTR

SEQ ID NO: 17 - DNA, 849 bp, Brasssica napus
  Napus_3A
atgtcaactaataccgtcgtccctctccgtcgcagatctaacggatatcacactaacggcgtggcctttaacggaatggataatattgtcaagaaaaccgacgactgctacaccaacggcaacggcaacggaggagtagagagaagcaaagcctcgtttctgacatggaccatgcgtgacgctgtctacgtagcgagataccattggataccgtgtttctttgcggtcggagttctgttctttatgggggttgagtacacgctccagatggttccggcgaagtctgagccgttcgatattgggtttgtggccacgcgctctctgaaccgcgtcttggcgagttcaccggatcttaacacccttttagcggctctaaacacggtattcgtagcgatgcaaacgacgtatattgtatggacatggttgatggaaggaagaccacgagccactatctcggcttgcttcatgtttacttgtcgcggcattcttggttactctactcagctccctctaccacaggattttttaggatcaggagttgattttccggtgggaaacgtctcattcttcctcttctattctggccacgtagccggttcaatgatcgcatccttggacatgaggagaatgcagaggttgagactagcgatgctttttgacatcctcaacatattacaatcgatcagactgctcgggacgagaggacactacacgatcgatcttgcggtcggagttggcgctgggattctctttgactcattggccgggaagtacgaagagatgatgagcaagagacacaatttagccaatggttttagtttgatttctaaagactcgctagtcaattaa

SEQ ID NO: 18 - protein, 282 aa, Brasssica napus
  Napus_3A
MSTNTVVPLRRRSNGYHTNGVAFNGMDNIVKKTDDCYTNGNGNGGVERSKASFLTWTMRDAVYVARYHWIPCFFAVGVLFFMGVEYTLQMVPAKSEPFDIGFVATRSLNRVLASSPDLNTLLAALNTVFVAMQTTYIVWTWLMEGRPRATISACFMFTCRGILGYSTQLPLPQDFLGSGVDFPVGNVSFFLFYSGHVAGSMIASLDMRRMQRLRLAMLFDILNILQSIRLLGTRGHYTIDLAVGVGAGILFDSLAGKYEEMMSKRHNLANGFSLISKDSLVN

SEQ ID NO: 19 - DNA, 852 bp, Brasssica napus
  Napus_5A
atgtcaactgaaactagcgtccctctccgtcgcagatctacctctcttaacggacatcactctaacgacgtcgcctttgacggaaccgtcccattaatggagaacaacattgttaagaaaacagacgacggctacgccaatggaggaggaaaggcgtcgtttatgacatggacggcgcgtgacgctatctacgtggcgagagtccattggataccgtgtgtgttcgcggttggagttctcttcttcatgggcgtcgagtatacgcttcaaatgattcccgcgaggtctgagccgttcgatattgggtttgtggtcacgcgctctctgaaccgcgtcttggcaaattcaccgggtcttaacaccgttttagccgcactaaacacggtgttcgtagggatgcaaactacgtatattgtatggacatggttgatggaaggaagaccacgagccaccatctcggcttgcttcatgtttacttgtcgcggtatccttggttactctactcagctccctctccctcaggagtttttaggatcaggagtcgattttcctgtgggaaacgtctcattcttccttttctactcgggtcacgtcgccggttcgatgatagcatccttagacatgaggagaatgcagaggttgagactagcaatgctttttgacatcctcaatgtattacaatcgatacggctgctcgggacgagaggacattacaccatcgatcttgcggtcggagttggcgctgggattctctttgactcgttggccgggaagtacgaagagatgatgagcaaaagacacaatttaggcaatggttttagtttgatttctaaagactcgctagacaattaa

SEQ ID NO: 20 - protein, 283 aa, Brasssica napus
  Napus_5A
MSTETSVPLRRRSTSLNGHHSNDVAFDGTVPLMENNIVKKTDDGYANGGGKASFMTWTARDAIYVARVHWIPCVFAVGVLFFMGVEYTLQMIPARSEPFDIGFVVTRSLNRVLANSPGLNTVLAALNTVFVGMQTTYIVWTWLMEGRPRATISACFMFTCRGILGYSTQLPLPQEFLGSGVDFPVGNVSFFLFYSGHVAGSMIASLDMRRMQRLRLAMLFDILNVLQSIRLLGTRGHYTIDLAVGVGAGILFDSLAGKYEEMMSKRHNLGNGFSLISKDSLDN

SEQ ID NO: 21 - DNA, 843 bp, Brassica carinata
  Carinata_3B
atgtcaactaataccgtcgtccctctccgtcgcagatctaacggatatcacagtaacggcgtggcctttaacggaatggagaacattgtcaagaaaacagacgactgctacaccaacggcaacggaggaggagggaagagcaaggcgtcgtttctgacatggaccatgcgcgacgctgtctacgtggcgagataccattggataccgtgtttctttgcggtcggagttctgttctttatgggcgttgagtatacgctccagatggttccggcgaagtctgagccgttcgatattgggtttgtggccacgcgctctctgaaccgcgtcttggcgagttcaccggatcttaacacccttttagcggctctaaacacggtattcgtagcgatgcaaacgacgtatattgtatggacatggttgatggaaggaagaccacgagccactatctctgcttgctttatgtttacttgtcgtggcattcttggttactctactcagctccctctcccacaggattttttaggatcaggagttgattttccagtgggaaacgtctcattcttcctcttctattctggtcacgtcgccggttcaatgatcgcatccttggacatgaggagaatgcggaggttgagactagcgatgctttttgacatcctcaacgtattacaatctatcaggctgctcgggacaagaggacattacacgattgatcttgcggtcggagttggcgctgggattctctttgactctttggccgggaagtacgaagagatgatgagcaagagacacaatttagccaatggttttagtttgatttcgaaagaatccctagtcaattaa

SEQ ID NO: 22 - protein, 280 aa, Brassica carinata
  Carinata_3B
MSTNTVVPLRRRSNGYHSNGVAFNGMENIVKKTDDCYTNGNGGGGKSKASFLTWTMRDAVYVARYHWIPCFFAVGVLFFMGVEYTLQMVPAKSEPFDIGFVATRSLNRVLASSPDLNTLLAALNTVFVAMQTTYIVWTWLMEGRPRATISACFMFTCRGILGYSTQLPLPQDFLGSGVDFPVGNVSFFLFYSGHVAGSMIASLDMRRMRRLRLAMLFDILNVLQSIRLLGTRGHYTIDLAVGVGAGILFDSLAGKYEEMMSKRHNLANGFSLISKESLVN

SEQ ID NO: 23 - DNA, 843 bp, Brassica carinata
  Carinata_3C
atgtcaactaataccgtcgtccctctccgtcgcagatctaacggatatcacactaacggcgtggccttcaacggaatggagaacattgtcaagaaaaccgacgactgctacaccaatggcaacggagtaggagggaagagcaaggcgtcgtttctgacatggaccatgcgtgacgctgtctacgtagcgagataccattggataccgtgtttctttgcggtcggagttctgttctttatgggggttgagtacacgctccagatggttccggcgaagtctgagccgttcgatattgggtttgtggccacgcgctctctgaaccgcgtcttggcgagttcaccggatcttaacacccttttagcggctctaaacacggtattcgtagcgatgcaaacgacgtatattgtatggacatggttgatggaaggaagaccacgagccactatctcggcttgcttcatgtttacttgtcgcggtattcttggttactctactcagctccctctaccacaggattttttaggatcaggagttgattttccggtgggaaacgtctcattcttcctcttctattctggccacgtagccggttcaatgatcgcatccttggacatgaggagaatgcagaggttgagactagcgatgctttttgacatcctcaacatattacaatcgatcaggctgctcgggacgagaggacactacacgatcgatcttgcggtcggagttggcgctgggattctctttgactcattggccgggaagtacgaagagatgatgagcaagagacacaatttagccaatggttttagtttgatttctaaagactcgctagtcaattaa

SEQ ID NO: 24 - protein, 280 aa, Brassica carinata
  Carinata_3C
MSTNTVVPLRRRSNGYHTNGVAFNGMENIVKKTDDCYTNGNGVGGKSKASFLTWTMRDAVYVARYHWIPCFFAVGVLFFMGVEYTLQMVPAKSEPFDIGFVATRSLNRVLASSPDLNTLLAALNTVFVAMQTTYIVWTWLMEGRPRATISACFMFTCRGILGYSTQLPLPQDFLGSGVDFPVGNVSFFLFYSGHVAGSMIASLDMRRMQRLRLAMLFDILNILQSIRLLGTRGHYTIDLAVGVGAGILFDSLAGKYEEMMSKRHNLANGFSLISKDSLVN

SEQ ID NO: 25 - DNA, 849 bp, Brassica carinata
  Carinata_5B
atgtcaactgaaactggcgtccctctccgtcgcagatctaactctcttaacggacatcacactaacggcgtcgcctctgacggaaacgtcccatcaatggagagcattgttaagagaacagacggaggaggaggagggatcagaagcaaggcgtcgtttatgacatggacggcgcgtgacgctatctacgtggcgagagtccattggataccgtgtgtgttcgcggtcggagttctgttcttcatgggcgtcgagtatacgcttcagatgattcccgcgaggtctgagccgttcgatattgggttcgtggccacgcgctctctgaaccgcgtcttggcaaactcaccggatcttaacaccgttttagccgcactaaacacggttttcgtagggatgcaaacgacgtatattgtatggacatggttgatggaaggacgaccacgagcgaccatctcggcttgctttatgtttacttgtcgcggcattcttggttactctactcagcttcctctccctcaggagtttttaggatcaggagtcgattttccggtgggaaacgtctcattcttcctcttctactcgggtcacgtcgccggttccatgatagcatccttggacatgaggagaatgcagaggttgagactagcgatgctttttgacatcctcaatgtactacaatccatcaggctgctcgggacgagaggacattacaccatcgatcttgcggtcggagttggcgctgggattctctttgactcgttggccgggaagtacgaagagatgatgaacaagagacacaatttaggcaatggttttagtttgatttctaaagactcgctagtcaattaa

SEQ ID NO: 26 - protein, 282 aa, Brassica carinata
  Carinata_5B
MSTETGVPLRRRSNSLNGHHTNGVASDGNVPSMESIVKRTDGGGGGIRSKASFMTWTARDAIYVARVHWIPCVFAVGVLFFMGVEYTLQMIPARSEPFDIGFVATRSLNRVLANSPDLNTVLAALNTVFVGMQTTYIVWTWLMEGRPRATISACFMFTCRGILGYSTQLPLPQEFLGSGVDFPVGNVSFFLFYSGHVAGSMIASLDMRRMQRLRLAMLFDILNVLQSIRLLGTRGHYTIDLAVGVGAGILFDSLAGKYEEMMNKRHNLGNGFSLISKDSLVN

SEQ ID NO: 27 - DNA, 852 bp, Brassica carinata
  Carinata_5C
atgtcaactgaaactggcgtccctctccgtcgcagatctaactctcttaacggacatcactctaacgacgtcgcctttgacggaaccgtcccatcaatggagaacaacattgttaagaaaacagacgacggctacgccaatggaggaggaaaggcgtcgtttatgacatggacggcgcgtgacgctatctacgtggcgagagtccattggataccgtgtgtgttcgcggttggagttctgttcttcatgggcgtcgagtatacgcttcagatgattcccgcgaggtctgagccgttcgatgttgggtttgtggccacgcgctctctgaacagcgtcttggcaaattcaccgggtcttaacaccgttttagccgcactaaacacggtgttcgtagggatgcaaactacgtatattgtatggacatggttgatggaaggaagaccacgagccaccatctcggcttgcttcatgtttacttgtcgcggtattcttggttactctactcagctccctctccctcaggagtttttaggatcaggagtcgattttcctgtgggaaacgtctcattcttccttttctactcgggtcacgtcgccggttcgatgatagcatccttagacatgaggagaatgcagaggttgagactagcaatgctttttgacatcctcaatgtattacaatcgataaggctgctcgggacgagaggacattacaccatcgatcttgcggtcggagttggcgctgggattctctttgactcgttggccgggaagtacgaagagatgatgagcaaaagacacaatttaggcaatggttttagtttgatttctaaagactcgctagtcaattaa

SEQ ID NO: 28 - protein, 283 aa, Brassica carinata
  Carinata_5C
MSTETGVPLRRRSNSLNGHHSNDVAFDGTVPSMENNIVKKTDDGYANGGGKASFMTWTARDAIYVARVHWIPCVFAVGVLFFMGVEYTLQMIPARSEPFDVGFVATRSLNSVLANSPGLNTVLAALNTVFVGMQTTYIVWTWLMEGRPRATISACFMFTCRGILGYSTQLPLPQEFLGSGVDFPVGNVSFFLFYSGHVAGSMIASLDMRRMQRLRLAMLFDILNVLQSIRLLGTRGHYTIDLAVGVGAGILFDSLAGKYEEMMSKRHNLGNGFSLISKDSLVN

SEQ ID NO: 29 - DNA, 818 bp, Brassica juncea
  BjROD1-A2
atgtcaactgaaactagcgtccctctccgtcgcagatctacctctcttaacggacatcactctaacgacgtcgcctttgacggaaccgtcccattaatggagaacaacattgttaagaaaacagacgacggctacgccaatggaggaggaaaggcgtcgtttatgacatggacggcgcgtgacgctatctacgtggcgagagtccattggataccgtgtgtgttcgcggttggagttctcttcttcatgggcgtcgagtatacgcttcaaatgattcccgcgaggtctgagccgttcgatattgggtttgtggtcacgcgctctctgaaccgcgtcttggcaaattcaccggctcttaacaccgttttagccgcactaaacacggtgttcgtagggatgcaaactacgtatattgtatggacatggttgatggaaggaagaccacgggccaccatctcggcttgcttcatgtttacttgtcgcgactctacccagcttcctctccctcaggagtttttaggatcaggagtcgattttccggtgggaaacgtctcattcttcctcttctactcgggtcacgtcgccggttccatgatagcatccttggacatgaggagaatgcagaggttgagactagcgatgctttttgacatcctcaatgtactacaatccatcaggctgctcgggacgagaggacattacaccatcgatcttgcggtcggagttggcgctgggattctctttgactcgttggccgggaagtacgaagagatgatgagcaaaagacacaatttaggcaatggttttagtttgatttc

SEQ ID NO: 30 - protein, 272 aa, Brassica juncea
  BjROD1-A2
MSTETSVPLRRRSTSLNGHHSNDVAFDGTVPLMENNIVKKTDDGYANGGGKASFMTWTARDAIYVARVHWIPCVFAVGVLFFMGVEYTLQMIPARSEPFDIGFVVTRSLNRVLANSPALNTVLAALNTVFVGMQTTYIVWTWLMEGRPRATISACFMFTCRDSTQLPLPQEFLGSGVDFPVGNVSFFLFYSGHVAGSMIASLDMRRMQRLRLAMLFDILNVLQSIRLLGTRGHYTIDLAVGVGAGILFDSLAGKYEEMMSKRHNLGNGFSLI

SEQ ID NO: 31 - DNA, 807 bp, Brassica juncea
  BjROD1-B2
atgtcaactgaaactggcgtccctctccgtcgcagatctaactctcttaacggacatcacactaacggcgtcgcctctgacggaacaaacgtcccattaatggagaaggcgtcgtttatgacatggacggcgcgtgacgctatctacgtggcgagagtccattggataccgtgtgtgttcgcggtcggagttctgttcttcatgggcgtcgagtatacgcttcagatgattcccgcgaggtctgagccgttcgatattgggttcgtggccacgcgctctctgaatcgcgtcttggcagattcaccggatcttaacaccgttttagctgcactaaacacggttttcgtagggatgcaaactacgtatattgtatggacatggttgatggaaggaagaccacgggccaccatctcggcttgcttcatgtttacttgtcgcggtattcttggttactctactcagctccctctccctcaggagtttttaggatcaggagtcgattttccggtgggaaacgtctcattcttcctcttctactcgggtcacgtcgccggttccatgatagcatccttggacatgaggagaatgcagaggttgagactagcgatgctttttgacatcctcaatgtactacaatccatcaggctgctcgggacgagaggacattacaccatcgatcttgcggtcggagttggcgctgggattctctttgactcgttggccgggaagtacgaagagatgatgagcaaaagacacaatttaggcaatggttttagtttgatttctaaagactcgctagtcaattaa

SEQ ID NO: 32 - protein, 268 aa, Brassica juncea
  BjROD1-B2
MSTETGVPLRRRSNSLNGHHTNGVASDGTNVPLMEKASFMTWTARDAIYVARVHWIPCVFAVGVLFFMGVEYTLQMIPARSEPFDIGFVATRSLNRVLADSPDLNTVLAALNTVFVGMQTTYIVWTWLMEGRPRATISACFMFTCRGILGYSTQLPLPQEFLGSGVDFPVGNVSFFLFYSGHVAGSMIASLDMRRMQRLRLAMLFDILNVLQSIRLLGTRGHYTIDLAVGVGAGILFDSLAGKYEEMMSKRHNLGNGFSLISKDSLVN

SEQ ID NO: 33 - DNA, 894 bp, Camelina sativa
  Camelina_C15(45897)
atgtcagtcgccgcagctaaacccgccgtctctcgccgtcacgtatctaacggaaacaacactaacaacgtcgccattgacgacgatcacaaccaccaacgccgcatcgtcggagataaaaacactcgaatggagatcgctgctaagaacaacggctacgccaacggtgtcatcggaggaggaggatggaggagcaaggcgtcgttcatgacgtggacgacgcgtgacgttgtctacgtggcgagacaccattggataccgtgcatgttcgctgccgggcttttgttcttcatgggggtcgagtacacgctccagatgattcccgcgagatctgagccgttcgatcttgggttcgtggccacgcgctctttgaatcgcgtcttagcatcttccccggatcttaacaccgttttagccgcactaaacacggtgttcgtattgatgcaaacaacgtatattgtatggacatggttagtggaaggacgagcacgagcaaccatctcggctttattcatgttcacgtgtcggggcattctcggctactctactcagcttcctctccctcaggattttttaggatcaggagttgattttccagtgggaaacgtctctttcttcctcttcttctcgggccacgttgccggctcgatgatcgcatcactggacatgaggagaatgcagaggtttaagctggcgagggtttttgacatcctcaatgtattacaatcgatcaggctgctcggtacaagaggacactacaccatcgaccttgcggttggagttggcgctgggattctctttgactcactggccgggaagtacgaagagatgagcagaagacaccacctaggaactggttttagtttgatatcgaaagactctctagtcaattaa

SEQ ID NO: 34 - protein, 297 aa, Camelina sativa
  Camelina_C15(45897)
MSVAAAKPAVSRRHVSNGNNTNNVAIDDDHNHQRRIVGDKNTRMEIAAKNNGYANGVIGGGGWRSKASFMTWTTRDVVYVARHHWIPCMFAAGLLFFMGVEYTLQMIPARSEPFDLGFVATRSLNRVLASSPDLNTVLAALNTVFVLMQTTYIVWTWLVEGRARATISALFMFTCRGILGYSTQLPLPQDFLGSGVDFPVGNVSFFLFFSGHVAGSMIASLDMRRMQRFKLARVFDILNVLQSIRLLGTRGHYTIDLAVGVGAGILFDSLAGKYEEMSRRHHLGTGFSLISKDSLVN

SEQ ID NO: 35 - DNA, 894 bp, Camelina sativa
  Camelina_C19(65416)
atgtcagccaccgcagctaaacccgctgtctctcgccgtcacgtatctaacggaaaccacactaacaacgtcgccattgacgacgatcacaaccaccaacgccccgtcgacgtcggagatacaaacactcgaatggagatcgctgctaagaacaacggctacgccaacggtgtcatcggaggaggatggaggagcaaggcgtcgttcatgacgtggacgacgcgtgacgttgtctacgtggcgagacaccattggataccgtgcatgttcgctgccgggcttttgttcttcatgggggtcgagtacacgctccagatgatacccgcgagatctgagccgttcgatcttgggtttgtagccacgcgctctttgaatcgcgtcttagcatcttccccggatctaaacactgttctagccgcactaaacacggtgttcgtattgatgcaaacaacgtatattgtatggacatggttagtggaaggacgagcacgagcaaccatctcggctttattcatgttcacgtgtcgcggcattctcggctactctactcagcttcctctccctcaggattttttaggatcaggagttgattttccggtgggaaacgtctctttcttcctcttcttctcgggccacgttgccggctcgatgatcgcatcgttggacatgaggagaatgcagaggtttaagctggcgagggtttttgacatcctcaatgtattacaatcgatcaggctgctcggtacaagaggacactacaccatcgaccttgcggttggagttggcgctgggattctttttgactcactggccggaaagtacgaagagatgagcagaagacaccacctaggaactggttttagtttgatatcgaaagactctctagtcaattaa

SEQ ID NO: 36 - protein, 297 aa, Camelina sativa
  Camelina_C19(65416)
MSATAAKPAVSRRHVSNGNHTNNVAIDDDHNHQRPVDVGDTNTRMEIAAKNNGYANGVIGGGWRSKASFMTWTTRDVVYVARHHWIPCMFAAGLLFFMGVEYTLQMIPARSEPFDLGFVATRSLNRVLASSPDLNTVLAALNTVFVLMQTTYIVWTWLVEGRARATISALFMFTCRGILGYSTQLPLPQDFLGSGVDFPVGNVSFFLFFSGHVAGSMIASLDMRRMQRFKLARVFDILNVLQSIRLLGTRGHYTIDLAVGVGAGILFDSLAGKYEEMSRRHHLGTGFSLISKDSLVN

SEQ ID NO: 37 - DNA, 891 bp, Camelina sativa
  Camelina_C1(80666)
atgtcagcctccgcagctaaacccgccgtctctcgccgtcacgtatctaacggaaaccacactaacaacgtcgccattgagaacgatcacaaccaccaacgccccgtcgccggagatacaaacactcgaatggagatcgctgctaagaacaacggctacgccaacggtgtcggaggaggaggatggaggagcaaggcgtcgttcatgacgtggacgacgcgtgacgttgtctacgtggcgagacaccattggataccgtgcatgttcgctgccgggcttttgttcttcatgggggtcgagtacacgctccagatgatacccgcgagatctgaaccgttcgatcttgggtttgtggccacgcgctctttaaatcgcgtcttagcatcttccccggatctaaacactgttctagccgcactaaacacggtgttcgtattgatgcaaacaacgtatattgtatggacatggttagtggaaggacgagcacgagcaaccatctcggctttattcatgttcacgtgtcgcggcattctcggctactctactcagcttcctctccctcaggattttttaggatcaggagttgattttccggtgggaaacgtctctttcttcctcttcttctcgggccacgttgccggctcgatgatcgcatcgttggacatgaggagaatgcaaaggtttaagctggcgagggtttttgacatcctcaatgtattacaatcgatcaggctgctcggtacaagaggacactacaccatcgaccttgcggttggagttggcgctgggattctctttgactcactggccgggaagtacgaagagatgagcagaagacaccacctaggaactggttttagtttgatatcgaaagactctctagtcaattaa

SEQ ID NO: 38 - protein, 296 aa, Camelina sativa
  Camelina_C1(80666)
MSASAAKPAVSRRHVSNGNHTNNVAIENDHNHQRPVAGDTNTRMEIAAKNNGYANGVGGGGWRSKASFMTWTTRDVVYVARHHWIPCMFAAGLLFFMGVEYTLQMIPARSEPFDLGFVATRSLNRVLASSPDLNTVLAALNTVFVLMQTTYIVWTWLVEGRARATISALFMFTCRGILGYSTQLPLPQDFLGSGVDFPVGNVSFFLFFSGHVAGSMIASLDMRRMQRFKLARVFDILNVLQSIRLLGTRGHYTIDLAVGVGAGILFDSLAGKYEEMSRRHHLGTGFSLISKDSLVN

SEQ ID NO: 39 - DNA, 870 bp, Brassica juncea
  BjROD1-B3
atgtcaactacaacaatcgtccctctccgtcggagttctaactctctcaatgaataccacactaacgcagtcgcctttgacggaatcgtcgggtcaacaagtactagccaaatggaggagattgttacgcaaatggacgaaggctacgccaaccccaacggagatggagggagaagcaaggtgtcgttcatgacgtggaggatgtgcagtgctgtccacgtggtgagagtccactggataccgtgtttgttagcggtaggagttctgttcttcacgggggtggaggagtacatgctccagatgattcccccgagttctgagccgttcgatattggttttgtggcgacgcgctctctctatcgcctcttggcttcttcaccggatctcaacaccgttttagccgctctcaacacggtgttcgtagggatgcaaacgacgtatattgtatggacatggttgatggaaggacgaccacgagcgaccatctcggcttgctttatgtttacatgtcgtggcattcttggttactctactcagctccctcttcctcaggattttctaggatcaggggtagactttcctgtaggaaacgtctccttcttcctcttctactcaggccatgtggcagggtcgacgatagcatccttggacatgaggagaatgaagaggttgagactagccttgctttttgacatcctcaatgtattacaatcgatcaggcttctcgggacgagaggacaatacacgatcgatctcgctgtcggagttggcgctggggttctctttgactcactggctggaaaatacgaagagatgatgagcaagagacacaatgtaggcaatggttttagtttgatttcgtctcgctag

SEQ ID NO: 40 - protein, 289 aa, Brassica juncea
  BjROD1-B3
MSTTTIVPLRRSSNSLNEYHTNAVAFDGIVGSTSTSQMEEIVTQMDEGYANPNGDGGRSKVSFMTWRMCSAVHVVRVHWIPCLLAVGVLFFTGVEEYMLQMIPPSSEPFDIGFVATRSLYRLLASSPDLNTVLAALNTVFVGMQTTYIVWTWLMEGRPRATISACFMFTCRGILGYSTQLPLPQDFLGSGVDFPVGNVSFFLFYSGHVAGSTIASLDMRRMKRLRLALLFDILNVLQSIRLLGTRGQYTIDLAVGVGAGVLFDSLAGKYEEMMSKRHNVGNGFSLISSR

SEQ ID NO: 41 - DNA, 870 bp, Brasssica napus
  Napus_1C
atgtcaactacaacaatcgtccctctccgtcgcagttctaactctctcaatgaataccacactaacgcagtcgcctttgacggaatcgtcgggtcaacaagtactagccaaatggaggagattgttacgcaaaccgacgactgctacgccaatcacaacggagatggagggagaagcaaggcatcgtttatgacgtggaggatgtgcaatcctgtccaggtggcgagagtccattggataccgtgtttgctagcggtaggagttctgttcttcacgggcgtagaggagtacatgctccagatgattccggcgagttctgagccgttcgatattggttttgtggcgacgcgctctctgtatcgactcttggcttcttcaccggatcttaataccgttttagctgctctcaacacggtgtttgtagggatgcaaacgacgtatattttatggacatggttggtggaaggacgaccacgagcgaccatctcggcttgcttcatgtttacttgtcgtggcattcttggttactctactcagctccctcttcctcaggattttctaggatcaggggtagattttccggtaggaaacgtctcgttcttcctcttctactcaggccatgtcgcagggtcgacgatagcatccttggatatgaggagaatgaagaggttgagactagccttgctttttgacatcctcaatgtattacaatcgatcaggcttctcgggacgagaggacaatacacgatcgatctcgctgtcggagttggcgctggggttctctttgactcactggctggaaaatacgaagagatgatgagcaagagacgcaatgtaggcaatggttttagtttgatttcgtctcgctag

SEQ ID NO: 42 - protein, 289 aa, Brasssica napus
  Napus_1C
MSTTTIVPLRRSSNSLNEYHTNAVAFDGIVGSTSTSQMEEIVTQTDDCYANHNGDGGRSKASFMTWRMCNPVQVARVHWIPCLLAVGVLFFTGVEEYMLQMIPASSEPFDIGFVATRSLYRLLASSPDLNTVLAALNTVFVGMQTTYILWTWLVEGRPRATISACFMFTCRGILGYSTQLPLPQDFLGSGVDFPVGNVSFFLFYSGHVAGSTIASLDMRRMKRLRLALLFDILNVLQSIRLLGTRGQYTIDLAVGVGAGVLFDSLAGKYEEMMSKRRNVGNGFSLISSR

SEQ ID NO: 43 - DNA, 708 bp, Brasssica napus
  Napus_2C
atgtctcaaatggacatttctacgagaactgaggaaggaggatggagaagcaagccttcgtttatgacgtggagagcgcgcgacgttgtctacgtgatgagacaccattggataccgtgtctgttcgcggccggattcttgttcgtcgtaagcgtggagtcctcgatcaagatggtttccgagagttctccaccgttcgatattgggtttgtggccacggagtctctgcatcatatcttggcttcttcaccggatctgaacaccggtttggccgctctaaactcggtgttaggagtgatgcaagtatcgtatattgcatggacatggttaatagaaggacggccccgagccaccatcacggctttattcctcttcacttgtcgcggtgttctcggttactgtactcagctccctctttcaaaggagtatctaggatcagcaatcgatttcccgctaggaaatctctcgttcttctattttttctcgggtcacgtggcaggcgcgaccatcgcatctttggacatgaggaggatgcagaggttgagatttgcgatggtttttgacatcctcaatgtattacagtcgatcaggctgcttgcgacgagaggacactacacgatcgatctcgcaggtggagttgccgctgcgattctctttgactcattggccggaaagtacgaagctaatacaagaaagaggcaattgtag

SEQ ID NO: 44 - protein, 235 aa, Brasssica napus
  Napus_2C
MSQMDISTRTEEGGWRSKPSFMTWRARDVVYVMRHHWIPCLFAAGFLFVVSVESSIKMVSESSPPFDIGFVATESLHHILASSPDLNTGLAALNSVLGVMQVSYIAWTWLIEGRPRATITALFLFTCRGVLGYCTQLPLSKEYLGSAIDFPLGNLSFFYFFSGHVAGATIASLDMRRMQRLRFAMVFDILNVLQSIRLLATRGHYTIDLAGGVAAAILFDSLAGKYEANTRKRQL

SEQ ID NO: 45 - DNA, 870 bp, Artificial
  Consensus PDCT1
atgtcaactacaacaatcgtccctctccgtcgcagttctaactctctcaatgaataccacactaacgcagtcgcctttgacggaatcgtcgggtcaacaagtactagccaaatggaggagattgttacgcaaaccgacgaaggctacgccaaccccaacggagatggagggagaagcaagccttcgtttatgacgtggaggatgtgcgacgttgtctacgtggtgagagtccattggataccgtgtttgttagcggtaggagttctgttcttcacgagcgtggaggagtacatgctccagatgattcccgagagttctgagccgttcgatattggttttgtggcgacgcgctctctgtatcgtctcttggcttcttcaccggatcttaacaccgttttagccgctctcaacacggtgtttgtagggatgcaaacgacgtatattgcatggacatggttgatagaaggacgaccacgagcgaccatctcggcttgcttcatgtttacttgtcgcggcattctcggttactctactcagctccctcttcctcaggattttctaggatcaggggtagattttccggtaggaaacgtctcgttcttcctcttctactcaggccacgtggcagggtcgacgatcgcatccttggacatgaggagaatgcagaggttgagacttgccttgctttttgacatcctcaatgtattacaatcgatcaggcttctcgggacgagaggacaatacacgatcgatctcgctgtcggagttggcgctggggttctctttgactcattggccggaaagtacgaagagatgatgagcaagagacaaaatgtaggcaatggttttagtttgatttcgtctcgctag

SEQ ID NO: 46 - protein, 289 aa, Artificial
  Consensus PDCT1
MSTTTIVPLRRSSNSLNEYHTNAVAFDGIVGSTSTSQMEEIVTQTDEGYANPNGDGGRSKPSFMTWRMCDVVYVVRVHWIPCLLAVGVLFFTSVEEYMLQMIPESSEPFDIGFVATRSLYRLLASSPDLNTVLAALNTVFVGMQTTYIAWTWLIEGRPRATISACFMFTCRGILGYSTQLPLPQDFLGSGVDFPVGNVSFFLFYSGHVAGSTIASLDMRRMQRLRLALLFDILNVLQSIRLLGTRGQYTIDLAVGVGAGVLFDSLAGKYEEMMSKRQNVGNGFSLISSR

SEQ ID NO: 47 - DNA, 894 bp, Artificial
  Consensus PDCT19
atgtcagcctccgcagctaaacccgctgtctctcgccgtcacgtatctaacggaaaccacactaacaacgtcgccattgacgacgatcacaaccaccaacgccccgtcgacgccggagatacaaacactcgaatggagatcgctgctaagaacaacggctacgccaacggtgtcggaggaggaggatggaggagcaaggcgtcgttcatgacgtggacgacgcgtgacgttgtctacgtggcgagacaccattggataccgtgcatgttcgctgccgggcttttgttcttcatgggggtcgagtacacgctccagatgatacccgcgagatctgagccgttcgatcttgggtttgtagccacgcgctctttgaatcgcgtcttagcatcttccccggatctaaacactgttctagccgcactaaacacggtgttcgtattgatgcaaacaacgtatattgtatggacatggttagtggaaggacgagcacgagcaaccatctcggctttattcatgttcacgtgtcgcggcattctcggctactctactcagcttcctctccctcaggattttttaggatcaggagttgattttccggtgggaaacgtctctttcttcctcttcttctcgggccacgttgccggctcgatgatcgcatcgttggacatgaggagaatgcagaggtttaagctggcgagggtttttgacatcctcaatgtattacaatcgatcaggctgctcggtacaagaggacactacaccatcgaccttgcggttggagttggcgctgggattctttttgactcactggccggaaagtacgaagagatgagcagaagacaccacctaggaactggttttagtttgatatcgaaagactctctagtcaattaa

SEQ ID NO: 48 - protein, 297 aa, Artificial
  Consensus PDCT19
MSASAAKPAVSRRHVSNGNHTNNVAIDDDHNHQRPVDAGDTNTRMEIAAKNNGYANGVGGGGWRSKASFMTWTTRDVVYVARHHWIPCMFAAGLLFFMGVEYTLQMIPARSEPFDLGFVATRSLNRVLASSPDLNTVLAALNTVFVLMQTTYIVWTWLVEGRARATISALFMFTCRGILGYSTQLPLPQDFLGSGVDFPVGNVSFFLFFSGHVAGSMIASLDMRRMQRFKLARVFDILNVLQSIRLLGTRGHYTIDLAVGVGAGILFDSLAGKYEEMSRRHHLGTGFSLISKDSLVN

SEQ ID NO: 49 - DNA, 843 bp, Brassica juncea
  BjROD1-B1
  misc_feature (476)..(477): n means a or g or c or t
atgtcaactaataccgtcgtccctctccgtcgcagatctaacggatatcacagtaacggcgtggcctttaacggaatggagaacattgtcaagaaaacagacgactgctacaccaacggcaacggaggaggagggaagagcaaggcgtcgtttctgacatggaccatgcgcgacgctgtctacgtggcgagataccattggataccgtgtttctttgcggtcggagttctgttctttatgggcgttgagtatacgctccagatggttccggcgaagtctgagccgttcgatattgggtttgtggccacgcgctctctgaaccgcgtcttggcgagttcaccggatcttaacacccttttagcggctctaaacacggtattcgtagcgatgcaaacgacgtatattgtatggacatggttgatggaaggaagaccacgagccactatctctgcttgctttatgtttacttgtcgcgnnattcttggttactctactcagctccctctcccacaggattttttaggatcaggagttgattttccagtgggaaacgtctcattcttcctcttctattctggtcacgtcgccggttcaatgatcgcatccttggacatgaggagaatgcggaggttgagactagcgatgctttttgacatcctcaacgtattacaatctatcaggctgctcgggacaagaggacattacacgattgatcttgcggtcggagttggcgctgggattctctttgactctttggccgggaagtacgaagagatgatgagcaagagacacaatttagccaatggttttagtttgatttcgaaagactcgctagtcaattaa

SEQ ID NO: 50 - protein, 280 aa, Brassica juncea
  BjROD1-B1
  misc_feature (159)..(159): Xaa can be Val, Ala, Asp, Glu, or Gly
MSTNTVVPLRRRSNGYHSNGVAFNGMENIVKKTDDCYTNGNGGGGKSKASFLTWTMRDAVYVARYHWIPCFFAVGVLFFMGVEYTLQMVPAKSEPFDIGFVATRSLNRVLASSPDLNTLLAALNTVFVAMQTTYIVWTWLMEGRPRATISACFMFTCRXILGYSTQLPLPQDFLGSGVDFPVGNVSFFLFYSGHVAGSMIASLDMRRMRRLRLAMLFDILNVLQSIRLLGTRGHYTIDLAVGVGAGILFDSLAGKYEEMMSKRHNLANGFSLISKDSLVN

SEQ ID NO: 51 - DNA, 843 bp, Brassica juncea
  BjROD1-A1
atgtcaactaataccgtcgtccctctccgtcgcagatctaacggaaatcacactaacggcgaggcctttaacggaatggagaacattgtcaagaaaaccgacgactgctacaccaacggcaacggaggagtagagagaagcaaagcctcgtttctgacatggaccatgcgtgacgctgtctacgtagcgagataccattggataccgtgtttctttgcggtcggagttctgttctttatgggggttgagtacacgctccagatggttccggcgaagtctgagccgttcgatattgggtttgtggccacgcgctctctgaaccgcgtcttggcgagttcaccggatcttaacacccttttagcggctctaaacacggtattcgtagcgatgcaaacgacgtatattgtatggacatggttgatggaaggaagaccacgagccactatctcggcttgcttcatgtttacttgtcgcggcattcttggttactctactcagctccctctaccacaggattttttaggatcaggagttgattttccggtgggaaacgtctcattcttcctcttctattctggccacgtagccggttcaatgatcgcatccttggacatgaggagaatgcagaggttgagactagcgatgctttttgacatcctcaacatattacaatcgatcagactgctcgggacgagaggacactacacgatcgatcttgcggtcggagttggcgctgggattctctttgactcattggccgggaagtacgaagagatgatgagcaagagacacaatttagccaatggttttagtttgatttctaaagactcgctagtcaattaa

SEQ ID NO: 52 - protein, 280 aa, Brassica juncea
  BjROD1-A1
MSTNTVVPLRRRSNGNHTNGEAFNGMENIVKKTDDCYTNGNGGVERSKASFLTWTMRDAVYVARYHWIPCFFAVGVLFFMGVEYTLQMVPAKSEPFDIGFVATRSLNRVLASSPDLNTLLAALNTVFVAMQTTYIVWTWLMEGRPRATISACFMFTCRGILGYSTQLPLPQDFLGSGVDFPVGNVSFFLFYSGHVAGSMIASLDMRRMQRLRLAMLFDILNILQSIRLLGTRGHYTIDLAVGVGAGILFDSLAGKYEEMMSKRHNLANGFSLISKDSLVN

SEQ ID NO: 53 - DNA, 843 bp, Brassica rapa
  BrROD1_SEQIDNO7
atgtcaactaataccgtcgtccctctccgtcgcagatctaacggatatcacactaacggcgtggcctttaacggaatggagaacattgtcaagaaaaccgacgactgctacaccaacggcaacggaggagtagagagaagcaaagcctcgtttctgacatggaccatgcgtgacgctgtctacgtagcgagataccattggataccgtgtttctttgcggtcggagttctgttctttatgggggttgagtacacgctccagatggttccggcgaagtctgagccgttcgatattgggtttgtggccacgcgctctctgaaccgcgtcttggcgagttcaccggatcttaacacccttttagcggctctaaacacggtattcgtagcgatgcagacgacgtatattgtatggacatggttgatggaaggaagaccacgagccactatctcggcttgcttcatgtttacttgtcgcggcattcttggttactctactcagctccctctaccacaggattttttaggatcaggagttgattttccggtgggaaacgtctcattcttcctcttctattctggccacgtagccggttcaatgatcgcatccttggacatgaggagaatgcagaggttgagactagcgatgctttttgacatcctcaacatattacaatcgatcagactgctcgggacgagaggacactacacgatcgatcttgcggtcggagttggcgctgggattctctttgactcattggccgggaagtacgaagagatgatgagcaagagacacaatttagccaatggttttagtttgatttctaaagactcgctagtcaattaa

SEQ ID NO: 54 - protein, 280 aa, Brassica rapa
  BrROD1_SEQIDNO7
MSTNTVVPLRRRSNGYHTNGVAFNGMENIVKKTDDCYTNGNGGVERSKASFLTWTMRDAVYVARYHWIPCFFAVGVLFFMGVEYTLQMVPAKSEPFDIGFVATRSLNRVLASSPDLNTLLAALNTVFVAMQTTYIVWTWLMEGRPRATISACFMFTCRGILGYSTQLPLPQDFLGSGVDFPVGNVSFFLFYSGHVAGSMIASLDMRRMQRLRLAMLFDILNILQSIRLLGTRGHYTIDLAVGVGAGILFDSLAGKYEEMMSKRHNLANGFSLISKDSLVN

SEQ ID NO: 55 - DNA, 852 bp, Brasssica napus
  Napus_5C
atgtcaactgaaactggcgtccctctccgtcgcagatctaactctcttaacggacatcactctaacgacgtcgcctttgacggaaccgtcccatcaatggagaacaacattgttaagaaaacagacgacggctacgccaatggaggaggaaaggcgtcgtttatgacatggacggcgcgtgacgctatctacgtggcgagagtccattggataccgtgtgtgttcgcggttggagttctgttcttcatgggcgtcgagtatacgcttcagatgattcccgcgaggtctgagccgttcgatgttgggtttgtggccacgcgctctctgaaccgcgtcttggcaaattcaccgggtcttaacaccgttttagccgcactaaacacggtgttcgtagggatgcaaactacgtatattgtatggacatggttgatggaaggaagaccacgagccaccatctcggcttgcttcatgtttacttgtcgcggtattcttggttactctactcagctccctctccctcaggagtttttaggatcaggagtcgattttcctgtgggaaacgtctcattcttccttttctactcgggtcacgtcgccggttcgatgatagcatccttagacatgaggagaatgcagaggttgagactagcaatgctttttgacatcctcaatgttttacaatcgataaggctgctcgggacgagaggacattacaccatcgatcttgcggtcggagttggcgctgggattctctttgactcgttggccgggaagtacgaagagatgatgagcaaaagacacaatttaggcaatggttttagtttgatttctaaagactcgctagtcaattaa

SEQ ID NO: 56 - protein, 283 aa, Brasssica napus
  Napus_5C
MSTETGVPLRRRSNSLNGHHSNDVAFDGTVPSMENNIVKKTDDGYANGGGKASFMTWTARDAIYVARVHWIPCVFAVGVLFFMGVEYTLQMIPARSEPFDVGFVATRSLNRVLANSPGLNTVLAALNTVFVGMQTTYIVWTWLMEGRPRATISACFMFTCRGILGYSTQLPLPQEFLGSGVDFPVGNVSFFLFYSGHVAGSMIASLDMRRMQRLRLAMLFDILNVLQSIRLLGTRGHYTIDLAVGVGAGILFDSLAGKYEEMMSKRHNLGNGFSLISKDSLVN

SEQ ID NO: 57 - DNA, 843 bp, Brasssica napus
  Napus_3C
atgtcaactaataccgtcgtccctctccgtcgcagatctaacggatatcacactaacggcgtggccttcaacggaatggagaacattgtcaagaaaaccgacgactgctacaccaatggcaacggagtaggagggaagagcaaggcgtcgtttctgacatggaccatgcgtgacgctgtctacgtagcgagataccattggataccgtgtttctttgcggtcggagttctgttctttatgggggttgagtacacgctccagatggttccggcgaagtctgagccgttcgatattgggtttgtggccacgcgctctctgaaccgcgtcttggcgagttcaccggatcttaacacccttttggcggctctaaacacggtattcgtagcgatgcaaacgacgtatattgtatggacatggttgatggaaggaagaccacgagccactatctcggcttgcttcatgtttacttgtcgcggtattcttggttactctactcagctccctctaccacaggattttttaggatcaggagttgattttccggtgggaaacgtctcattcttcctcttctattctggccacgtagccggttcaatgatcgcatccttggacatgaggagaatgcagaggttgagactagcgatgctttttgacatcctcaacatattacaatcgatcaggctgctcgggacgagaggacactacacgatcgatcttgcggtcggagttggcgctgggattctctttgactcattggccgggaagtacgaagagatgatgagcaagagacacaatttagccaatggttttagtttgatttctaaagactcgctagtcaattaa

SEQ ID NO: 58 - protein, 280 aa, Brasssica napus; identical to SEQ ID NO: 24
  Napus_3C

SEQ ID NO: 59 - DNA, 843 bp, Artificial
  Consensus PDCT3/5
atgtcaactaataccgtcgtccctctccgtcgcagatctaacggacatcacactaacggcgtggcctttaacggaatggagaacattgtcaagaaaacagacgactgctacaccaatggcaacggaggaggagggaagagcaaggcgtcgtttctgacatggaccatgcgtgacgctgtctacgtggcgagataccattggataccgtgtttctttgcggtcggagttctgttctttatgggcgttgagtatacgctccagatggttccggcgaagtctgagccgttcgatattgggtttgtggccacgcgctctctgaaccgcgtcttggcgagttcaccggatcttaacacccttttagcggctctaaacacggtattcgtagcgatgcaaacgacgtatattgtatggacatggttgatggaaggaagaccacgagccactatctcggcttgcttcatgtttacttgtcgcggtattcttggttactctactcagctccctctcccacaggattttttaggatcaggagttgattttccggtgggaaacgtctcattcttcctcttctattctggtcacgtcgccggttcaatgatcgcatccttggacatgaggagaatgcagaggttgagactagcgatgctttttgacatcctcaacgtattacaatcgatcaggctgctcgggacgagaggacattacacgatcgatcttgcggtcggagttggcgctgggattctctttgactcgttggccgggaagtacgaagagatgatgagcaagagacacaatttagccaatggttttagtttgatttctaaagactcgctagtcaattaa

SEQ ID NO: 60 - protein, 280 aa, Artificial
  Consensus PDCT3/5
MSTNTVVPLRRRSNGHHTNGVAFNGMENIVKKTDDCYTNGNGGGGKSKASFLTWTMRDAVYVARYHWIPCFFAVGVLFFMGVEYTLQMVPAKSEPFDIGFVATRSLNRVLASSPDLNTLLAALNTVFVAMQTTYIVWTWLMEGRPRATISACFMFTCRGILGYSTQLPLPQDFLGSGVDFPVGNVSFFLFYSGHVAGSMIASLDMRRMQRLRLAMLFDILNVLQSIRLLGTRGHYTIDLAVGVGAGILFDSLAGKYEEMMSKRHNLANGFSLISKDSLVN

SEQ ID NO: 61 - DNA, 906 bp, Arabidopsis thaliana
  AtRodD1
atgtcagccgccgcagctgaaaccgacgtctctctccgtcgcagatctaactctcttaacggaaaccacactaacggcgtcgccattgacggaaccctagacaacaacaaccgtcgcgtcggagatacaaacactcacatggatatatctgctaagaaaactgacaacggctacgccaatggtgtcggaggaggaggatggagaagcaaagcgtcgttcacgacgtggacggcgcgtgatatcgtctacgtggtgagataccattggataccgtgcatgttcgctgccggacttctgttcttcatgggcgtggagtacacgcttcagatgattcccgcgagatctgagccgttcgatcttgggtttgtggtcacgcgctctttgaatcgcgtattagcatcttcaccggatcttaacactgttttagccgcactaaacacggtgttcgtagggatgcaaacaacgtatattgtatggacatggttagtggaaggacgagcacgagccaccatcgcggctttattcatgttcacttgtcgcggcattctcggctactctactcagcttcctctccctcaggactttctaggatcaggggttgattttccggtgggaaatgtctctttcttcctcttcttctctggccatgtcgccggctcgatgatcgcatcattggacatgagaagaatgcagaggttgagacttgcaatggtctttgacatcctcaatgtattacagtcgatcagactgctcggtacaagaggacattacacaatcgaccttgcggttggagttggcgctgggattctcttcgactcattggccggaaagtacgaagagatgatgagcaagagacatttaggcactggttttagtttgatttcgaaagactctctagtcaattaa

SEQ ID NO: 62 - protein, 301 aa, Arabidopsis thaliana
  AtRodD1
MSAAAAETDVSLRRRSNSLNGNHTNGVAIDGTLDNNNRRVGDTNTHMDISAKKTDNGYANGVGGGGWRSKASFTTWTARDIVYVVRYHWIPCMFAAGLLFFMGVEYTLQMIPARSEPFDLGFVVTRSLNRVLASSPDLNTVLAALNTVFVGMQTTYIVWTWLVEGRARATIAALFMFTCRGILGYSTQLPLPQDFLGSGVDFPVGNVSFFLFFSGHVAGSMIASLDMRRMQRLRLAMVFDILNVLQSIRLLGTRGHYTIDLAVGVGAGILFDSLAGKYEEMMSKRHLGTGFSLISKDSLVN

SEQ ID NO: 63 - DNA, 804 bp, Glycine max
  GmROD1-1
atgaatggcggcgctgaggcctccctcaatcacaggcgcaaacaccaaacagctcccgccgacggcgctaaaggcgttaaggtagcaaacggagccatggggaagccgtcctcttccaagcactcctgcggcgcgtcgttcatgaaatggaccgtggctgacgctgtccacgtggtgacgcaccattggatgccgtgcttgttcgcattggggcttctcttcttcatggccgtggagtacacgcttctcatggtgccgccgtcgtcgccgcccttcgaccttggcttcatcgccacacgctccctccacgcgctcctcgagtcgtcgccgaatctcaacacgctcttcgccgggctcaatacggtgtttgtggggatgcaaacgagttatatcttatggacgtggctgattgaaggacgccccagagccacaatttcagcattgttcatgttcacatgccgtgggattttaggctactccacccagctcccattgcctcaggggtttttgggctcgggtgtggacttccctgttgggaacgtgtcttttttcttgtttttttctgggcacgttgcagggtcagtgattgcttcattggacatgaggaggatgcagaggtgggaactggcttggacttttgatgtgctcaatgttttgcaagctgtgaggttgctgggtacaagaggccattacactattgatttggccgtaggggttggtgctggaattctctttgattctttagctggcaagtacgaagatagcaaaaggaatggtgctctcaaacacaatttgattgcgtga

SEQ ID NO: 64 - protein, 267 aa, Glycine max
  GmROD1-1
MNGGAEASLNHRRKHQTAPADGAKGVKVANGAMGKPSSSKHSCGASFMKWTVADAVHVVTHHWMPCLFALGLLFFMAVEYTLLMVPPSSPPFDLGFIATRSLHALLESSPNLNTLFAGLNTVFVGMQTSYILWTWLIEGRPRATISALFMFTCRGILGYSTQLPLPQGFLGSGVDFPVGNVSFFLFFSGHVAGSVIASLDMRRMQRWELAWTFDVLNVLQAVRLLGTRGHYTIDLAVGVGAGILFDSLAGKYEDSKRNGALKHNLIA

SEQ ID NO: 65 - DNA, 840 bp, Glycine max
  GmROD1-2
atgaacggcggcgctgaggcctccgtcaatcacaggcgcagacaccaagcagcttccgctaacggcgttaagatagcaaacggggccatggcgaagccgtcctcgacgctctgctacgacgcctcgttcatgaaatggaccgtggcggatgctgtccacgtggcgacgcatcattggatgccgtgcttattcgcattagggcttctcttcttcatggccgtggaatacacgctcctcatggttccgccgtcgtcgccgcctttcgatctgggcttcattgccacgcgttccctccacgcactcctcgagtcatcgccgaatctcaacacgctcttcgccgggctcaatacggtgtttgtggggatgcaaacgagttatatcttatggacgtggctgattgaaggacgccccagagccacgatttcagcattgttcatgttcacatgccgtggaattttagggtactccacccagctcccattgcctcagggatttttgggctcgggtgtggatttcccagttgggaacgtgtcgtttttcttgtttttttcggggcatgttgcgggttcagtgattgcttccttggacatgaggaggatgcagaggtgggaactggcttggacttttgatgtgctcaatgttttgcaagctgtgaggttgctgggtacaagaggacattacactattgatttggccgtaggggttggtgctggaattctctttgattctttagctggcaagtacgaagatagcaaaaggaatgctgctctatccacaacccacagagcacaatttgattgcgtcaacaatgtggatatagctaaaaaaattaacaaatga

SEQ ID NO: 66 - protein, 279 aa, Glycine max
  GmROD1-2
MNGGAEASVNHRRRHQAASANGVKIANGAMAKPSSTLCYDASFMKWTVADAVHVATHHWMPCLFALGLLFFMAVEYTLLMVPPSSPPFDLGFIATRSLHALLESSPNLNTLFAGLNTVFVGMQTSYILWTWLIEGRPRATISALFMFTCRGILGYSTQLPLPQGFLGSGVDFPVGNVSFFLFFSGHVAGSVIASLDMRRMQRWELAWTFDVLNVLQAVRLLGTRGHYTIDLAVGVGAGILFDSLAGKYEDSKRNAALSTTHRAQFDCVNNVDIAKKINK

SEQ ID NO: 67 - DNA, 858 bp, Ricinis communis
  RcPDCT
atgaaatccaccgtccctcccaccaccacaaccacaaccacaactactctttacaagcgcaagaaagacatcaacttgacctccgtcaacgacagtgttgacatggttagcaacaagaactttgctaacggtaatgttaatggtggtgggggctacactgcttttaataggtttgacccatcgttcatgaaatggacgactcatgatgtggtcaatgtagtcaagtttcattggttaccgtgtgtttttggacttgggttgctattctttatggccgttgaatacactcttcgcatggttccggcttcttctccgccttttgatttggggtttctggttacgcgccaccttcatctcttgctttcttcttggccggcgctcaacactttgttggcttttcttaatacggtgtttgttttgatgcaaaccgcatatatattgtggacgtggctaatagagggcagaccaagagctacaatttcggctttattcatgttcacttgccgtgggattcttggctactccactcagcttccacttcctgagggatttctgggatcaggagttgattttccagtaggaaatgtgtcattcttcctgtttttctccggccatgtcgcggggtctgtgatagcatcgctcgatatgagaagaatgcagagatgggaattggcatggacatatgatgtgcttaatgttctacaagctgtgaggctactaggcactagaggccactatacaatcgacttagcaactggtgtaggtgctggcattctgtttgattcacttgcggggaaatatgaagagagcaagagaaaacaggctgttgttgctaaagagtcttctttgtttagttaa

SEQ ID NO: 68 - protein, 285 aa, Ricinis communis
  RcPDCT
MKSTVPPTTTTTTTTTLYKRKKDINLTSVNDSVDMVSNKNFANGNVNGGGGYTAFNRFDPSFMKWTTHDVVNVVKFHWLPCVFGLGLLFFMAVEYTLRMVPASSPPFDLGFLVTRHLHLLLSSWPALNTLLAFLNTVFVLMQTAYILWTWLIEGRPRATISALFMFTCRGILGYSTQLPLPEGFLGSGVDFPVGNVSFFLFFSGHVAGSVIASLDMRRMQRWELAWTYDVLNVLQAVRLLGTRGHYTIDLATGVGAGILFDSLAGKYEESKRKQAVVAKESSLFS

SEQ ID NO: 69 - DNA, 858 bp, Ricinis communis; identical to SEQ ID NO: 67
  RcROD1_SEQIDNO9

SEQ ID NO: 70 - protein, 285 aa, Ricinis communis; identical to SEQ ID NO: 68
  RcROD1_SEQIDNO9

SEQ ID NO: 71 - DNA, 831 bp, Linum usitatissimum
  LuPDCT1
atgtctgccgcacccaccaccaccgtggcagccaccgccattccgttgaagcgcaagaaaacggatgctgctgctaaaacacagaaaaacaaccagggaaatgaggacgaacaggaggcaatagtggagagcgtggcgccggcgtttacgaaatggaggaggagagatgcgatgaatgcggttagggagcattggttgcctgtcatattgggaggtgccctgttgtttttcatgtgggttgagtacaccctcaggatggtcccgacctcctcacaaccctttgatttagggttcgtggccacccgagctctccatcgccttctttcttcctcgccggagctcaactccgtcctcgccgctctcaacacggtgttcgtgggaatgcagacaagctacatactgtggacgtttgtggtggagggaagggggagacccactatctctgcacttttcatgttcacttgccgtggaattcttggctattccactcagcttcctctccccgagggatatttggggtcgggagtggacttcccagtagggaacgtgtcgtttttcctgttcttctccgggcacgtggcgggatcagtaatagcatcacaggatatgaggagaatgcaaaggtgggagcttgccttgggatttgaattcctcaacgccttacaagtggtcaggctactcgccaccagaggtcactacaccattgatttggccgctggttatgccgccggtctactctttgactcacttgccggacgttatctccaaaccaagaccgccactgctgctgctcttcttaccacttctcccagaaaatttgtcaccaactag

SEQ ID NO: 72 - protein, 276 aa, Linum usitatissimum
  LuPDCT1
MSAAPTTTVAATAIPLKRKKTDAAAKTQKNNQGNEDEQEAIVESVAPAFTKWRRRDAMNAVREHWLPVILGGALLFFMWVEYTLRMVPTSSQPFDLGFVATRALHRLLSSSPELNSVLAALNTVFVGMQTSYILWTFVVEGRGRPTISALFMFTCRGILGYSTQLPLPEGYLGSGVDFPVGNVSFFLFFSGHVAGSVIASQDMRRMQRWELALGFEFLNALQVVRLLATRGHYTIDLAAGYAAGLLFDSLAGRYLQTKTATAAALLTTSPRKFVTN

SEQ ID NO: 73 - DNA, 831 bp, Linum usitatissimum
  LuPDCT2
atgtctgccacacccaccgccgccgtggcagccaccgccattccgttgaagcgcaagaaaacggatgctgctgctaaaacacagaaaaacaaccagggaaatgaggacgaacaggaggcaatagtggagagcgtggcgccggcgtttacgaaatggaggaggagagatgcgatgaatgcggtcagggagcattggttgcctgtcatgttgggaggtgccctgttgtttttcatgtgggttgagtacaccctcaggatggtcccgacctcctcacagccctttgatttagggttcgtggccacccgagctctccatcgccttctttcttcctcgccggagctcaactccgtcctcgccgctctcaacacggtgttcgtgggaatgcagacaagctacatattgtggacgtttgtggtggagggaagggggagacccactatctctgcacttttcatgttcacttgccgtggaattcttggctattccactcagcttcctctcccagagggatatttggggtcgggagtggacttcccagtagggaacgtgtcgtttttcctctttttctcgggacacgtggcgggatcagtgatagcatcacaggatatgaggagaatgcaaaggtgggagctggccttgggatttgaattcctcaacgccttacaagtagtcaggctactcgccaccagaggtcactacaccattgatttggccgccggttatgccgccggtctgctctttgactcacttgccggacgttacctccaaaccaagaccgccactgctgctgctcttcttaccacctctcccagaaaatttgtcaccaactag

SEQ ID NO: 74 - protein, 276 aa, Linum usitatissimum
  LuPDCT2
MSATPTAAVAATAIPLKRKKTDAAAKTQKNNQGNEDEQEAIVESVAPAFTKWRRRDAMNAVREHWLPVMLGGALLFFMWVEYTLRMVPTSSQPFDLGFVATRALHRLLSSSPELNSVLAALNTVFVGMQTSYILWTFVVEGRGRPTISALFMFTCRGILGYSTQLPLPEGYLGSGVDFPVGNVSFFLFFSGHVAGSVIASQDMRRMQRWELALGFEFLNALQVVRLLATRGHYTIDLAAGYAAGLLFDSLAGRYLQTKTATAAALLTTSPRKFVTN

SEQ ID NO: 75 - DNA, 924 bp, Oryza sativa
  OsROD1_SEQIDNO11
atgccgccgccgccgccgcccagcctcacggccaacaccgcatcctccatgggcaacgccgaggccgtcgtggtgctgcccgcgaacggcggcgcgcggcggcgcgccgacaaggtcgtccacccggcgccgatgccggacagagcagctggtggcgcgatggagagggaaggcggcggcgtcggcggcggcggcgaggtgggtgggtggaggaggccggagtggtgctcggcggcgggggtggcgggggtcctgcggcggcacccggcggcggcggcgttcgggtgcgggctgctgctgttcatggccgtggagtacaccatccccatggtgccgcccgccgcgccgccggtcgacctcggcttcgccgccaccgccgcgctccacgccgggatcgccgcccgcccatggctcaactcgctcctcgccgcgctcaacacggtgttcgtggcgatgcaggcggcgtacatcctgtgggcgatcctcggcgagggccggccgcgcgccgccgtggcggcgatgatgatgttcacctgccgcggcgcgctcggctgcgccacgcagctgccgctgccggccgagttcctgggctccggcatggacttccccgtcggcaacgtctccttcttcctcttcttctccggccacgtcgccggcgcggtgatcgccgccgaggacatgcgccgcgcggggcgccgcggcatggcgcgcctctacgacgcgctcaacctgctccagggcgtcaggctgctcgcctgcaggggccactacaccatcgacctcgccgtcggcgtcggcgccggcctcctcttcgacatgctcgccggcaggtacctggacggcaagaacaccgtcgacggcggcgccgccgtggcgccggggagccggtgctgcagctgccacaaggctctcttgtcacagtag

SEQ ID NO: 76 - protein, 307 aa, Oryza sativa
  OsROD1_SEQIDNO11
MPPPPPPSLTANTASSMGNAEAVVVLPANGGARRRADKVVHPAPMPDRAAGGAMEREGGGVGGGGEVGGWRRPEWCSAAGVAGVLRRHPAAAAFGCGLLLFMAVEYTIPMVPPAAPPVDLGFAATAALHAGIAARPWLNSLLAALNTVFVAMQAAYILWAILGEGRPRAAVAAMMMFTCRGALGCATQLPLPAEFLGSGMDFPVGNVSFFLFFSGHVAGAVIAAEDMRRAGRRGMARLYDALNLLQGVRLLACRGHYTIDLAVGVGAGLLFDMLAGRYLDGKNTVDGGAAVAPGSRCCSCHKALLSQ

SEQ ID NO: 77 - DNA, 849 bp, Zea mays
  ZmROD1_GRMZM2G015040
  misc_feature (726)..(726): s means g or c
atgccgccgcccagcctcaccgccgccggcaccaccaccaccacaacccgccgccgcaacgaccgagccgcgaaggtccaccaggtactgggcgaaggcgcggggacggaggagatgggcgcggtggcggacgggtggacgcggcccgagtggtgctcggcggcgggcgtcgcgggcgtgctgcggcggcacccggcgcccgcgctcttcgggtgcggcctcctgctcttcatggccgtcgagtacaccatccccatggtcaagccggacgcgccgccgctcgacctaggcttcctcgccaccgcgggcatgcacgccgccatcgccgcgaggccctggcttaactcgctcctcgccgcgctcaacacggtcttcgtcgcgatgcaggcggcgtacatcctgtgggccatcctcgccgagcagcggccgcgcgcggccgtcgccgcgctcatgatgttcacttgccggggcgtgctgggctgcgccacccagctcccgctgccggaggagttcctgggctccgggatggacttccccgtgggcaacgtctccttcttcctcttcttctcgggccacgtcgcgggcgcggtgatcgcggcggccgacatgcggcgcgaggggcggctggcgctggcgcgcctcttcgactcgctcaacgtgctccaggtggtcaggctgctcgcgtgcaggggacactacaccattgacctggctgttggcgttggsgcgggcatcctcttcgacacgctctccggatggtacttcgacgccaagaacggcgatagcagcaacgcaccggagaagcagtgccggagctgccagtgccacaaggccctcctttcacactag

SEQ ID NO: 78 - protein, 282 aa, Zea mays
  ZmROD1_GRMZM2G015040
MPPPSLTAAGTTTTTTRRRNDRAAKVHQVLGEGAGTEEMGAVADGWTRPEWCSAAGVAGVLRRHPAPALFGCGLLLFMAVEYTIPMVKPDAPPLDLGFLATAGMHAAIAARPWLNSLLAALNTVFVAMQAAYILWAILAEQRPRAAVAALMMFTCRGVLGCATQLPLPEEFLGSGMDFPVGNVSFFLFFSGHVAGAVIAAADMRREGRLALARLFDSLNVLQVVRLLACRGHYTIDLAVGVGAGILFDTLSGWYFDAKNGDSSNAPEKQCRSCQCHKALLSH

SEQ ID NO: 79 - DNA, 825 bp, Zea mays
  ZmROD1_GRMZM2G087896
  misc_feature (412)..(412): m means a or c
  misc_feature (635)..(635): w means a or t
atgccgccgcccagcctcaccgtggcgcgcgacccggccgccgccgccaccacgcggcaccgcaaggtccacccgacgccgggcggaggcgcagggacgacgaaggagatgggcgcggcggaggggtgggcgcggcccgagtggtgctcggcggcgggcgccgcggccgtgctgcggcggcacccggcgcccgcgctcttcgggtgcggcctcctgctcttcatggccgtcgagtacaccatccccatggtcaggccggacgcgccgccgctcgacctgggcttcgtcgccaccaggggcttgcacgccgccgtcgccgcgaggccctggctcaactcgctcctcgccgcgctcaacacggtcttcgtcgcgatgcaggcggcgtacatcctgtgggccatcctcggcgagmagcggccgcgcgccgccgtcgccacgctcatgatgttcacctgccggggcgcgctgggctgcgccacccagctcccgctgccggaggagttcctggggtccgggatggacttccccgtgggcaacgtctccttcttcctcttcttctcgggccacgtcgcgggcgcggtcatcgcggccgccgacatgcgccgcgagggccggctggcgctggcgcgcctctwcgacgcgctcaacgcgctccagggggtcaggctgctggcgtgcaggggacactacaccatcgacctggccgtcggtgtcggagcggggatcctcttcgatacgctctccggatggtacttccacgccaagaacgcaccggagaagcactgccgcagctgccagtgccacaaggctctcctttcacgctag

SEQ ID NO: 80 - protein, 274 aa, Zea mays
  ZmROD1_GRMZM2G087896
  misc_feature (138)..(138): Xaa can be Lys or Gln
  misc_feature (212)..(212): Xaa can be Tyr or Phe
MPPPSLTVARDPAAAATTRHRKVHPTPGGGAGTTKEMGAAEGWARPEWCSAAGAAAVLRRHPAPALFGCGLLLFMAVEYTIPMVRPDAPPLDLGFVATRGLHAAVAARPWLNSLLAALNTVFVAMQAAYILWAILGEXRPRAAVATLMMFTCRGALGCATQLPLPEEFLGSGMDFPVGNVSFFLFFSGHVAGAVIAAADMRREGRLALARLXDALNALQGVRLLACRGHYTIDLAVGVGAGILFDTLSGWYFHAKNAPEKHCRSCQCHKALLSR

(19) United States
(12) Patent Application Publication
(10) Pub. No.: US 2021/0317467 A1
(43) Pub. Date: Oct. 14, 2021

(54) IMPROVED METHOD FOR THE PRODUCTION OF HIGH LEVELS OF PUFA IN PLANTS

(71) Applicant: BASF PLANT SCIENCE COPMANY GMBH, Ludwigshafen (DE)

(72) Inventors: Toralf Senger, Research Triangle Park, NC (US); Hui Yang, Saskatoon (CA); Carl Andre, Research Triangle Park, NC (US); Patricia Vrinten, Saskatoon (CA)

(21) Appl. No.: 17/273,750

(22) PCT Filed: Sep. 6, 2019
(86) PCT No.: PCT/EP2019/073841

§ 371 Date: Mar. 5, 2021

(30) Foreign Application Priority Data
Sep. 7, 2018 (EP) 18193288.0
Sep. 11, 2018 (EP) 18193828.3

(51) Int. Cl. C12N 15/82; C11B 1/00; C12N 9/12; C07K 16/40; A23D 9/00; A23L 33/12; A23K 20/158

(52) U.S. Cl. CPC C12N 15/8247; C11B 1/00; C12N 9/1288; C12Y 207/08002; A23V 2002/00; A23D 9/00; A23L 33/12; A23K 20/158; C07K 16/40

(57) ABSTRACT

The present invention is concerned with materials and methods for the production of genetically modified plants, particularly where the plants are for the production of at least one unsaturated or polyunsaturated fatty acid. The invention is also concerned with identification of genes conveying an unsaturated fatty acid metabolic property to a plant or plant cell, and generally relates to the field of phosphotidylcholine:diacylglycerol cholinephosphotransferase (PDCT).

The present invention is concerned with materials and methods for the production of genetically modified plants, particularly where the plants are for the production of at least one unsaturated or polyunsaturated fatty acid. The invention is also concerned with identification of genes conveying an unsaturated fatty acid metabolic property to a plant or plant cell, and generally relates to the field of phosphotidylcholine:diacylglycerol cholinephosphotransferase (PDCT).

Very long chain polyunsaturated fatty acids (VLC-PUFAs), such as arachidonic acid (ARA; 20:4 ω6), eicosapentaenoic acid (EPA; 20:5ω3) and docosahexaenoic acid (DHA; 22:6ω3), have demonstrable benefits for human health (Swanson et al., 2012; Haslam et al., 2013), but humans are unable to synthesize these fatty acids in sufficient quantities. Transgenic oilseed crops are an alternative source for VLC-PUFAs: such systems minimally require two desaturation steps and one elongation to convert plant-derived linoleic acid (LA; 18:2ω6) and ALA to VLC-PUFAs (Venegas-Caleron et al., 2010).

In the production of unusual fatty acids in plants, improving the flux of fatty acids through pools such as acyl-CoA PC, DAG and TAG is of particular interest (Wu et al., 2005;)

Brassica carinata has been shown to have potential as a host plant for VLC-PUFA production (Cheng at al., 2010). Ruiz-Lopez et al (2014) demonstrated that Camelina sativa also functions well as a host plant, and were able to demonstrate production of VLC-PUFA levels similar to those found in fish oils. Brassica juncea (Wu et al 2005), and Brassica napus has also been used as a host plant by various groups for the production of various fatty acids, including VLC-PUFAs, γ-linolenic acid (GLA), and stearidonic acid (SDA) (Petrie et al, 2014; Ursin et al, 2003, Liu et al, 2001).

Differences in VLC-PUFA production have been observed among these plants when enzymes involved in EPA and DHA biosynthesis (and their various pre-cursors) have been ectopically expressed, which may be partly due to differences in endogenous enzymes functioning in the fatty acid synthesis pathway (Cheng et al, 2010). Such differences may be reflected in the fatty acid profile of these plants; for example, Camelina seed oil is high in ALA (18:3), with levels of around 30% (Iskandarov et al. 2014, while B. napus generally has levels around 10% (Singer et al. 2014) and B. carinata seed oil averages 18% (Genet et al. 2004). A better understanding of the endogenous metabolism that impacts the production of EPA and DHA will lead to strategies to improve the production of these fatty acids in any host plant.

The identification of the phosphotidylcholine:diacylglycerol cholinephosphotransferase (PDCT) encoded by the Arabidopsis (Arabidopsis thaliana) ROD1 gene (Lu et al., 2009) led to an improved understanding of the incorporation of polyunsaturated fatty acids (PUFAs) into triacylglycerols (TAGs). PDCT acts through the exchange of phosphocholine headgroups between de-novo synthesized diacylglycerols (DAG) and phosphatidylcholine (PC); PC can then be converted back to DAG and sequentially to TAG (Lu et al., 2009). Such exchanges contribute significantly to the flux of PUFAs into the TAG pool in Arabidopsis seeds (Bates et al., 2012).

To make possible the fortification of food and/or of feed with polyunsaturated omega-3-fatty acids, there is still a great need for a simple, inexpensive process for the production of each of the aforementioned long chain polyunsaturated fatty acids, especially in eukaryotic systems.

The invention is thus concerned with providing a reliable source for easy manufacture of VLC-PUFAs. To this end the invention is also concerned with providing plants reliably producing VLC-PUFAS, preferably EPA and/or DHA. The invention is also concerned with providing means and methods for obtaining, improving and farming such plants, and also with VLC-PUFA containing oil obtainable from such plants, particularly from the seeds thereof. Also, the invention provides uses for such plants and parts thereof.

The complementation of Arabidopsis rod1 mutants with flax PDCT (Wickramarathna et al., 2015) and castor PDCT (Hu et al., 2012) restored the fatty acid profiles of Arabidopsis seeds, showed that PDCT from different species function through similar mechanisms.

B. napus, B. carinata, and C. sativa are polyploid species, each having more than one copy of the PDCT gene. Differences in the PDCT genes within and between these three species may effect the production of polyunsaturated fatty acids in transgenic plants. Using Arabidopsis as a model system to examine the influence of PDCTs from B. napus, B. carinata, and C. sativa on the production of PUFAs in seeds it was found that individual PDCT′ groups have distinct functional properties that influence the production of PUFAs in seeds.

It has now surprisingly been found that the increased activity of the phosphotidylcholine:diacylglycerol cholinephosphotransferase (PDCT) of the present invention, e.g. of a PDCT1 as described herein, in a plant, plant cell or plant seed can increase the level of new PUFAs in the plant, plant cell, or seed, that is capable to produce DPA, DHA and/or EPA and expressing a d6elo and a d6des.

With the “level of PUFA” is meant the level of PUFAs as a percentage of the total fatty acids found in seeds or seed oil, preferablyas percent of weight

Further, it was found that the increased expression, the increase in cellular activity or the de novo expression of a PDCT of the present invention, e.g. of a PDCT1 , results in the production of a plant, a part thereof, a plant cell, plant seed or plant seed oil, wherein the combined ALA and LA level (ALA plus LA level) is less than the combined level of C18, C20 and C22 PUFAs.

Furthermore, surprisingly, it was observed that the increased expression, the increase in cellular activity or the de novo expression a PDCT of the present invention, e.g. of a PDCT1 , in a plant, plant cell and/or plant seed can increase the Delta-6 elongase conversion efficiency in a plant, plant cell and/or plant seed that produces C18, C20, and/or C22 fatty acids and that expresses a Delta-6 elongase.

Further, the present invention relates to a method for the production of a composition comprising the fatty acid GLA in a plant, or part thereof, like a plant cell, and/or part seed, or part thereof,

wherein the level of the 18:2 fatty acid in % (w/w) in the triacylglycerol (TAG) fraction is around the same level as the 18:2 fatty acid level in % (w/w) in the diacylglyerol (DAG) fraction, comprising,

providing a plant cable to produce GLA and having an increased activity or expression of one or more PDCT compared to the wild type, the PDCT selected from the group consisting of:

(a) a PDCT1 having at least 80% sequence identity with SEQ ID NO: 2, 4, 6, 8, 10, 12, 14, 16, 40, 42, 44, and/or 46;

(b) a PDCT1 encoded by a polynucleotide having at least 80% sequence identity with SEQ ID NO: 1, 3, 5, 7, 9, 11, 13, 15, 39, 41, 43, and/or 45;

(c) a PDCT1 encoded by a polynucleotide that hybridizes under high stringency conditions with (i) a polynucleotide that encodes the amino acid sequence of SEQ ID NO: 2, 4, 6, 8, 10, 12, 14, 16, 40, 42, 44, and/or 46, or (ii) the full-length complement of (i);

(d) a variant of the PDCT1 of SEQ ID NO: 2, 4, 6, 8, 10, 12, 14, 16, 40, 42, 44, and/or 46 comprising a substitution, preferably a conservative substitution, deletion, and/or insertion at one or more positions and having PDCT1 activity;

(e) a PDCT1 encoded by a polynucleotide that differs from SEQ ID NO: 1, 3, 5, 7, 9, 11, 13, 15, 39, 41, 43, and/or 45 due to the degeneracy of the genetic code; and

(f) a fragment of the PDCT1 of (a), (b), (c), (d) or (e) having PDCT1 activity;

Optionally, the seed oil is isolated.

Further, the present invention relates to a method for the production of a composition comprising the fatty acid 22:0 in a plant, or part thereof, like a plant cell, and/or part seed, or part thereof,

wherein the level of the 22:0 fatty acid in % (w/w) in the triacylglycerol fraction is higher than the 22:0 fatty acid level in % (w/w) in the diacylglyerol fraction, comprising, providing a plant cable to produce GLA and having an increased activity or expression of one or more PDCT compared to the wild type, the PDCT selected from the group consisting of:

(a) a PDCT1 having at least 80% sequence identity with SEQ ID NO: 2, 4, 6, 8, 10, 12, 14, 16, 40, 42, 44, and/or 46;

(b) a PDCT1 encoded by a polynucleotide having at least 80% sequence identity with SEQ ID NO: 1, 3, 5, 7, 9, 11, 13, 15, 39, 41, 43, and/or 45;

(c) a PDCT1 encoded by a polynucleotide that hybridizes under high stringency conditions with (i) a polynucleotide that encodes the amino acid sequence of SEQ ID NO: 2, 4, 6, 8, 10, 12, 14, 16, 40, 42, 44, and/or 46, or (ii) the full-length complement of (i);

(d) a variant of the PDCT1 of SEQ ID NO: 2, 4, 6, 8, 10, 12, 14, 16, 40, 42, 44, and/or 46 comprising a substitution, preferably a conservative substitution, deletion, and/or insertion at one or more positions and having PDCT1 activity;

(e) a PDCT1 encoded by a polynucleotide that differs from SEQ ID NO: 1, 3, 5, 7, 9, 11, 13, 15, 39, 41, 43, and/or 45 due to the degeneracy of the genetic code; and

(f) a fragment of the PDCT1 of (a), (b), (c), (d) or (e) having PDCT1 activity;

Further, the present invention relates to a method for the production of a composition comprising the fatty acid SDA in a plant, or part thereof, like a plant cell, and/or part seed, or part thereof,

wherein the level of SDA in % (w/w) in the phosphatyidylcholine (PC) fraction is higher than the SDA level in % (w/w) in the triacylglycerol fraction, comprising,

providing a plant cable to produce SDA and having an increased activity or expression of one or more PDCT compared to the wild type, the PDCT selected from the group consisting of:

(a) a PDCT1 having at least 80% sequence identity with SEQ ID NO: 2, 4, 6, 8, 10, 12, 14, 16, 40, 42, 44, and/or 46;

(b) a PDCT1 encoded by a polynucleotide having at least 80% sequence identity with SEQ ID NO: 1, 3, 5, 7, 9, 11, 13, 15, 39, 41, 43, and/or 45;

(c) a PDCT1 encoded by a polynucleotide that hybridizes under high stringency conditions with (i) a polynucleotide that encodes the amino acid sequence of SEQ ID NO: 2, 4, 6, 8, 10, 12, 14, 16, 40, 42, 44, and/or 46, or (ii) the full-length complement of (i);

(d) a variant of the PDCT1 of SEQ ID NO: 2, 4, 6, 8, 10, 12, 14, 16, 40, 42, 44, and/or 46 comprising a substitution, preferably a conservative substitution, deletion, and/or insertion at one or more positions and having PDCT1 activity;

(e) a PDCT1 encoded by a polynucleotide that differs from SEQ ID NO: 1, 3, 5, 7, 9, 11, 13, 15, 39, 41, 43, and/or 45 due to the degeneracy of the genetic code; and

(f) a fragment of the PDCT1 of (a), (b), (c), (d) or (e) having PDCT1 activity;

Further, the present invention relates to a method to produce a plant or a part thereof, the plant cell, and/or the plant seed that comprises an oil comprising PUFAs, e.g. vlcPUFAs, as for example, EPA, DHA, and/or DPA and that is further characterized by
i. the level of the 18:2 fatty acid in % (w/w) in the triacylglycerol (TAG) fraction that is between 80 and 120% of the level as the 18:2 fatty acid level in % (w/w) in the diacylglyerol (DAG) fraction
ii. increased total PUFA level,
iii, a ratio of 18:1 fatty acid to total fatty acid content (w/w) is between 10% and 50% less compared to the control and/or wherein ratio ALA to total fatty acid content is reduced by between 10% and 50%,
Iv: an increased amount of GLA,
v. an increased ratio of C20 fatty acids to C18,
vi. the level of the 22:1 fatty acid in % (w/w) in the triacylglycerol fraction is higher than the 22:1 fatty acid level in % (w/w) in the diacylglyerol fraction, and/or
vii. the level of SDA in % (w/w) in the phosphatyidylcholine (PC) fraction is higher than the SDA level in % (w/w) in the triacylglycerol fraction.

Consequently, the present invention also relates to a plant raw oil comprising PUFAs, e.g. vIcPUFAs, as for example, EPA, DHA, and/or DPA that further comprises
i. the level of the 18:2 fatty acid in % (w/w) in the triacylglycerol (TAG) fraction that is between 80 and 120% of the level as the 18:2 fatty acid level in % (w/w) in the diacylglyerol (DAG) fraction
ii. increased total PUFA level,
iii. ratio of 18:1 fatty acid to total fatty acid content (w/w) is between 10% and 50% less compared to the control and/or wherein ratio ALA to total fatty acid content is reduced by between 10% and 50%,
Iv: an increased amount of GLA,
v. an increased ratio of C20 fatty acids to C18,
vi. the level of the 22:1 fatty acid in % (w/w) in the triacylglycerol fraction is higher than the 22:1 fatty acid level in % (w/w) in the diacylglyerol fraction, and/or
vii. the level of SDA in % (w/w) in the phosphatyidylcholine (PC) fraction is higher than the SDA level in % (w/w) in the triacylglycerol fraction.

The level of 18:2 fatty acid in the triacylglycerol fraction is more than 70% and less than 140% of the 18:2 fatty acid level in the diacylglyerol fraction,e g. 80%, 90%, or more, for example, around 95% and less than 130%, 120%, 110% or 100%.

The level of 22:1 in the triacylglycerol fraction is more than 130% of the 22:1 fatty acid level in the diacylglyerol fraction, e.g. 150%, 200%, 250% or 280% or more, for example, between 280% and 350%, e.g. less than 500%, or 400%, 350% or less. It was found that in the control, the level of 22:1 in the triacylglycerol fraction is less the level of 22:1 in the diacylglyerol fraction.

The level of SDA in the phosphatyidylcholine fraction is more than 100% of the level of SDA in the triacylglycerol fraction, e.g. 105%, 110%, 120% or 130% or more, for example, around 150% and less than 250, or 200% or less.

Thus, by making use of the PDCT of the present invention it is possible to improve the conversion efficiency of a Delta-6 elongase in plants, produce plants with an increased conversion rate of oleic acid to combined level C18 to C22 fatty acids in the cell and/or seed, and thus, to increase the production of PUFAs in a plant, cell or seed compared to a control that does not express PDCT of the invention, e.g. the PDCT1 or does have a reduced activity compared to the cell, plant or seed used in the method of the invention.

Preferably, the plant, plant cell and/or the seed is also expressing a Delta-6 desaturase and/or a Delta-6 elongase.

The invention also provides an improved method for the production of total PUFA in a plant, plant cell, seed or a part thereof, which comprises providing a plant, seed, or plant cell capable to produce SDA, ETA, GLA HG LA, EPA, DHA, and/or DPA and the plant, seed, and/or plant cell functionally expressing:

at least a nucleic acid sequence which encodes a Delta-12 desaturase activity

at least a nucleic acid sequence which encodes a omega 3 desaturase activity,

at least a nucleic acid sequence which encodes a Delta-6-desaturase activity, and

at least a nucleic acid sequence which encodes a Delta-6 elongase activity, and

at least a nucleic acid sequence which encodes a Delta-5 desaturase activity, and

at least a nucleic acid sequence which encodes a Delta-5 elongase activity, and

at least a nucleic acid sequence which encodes a Delta-4 desaturase activity, and

whereby preferably, at least one desaturase uses Acyl-CoA as a substrate, and whereby the plant has an increased activity of one or more PDCT of the invention, e.g. PDCT1. Thus, the present invention provides a method of the invention comprising providing or producing a plant, a part thereof, a plant cell, and/or plant seed with an increased activity or de novo expression of one or more PDCT selected from the group consisting of:

(a) a PDCT1 having at least 80% sequence identity with SEQ ID NO: 2, 4, 6, 8, 10, 12, 14, 16, 40, 42, 44, and/or 46;

(b) a PDCT1 encoded by a polynucleotide having at least 80% sequence identity with SEQ ID NO: 1, 3, 5, 7, 9, 11, 13, 15, 39, 41, 43, and/or 45;

(c) a PDCT1 encoded by a polynucleotide that hybridizes under high stringency conditions with (i) a polynucleotide that encodes the amino acid sequence of SEQ ID NO: 2, 4, 6, 8, 10, 12, 14, 16, 40, 42, 44, and/or 46, or (ii) the full-length complement of (i);

(d) a variant of the PDCT1 of SEQ ID NO: 2, 4, 6, 8, 10, 12, 14, 16, 40, 42, 44, and/or 46 comprising a substitution, preferably a conservative substitution, deletion, and/or insertion at one or more positions and having PDCT1 activity;

(e) a PDCT1 encoded by a polynucleotide that differs from SEQ ID NO: 1, 3, 5, 7, 9, 11, 13, 15, 39, 41, 43, and/or 45 due to the degeneracy of the genetic code; and

(f) a fragment of the PDCT1 of (a), (b), (c), (d) or (e) having PDCT1 activity;

According to the invention, the activity of a PDCT1 can be increase, e.g. by de novo expression, for example after transformation with a corresponding expression construct, or by increasing the endogenous activity. Thus, the method of the invention comprises also increasing the endogenous activity of at least one endogenous PDCT1.

According to this invention, the PDCT1 activity can be increased in B. carinata by introducing and expressing a expression construct encoding for a PDCT1 as described herein. For example, the PDCT1 activity can be a PDCT1 gene from B. napus or of B. carinata or of B. juncea as described in Table 1. In one embodiment, the PDCT1 activity in B. napus is increased by increasing the activity of a B. napus PDCT1 as shown in Table 1. Further, the PDCT1 activity can be increased in B. napus by increasing the activity of an non-endogenous PDCT1 as described in Table 1, e.g. a PDCT from B. juncea or B. carinata. In one embodiment, the PDCT1 activity in B. juncea is increased by increasing the activity of a B. juncea PDCT1 as shown in Table 1. Further, the PDCT1 activity can be increased in B. juncea by increasing the activity of n non-endogenous PDCT1 as described in Table 1, e.g. a PDCT from B. napus or B. carinata. In one embodiment, the PDCT1 activity in B. carinata is increased by increasing the activity of a B. carinata PDCT1 as shown in Table 1. Further, the PDCT1 activity can be increased in B. carinata by increasing the activity of an non-endogenous PDCT1 as described in Table 1, e.g. a PDCT from B. juncea or B. napus.

According to the invention, also the activity of a PDCT19 can be increase, e.g. by de novo expression, for example after transformation with a corresponding expression construct, or by increasing the endogenous activity. Thus, the method of the invention comprises also increasing the activity of at least one PDCT19 in the plant, plant cell or plant seed, whereby the PDCT19 is selected from:

(a) a PDCT19 having at least 80% sequence identity with SEQ ID NO: 36, 38, and/or 48;

(b) a PDCT19 encoded by a polynucleotide having at least 80% sequence identity with SEQ ID NO: 35, 37, and/or 47;

(c) a PDCT19 encoded by a polynucleotide that hybridizes under high stringency conditions with (i) a polynucleotide that encodes the amino acid sequence of SEQ ID NO: 36, 38, and/or 48, or (ii) the full-length complement of (i);

(d) a variant of the PDCT19 of SEQ ID NO: 36, 38, and/or 48 comprising a substitution, preferably a conservative substitution, deletion, and/or insertion at one or more positions and having PDCT19 activity;

(e) a PDCT19 encoded by a polynucleotide that differs from SEQ ID NO: 35, 37, and/or 47 due to the degeneracy of the genetic code; and

(f) a fragment of the PDCT19 of (a), (b), (c), (d) or (e) having PDCT19 activity.

Further, according to the method of the invention also the activity of a PDCT3 and/or PDCT5 can be reduced. The PDCT3 can be selected from the group of

(a) a PDCT3 and/or PDCT5 and having at least 80% sequence identity with SEQ ID NO: 18, 20, 22, 24, 26, 28, 30, 32, 50, 52, 54, 56, 58, and/or 60;

(b) a PDCT3 and/or PDCT5 encoded by a polynucleotide having at least 80% sequence identity with SEQ ID NO: 17, 19, 21, 23, 27, 29, 31, 49, 51, 53, 55, and/or 57;

(c) a PDCT3 and/or PDCT5 encoded by a polynucleotide that hybridizes under high stringency conditions with (i) a polynucleotide that encodes the amino acid sequence of SEQ ID NO: 18, 20, 22, 24, 26, 28, 30, 32, 50, 52, 54, 56, 58, and/or 60, or (ii) the full-length complement of (i);

(d) a variant of the PDCT3 and/or PDCT5 of SEQ ID NO: 18, 20, 22, 24, 26, 28, 30, 32, 50, 52, 54, 56, 58, and/or 60 comprising a substitution, preferably a conservative substitution, deletion, and/or insertion at one or more positions and having PDCT activity;

(e) a PDCT3 and/or PDCT5 encoded by a polynucleotide that differs from SEQ ID NO: 17, 19, 21, 23, 27, 29, 31, 49, 51, 53, 55, and/or 57 due to the degeneracy of the genetic code; and

(f) a fragment of the PDCT of (a), (b), (c), (d) or (e) having PDCT3 and/or PDCT5 activity.

According to the invention, the activity of a PDCT3 and/or PDCT5 is decrease in the method of the invention, e.g. by expression of any expression reducing or inhibiting agent, like a transcription factor, ribozyme, microRNA, or antisense molecule, or by integrating into the genes or regulatory elements that encodes or regulate the expression or activity of the PDCT3 or PDCT5 a sequence or mutating the genes or regulatory elements that encode or regulate the expression or activity of the PDCT3 or PDCT 5, whereby the measures results in the inhibition of an active PDCT3 or PDCT5 or results in no expression of a polypeptide from that gene with the insert at all or results in the expression of an inactive polypeptide form the gene that in a control or wild type cell encodes for a PDCT3 or PDCT5.

Thus, according to method of the invention depleting, inhibiting, reducing or decreasing or blocking the activity of at least one PDCT3 and/or PDCT5 in the plant, plant cell or seed used in the method of the invention is independent on the method that is used to achieve the decrease, depletion, inhibition, reduction or block of the activity.

Accordingly, the term “reduced” in context of the activity or expression of a PDCT3 and/or PDCT5 means herein that the activity of the PDCT3 and/or PDCT5 in a plant, cell, seed or a part thereof is reduced, blocked, depleted or inhibited compared to a control as described herein. For example, in the assay described herein no or a reduced PDCT3 and/or PDCT5 activity can be measured. For example, the term “reduced” also encompasses a mutation or a knock out of a gene encoding the PDCT3 or PDCT5 in a plant, plant cell or seed. Thus, the term “reduced” also comprises the mutation or knock out of the PDCT3 and/or PDCT5 of an oil seed crop producing PUFA, e.g. a B. napus, B. carrinata, B. rapa, C. sativa or B. juncea or the expression of antisense RNA, ribozyme or microRNA molecules that target for the PDCT3 and/or PDCT5 in said plants, e.g. genes comprising the B. napus, C. sativa or B. juncea sequences as shown in the sequence listing

Optionally, the method of the invention comprises the step of isolating the oil from the plant, plant seed or plant cell.

Accordingly, a phosphotidylcholine:diacylglycerol cholinephosphotransferase (PDCT) enzyme is considered as a PDCT activity of the invention or “PDCT1” if it has a phosphotidylcholine:diacylglycerol cholinephosphotransferase (PDCT) activity and further in a functionality assay comprising the expression of the PDCT in an A. thaliana ROD1k.o. mutant expressing a delta 6 elongase and a delta 6 desaturase the ALA and LA level is less than the level of C18, C20 and C22 PUFAs and the conversion rate of a delta 6 desaturase being increased. An example for a corresponding functionality test is shown in the examples. Such an activity herein is described as the “PDCT activity of the invention” or the “PDCT1 activity”. Preferably the PDCT of the invention has 80% or higher identity to SEQ ID NO. 2, 4, 6, 8, 10, 12, 14, 16, 40, 42, 44, and/or 46. Preferably, the PDCT is not a Camelina C15 polypeptide, e.g. as shown in SEQ ID NO: 34. For example, the Delta-6 desaturase is phospholipid-dependent.

Further, according to this invention, a PDCT is considered as a “PDCT1” if in a functionality assay comprising the expression the PDCT in A. thaliana ROD1 k.o. mutant expressing a delta 6 elongase and a delta 6 desaturase and the PDCT having phosphotidylcholine:diacylglycerol cholinephosphotransferase (PDCT) activity, whereby the conversion rate of a delta 6 elongase is increased. Preferably the total PUFA level is increased. Preferably the PDCT1 has 80% or higher identity to SEQ ID NO.2, and/or 4, preferably also to 6, 8, 10 and/or 12. Even more preferred is an identity of 80% also to 14 or 16 or 40. Preferably the Delta-6 desaturase is Acyl-CoA dependent.

Further, according to this invention, a PDCT is considered as a “PDCT3” or a “PDCT5” if in an functionality assay comprising the expression the PDCT in A. thaliana k.o. ROD1 mutant expressing a delta 6 elongase and a delta 6 desaturase and the PDCT having phosphotidylcholine:diacylglycerol cholinephosphotransferase (PDCT) activity, and whereby the conversion rate of a delta 6 elongase is decreased. Preferably, also the ETA level is reduced. Preferably the PDCT3 and/or PDCT5 has 80% or higher identity to 18, 20, 22, 24, 26, 28, 30, 32, 50, 52, 54, 56, 58, and/or 60. Preferably a PDCT3 has an identity of at least 80% to SEQ ID NO. 18, 22, or 24. Preferably, a PDCT5 has an identity of at least 80% to SEQ ID NO. 20, 26 or 28. Preferably the Delta-6 desaturase is Acyl-CoA dependent.

An increase in the level or the increase of a fatty acid or the increase of a combination of fatty acids or the increase of PUFAs or the increase of total PUFAs or similar expressions refer to an increase of the specific compound or the combination of compounds compared to a control. For example, the increase of said compound or combination of compound is an relative increase within the corresponding extract from plants, plant cells or plant seeds. According to the invention, the increase of a fatty acid or a combination of fatty acids, e.g. of a PUFA or of PUFAs, like vIcPUFAs, is measured in the oil or the fatty acids extracted from the plant, plant cell or plant seed in percent per volume or percent per weight, preferably percent of weight. For example, the content and composition of an extract from a plant, plant cell or plant seed or from plants, plant cells or plant seeds can be measured as shown in the examples.

“Total PUFA” as used in this invention refers to the level of GLA 18:3n-6, SDA 18:4n-3, DGLA 20:3n-6, EtrA 20:3n-3, ETA 20:4n-3, ARA 20:4n-6, EPA 20:5 n-3, DPA 22″5n-3, and DHA 22:6n-3.

With the level of “total” or “new” PUFA is meant the level of GLA 18:3n-6, SDA 18:4n-3, DGLA 20:3n-6, EtrA 20:3n-3, ETA 20:4n-3, ARA 20:4n-6, EPA 20:5 n-3, DPA 22″5n-3, and DHA 22:6n-3. For example, the term does not include (18:2n-6) and ALA (18:3n-3).

According to the present invention, unsaturated fatty acids preferably are polyunsaturated fatty acids, that is fatty acids comprising at least two, more preferably at least three and even more preferably at least or exactly 4 carbon-carbon double bonds. Unsaturated fatty acids including polyunsaturated fatty acids are generally known to the skilled person, important unsaturated fatty acids are categorised into a omega-3, omega-6 and omega-9 series, without any limitation intended. Unsaturated fatty acids of the omega-6 series include, for example, and without limitation, gamma-linolenic acid (18:3 n-6; GLA), di-homo-gamma-linolenic acid (C20:3 n-6; DGLA), arachidonic acid (C20:4 n-6; ARA), adrenic acid (also called docosatetraenoic acid or DTA; C22:4 n-6) and docosapentaenoic acid (C22:5 n-6). Unsaturated fatty acids of the omega-3 series include, for example and without limitation, stearidonic acid (18:4 n-3; STA or SDA), eicosatrienoic acid (C20:3 n-3; ETA), eicosatetraenoic acid (C20:4 n-3; ETA), eicosapentaenoic acid (C20:5 n-3; EPA), docosapentaenoic acid (C22:5 n-3; DPA) and docosahexaenoic acid (C22:6 n-3; DHA). Unsaturated fatty acids also include fatty acids with greater than 22 carbons and 4 or more double bonds, for example and without limitation, C28:8 (n-3). Unsaturated fatty acids of the omega-9 series include, for example, and without limitation, mead acid (20:3 n-9; 5,8,11-eicosatrienoic acid), erucic acid (22:1 n-9; 13-docosenoic acid) and nervonic acid (24:1 n-9; 15-tetracosenoic acid). Further unsaturated fatty acids are eicosadienoic acid (C20:2d11, 14; EDA) and eicosatrienoic acid (20:3d11, 14, 17; ETrA).

In the method of the invention a number of VLC-PUFA and intermediates are produced that are non-naturally occurring in wild type crop plant, in particular not in oil seed crop plants, though they VLC-PUFA and intermediates may occur in various other organisms. These fatty acids include but are not limited to 18:2n-9, GLA, SDA, 20:2n-9, 20:3n-9, 20:3 n-6, 20:4n-6, 22:2n-6, 22:5n-6, 22:4n-3, 22:5n-3, and 22:6n-3.

According to the present invention, the metabolic property preferably is the production and particularly preferably the yield of an omega-6 type and/or an omega-3 type unsaturated fatty acid. Such yield is preferably defined as the percentage of said fatty acid relative to the total fatty acids of an extract, preferably of a plant or seed oil. Thus, preferably the assay method of the present invention entails measuring the amount and/or concentration of an unsaturated fatty acid, preferably of an unsaturated fatty acid having at least 20 carbon atoms length, for example 18, 20 and 22 carbon atoms length, and belonging to the omiga-3 or omega-6 series.

Preferably, the DPA, DHA and/or EPA level is increased in lipids or oil or in an composition of fatty acids derived or isolated from the plant, plant cell or seed provided according to the method of the invention.

The amount and/or concentration is determined on a plant extract, preferably a plant oil or plant lipids. The term “lipids” refers to a complex mixture of molecules comprising compounds such as sterols, waxes, fat soluble vitamins such as tocopherols and carotenoid/retinoids, sphingolipids, phosphoglycerides, glycolipids such as glycosphingolipids, phospholipids such as phosphatidylethanolamine, phosphatidylcholine, phosphatidylserine, phosphatidylglycerol, phosphatidylinositol or diphosphatidylglycerol, monoacylglycerides, diacylglycerides, triacylglycerides or other fatty acid esters such as acetylcoenzyme A esters. “Lipids” can be obtained from biological samples, such as fungi, algae, plants, leaves, seeds, or extracts thereof, by solvent extraction using protocols well known to those skilled in the art (for example, as described in Bligh, E. G., and Dyer, J. J. (1959) Can J. Biochem. Physiol. 37: 911-918).

The term “oil” refers to a fatty acid mixture comprising unsaturated and/or saturated fatty acids which are esterified to triglycerides. The oil may further comprise free fatty acids. Fatty acid content can be, e.g., determined by GC analysis after converting the fatty acids into the methyl esters by transesterification. The content of the various fatty acids in the oil or fat can vary, in particular depending on the source. It is known that most of the fatty acids in plant oil are esterified in triacylglycerides. In addition the oil of the invention may comprise other molecular species, such as monoacylglycerides, diacylglycerides, phospholipids, or any the molecules comprising lipids. Moreover, oil may comprise minor amounts of the polynucleotide or vector of the invention. Such low amounts, however, can be detected only by highly sensitive techniques such as PCR. Oil can be obtained by extraction of lipids from any lipid containing biological tissue and the amount of oil recovered is dependent on the amount of triacylglycerides present in the tissue. Extraction of oil from biological material can be achieved in a variety of ways, including solvent and mechanical extraction. Specifically, extraction of canola oil typically involves both solvent and mechanical extraction, the products of which are combined to form crude oil. The crude canola oil is further purified to remove phospholipids, free fatty acids, pigments and metals, and odifierous compounds by sequential degumming, refining, bleaching, and deoderorizing. The final product after these steps is a refined, bleached, and deodorized oil comprising predominantly fatty acids in the form of triglycerides.

The method of the present invention comprises the step of providing and/or producing a plant. According to the present invention, the term “plant” shall mean a plant or part thereof in any developmental stage. Particularly, the term “plant” herein is to be understood to indicate a callus, shoots, root, stem, branch, leaf, flower, pollen and/or seed, and/or any part thereof. The plant can be monocotyledonous or dicotyledonous and preferably is a crop plant. Crop plants include Brassica species, corn, alfalfa, sunflower, soybean, cotton, safflower, peanut, sorghum, wheat, millet and tobacco. The plant preferably is an oil plant. Preferred plants are of order Brassicales, particularly preferred of family Brassicaceae.

Even more preferred are plants of oil seed crops, e.g. Camelina sativa, Brassica sp., Brassica aucheri, Brassica balearica, Brassica barrelieri, Brassica carinata, Brassica carinata x Brassica napus, Brassica carinata x Brassica rapa, Brassica carinata x Brassica juncea, Brassica cretica, Brassica deflexa, Brassica desnottesii, Brassica drepanensis, Brassica elongata, Brassica fruticulosa, Brassica gravinae, Brassica hilarionis, Brassica incana, Brassica insularis, Brassica juncea, Brassica macrocarpa, Brassica maurorum, Brassica montana, Brassica napus, Brassica napus x Brassica juncea, Brassica napus x Brassica nigra, Brassica nigra, Brassica oleracea, Brassica oxyrrhina, Brassica procumbens, Brassica rapa, Brassica repanda, Brassica rupestris, Brassica ruvo, Brassica souliei, Brassica spinescens, Brassica tournefortii or Brassica villosa.

The plant of the method of the present invention is capable of expressing a PDCT as defined herein, in particular a PDCT1. The plant can be provided by any appropriate means. For example, the plant can be provided by transforming a plant cell with a nucleic acid comprising a gene coding for the PDCT of the invention, in particular a PDCT1 and raising such transformed plant cell to a plant sufficiently developed for measuring the plant metabolic property. According to the invention, a plant can also be provided in the form of an offspring of such transformed plant. Such offspring may be produced vegetatively from material of a parent plant, or may be produced by crossing a plant with another plant, preferably by inbreeding.

The plant is capable of expressing a PDCT of the invention, in particular a PDCT1. According to the invention, the term “capable of expressing a gene product” means that a cell will produce the gene product provided that the growth conditions of the sale are sufficient for production of said gene product. For example, a plant is capable of expressing a PDCT of the invention, in particular a PDCT1 is a cell of said plant during any developmental stage of said plant will produce the corresponding PDCT of the invention, in particular a PDCT1. It goes without saying that where expression depends on human intervention, for example the application of an inductor, a plant is likewise considered capable of expressing the PDCT of the invention, in particular a PDCT1.

A PDCT having this desired sequence identity and/or sequence similarity and functionality is also called a PDCT of the present invention. The action of a PDCT is shown in FIG. 5.

For a metabolic pathway for the production of unsaturated and polyunsaturated fatty acids, see for example FIG. 4 or FIG. 1 of W02006100241.

Examples of PDCT referred to herein shown in the Examples, Figures and Tables, e.g. in Tables 5 or 6:

According to the invention, the plant is capable of expressing a PDCT of the invention, in particular a PDCT1, wherein said PDCT of the invention, in particular a PDCT1 has at least, the PDCT1 50, 70, 80, 85, 87, 88, 90, 91, 92, 92, 94, 95, 96, 97, 98, 99, or 100% sequence identity with SEQ ID NO: 2, 4, 6, 8, 10, 12, 14, 16, 40, 42, 44, and/or 46. For example, the PDCT of said method has at least 50, 70, 80, 85, 87, 88, 90, 91, 92, 92, 94, 95, 96, 97, 98, 99, or 100% sequence identity with SEQ ID NO: 2 or 6. Further, for example, the PDCT of said method has at least 50, 70, 80, 85, 87, 88, 90, 91, 92, 92, 94, 95, 96, 97, 98, 99, or 100% sequence identity with SEQ ID NO: 4 or 8. Likewise, for example, the PDCT of said method has at least 50, 70, 80, 85, 87, 88, 90, 91, 92, 92, 94, 95, 96, 97, 98, 99, or 100% sequence identity with SEQ ID NO: 46.

The plant of the method of the present invention may also be capable of expressing an other PDCT as defined herein, in particular a PDCT19. The plant can be provided by any appropriate means. For example, the plant can be provided by transforming a plant cell with a nucleic acid comprising a gene coding for a PDCT19 and raising such transformed plant cell to a plant sufficiently developed for measuring the plant metabolic property.

The plant is capable of expressing a PDCT, in particular a PDCT1 and a PDCT19. According to the invention, the term “capable of expressing a gene product” means that a cell will produce the gene product provided that the growth conditions of the sale are sufficient for production of said gene product. For example, a plant is capable of expressing a PDCT1 is a cell of said plant during any developmental stage of said plant will produce the PDCT1. It goes without saying that where expression depends on human intervention, for example the application of an inductor, a plant is likewise considered capable of expressing a PDCT1, for example PDCT1 and PDCT19.

According to the invention, a PDCT19 can have 50, 70, 80, 85, 87, 88, 90, 91, 92, 92, 94, 95, 96, 97, 98, 99, or 100% sequence identity with SEQ ID NO: 36, 38, and/or 48. For example, a PDCT19 as used in the method of the invention, e.g. in combination with the PDCT of the invention, e.g. with a PDCT1, has at least 50, 70, 80, 85, 87, 88, 90, 91, 92, 92, 94, 95, 96, 97, 98, 99, or 100% sequence identity with SEQ ID NO: 36. Further, for example, the PDCT19 as used in the method of the invention, e.g. in combination with the PDCT of the invention, e.g. with a PDCT1, has at least 50, 70, 80, 85, 87, 88, 90, 91, 92, 92, 94, 95, 96, 97, 98, 99, or 100% sequence identity with SEQ ID NO: 38. Likewise, for example, the PDCT19 as used in the method of the invention, e.g. in combination with the PDCT of the invention, e.g. with a PDCT1, has at least 50, 70, 80, 85, 87, 88, 90, 91, 92, 92, 94, 95, 96, 97, 98, 99, or 100% sequence identity with SEQ ID NO: 48.

According to the invention, a nucleic acid sequence encoding a PDCT19 can have 50, 70, 80, 85, 87, 88, 90, 91, 92, 92, 94, 95, 96, 97, 98, 99, or 100% sequence identity with SEQ ID NO: 35, 37, and/or 47.

The plant of the method of the present invention may also be capable of expressing an other PDCT as defined herein, in particular a PDCT3 or a PDCT5. Surprisingly, it was found that the reduction, depletion, inhibition or deletion of the activity of an endogenous PDCT3 and/or PDCT5 leads to an improved production of PUFAs, in particular of EPA, DHA and/or DPA. The plant, plant cell or plant seed, in which the endogenous activity and/or expression had been reduced, depleted, inhibited or deleted compared to a control can be provided by any appropriate means. For example, the plant can be provided by transforming a plant cell with a nucleic acid comprising an inhibitor of expression or activity of the PDCT3 and/or PDCT5, e.g. a microRNA, antisense, ribozyme, antibody, inhibitor, knock-out etc, and raising such transformed plant cell to a plant sufficiently developed for measuring the plant metabolic property. According to the invention, a plant can also be provided in the form of an offspring of such transformed plant. Such offspring may be produced vegetatively from material of a parent plant, or may be produced by crossing a plant with another plant, preferably by inbreeding.

For example, in the method of the invention, the plant is not capable of expressing an endogenous PDCT3 and/or 5 or has a reduced expression of a PDCT3 or 5, compared to the control, and still has an increased activity of PDCT1 and/or a PDCT19. For example, a plant is not capable of expressing a PDCT3 and/or PDCT 5 is a cell of said plant during any developmental stage of said plant will not produce the PDCT3 and/or PDCT5. It goes without saying that where reduction of expression or activity depends on human intervention, for example the application of an repressor, e.g. a microRNA, antisense, ribozyme, antibody, inhibitor, knock out, etc, with a partial or full repression of the endogenous activity of the PDCT3 and/or PDCT5 in a plant, plant cell or seed can still be capable of expressing a PDCT1 and/or PDCT19.

According to the invention, a PDCT3 and/or PDCT5 can have 50, 70, 80, 85, 87, 88, 90, 91, 92, 92, 94, 95, 96, 97, 98, 99, or 100% sequence identity with SEQ ID NO: 18, 20, 22, 24, 26, 28, 30, 32, 50, 52, 54, 56, 58, and/or 60. According to the invention, a nucleic acid sequence encoding a

PDCT3 and/or PDCT5 can have 50, 70, 80, 85, 87, 88, 90, 91, 92, 92, 94, 95, 96, 97, 98, 99, or 100% sequence identity with SEQ ID NO: 1, 3, 5, 7, 9, 11, 13, 15, 39, 41, 43,and/or 45.

According to the invention, a plant can also be provided in the form of an offspring of such transformed plant. Such offspring may be produced vegetatively from material of a parent plant, or may be produced by crossing a plant with another plant, preferably by inbreeding.

A gene coding for a PDCT of the present invention can be obtained by de novo synthesis. Starting from any of the amino acid sequences SEQ ID NO: 2, 4, 6, 8, 10, 12, 14, 16, 40, 42, 44, and/or 46, the skilled person can reverse-translate the selected sequence into a nucleic acid sequence and have the sequence synthesised. As described herein, the skilled person can also introduce one or more mutations, including insertions, substitutions and deletions to the amino acid sequence chosen or the corresponding nucleic acid sequence. For reverse translation, the skilled person can and should use nucleic acid codons such as to reflect codon frequency of the plant intended for expression of said PDCT of the present invention. By using any of the amino acid sequences according to SEQ ID NO: 2, 4, 6, 8, 10, 12, 14, 16, 40, 42, 44, and/or 46 as such or one or more mutations, the person can obtain using routine techniques and standard equipment, a PDCT having the beneficial properties described herein and exhibiting these beneficial properties in numerous plant species.

The amino acid sequence of the PDCT of the present invention may be identical to any of the sequences according to SEQ ID NO: 2, 4, 6, 8, 10, 12, 14, 16, 40, 42, 44, and/or 46. However, in certain embodiments it is preferred that the amino acid sequence of the PDCT of the present invention is not the sequence encoding a PDCT 3 and/or a PDCT5, e.g. SEQ ID NO: 18, 20, 22, 24, 26, 28, 30, 32, 50, 52, 54, 56, or 58. Where the skilled person for any reason wants to avoid any one or more of the amino acid sequences according to SEQ ID NO: 18, 20, 22, 24, 26, 28, 30, 32, 50, 52, 54, 56, or 58, the skilled person can use any of the remaining sequences of this set of sequences. However, the skilled person can also make up a new amino acid and corresponding nucleic acid sequence by selecting a base sequence from the set of amino acid sequences according to SEQ ID NO: 2, 4, 6, 8, 10, 12, 14, 16, 40, 42, 44, and/or 46 and introducing one or more mutations (insertions, substitutions and/or deletions) at appropriate positions of the base sequence to obtain a derived sequence. Generally, the skilled person will take into account that the higher the sequence identity and/or similarity between base sequence and derived sequence, the more will the corresponding derived PDCT resemble the PDCT activity that corresponds to the PDCT of the base sequence or the PDCT activity of the invention. Thus, if the skilled person uses a mutated PDCT according to the present invention and such mutated PDCT unexpectedly does not convey the benefits of a PDCT of the present invention, e.g. a PDCT1 with the PDCT activity of the invention, the skilled person should reduce the number of differences of the PDCT sequence to increase resemblance of any of the sequences according to SEQ ID NO: 2, 4, 6, 8, 10, 12, 14, 16, 40, 42, 44, and/or 46.

For substituting amino acids of a base sequence selected from any of the sequences SEQ ID NO: 2, 4, 6, 8, 10, 12, 14, 16, 40, 42, 44, and/or 46 without regard to the occurrence of amino acid in other of these sequences, the following applies, wherein letters indicate L amino acids using their common abbreviation and bracketed numbers indicate preference of replacement (higher numbers indicate higher preference), as long as the PDCT activity of the invention is maintained: A may be replaced by any amino acid selected from S (1), C (0), G (0), T (0) or V (0). C may be replaced by A (0). D may be replaced by any amino acid selected from E (2), N (1), Q (0) or S (0). E may be replaced by any amino acid selected from D (2), Q (2), K (1), H (0), N (0), R (0) or S (0). F may be replaced by any amino acid selected from Y (3), W (1), I (0), L (0) or M (0). G may be replaced by any amino acid selected from A (0), N (0) or S (0). H may be replaced by any amino acid selected from Y (2), N (1), E (0), Q (0) or R (0). I may be replaced by any amino acid selected from V (3), L (2), M (1) or F (0). K may be replaced by any amino acid selected from R (2), E (1), Q (1), N (0) or S (0). L may be replaced by any amino acid selected from I (2), M (2), V (1) or F (0). M may be replaced by any amino acid selected from L (2), I (1), V (1), F (0) or Q (0). N may be replaced by any amino acid selected from D (1), H (1), S (1), E (0), G (0), K (0), Q (0), R (0) or T (0). Q may be replaced by any amino acid selected from E (2), K (1), R (1), D (0), H (0), M (0), N (0) or S (0). R may be replaced by any amino acid selected from K (2), Q (1), E (0), H (0) or N (0). S may be replaced by any amino acid selected from A (1), N (1), T (1), D (0), E (0), G (0), K (0) or Q (0). T may be replaced by any amino acid selected from S (1), A (0), N (0) or V (0). V may be replaced by any amino acid selected from I (3), L (1), M (1), A (0) or T (0). W may be replaced by any amino acid selected from Y (2) or F (1). Y may be replaced by any amino acid selected from F (3), H (2) or W (2).

Enzyme variants may be defined by their sequence identity when compared to a parent enzyme. Sequence identity usually is provided as “% sequence identity” or “% identity”. To determine the percent-identity between two amino acid sequences in a first step a pairwise sequence alignment is generated between those two sequences, wherein the two sequences are aligned over their complete length (i.e., a pairwise global alignment). The alignment is generated with a program implementing the Needleman and Wunsch algorithm (J. Mol. Biol. (1979) 48, p. 443-453), preferably by using the program “NEEDLE” (The European Molecular Biology Open Software Suite (EMBOSS)) with the programs default parameters (gapopen=10.0, gapextend=0.5 and matrix=EBLOSUM62). The preferred alignment for the purpose of this invention is that alignment, from which the highest sequence identity can be determined.

The following example is meant to illustrate two nucleotide sequences, but the same calculations apply to protein sequences:

Seq A: AAGATACTG length: 9 bases
Seq B: GATCTGA length: 7 bases

Hence, the shorter sequence is sequence B.

Producing a pairwise global alignment which is showing both sequences over their complete lengths results in

Seq A: AAGATACTG-
||| |||
Seq B: --GAT-CTGA

The “I” symbol in the alignment indicates identical residues (which means bases for DNA or amino acids for proteins). The number of identical residues is 6.

The “-” symbol in the alignment indicates gaps. The number of gaps introduced by alignment within the Seq B is 1. The number of gaps introduced by alignment at borders of Seq B is 2, and at borders of Seq A is 1.

The alignment length showing the aligned sequences over their complete length is 10.

Producing a pairwise alignment which is showing the shorter sequence over its complete length according to the invention consequently results in:

Seq A: GATACTG-
||| |||
Seq B: GAT-CTGA

Producing a pairwise alignment which is showing sequence A over its complete length according to the invention consequently results in:

Seq A: AAGATACTG
||| |||
Seq B: --GAT-CTG

Producing a pairwise alignment which is showing sequence B over its complete length according to the invention consequently results in:

Seq A: GATACTG-
||| |||
Seq B: GAT-CTGA

The alignment length showing the shorter sequence over its complete length is 8 (one gap is present which is factored in the alignment length of the shorter sequence).

Accordingly, the alignment length showing Seq A over its complete length would be 9 (meaning Seq A is the sequence of the invention).

Accordingly, the alignment length showing Seq B over its complete length would be 8 (meaning Seq B is the sequence of the invention).

After aligning two sequences, in a second step, an identity value is determined from the alignment produced. For purposes of this description, percent identity is calculated by %-identity=(identical residues/length of the alignment region which is showing the two aligned sequences over their complete length)*100. Thus, sequence identity in relation to comparison of two amino acid sequences according to this embodiment is calculated by dividing the number of identical residues by the length of the alignment region which is showing the two aligned sequences over their complete length. This value is multiplied with 100 to give “%-identity”. According to the example provided above, %-identity is: (6/10)*100=60%.

Moreover, the preferred alignment program implementing the Needleman and Wunsch algorithm (J. Mol. Biol. (1979) 48, p. 443-453) is “NEEDLE” (The European Molecular Biology Open Software Suite (EMBOSS)) with the programs default parameters (gapopen=10.0, gapextend=0.5 and matrix=EDNAFULL).

In table 6, the identities between PDCTs used in the method of the invention and other PDCTs calculated as described herein are shown.

The PDCT of the present invention preferably has at least 50% amino acid sequence identity to any of the sequences SEQ ID NO: 2, 4, 6, 8, 10, 12, 14, 16, 40, 42, 44, and/or 46. Most preferably, the PDCT of the present invention has at least 50% amino acid sequence identity to sequence SEQ ID NO. 2 or 4. This PDCT can be shown to be functional, it is easy to obtain and conveys the benefits of the PDCT of the present invention. Preferably, the PDCT of the present invention has at least 55% amino acid sequence identity to any of the sequences SEQ ID NO: 6, 8, 10, 12, 14, 16 or 46, wherein identity to SEQ ID NO. 2, 4, 6 or 8 are particularly preferred, even more preferably at least 65%, even more preferably at least 72%, even more preferably at least 78%, even more preferably at least 80%, even more preferably at least 82%, even more preferably at least 89%, even more preferably at least 91%, even more preferably at least 96%. The PDCT of the present invention preferably has at least 50% amino acid sequence identity to any of the sequences SEQ ID NO. 6 or 8 or 10 or 12. Preferably, the PDCT of the present invention has at least 50% amino acid sequence identity to sequence SEQ ID NO. 6 or 8 or 10 or 12. This PDCT can be shown to be functional, it is easy to obtain and conveys the benefits of the PDCT of the present invention. Preferably, the PDCT of the present invention has at least 60% amino acid sequence identity to any of the sequences SEQ ID NO. 6 or 8 or 10 or 12, where similarity to SEQ ID NO. 2 or 4 is particularly preferred, even more preferably at least 73%, even more preferably at least 75%, even more preferably at least 89%, even more preferably at least 95%, even more preferably at least 96%, even more preferably at least 97%, even more preferably at least 98%, even more preferably at least 99%. Preferably, the PDCT of the present invention has both the required or preferred minimal identity and the required or preferred minimal similarity. The higher the similarity and identity between the amino acid sequence of the PDCT of the present invention and the amino acid sequence according to SEQ ID NO: 2, 4, 6, 8, 10, 12, 14, 16, 40, 42, 44, and/or 46 , the more reliable will the PDCT of the present invention exhibit PDCT activity in a plant cell, plant or seed as described herein and convey the benefits of the present invention. Preferably, the PDCT of the present invention is not a PDCT3 or a PDCT 5 and has not any of the sequences SEQ ID NO. 18, 20, 22, 24, 26, 28, 30, 32, 50, 52, 54, 56, 58, and/or 60.

Preferably, the amino acid sequence of the PDCT of the present invention differs from the amino acid sequences according to any of SEQ ID NO: 2, 4, 6, 8, 10, 12, 14, 16, 40, 42, 44, and/or 46 only at such one or more positions where according to FIG. 1 at least one of the amino acid sequences of SEQ ID NO. 2, 4, 6 or 8 differs from at least one other of the sequences SEQ ID NO. 2, 4, 6, or 8, preferably not allowing any amino acid insertion or deletion. FIG. 1 shows an alignment of the amino acid sequences of PDCT of the present invention. Preferably, the amino acid sequence of the PDCT of the invention can be thought to be the result of exchanging selected amino acids from one chosen base sequence of the sequences SEQ ID NO: 2, 4, 6, 8, 10, 12, 14, 16, 40, 42, 44, and/or 46 for the corresponding amino acid at the respective positions of any other of the sequences SEQ ID NO: 2, 4, 6, 8, 10, 12, 14, 16, 40, 42, 44, and/or 46. Also, preferably, any mutation should increase the similarity, or, even more preferably, the identity, of the amino acid sequence of the PDCT of the present invention to that of a sequence according to SEQ ID NO: 2, 4, 6, 8, 10, 12, 14, 16, 40, 42, 44, and/or 46 and reduce the similarity or, even more preferably, the identity, to an amino acid sequence according to SEQ ID NO: 2, 4, 6, 8, 10, 12, 14, 16, 40, 42, 44, and/or 46.

For the reasons indicated above, the PDCT of the present invention preferably consists of the amino acid sequence SEQ ID NO. 2, 4, 6 and/or 8. More preferably, the PDCT of the present invention does not differ from the amino acid sequence of SEQ ID NO. 2, 4, 6, and/or 8 by an insertion or deletion and thus only comprises one or more substitutions. Even more preferably, the PDCT of the present invention consists of an amino acid sequence that differs from SEQ ID NO. 2, 4, 6, and/or 8 only by amino acids found at the corresponding position of amino acid sequence SEQ ID NO. 2, 4, 6, and/or 8.

The plant of the present invention is further capable of expressing at least one or more enzymes of unsaturated fatty acid metabolism. Preferably, such enzymes are capable of using an unsaturated fatty acid of the omega-6 and/or, more preferably, of the omega-3 series as a substrate. Preferred activities of the enzymes are: desaturase, elongase, ACS, acylglycerol-3-phosphate acyltransferase (AGPAT), choline phosphotransferase (CPT), diacylglycerol acyltransferase (DGAT), glycerol-3-phosphate acyltransferase (GPAT), lysophosphatidate acyltransferase (LPAT), lysophosphatidylcholine acyltransferase (LPCAT),lysophosphatidylethanolamine acyltransferase (LPEAT), lysophospholipid acyltransferase (LPLAT), phosphatidate phosphatase (PAP), phospholipid:diacylglycerol acyltransferase (PDAT), phosphatidylcholine:diacylglycerol choline phosphotransferase (PDCT), particularly Delta-12 desaturase, Delta-8 desaturase, Delta-6 desaturase, Delta-5 desaturase, Delta-4 desaturase, Delta-9 elongase, Delta-6 elongase, Delta-5 elongase, omega-3 desaturase.

At least one of the enzymes is capable of using linoleic acid as substrate. Such enzymes are known to the skilled person as omega-3 desaturases, Delta-15 desaturases, Delta-9 desaturase and Delta-6 desaturases. It is possible that one or more enzymes of unsaturated fatty acid metabolism can have more than one activity. For example, it is common for omega-3 desaturases to be also Delta-15 desaturases and/or Delta-17 desaturases and/or Delta-19 desaturases. Further preferred enzymes of unsaturated fatty acid metabolic is our Delta-12 desaturases, omega-3 desaturases, Delta-6 desaturases, Delta-6 elongases, Delta-5 desaturases, Delta-5 elongase and Delta-4 desaturases. At least one of these enzymes is supposedly connected to a plant metabolic property. Preferably, the metabolic property is the presence and/or concentration of the product of the respective enzyme. Thus, preferably the plant metabolic property is the presence and/or concentration of any of GL a, SDA, EDA, ETrA, the GLA, EDTA, ARA, EPA, DTA, DPA and DHA, wherein particularly preferred are the concentration of ARA, EPA and DHA.

In the method of the present invention, the plant is capable of expressing the PDCT of the present invention and at least one more enzyme of the unsaturated fatty acid metabolic pathway during the plant is grown. “Growing” for the present invention means to nurture plant material, preferably a plant can use, embryo or seed, such that cells of said plant material can develop and preferably multiply, such that at least one cell of the developed plant material can be expected to exhibit the plant metabolic property. For example, where the expression of a gene coding for an enzyme of unsaturated fatty acid metabolism, for example a desaturase or elongates, is under the control of a tissue-specific promoter, the plant material is grown such that the corresponding tissue develops.

The plant metabolic property is then measured by any suitable means. For example, the concentration of fatty acids in the form of free fatty acids or in the form of mono-, di- or triglycerides can be measured from extracts of plant material, preferably of plant seeds and most preferably from seed oil.

The method of the present invention preferably is not performed only on one plant but on a group of plants. This way, the measured plant metabolic properties will be statistically more significant than measurements taken only on plant material of a single plant, for example a single seed.

Even though assay methods of the present invention preferably are performed on plant groups, assay methods of the present invention performed on single plants are also useful and beneficial. Such methods allow for a fast screening plants and thus are particularly suitable for high throughput evaluation of genes and gene combinations coding for enzymes of unsaturated fatty acid metabolism.

According to the method of the invention, the activity of a PDCT which activity is increased in the method of the invention can be increased by de novo expression of the PDCT in the plant, plant cell or seed or by increasing the expression or activity of an endogenous PDCT.

The gene coding for the PDCT of the present invention or used in the method of the present invention preferably is operably linked to an expression control sequence to allow constitutive or non-constitutive expression of said gene. Expression control sequences according to the present invention are known to the skilled person as promoters, transcription factor binding sites and regulatory nucleic acids like for example RNAi. Preferably, the expression control sequence directs expression of the gene in a tissue-specific manner. Where the plant is an oil seed plant, preferably of a Brassica species, expression of the gene preferably is specific to plant seeds in one or more of their developmental stages. According to the present invention, tissue-specific expression does not require the total absence of gene expression in any other tissue. However, tissue-specific expression for a selected tissue means that the maximum amount of mRNA transcript in this tissue is at least 2-fold, preferably at least 5-fold, even more preferably at least 10-fold, even more preferably at least 20-fold, even more preferably at least 50-fold and most preferably at least 100-fold the maximum amount of said mRNA in the other tissues. Furthermore, expression control sequences are known to the skilled person which allow induction or repression of expression by a signal applied by a user, for example application of an inductor like IPTG.

The PDCT of the present invention or the PDCT or used in the method of the present invention can be present in the cell, the plant or seed of the method of the present invention as a single copy gene or in multiple gene copies.

The PDCT of the present invention or used in the method of the present invention preferably is expressed in the same plant cell also expressing the other at least one or more enzymes of unsaturated fatty acid metabolism. It is possible but not necessary that the PDCT of the present invention or used in the method of the present invention is expressed at the same time as one, some or all of said other genes of unsaturated fatty acid metabolism.

In case, the plant, plant cell or seed expresses a Delta-6 elongase, the increased activity of the PDCT of the invention in the cell, plant or seed, whereby the PDCT preferably can be selected from the group consisting of:

(a) a PDCT1 having at least 80% sequence identity with SEQ ID NO: 2, 4, 6, 8, 10, 12, 14, 16, 40, 42, 44, and/or 46;

(b) a PDCT1 encoded by a polynucleotide having at least 80% sequence identity with SEQ ID NO: 1, 3, 5, 7, 9, 11, 13, 15, 39, 41, 43, and/or 45;

(c) a PDCT1 encoded by a polynucleotide that hybridizes under high stringency conditions with (i) a polynucleotide that encodes the amino acid sequence of SEQ ID NO: 2, 4, 6, 8, 10, 12, 14, 16, 40, 42, 44, and/or 46, or (ii) the full-length complement of (i);

(d) a variant of the PDCT1 of SEQ ID NO: 2, 4, 6, 8, 10, 12, 14, 16, 40, 42, 44, and/or 46 comprising a substitution, preferably a conservative substitution, deletion, and/or insertion at one or more positions and having PDCT1 activity;

(e) a PDCT1 encoded by a polynucleotide that differs from SEQ ID NO: 1, 3, 5, 7, 9, 11, 13, 15, 39, 41, 43, and/or 45 due to the degeneracy of the genetic code; and

(f) a fragment of the PDCT1 of (a), (b), (c), (d) or (e) having PDCT1 activity; leads to an increase in the conversion efficiency of a Delta-6 elongase.

The activity of the PDCT may be increased as result of a de novo expression due to a stable transformation with an expression construct comprising a nucleic acid molecule encoding and providing expressing a PDCT1 or by increasing the endogenous activity of the PDCT of the invention if already present in the wildtype or in the control.

The contribution from each desaturase and elongase gene present in the T-DNA to the amount of VLC-PUFA is difficult to assess, but it is possible to calculate conversion efficiencies for each pathway step, for example by using the equations shown in FIG. 7. The calculations are based on fatty acid composition of the tissue or oil in question and indicate the amount of product fatty acid (and downstream products) formed from the subatrate of a particular enzyme. The conversion efficiencies are sometimes referred to as “apparent” conversion efficiencies because for some of the calculations it is recognized that the calculations do not take into account all factors that could be influencing the reaction. Nevertheless, conversion efficiency values can be used to assess contribution of each desaturase or elongase reaction to the overall production of VLC-PUFA. By comparing conversion efficiencies, one can compare the relative effectiveness of a given enzymatic step between different individual seeds, plants, bulk seed batches, events, Brassica germplasm, or transgenic constructs.

The activity of a PDCT can be measured as described in the Examples e.g. by expressing the PDCT in plants, as described in the examples.

Preferably, the PDCT of the invention is expressed in oil seed crop, e.g. in Camelina or Brassica sp as described herein, e.g. by transforming the plant stably with the PDCT of the invention, e.g. with the PDCT preferably selected from the group consisting of:

(a) a PDCT1 having at least 80% sequence identity with SEQ ID NO: 2, 4, 6, 8, 10, 12, 14, 16, 40, 42, 44, and/or 46;

(b) a PDCT1 encoded by a polynucleotide having at least 80% sequence identity with SEQ ID NO: 1, 3, 5, 7, 9, 11, 13, 15, 39, 41, 43, and/or 45;

(c) a PDCT1 encoded by a polynucleotide that hybridizes under high stringency conditions with (i) a polynucleotide that encodes the amino acid sequence of SEQ ID NO: 2, 4, 6, 8, 10, 12, 14, 16, 40, 42, 44, and/or 46, or (ii) the full-length complement of (i);

(d) a variant of the PDCT1 of SEQ ID NO: 2, 4, 6, 8, 10, 12, 14, 16, 40, 42, 44, and/or 46 comprising a substitution, preferably a conservative substitution, deletion, and/or insertion at one or more positions and having PDCT1 activity;

(e) a PDCT1 encoded by a polynucleotide that differs from SEQ ID NO: 1, 3, 5, 7, 9, 11, 13, 15, 39, 41, 43, and/or 45 due to the degeneracy of the genetic code; and

(f) a fragment of the PDCT1 of (a), (b), (c), (d) or (e) having PDCT1 activity;

The resulting oil is enriched in EPA, DHA and/or EPA. The method of the invention for example results preferably in an increase of the total PUFAs, compared to a control. According to the invention, the Delta-6 desaturase is preferably Acyl-CoA dependent.

In one embodiment, in the method of the invention, the plant, plant cell, and/or seed, for example, expresses none, one or more Acyl-CoA dependent desaturase, e.g. an Acyl-CoA dependent Delta-4 desaturase, Delta-5 desaturase, Delta-6 Desaturase, Delta-12 Desaturase, and/or Omega-3 desaturase, for example a Phospholipid-dependent or an Acyl-CoA dependent Delta-6 desaturase as described herein. Preferably a Acyl-CoA dependent Destaurase, e.g. a Acyl-CoA Delta-6 desaturase is used in the method of the present invention.

Further, for example, in the method of the invention, the plant, plant cell, and/or seed, for example, expresses none, one or more phospholipid dependent desaturases.

According to the invention, for example, none, or one or more desaturase from the group above uses Acyl-CoA as substrate. So, for example, at least one desaturase uses phophplipids and one uses Acyl-CoA as substrate. Preferably, the Desaturase is selected from the group Delta-4 desaturase, Delta-5 desaturase, Delta-6 desaturase, omega.3 desaturase, or Delta-12 desaturase. So, for example, in the method of the present invention uses a Delta-6 desaturase with Acyl-CoA as substrate and a Delta-6 elongase, e.g. together with an another desaturase that uses Acyl-CoA as substrate. So, for example, in the method of the present invention uses a Delta-6 desaturase with Phospholipid as substrate and a Delta-6 elongase, e.g. together with an another desaturase that uses Acyl-CoA as substrate.

Preferably, at least one of the desaturases used in the method of the invention Acyl-CoA as substrate, in particular one desaturase selected from the groups consisting of Delta-4 desaturase, Delta-5 desaturase, Delta-6 desaturase, omega.3 desaturase, Delta 5/Delta 6-desaturase, Delta-8 desaturase or Delta-9 desaturase, Delta-8/9 desaturase, Delta-12 desaturase uses as substrate phospholipids.

Preferably, at least one desaturase from the group uses Acyl-CoA as substrate.

Thus, in the method of the invention, the plant, plant cell and/or seed, for example expresses Delta-4 desaturase, Delta-5 desaturase, Delta-6 Desaturase, Delta-12 Desaturase, and/or Omega-3 desaturase, whereby none, one or more desaturases use Acyl-CoA-activated fatty acids as substrate, and/or whereby none, one or more desaturases uses phospholipid activated fatty acids as substrate. Thus, in the method of the invention, the plant, plant cell and/or seed, for example expresses one or more Delta-4 desaturase, Delta-5 desaturase, Delta-6 Desaturase, Delta-12 Desaturase, and/or Omega-3 desaturase, that use Acyl-CoA-activated fatty acids as substrate, and one or more Delta-4 desaturase, Delta-5 desaturase, Delta-6 Desaturase, Delta-12 Desaturase, and/or Omega-3 desaturase, that use phospholipid-activated fatty acids as substrate

So, for example, at least one desaturase uses phosphoplipids and one uses Acyl-CoA as substrate. Preferably, the desaturase is selected from the group Delta-4 desaturase, Delta-5 desaturase, Delta-6 desaturase, Omega.3 desaturase, or Delta-12 desaturase. So, for example, in the method of the present invention a Delta-6 desaturase uses phospholipids as substrate.

The invention also provides a method of increasing the PDCT of the invention, e.g. the PDCT1, activity and/or of stabilising PDCT of the invention, e.g. the PDCT1 , activity in a plant or part thereof or during developmental stages of a plant or part thereof, preferably during seed development, which methods comprise growing a plant expressing a PDCT of the present invention.

Thus, the invention also provides a method of producing one or more desired unsaturated fatty acids in a plant, comprising growing a plant, said plant expressing, at least temporarily, a PDCTof the present invention and one or more further genes to convert linoleic acid to said one or more desired unsaturated fatty acids. As indicated above, the one or more further genes coding for enzymes for the production of unsaturated fatty acids preferably comprise desaturases and elongases.

The invention also provides a nucleic acid comprising a gene coding for a PDCT of the present invention, wherein the gene does not code for a PDCT of any of the exact sequences SEQ ID NO. 2, 4, 6, 8, 10, 12, 14, 16, 40, 42, 44, and/or 46. Thus, the present invention provides a nucleic acid comprising a gene coding for a PDCT, wherein said PDCT has at least 50% total amino acid sequence identity to any of the sequences SEQ ID NO. 2, 4, 6, 8, 10, 12, 14, 16, 40, 42, 44, and/or 46 and/or at least 60% total amino acid sequence similarity to any of the sequences SEQ ID NO. 2, 4, 6, 8, 10, 12, 14, 16, 40, 42, 44, and/or 46 , and wherein the sequence is not any of the sequences SEQ ID NO. 18, 20, 22, 24, 26, 28, 30, 32, 50, 52, 54, 56, 58, and/or 60. Preferably, the nucleic acid molecule of the invention or (over)expressed in the method of the invention does not encode a PDCT3 or PDCT5.

The invention also provides a nucleic acid comprising a gene coding for a PDCT of the present invention, wherein the gene is operably linked to an expression control sequence, and wherein the expression control sequence is heterologous to said gene if the gene codes for any of the exact sequences according to SEQ ID NO. 2, 4, 6, 8, 10, 12, 14, 16, 40, 42, 44, and/or 46. Thus, the invention particularly provides combinations of promoters and genes not found in nature.

The nucleic acids of the present invention preferably are expression vectors transformation constructs or expression constructs useful for transforming a plant cell and causing the PDCT gene of the present invention to be expressed at least temporarily, preferably stable during plant or plant cell or seed development. Thus, the nucleic acids of the present invention facilitate to materialise the benefits conveyed by the present invention as described herein. Also, the invention provides purified PDCT polypeptides coded by any of the nucleic acids of the present invention as well as antibodies specifically binding the PDCT polypeptide of the invention, e.g. monoclonale Antibodies or fragments thereof, as long as the fragments specifically bind the PDCT of the invention.

According to the invention, there is also provided a plant cell comprising a non-native gene coding for a PDCT of the present invention. Such plant cells can be obtained, as described above, by transformation of wild-type plant cells or offspring thereof, for example by crossing a plant comprising a gene coding for a PDCT of the invention with a plant not comprising such gene and selecting offspring, preferably seeds, which comprise said gene. This way it is easily possible to transfer the gene coding for a PDCT of the present invention from one germplasm to another. The plant cell of the present invention preferably comprises a gene coding for one of the preferred PDCT of the present invention to materialise the benefits conveyed by such preferred PDCT. Also as described above, the gene coding for the PDCT of the present invention preferably is operably linked to an expression control sequence, and it is particularly preferred that said expression control sequence directs expression to certain tissues and certain times of plant development, for example to developing seed tissue and the above indicated preferred times after flowering.

Preferably the plant cell, plant or seed comprising the polynucleotide of the invention, e.g. the PDCT1, is a Camelia or Brassica species, preferably B. napus, B. juncea, B. carrinata or Camelina sativa.

As the present invention provides an assay method which can, also be used for screening and comparison purposes, the present invention also provides a plant set comprising at least 2 plant groups, each consisting of one or more plants, wherein the plant or plants of each group are capable of expressing a PDCT of the present invention, and wherein the plant or plants of said groups comprise one or more genes coding for at least one or more enzymes of unsaturated fatty acid metabolism, of which enzymes at least one is capable of using linoleic acid as a substrate, and of which enzymes at least one is supposedly connected to a plant metabolic property, and wherein the plant or plants of said groups differ in the expression of at least one of the enzymes of unsaturated fatty acid metabolism. To differ in expression of at least one of the enzymes of unsaturated fatty acid metabolism, one gene present in the plant or plants of one group may be missing in the plant or plants of another group, or may be expressed at different times or in different tissues or in differing intensities. For example, the plants of 2 groups may both comprise a gene coding for a Delta-4 desaturase under the control of identical expression control sequences, but the Delta-4 desaturase nucleic acid sequences are derived from different organisms such that the amino acid sequences of the respective Delta-4 desaturases are unique for the plants of each of the groups. Instead of or additional to differing in the genes for Delta-4 desaturases, the groups can also differ in any other nucleic acid sequence coding for an enzyme of unsaturated fatty acid metabolism, included but not limited to omega-3 desaturases, Delta-6 desaturases, Delta-9 elongases, Delta-6 elongases, Delta-8 desaturases, Delta-5 desaturases and Delta-5 elongases.

Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in M. Green & J. Sambrook (2012) Molecular Cloning: a laboratory manual, 4th Edition Cold Spring Harbor Laboratory Press, CSH, New York; Ausubel et al., Current Protocols in Molecular Biology, Wiley Online Library; Maniatis et al., 1982 Molecular Cloning, Cold Spring Harbor Laboratory, Plainview, N.Y.; Wu (Ed.) 1993 Meth. Enzymol. 218, Part I; Wu (Ed.) 1979 Meth Enzymol. 68; Wu et al., (Eds.) 1983 Meth. Enzymol. 100 and 101; Grossman and Moldave (Eds.) 1980 Meth. Enzymol. 65; Miller (Ed.) 1972 Experiments in Molecular Genetics, Cold Spring Harbor Laboratory, Cold Spring Harbor, N.Y.; Old and Primrose, 1981 Principles of Gene Manipulation, University of California Press, Berkeley; Schleif and Wensink, 1982 Practical Methods in Molecular Biology; Glover (Ed.) 1985 DNA Cloning Vol. I and II, IRL Press, Oxford, UK; Hames and Higgins (Eds.) 1985 Nucleic Acid Hybridization, IRL Press, Oxford, UK; and Setlow and Hollaender 1979 Genetic Engineering: Principles and Methods, Vols. 1-4, Plenum Press, New York.

The term “cultivating” as used herein refers to maintaining and growing the transgenic plant under culture conditions which allow the cells to produce the said polyunsaturated fatty acids, i.e. the PUFAs and/or VLC-PUFAs referred to above. This implies that the polynucleotide of the present invention is expressed in the transgenic plant so that the desaturase, elongase as also the keto-acyl-CoA-synthase, keto-acyl-CoA-reductase, dehydratase and enoyl-CoA-reductase activity is present. Suitable culture conditions for cultivating the host cell are described in more detail below.

The term “obtaining” as used herein encompasses the provision of the cell culture including the host cells and the culture medium or the plant or plant part, particularly the seed, of the current invention, as well as the provision of purified or partially purified preparations thereof comprising the polyunsaturated fatty acids, preferably, ARA, EPA, DHA, in free or in CoA bound form, as membrane phospholipids or as triacylglyceride esters. More preferably, the PUFA and VLC-PUFA are to be obtained as triglyceride esters, e.g., in form of an oil. More details on purification techniques can be found elsewhere herein below.

The term “polynucleotide” according to the present invention refers to a desoxyribonucleic acid or ribonucleic acid. Unless stated otherwise, “polynucleotide” herein refers to a single strand of a DNA polynucleotide or to a double stranded DNA polynucleotide. The length of a polynucleotide is designated according to the invention by the specification of a number of basebairs (“bp”) or nucleotides (“nt”). According to the invention, both specifications are used interchangeably, regardless whether or not the respective nucleic acid is a single or double stranded nucleic acid. Also, as polynucleotides are defined by their respective nucleotide sequence, the terms nucleotide/polynucleotide and nucleotide sequence/polynucleotide sequence are used interchangeably, thus that a reference to a nucleic acid sequence also is meant to define a nucleic acid comprising or consisting of a nucleic acid stretch the sequence of which is identical to the nucleic acid sequence.

In particular, the term “polynucleotide” as used in accordance with the present invention as far as it relates to a desaturase or elongase gene relates to a polynucleotide comprising a nucleic acid sequence which encodes a polypeptide having desaturase or elongase activity. Preferably, the polypeptide encoded by the polynucleotide of the present invention having desaturase, or elongase activity upon expression in a plant shall be capable of increasing the amount of PUFA and, in particular, VLC-PUFA in, e.g., seed oils or an entire plant or parts thereof. Whether an increase is statistically significant can be determined by statistical tests well known in the art including, e.g., Student's t-test with a confidentiality level of at least 90%, preferably of at least 95% and even more preferably of at least 98%. More preferably, the increase is an increase of the amount of triglycerides containing VLC-PUFA of at least 5%, at least 10%, at least 15%, at least 20% or at least 30% compared to wildtype control (preferably by weight), in particular compared to seeds, seed oil, extracted seed oil, crude oil, or refined oil from a wild-type control. Preferably, the VLC-PUFA referred to before is a polyunsaturated fatty acid having a C20, C22 or C24 fatty acid body, more preferably EPA or DHA. Lipid analysis of oil samples are shown in the accompanying Examples.

In the plants of the present invention, in particular in the oil obtained or obtainable from the plant of the present invention, the content of certain fatty as shall be decreased or, in particular, increased as compared to the oil obtained or obtainable from a control plant. In particular decreased or increased as compared to seeds, seed oil, crude oil, or refined oil from a control plant. The choice of suitable control plants is a routine part of an experimental setup and may include corresponding wild type plants or corresponding plants without the polynucleotides as encoding desaturases and elongase as referred to herein. The control plant is typically of the same plant species or even of the same variety as the plant to be assessed. The control plant may also be a nullizygote of the plant to be assessed. Nullizygotes (or null control plants) are individuals missing the transgene by segregation. Further, control plants are grown under the same or essentially the same growing conditions to the growing conditions of the plants of the invention, i.e. in the vicinity of, and simultaneously with, the plants of the invention. A “control plant” as used herein preferably refers not only to whole plants, but also to plant parts, including seeds and seed parts. The control could also be the oil from a control plant.

Preferably, the control plant is an isogenic control plant. Thus, e.g. the control oil or seed shall be from an isogenic control plant.

The fatty acid esters with polyunsaturated C20- and/or C22-fatty acid molecules can be isolated in the form of an oil or lipid, for example, in the form of compounds such as sphingolipids, phosphoglycerides, lipids, glycolipids such as glycosphingolipids, phos-pholipids such as phosphatidylethanolamine, phosphatidylcholine, phosphatidylserine, phosphatidylglycerol, phosphatidylinositol or diphosphatidylglycerol, monoacylglycerides, diacylglycerides, triacylglycerides or other fatty acid esters such as the acetylcoenzyme A esters which comprise the polyunsaturated fatty acids with at least two, three, four, five or six, preferably five or six, double bonds, from the organisms which were used for the preparation of the fatty acid esters. Preferably, they are isolated in the form of their diacylglycerides, triacylglycerides and/or in the form of phosphatidylcholine, especially preferably in the form of the triacylglycerides. In addition to these esters, the polyunsaturated fatty acids are also present in the non-human transgenic organisms or host cells, preferably in the plants, as free fatty acids or bound in other compounds. As a rule, the various abovementioned compounds (fatty acid esters and free fatty acids) are present in the organisms with an approximate distribution of 80 to 90% by weight of triglycerides, 2 to 5% by weight of diglycerides, 5 to 10% by weight of monoglycerides, 1 to 5% by weight of free fatty acids, 2 to 8% by weight of phospholipids, the total of the various compounds amounting to 100% by weight. In the process of the invention, the VLC-PUFAs which have been produced are produced in a content as for DHA of at least 5.5% by weight, at least 6% by weight, at least 7% by weight, advantageously at least 8% by weight, preferably at least 9% by weight, especially preferably at least 10.5% by weight, very especially preferably at least 20% by weight, as for EPA of at least 9.5% by weight, at least 10% by weight, at least 11% by weight, advantageously at least 12% by weight, preferably at least 13% by weight, especially preferably at least 14.5% by weight, very especially preferably at least 30% by weight based on the total fatty acids in the non-human transgenic organisms or the host cell referred to above. The fatty acids are, preferably, produced in bound form. It is possible, with the aid of the polynucleotides and polypeptides of the present invention, for these unsaturated fatty acids to be positioned at the sn1, sn2 and/or sn3 position of the triglycerides which are, preferably, to be produced.

In a method or manufacturing process of the present invention the polynucleotides and polypeptides of the present invention may be used with at least one further polynucleotide encoding an enzyme of the fatty acid or lipid biosynthesis. Preferred enzymes are in this context the desaturases and elongases as mentioned above, but also polynucleotide encoding an enzyme having delta-8-desaturase and/or delta-9-elongase activity. All these enzymes reflect the individual steps according to which the end products of the method of the present invention, for example EPA or DHA are produced from the starting compounds linoleic acid (C18:2) or linolenic acid (C18:3). As a rule, these compounds are not generated as essentially pure products. Rather, small traces of the precursors may be also present in the end product. If, for example, both linoleic acid and linolenic acid are present in the starting host cell, organism, or the starting plant, the end products, such as EPA or DHA, are present as mixtures. The precursors should advantageously not amount to more than 20% by weight, preferably not to more than 15% by weight, more preferably, not to more than 10% by weight, most preferably not to more than 5% by weight, based on the amount of the end product in question. Advantageously, only EPA or more preferably only DHA, bound or as free acids, is/are produced as end product(s) in the process of the invention in a host cell. If the compounds EPA and DHA are produced simultaneously, they are, preferably, produced in a ratio of at least 1:2 (DHA:EPA), more preferably, the ratios are at least 1:5 and, most preferably, 1:8. Fatty acid esters or fatty acid mixtures produced by the invention, preferably, comprise 6 to 15% of palmitic acid, 1 to 6% of stearic acid, 7-85% of oleic acid, 0.5 to 8% of vaccenic acid, 0.1 to 1% of arachidic acid, 7 to 25% of saturated fatty acids, 8 to 85% of monounsaturated fatty acids and 60 to 85% of polyunsaturated fatty acids, in each case based on 100% and on the total fatty acid content of the organisms. DHA as a preferred long chain polyunsaturated fatty acid is present in the fatty acid esters or fatty acid mixtures in a concentration of, preferably, at least 0.1; 0.2; 0.3; 0.4; 0.5; 0.6; 0.7; 0.8; 0.9 or 1%, based on the total fatty acid content.

Chemically pure VLC-PUFAs or fatty acid compositions can also be synthesized by the methods described herein. To this end, the fatty acids or the fatty acid compositions are isolated from a corresponding sample via extraction, distillation, crystallization, chromatography or a combination of these methods. These chemically pure fatty acids or fatty acid compositions are advantageous for applications in the food industry sector, the cosmetic sector and especially the pharmacological industry sector.

The terms “essentially”, “about”, “approximately”, “substantially” and the like in connection with an attribute or a value, particularly also define exactly the attribute or exactly the value, respectively. The term “substantially” in the context of the same functional activity or substantially the same function means a difference in function preferably within a range of 20%, more preferably within a range of 10%, most preferably within a range of 5% or less compared to the reference function. In context of formulations or compositions, the term “substantially” (e.g., “composition substantially consisting of compound X”) may be used herein as containing substantially the referenced compound having a given effect within the formulation or composition, and no further compound with such effect or at most amounts of such compounds which do not exhibit a measurable or relevant effect. The term “about” in the context of a given numeric value or range relates in particular to a value or range that is within 20%, within 10%, or within 5% of the value or range given. As used herein, the term “comprising” also encompasses the term “consisting of”.

The term “isolated” means that the material is substantially free from at least one other component with which it is naturally associated within its original environment. For example, a naturally-occurring polynucleotide, polypeptide, or enzyme present in a living animal is not isolated, but the same polynucleotide, polypeptide, or enzyme, separated from some or all of the coexisting materials in the natural system, is isolated. As further example, an isolated nucleic acid, e.g., a DNA or RNA molecule, is one that is not immediately contiguous with the 5′ and 3′ flanking sequences with which it normally is immediately contiguous when present in the naturally occurring genome of the organism from which it is derived. Such polynucleotides could be part of a vector, incorporated into a genome of a cell with an unrelated genetic background (or into the genome of a cell with an essentially similar genetic background, but at a site different from that at which it naturally occurs), or produced by PCR amplification or restriction enzyme digestion, or an RNA molecule produced by in vitro transcription, and/or such polynucleotides, polypeptides, or enzymes could be part of a composition, and still be isolated in that such vector or composition is not part of its natural environment.

Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in M. Green & J. Sambrook (2012) Molecular Cloning: a laboratory manual, 4th Edition Cold Spring Harbor Laboratory Press, CSH, New York; Ausubel et al., Current Protocols in Molecular Biology, Wiley Online Library; Maniatis et al., 1982 Molecular Cloning, Cold Spring Harbor Laboratory, Plainview, N.Y.; Wu (Ed.) 1993 Meth. Enzymol. 218, Part I; Wu (Ed.) 1979 Meth Enzymol. 68; Wu et al., (Eds.) 1983 Meth. Enzymol. 100 and 101; Grossman and Moldave (Eds.) 1980 Meth. Enzymol. 65; Miller (Ed.) 1972 Experiments in Molecular Genetics, Cold Spring Harbor Laboratory, Cold Spring Harbor, N.Y.; Old and Primrose, 1981 Principles of Gene Manipulation, University of California Press, Berkeley; Schleif and Wensink, 1982 Practical Methods in Molecular Biology; Glover (Ed.) 1985 DNA Cloning Vol. I and II, IRL Press, Oxford, UK; Hames and Higgins (Eds.) 1985 Nucleic Acid Hybridization, IRL Press, Oxford, UK; and Setlow and Hollaender 1979 Genetic Engineering: Principles and Methods, Vols. 1-4, Plenum Press, New York.

Unless otherwise noted, the terms used herein are to be understood according to conventional usage by those of ordinary skill in the relevant art. In addition to the definitions of terms provided herein, definitions of common terms in molecular biology may also be found in Rieger et al., 1991 Glossary of genetics: classical and molecular, 5th Ed., Berlin: Springer-Verlag; and in Current Protocols in Molecular Biology, F. M. Ausubel et al., Eds., Current Protocols, a joint venture between Greene Publishing Associates, Inc. and John Wiley & Sons, Inc., (1998 Supplement).

It is to be understood that as used in the specification and in the claims, “a” or “an” can mean one or more, depending upon the context in which it is used. Thus, for example, reference to “a cell” can mean that at least one cell can be utilized. It is to be understood that the terminology used herein is for the purpose of describing specific embodiments only and is not intended to be limiting.

“Purified” means that the material is in a relatively pure state, e.g., at least about 90% pure, at least about 95% pure, or at least about 98% or 99% pure. Preferably “purified” means that the material is in a 100% pure state.

The term “non-naturally occurring” refers to a (poly)nucleotide, amino acid, (poly)peptide, enzyme, protein, cell, organism, or other material that is not present in its original environment or source, although it may be initially derived from its original environment or source and then reproduced by other means. Such non-naturally occurring (poly)nucleotide, amino acid, (poly)peptide, enzyme, protein, cell, organism, or other material may be structurally and/or functionally similar to or the same as its natural counterpart.

The term “native” (or “wildtype” or “endogenous”) cell or organism and “native” (or wildtype or endogenous) polynucleotide or polypeptide refers to the cell or organism as found in nature and to the polynucleotide or polypeptide in question as found in a cell in its natural form and genetic environment, respectively (i.e., without there being any human intervention).

The term “heterologous” (or exogenous or foreign or recombinant) polypeptide is defined herein as:

a polypeptide that is not native to the host cell. The protein sequence of such a heterologous polypeptide is a synthetic, non-naturally occurring, “man made” protein sequence;

a polypeptide native to the host cell but structural modifications, e.g., deletions, substitutions, and/or insertions, are included as a result of manipulation of the DNA of the host cell by recombinant DNA techniques to alter the native polypeptide; or

a polypeptide native to the host cell whose expression is quantitatively altered or whose expression is directed from a genomic location different from the native host cell as a result of manipulation of the DNA of the host cell by recombinant DNA techniques, e.g., a stronger promoter.

Descriptions b) and c), above, refer to a sequence in its natural form but not naturally expressed by the cell used for its production. The produced polypeptide is therefore more precisely defined as a “recombinantly expressed endogenous polypeptide”, which is not in contradiction to the above definition but reflects the specific situation that it's not the sequence of a protein being synthetic or manipulated but the way the polypeptide molecule is produced.

Similarly, the term “heterologous” (or exogenous or foreign or recombinant) polynucleotide refers:

to a polynucleotide that is not native to the host cell;

a polynucleotide native to the host cell but structural modifications, e.g., deletions, substitutions, and/or insertions, are included as a result of manipulation of the DNA of the host cell by recombinant DNA techniques to alter the native polynucleotide;

a polynucleotide native to the host cell whose expression is quantitatively altered as a result of manipulation of the regulatory elements of the polynucleotide by recombinant DNA techniques, e.g., a stronger promoter; or

a polynucleotide native to the host cell, but integrated not within its natural genetic environment as a result of genetic manipulation by recombinant DNA techniques.

With respect to two or more polynucleotide sequences or two or more amino acid sequences, the term “heterologous” is used to characterize that the two or more polynucleotide sequences or two or more amino acid sequences do not occur naturally in the specific combination with each other.

The term “gene” means the segment of DNA involved in producing a polypeptide chain; it includes regions preceding and following the coding region (leader and trailer) as well as intervening sequences (introns) between individual coding segments (exons).

The term “gene” means a segment of DNA containing hereditary information that is passed on from parent to offspring and that contributes to the phenotype of an organism. The influence of a gene on the form and function of an organism is mediated through the transcription into RNA (tRNA, rRNA, mRNA, non-coding RNA) and in the case of mRNA through translation into peptides and proteins.

The term hybridization according to this invention means, that hybridization must occur over the complete length of the sequence of the invention.

The term “hybridisation” as defined herein is a process wherein substantially complementary nucleotide sequences anneal to each other. The hybridisation process can occur entirely in solution, i.e. both complementary nucleic acids are in solution. The hybridisation process can also occur with one of the complementary nucleic acids immobilised to a matrix such as magnetic beads, Sepharose beads or any other resin. The hybridisation process can furthermore occur with one of the complementary nucleic acids immobilised to a solid support such as a nitro-cellulose or nylon membrane or immobilised by e.g. photolithography to, for example, a siliceous glass support (the latter known as nucleic acid arrays or microarrays or as nucleic acid chips). In order to allow hybridisation to occur, the nucleic acid molecules are generally thermally or chemically denatured to melt a double strand into two single strands and/or to remove hairpins or other secondary structures from single stranded nucleic acids.

The term “stringency” refers to the conditions under which a hybridisation takes place. The stringency of hybridisation is influenced by conditions such as temperature, salt concentration, ionic strength and hybridisation buffer composition. Generally, low stringency conditions are selected to be about 30° C. lower than the thermal melting point (Tm) for the specific sequence at a defined ionic strength and pH. Medium stringency conditions are when the temperature is 20° C. below Tm, and high stringency conditions are when the temperature is 10° C. below Tm. High stringency hybridisation conditions are typically used for isolating hybridising sequences that have high sequence similarity to the target nucleic acid sequence. However, nucleic acids may deviate in sequence and still encode a substantially identical polypeptide, due to the degeneracy of the genetic code. Therefore, medium stringency hybridisation conditions may sometimes be needed to identify such nucleic acid molecules.

The “Tm” is the temperature under defined ionic strength and pH, at which 50% of the target sequence hybridises to a perfectly matched probe. The Tm is dependent upon the solution conditions and the base composition and length of the probe. For example, longer sequences hybridise specifically at higher temperatures. The maximum rate of hybridisation is obtained from about 16° C. up to 32° C. below Tm. The presence of monovalent cations in the hybridisation solution reduce the electrostatic repulsion between the two nucleic acid strands thereby promoting hybrid formation; this effect is visible for sodium concentrations of up to 0.4M (for higher concentrations, this effect may be ignored). Formamide reduces the melting temperature of DNA-DNA and DNA-RNA duplexes with 0.6 to 0.7° C. for each percent formamide, and addition of 50% formamide allows hybridisation to be performed at 30 to 45° C., though the rate of hybridisation will be lowered. Base pair mismatches reduce the hybridisation rate and the thermal stability of the duplexes. On average and for large probes, the Tm decreases about 1° C. per % base mismatch. The Tm may be calculated using the following equations, depending on the types of hybrids:

DNA-DNA hybrids (Meinkoth and Wahl, Anal. Biochem., 138: 267-284, 1984):

Tm=81.5° C.+16.6×log[Na+]a+0.41×%[G/Cb]−500×[Lc]−1−0.61×% formamide

DNA-RNA or RNA-RNA hybrids:

Tm=79.8+18.5 (log 10[Na+]a)+0.58 (% G/Cb)+11.8 (% G/Cb)2−820/Lc

oligo-DNA or oligo-RNAd hybrids:

For <20 nucleotides: Tm=2 (In)

For 20-35 nucleotides: Tm=22+1.46 (In)

a or for other monovalent cation, but only accurate in the 0.01-0.4 M range.

b only accurate for % GC in the 30% to 75% range.

c L=length of duplex in base pairs.

d Oligo, oligonucleotide; In, effective length of primer=2×(no. of G/C)+(no. of A/T).

Non-specific binding may be controlled using any one of a number of known techniques such as, for example, blocking the membrane with protein containing solutions, additions of heterologous RNA, DNA, and SDS to the hybridisation buffer, and treatment with Rnase. For non-related probes, a series of hybridizations may be performed by varying one of (i) progressively lowering the annealing temperature (for example from 68° C. to 42° C.) or (ii) progressively lowering the formamide concentration (for example from 50% to 0%). The skilled artisan is aware of various parameters which may be altered during hybridisation and which will either maintain or change the stringency conditions.

Besides the hybridisation conditions, specificity of hybridisation typically also depends on the function of post-hybridisation washes. To remove background resulting from non-specific hybridisation, samples are washed with dilute salt solutions. Critical factors of such washes include the ionic strength and temperature of the final wash solution: the lower the salt concentration and the higher the wash temperature, the higher the stringency of the wash. Wash conditions are typically performed at or below hybridisation stringency. A positive hybridisation gives a signal that is at least twice of that of the background. Generally, suitable stringent conditions for nucleic acid hybridisation assays or gene amplification detection procedures are as set forth above. More or less stringent conditions may also be selected. The skilled artisan is aware of various parameters which may be altered during washing and which will either maintain or change the stringency conditions.

For example, typical high stringency hybridisation conditions for DNA hybrids longer than 50 nucleotides encompass hybridisation at 65° C. in 1×SSC or at 42° C. in 1×SSC and 50% formamide, followed by washing at 65° C. in 0.3×SSC. Examples of medium stringency hybridisation conditions for DNA hybrids longer than 50 nucleotides encompass hybridisation at 50° C. in 4×SSC or at 40° C. in 6×SSC and 50% formamide, followed by washing at 50° C. in 2× SSC. The length of the hybrid is the anticipated length for the hybridising nucleic acid. When nucleic acids of known sequence are hybridised, the hybrid length may be determined by aligning the sequences and identifying the conserved regions described herein. 1×SSC is 0.15M NaCI and 15 mM sodium citrate; the hybridisation solution and wash solutions may additionally include 5×Denhardt's reagent, 0.5-1.0% SDS, 100 μg/ml denatured, fragmented salmon sperm DNA, 0.5% sodium pyrophosphate. Another example of high stringency conditions is hybridisation at 65° C. in 0.1×SSC comprising 0.1 SDS and optionally 5×Denhardt's reagent, 100 μg/ml denatured, fragmented salmon sperm DNA, 0.5% sodium pyrophosphate, followed by the washing at 65° C. in 0.3×SSC.

For the purposes of defining the level of stringency, reference can be made to Sambrook et al. (2001) Molecular Cloning: a laboratory manual, 3rd Edition, Cold Spring Harbor Laboratory Press, CSH, New York or to Current Protocols in Molecular Biology, John Wiley & Sons, N.Y. (1989 and yearly updates).

The hybridisation process can occur entirely in solution, i.e. both complementary nucleic acids are in solution. The hybridisation process can also occur with one of the complementary nucleic acids immobilised to a matrix such as magnetic beads, Sepharose beads or any other resin. The hybridisation process can furthermore occur with one of the complementary nucleic acids immobilised to a solid support such as a nitro-cellulose or nylon membrane or immobilised by e.g. photolithography to, for example, a siliceous glass support (the latter known as nucleic acid arrays or microarrays or as nucleic acid chips). In order to allow hybridisation to occur, the nucleic acid molecules are generally thermally or chemically denatured to melt a double strand into two single strands and/or to remove hairpins or other secondary structures from single stranded nucleic acids.

A typical hybridisation experiment is done by an initial hybridisation step, which is followed by one to several washing steps. The solutions used for these steps may contain additional components, which are preventing the degradation of the analyzed sequences and/or prevent unspecific background binding of the probe, like EDTA, SDS, fragmented sperm DNA or similar reagents, which are known to a person skilled in the art (Sambrook et al. (2001) Molecular Cloning: a laboratory manual, 3rd Edition, Cold Spring Harbor Laboratory Press, CSH, New York or to Current Protocols in Molecular Biology, John Wiley & Sons, N.Y. (1989 and yearly updates).

A typical probe for a hybridisation experiment is for example generated by the random-primed-labeling method, which was initially developed by Feinberg and Vogelstein (Anal. Biochem., 132 (1), 6-13 (1983); Anal. Biochem., 137 (1), 266-7 (1984) and is based on the hybridisation of a mixture of all possible hexanucleotides to the DNA to be labeled. The labeled probe product will actually be a collection of fragments of variable length, typically ranging in sizes of 100-1000 nucleotides in length, with the highest fragment concentration typically around 200 to 400 bp. The actual size range of the probe fragments, which are finally used as probes for the hybridisation experiment, can for example also be influenced by the used labeling method parameter, subsequent purification of the generated probe (e.g. agarose gel), and the size of the used template DNA which is used for labeling (large templates can e.g. be restriction digested using a 4 bp cutter, e.g. Haelll, prior labeling).

“Recombinant” (or transgenic) with regard to a cell or an organism means that the cell or organism contains an exogenous polynucleotide which is introduced by gene technology and with regard to a polynucleotide means all those constructions brought about by gene technology/recombinant DNA techniques in which either

(a) the sequence of the polynucleotide or a part thereof, or

(b) one or more genetic control sequences which are operably linked with the polynucleotide, for example a promoter, or

(c) both a) and b)

are not located in their wildtype genetic environment or have been modified.

It shall further be noted that the term “isolated nucleic acid” or “isolated polypeptide” may in some instances be considered as a synonym for a “recombinant nucleic acid” or a “recombinant polypeptide”, respectively and refers to a nucleic acid or polypeptide that is not located in its natural genetic environment or cellular environment, respectively, and/or that has been modified by recombinant methods. An isolated nucleic acid sequence or isolated nucleic acid molecule is one that is not in its native surrounding or its native nucleic acid neighborhood, yet it is physically and functionally connected to other nucleic acid sequences or nucleic acid molecules and is found as part of a nucleic acid construct, vector sequence or chromosome. Typically, the isolated nucleic acid is obtained by isolating RNA from cells under laboratory conditions and converting it in copy-DNA (cDNA).

The term “control” polypeptide or the “control” polynucleotide, e.g. for use in an assay to identify the polypeptide that can be used in the method of the invention, is defined herein to include all sequences effecting for the expression of a polynucleotide, including but not limited thereto, the expression of a polynucleotide encoding a polypeptide. Each control sequence may be native or foreign to the polynucleotide or native or foreign to each other. Such control sequences include, but are not limited to, a leader, polyadenylation sequence, propeptide sequence, promoter, 5′-UTR, ribosomal binding site (RBS, shine dalgarno sequence), 3′-UTR, signal peptide sequence, and transcription terminator. At a minimum, the control sequence includes a promoter and transcriptional start and stop signals.

The control plant is typically of the same plant species or even of the same variety as the plant to be assessed. The control plant may also be a nullizygote of the plant to be assessed. A nullizygote (or null control plant) is progeny of T0 transformants and misses the transgene by segregation. Further, control plants are grown under equal growing conditions to the growing conditions of the plants of the invention, i.e. in the vicinity of, and simultaneously with, the plants of the invention. A “control plant” as used herein refers not only to whole plants, but also to plant parts, including seeds and seed parts.

The term “operably linked” means that the described components are in a relationship permitting them to function in their intended manner. For example, a regulatory sequence operably linked to a coding sequence is ligated in such a way that expression of the coding sequence is achieved under condition compatible with the control sequences.

Gene editing or genome editing is a type of genetic engineering in which DNA is inserted, replaced, or removed from a genome and which can be obtained by using a variety of techniques such as “gene shuffling” or “directed evolution” consisting of iterations of DNA shuffling followed by appropriate screening and/or selection to generate variants of nucleic acids or portions thereof encoding proteins having a modified biological activity (Castle et al., (2004) Science 304(5674): 1151-4; U.S. Pat. Nos. 5,811,238 and 6,395,547), or with “T-DNA activation” tagging (Hayashi et al. Science (1992) 1350-1353), where the resulting transgenic organisms show dominant phenotypes due to modified expression of genes close to the introduced promoter, or with “TILLING” (Targeted Induced Local Lesions In Genomes) and refers to a mutagenesis technology useful to generate and/or identify nucleic acids encoding proteins with modified expression and/or activity. TILLING also allows selection of organisms carrying such mutant variants. Methods for TILLING are well known in the art (McCallum et al., (2000) Nat Biotechnol 18: 455-457; reviewed by Stemple (2004) Nat Rev Genet 5(2): 145-50). Another technique uses artificially engineered nucleases like Zinc finger nucleases, Transcription Activator-Like Effector Nucleases (TALENs), the CRISPR/Cas system, and engineered meganuclease such as re-engineered homing endonucleases (Esvelt, K M.; Wang, H H. (2013), Mol Syst Biol 9 (1): 641; Tan, W S. et al. (2012), Adv Genet 80: 37-97; Puchta, H.; Fauser, F. (2013), Int. J. Dev. Biol 57: 629-637).

DNA and the proteins that they encoded can be modified using various techniques known in molecular biology to generate variant proteins or enzymes with new or altered properties. For example, random PCR mutagenesis, see, e.g., Rice (1992) Proc. Natl. Acad. Sci. USA 89:5467-5471; or, combinatorial multiple cassette mutagenesis, see, e.g., Crameri (1995) Biotechniques 18:194-196.

Alternatively, nucleic acids, e.g., genes, can be reassembled after random, or “stochastic,” fragmentation, see, e.g., U.S. Pat. Nos. 6,291,242; 6,287,862; 6,287,861; 5,955,358; 5,830,721; 5,824,514; 5,811,238; 5,605,793.

Alternatively, modifications, additions or deletions are introduced by error-prone PCR, shuffling, site-directed mutagenesis, assembly PCR, sexual PCR mutagenesis, in vivo mutagenesis (phage-assisted continuous evolution, in vivo continuous evolution), cassette mutagenesis, recursive ensemble mutagenesis, exponential ensemble mutagenesis, site-specific mutagenesis, gene reassembly, gene site saturation mutagenesis (GSSM), synthetic ligation reassembly (SLR), recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation, and/or a combination of these and other methods.

Alternatively,“gene site saturation mutagenesis” or “GSSM” includes a method that uses degenerate oligonucleotide primers to introduce point mutations into a polynucleotide, as described in detail in U.S. Pat. Nos. 6,171,820 and 6,764,835.

Alternatively, Synthetic Ligation Reassembly (SLR) includes methods of ligating oligonucleotide building blocks together non-stochastically (as disclosed in, e.g., U.S. Pat. No. 6,537,776).

Alternatively, Tailored multi-site combinatorial assembly (“TMSCA”) is a method of producing a plurality of progeny polynucleotides having different combinations of various mutations at multiple sites by using at least two mutagenic non-overlapping oligonucleotide primers in a single reaction. (as described in. PCT Pub. No. WO 2009/018449).

The term “substrate specificity” reflects the range of substrates that can be catalytically converted by an enzyme.

“Enzyme properties” include, but are not limited to catalytic activity as such, substrate/cofactor specificity, product specificity, increased stability during the course of time, thermostability, pH stability, chemical stability, and improved stability under storage conditions.

“Enzymatic activity” means at least one catalytic effect exerted by an enzyme. In one embodiment, enzymatic activity is expressed as units per milligram of enzyme (specific activity) or molecules of substrate transformed per minute per molecule of enzyme (molecular activity). Enzymatic activity can be specified by the enzymes actual function, e.g. proteases exerting proteolytic activity by catalyzing hydrolytic cleavage of peptide bonds, lipases exerting lipolytic activity by hydrolytic cleavage of ester bonds, etc

The term “recombinant organism” refers to a eukaryotic organism (yeast, fungus, alga, plant, animal) or to a prokaryotic microorganism (e.g., bacteria) which has been genetically altered, modified or engineered such that it exhibits an altered, modified or different genotype as compared to the wild-type organism which it was derived from. Preferably, the “recombinant organism” comprises an exogenous nucleic acid. “Recombinant organism”, “genetically modified organism” and “transgenic organism” are used herein interchangeably. The exogenous nucleic acid can be located on an extrachromosomal piece of DNA (such as plasmids) or can be integrated in the chromosomal DNA of the organism. In the case of a recombinant eukaryotic organism, it is understood as meaning that the nucleic acid(s) used are not present in, or originating from, the genome of said organism, or are present in the genome of said organism but not at their natural locus in the genome of said organism, it being possible for the nucleic acids to be expressed under the control of one or more endogenous and/or exogenous control element.

Host cells may be any cell selected from bacterial cells, yeast cells, fungal, algal or cyanobacterial cells, non-human animal or mammalian cells, or plant cells. The skilled artisan is well aware of the genetic elements that must be present on the genetic construct to successfully transform, select and propagate host cells containing the sequence of interest

The term “plant” as used herein refers to a photosynthetic, eukaryotic multicellular organism. Plants encompass green algae (Chlorophyta), red algae (Rhodophyta), Glaucophyta, mosses and liverworts (bryophytes), seedless vascular plants (horsetails, club mosses, ferns) and seed plants (angiosperms and gymnosperms). The term “plant” encompasses whole plants, ancestors and progeny of the plants and plant parts, including seeds, shoots, stems, leaves, roots, flowers, and tissues and organs, wherein each of the aforementioned comprise the gene/nucleic acid of interest. The term “plant” also encompasses plant cells, suspension cultures, callus tissue, embryos, meristematic regions, gametophytes, sporophytes, pollen, microspores and propagules, again wherein each of the aforementioned comprises the gene/nucleic acid of interest.

The term “plant parts” as used herein encompasses seeds, shoots, stems, leaves, roots, flowers, and tissues and organs, plant cells, suspension cultures, callus tissue, embryos, meristematic regions, gametophytes, sporophytes, pollen, microspores and propagules

“Propagule” is any kind of organ, tissue, or cell of a plant capable of developing into a complete plant. A propagule can be based on vegetative reproduction (also known as vegetative propagation, vegetative multiplication, or vegetative cloning) or sexual reproduction. A propagule can therefore be seeds or parts of the non-reproductive organs, like stem or leave. In particular, with respect to Poaceae, suitable propagules can also be sections of the stem, i.e., stem cuttings.

The terms “increase”, “improve” or “enhance” in the context of a yield-related trait are interchangeable and shall mean in the sense of the application at least a 3%, 4%, 5%, 6%, 7%, 8%, 9% or 10%, preferably at least 15% or 20%, more preferably 25%, 30%, 35% or 40% increase in the yield-related trait(s) (such as but not limited to more yield and/or growth) in comparison to control plants as defined herein.

The term “expression” or “gene expression” includes the transcription of a specific gene or specific genes or specific genetic construct. The term “expression” or “gene expression” in particular means the transcription of a gene or genes or genetic construct into structural RNA (rRNA, tRNA) or mRNA with or without subsequent translation of the latter into a protein. The process includes transcription of DNA and processing of the resulting mRNA product. Yet, the term “expression” as used herein may also include the translation of process of an mRNA molecule where a polypeptide is formed. Thus, the term “expression” may include the transcription process alone, the translation process alone, or both processes combined.

The term “increased expression”, “enhanced expression” or “overexpression” as used herein means any form of expression that is additional to the original wild-type expression level (which can be absence of expression or immeasurable expression as well). Reference herein to “increased expression”, “enhanced expression” or “overexpression” is taken to mean an increase in gene expression and/or, as far as referring to polypeptides, increased polypeptide levels and/or increased polypeptide activity, relative to control plants. The increase in expression, polypeptide levels or polypeptide activity is in increasing order of preference at least 5%, 10%, 20%, 30%, 40% or 50%, 60%, 70%, 80%, 85%, 90%, or 100% or even more compared to that of control plants.

Methods for increasing expression of genes or gene products are well documented in the art and include, for example, overexpression driven by appropriate promoters, the use of transcription enhancers or translation enhancers. Isolated nucleic acids which serve as promoter or enhancer elements may be introduced in an appropriate position (typically upstream) of a non-heterologous form of a polynucleotide so as to increase expression of a nucleic acid encoding the polypeptide of interest. For example, endogenous promoters may be altered in vivo by mutation, deletion, and/or substitution (see, Kmiec, U.S. Pat. No. 5,565,350; Zarling et al., WO9322443), or isolated promoters may be introduced into a plant cell in the proper orientation and distance from a gene of the present description so as to control the expression of the gene.

If polypeptide expression is desired, it is generally desirable to include a polyadenylation region at the 3′-end of a coding polynucleotide region.

An intron sequence may also be added to the 5′ untranslated region (UTR) or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg (1988) Mol. Cell biol. 8: 4395-4405; Callis et al. (1987) Genes Dev 1:1183-1200). Such intron enhancement of gene expression is typically greatest when placed near the 5′ end of the transcription unit. Use of the maize introns Adh1-5 intron 1, 2, and 6, the Bronze-1 intron are known in the art. For general information see: The Maize Handbook, Chapter 116, Freeling and Walbot, Eds., Springer, N.Y. (1994).

To obtain increased expression or overexpression of a polypeptide most commonly the nucleic acid encoding this polypeptide is overexpressed in sense orientation with a polyadenylation signal. Introns or other enhancing elements may be used in addition to a promoter suitable for driving expression with the intended expression pattern.

The term “vector” as used herein comprises any kind of construct suitable to carry foreign polynucleotide sequences for transfer to another cell, or for stable or transient expression within a given cell. The term “vector” as used herein encompasses any kind of cloning vehicles, such as but not limited to plasmids, phagemids, viral vectors (e.g., phages), bacteriophage, baculoviruses, cosmids, fosmids, artificial chromosomes, or and any other vectors specific for specific hosts of interest. Low copy number or high copy number vectors are also included. Foreign polynucleotide sequences usually comprise a coding sequence which may be referred to herein as “gene of interest”. The gene of interest may comprise introns and exons, depending on the kind of origin or destination of host cell.

Vectors thus are polynucleotide sequences—artificial in part or total or artificial in the arrangement of the genetic elements contained—capable of replication in a host cell and are used for introduction of a polynucleotide sequence of interest into a host cell or host organism (such as but, not limited to plasmids or viral polynucleotide sequences). A vector may be a construct or may comprise at least one construct, typically the vector comprises at least one expression cassette. A vector as used herein may provide segments for its transcription and translation upon transformation into a host cell or host cell organelles. Such additional segments may include regulatory nucleotide sequences, one or more origins of replication required for its maintenance and/or replication in a specific cell type, one or more selectable markers, a polyadenylation signal, a suitable site for the insertion of foreign coding sequences such as a multiple cloning site, etc. One example is when a vector is required to be maintained in a bacterial cell as an episomal genetic element (e.g. plasmid or cosmid molecule). Preferred origins of replication include, but are not limited to, the f1-ori and colE1. A vector may replicate without integrating into the genome of a host cell, e.g. as a plasmid in a bacterial host cell, or it may integrate part or all of its DNA into the genome of the host cell and thus lead to replication and expression of its DNA. The skilled artisan is well aware of the genetic elements that must be present on the genetic construct to successfully transform, select and propagate host cells containing the gene of interest.

Foreign nucleic acid may be introduced into a vector by means of cloning. Cloning may mean that by cleavage of the vector by suitable means and methods (e.g., restriction enzymes) e.g. within the multiple cloning site and the foreign nucleic acid comprising a coding sequence with appropriate means such as, e.g., restriction enzymes, fitting structures within the individual nucleic acids are created that enable the controlled fusion of said foreign nucleic acid and the vector.

Once introduced into the vector, the foreign nucleic acid comprising a coding sequence may be suitable to be introduced (transformed, transduced, transfected, etc.) into a host cell or host cell organelles. A cloning vector may be chosen for transport into a desired host cell or host cell organelles. A cloning vector may be chosen for expression of the foreign polynucleotide sequence in the host cell or host cell organelles. Suitability for expression normally requires that regulatory nucleotide sequences are operatively linked to the foreign polynucleotide sequence such that expression of the foreign polynucleotide sequence in the host cell or host cell organelle is possible. Such a vector may be called expression vector.

Expression vectors are generally derived from yeast or bacterial genomic or plasmid polynucleotide sequences, viral polynucleotide sequences, or artificial polynucleotide sequences, or may contain elements of two or more thereof. As already set forth, a vector may comprise one or more “origins of replication” which normally indicates a particular nucleotide sequence at which replication is initiated. Usually a origin of replication binds a protein complex that recognizes, unwinds, and begins to copy the polynucleotide sequence. Different origins of replication may be selected for different host cells or host cell organelles. The one skilled in the art is familiar with such a selection.

For the detection of the successful transfer of the nucleic acid sequences and/or selection of transgenic organisms or plants comprising these nucleic acids, it is advantageous to use marker genes (or reporter genes). Therefore, the vector may optionally comprise a selectable marker gene.

The term “terminator” encompasses a control sequence which is a DNA sequence at the end of a transcriptional unit which signals 3′ processing and polyadenylation of a primary transcript and termination of transcription. The terminator can be derived from the natural gene, from a variety of other plant genes, or from T-DNA. The terminator to be added may be derived from, for example, the nopaline synthase or octopine synthase genes, or alternatively from another plant gene, or less preferably from any other eukaryotic gene

“Construct”, “genetic construct” or “expression cassette” (used interchangeably) as used herein, is a DNA molecule composed of at least one sequence of interest to be expressed, operably linked to one or more control sequences (at least to a promoter) as described herein. Typically, the expression cassette comprises three elements: a promoter sequence, an open reading frame, and a 3′ untranslated region that, in eukaryotes, usually contains a polyadenylation site. Additional regulatory elements may include transcriptional as well as translational enhancers. An intron sequence may also be added to the 5′ untranslated region (UTR) or in the coding sequence to increase the amount of the mature message that accumulates in the cytosol. The skilled artisan is well aware of the genetic elements that must be present in the expression cassette to be successfully expressed. Preferably, at least part of the DNA or the arrangement of the genetic elements forming the expression cassette is artificial. The expression cassette may be part of a vector or may be integrated into the genome of a host cell and replicated together with the genome of its host cell. The expression cassette is capable of increasing or decreasing the expression of DNA and/or protein of interest.

The term “functional linkage” or “operably linked” means that the described components are in a relationship permitting them to function in their intended manner. For example, a regulatory sequence operably linked to a coding sequence is ligated in such a way that expression of the coding sequence is achieved under conditions compatible with the control sequences. Further, with respect to regulatory elements, is to be understood as meaning the sequential arrangement of a regulatory element (e.g. a promoter) with a nucleic acid sequence to be expressed and, if appropriate, further regulatory elements (such as e.g., a terminator) in such a way that each of the regulatory elements can fulfil its intended function to allow, modify, facilitate or otherwise influence expression of said nucleic acid sequence. The expression may result, depending on the arrangement of the nucleic acid sequences, in sense or antisense RNA. Preferred arrangements are those in which the nucleic acid sequence to be expressed recombinantly is positioned behind the sequence acting as promoter, so that the two sequences are linked covalently to each other. In a preferred arrangement, the nucleic acid sequence to be transcribed is located behind the promoter in such a way that the transcription start is identical with the desired beginning of the RNA. Functional linkage, and an expression construct, can be generated by means of customary recombination and cloning techniques as described (e.g., in Maniatis T, Fritsch E F and Sambrook J (1989) Molecular Cloning: A Laboratory Manual, 2nd Ed., Cold Spring Harbor Laboratory, Cold Spring Harbor (NY); Silhavy et al. (1984) Experiments with Gene Fusions, Cold Spring Harbor Laboratory, Cold Spring Harbor (NY); Ausubel et al. (1987) Current Protocols in Molecular Biology, Greene Publishing Assoc. and Wiley Interscience; Gelvin et al. (Eds) (1990) Plant Molecular Biology Manual; Kluwer Academic Publisher, Dordrecht, The Netherlands; Plant Molecular Biology Labfax (1993) by R. D. D. Croy, published by BIOS Scientific Publications Ltd (UK) and Blackwell Scientific Publications (UK)). However, further sequences, which, for example, act as a linker with specific cleavage sites for restriction enzymes, or as a signal peptide, may also be positioned between the two sequences. The insertion of sequences may also lead to the expression of fusion proteins. Preferably, the expression construct, consisting of a linkage of a regulatory region for example a promoter and nucleic acid sequence to be expressed, can exist in a vector-integrated form and be inserted into a plant genome, for example by transformation.

The term “introduction” or “transformation” as referred to herein encompasses the transfer of an exogenous polynucleotide into a host cell, irrespective of the method used for transfer. That is, the term “transformation” as used herein is independent from vector, shuttle system, or host cell, and it not only relates to the polynucleotide transfer method of transformation as known in the art (cf., for example, Sambrook, J. et al. (1989) Molecular Cloning: A Laboratory Manual, 2nd Ed., Cold Spring Harbor Laboratory Press, Cold Spring Harbor, NY), but it encompasses any further kind polynucleotide transfer methods such as, but not limited to, transduction or transfection. Plant tissue capable of subsequent clonal propagation, whether by organogenesis or embryogenesis, may be transformed with a genetic construct and a whole plant regenerated therefrom). The particular tissue chosen will vary depending on the clonal propagation systems available for, and best suited to, the particular species being transformed. The polynucleotide may be transiently or stably introduced into a host cell and may be maintained non-integrated, for example, as a plasmid. “Stable transformation” may mean that the transformed cell or cell organelle passes the nucleic acid comprising the foreign coding sequence on to the next generations of the cell or cell organelles. Usually stable transformation is due to integration of nucleic acid comprising a foreign coding sequence into the chromosomes or as an episome (separate piece of nuclear DNA).

“Transient transformation” may mean that the cell or cell organelle once transformed expresses the foreign nucleic acid sequence for a certain time—mostly within one generation. Usually transient transformation is due to nucleic acid comprising a foreign nucleic acid sequence is not integrated into the chromosomes or as an episome.

Alternatively, it may be integrated into the host genome. The resulting transformed plant cell may then be used to regenerate a transformed plant in a manner known to persons skilled in the art.

Transformation methods may be selected from the calcium/polyethylene glycol method for protoplasts (Krens, F. A. et al., (1982) Nature 296, 72-74; Negrutiu I et al. (1987) Plant Mol Biol 8: 363-373); electroporation of protoplasts (Shillito R. D. et al. (1985) Bio/Technol 3, 1099-1102); microinjection into plant material (Crossway A et al., (1986) Mol. Gen Genet 202: 179-185); DNA or RNA-coated particle bombardment (Klein T M et al., (1987) Nature 327: 70) infection with (non-integrative) viruses and the like. Transgenic plants, including transgenic crop plants, are preferably produced via Agrobacterium-mediated transformation. An advantageous transformation method is the transformation in planta. To this end, it is possible, for example, to allow the agrobacteria to act on plant seeds, on the intact plant or at least on the flower primordia, or to inoculate the plant meristem with agrobacteria. Methods for Agrobacterium-mediated transformation of rice include well known methods for rice transformation, such as those described in: European patent application EP 1198985 A1, Aldemita and Hodges (Planta 199: 612-617, 1996); Chan et al. (Plant Mol Biol 22 (3): 491-506, 1993), Hiei et al. (Plant J 6 (2): 271-282, 1994). In the case of corn transformation, the preferred method is as described in either Ishida et al. (Nat. Biotechnol 14(6): 745-50, 1996) or Frame et al. (Plant Physiol 129(1): 13-22, 2002). Said methods are further described by way of example in B. Jenes et al., Techniques for Gene Transfer, in: Transgenic Plants, Vol. 1, Engineering and Utilization, eds. S. D. Kung and R. Wu, Academic Press (1993) 128-143 and in Potrykus Annu. Rev. Plant Physiol. Plant Molec. Biol. 42 (1991) 205-225). The nucleic acids or the construct to be expressed is preferably cloned into a vector, which is suitable for transforming Agrobacterium tumefaciens, for example pBin19 (Bevan et al., Nucl. Acids Res. 12 (1984) 8711). Agrobacteria transformed by such a vector can then be used in known manner for the transformation of plants. The transformation of plants by means of Agrobacterium tumefaciens is described, for example, by Hofgen and Willmitzer in Nucl. Acid Res. (1988) 16, 9877 or is known inter alia from F. F. White, Vectors for Gene Transfer in Higher Plants; in Transgenic Plants, Vol. 1, Engineering and Utilization, eds. S. D. Kung and R. Wu, Academic Press, 1993, pp. 15-38.

Cotyledonary petioles and hypocotyls of 5-6 day old young seedling are used as explants for tissue culture and transformed according to Babic et al. (1998, Plant Cell Rep 17: 183-188). The commercial cultivar Westar (Agriculture Canada) is the standard variety used for transformation, but other varieties can also be used.

The terms “regulatory element”, “control sequence” and “promoter” are all used interchangeably herein and are to be taken in a broad context to refer to regulatory nucleic acid sequences capable of effecting expression of the sequences to which they are associated. “Regulatory elements” or “regulatory nucleotide sequences” herein may mean pieces of nucleic acid which drive expression of a nucleic acid sequence. one upon transformation into a host cell or cell organelle had occurred. Regulatory nucleotide sequences may include any nucleotide sequence having a function or purpose individually and within a particular arrangement or grouping of other elements or sequences within the arrangement. Examples of regulatory nucleotide sequences include but are not limited to transcription control elements such as promoters, enhancers, and termination elements. Regulatory nucleotide sequences may be native (i.e. from the same gene) or foreign (i.e. from a different gene) to a nucleotide sequence to be expressed.

The term “promoter” typically refers to a nucleic acid control sequence located upstream from the transcriptional start of a gene and is involved in recognising and binding of RNA polymerase and other proteins, thereby directing transcription of an operably linked nucleic acid. “Promoter” herein may further include any nucleic acid sequence capable of driving transcription of a coding sequence. In particular, the term “promoter” as used herein may refer to a polynucleotide sequence generally described as the 5′ regulator region of a gene, located proximal to the start codon. The transcription of one or more coding sequence is initiated at the promoter region. The term promoter may also include fragments of a promoter that are functional in initiating transcription of the gene. Promoter may also be called “transcription start site” (TSS).

Encompassed by the aforementioned terms are further transcriptional regulatory sequences derived from a classical eukaryotic genomic gene (including the TATA box which is required for accurate transcription initiation, with or without a CCAAT box sequence) and additional regulatory elements (i.e. upstream activating sequences, enhancers and silencers) which alter gene expression in response to developmental and/or external stimuli, or in a tissue-specific manner.

For example, enhancers as known in the art and as used herein are normally short DNA segments (e.g. 50-1500 bp) which may be bound by proteins such as transcription factors to increase the likelihood that transcription of a coding sequence will occur.

Also included within the term is a transcriptional regulatory sequence of a classical prokaryotic gene, in which case it may include a −35 box sequence and/or −10 box transcriptional regulatory sequences. The term “regulatory element” also encompasses a synthetic fusion molecule or derivative that confers, activates or enhances expression of a nucleic acid molecule in a cell, tissue or organ. A promoter can be modified by one or more nucleotide substitution(s), insertion(s) and/or deletion(s) without interfering with functionality or activity, but it is also possible to increase the activity by modification of its sequence.

Further elements may be “transcription termination elements” which include pieces of nucleic acid sequences marking the end of a gene and mediating the transcriptional termination by providing signals within mRNA that initiates the release of the mRNA from the transcriptional complex. Transcriptional termination in prokaryotes usually is initiated by Rho-dependent or Rho-independent terminators. In eukaryotes transcription termination usually occurs through recognition of termination by proteins associated with RNA polymerase II.

A “plant promoter” comprises regulatory elements, which mediate the expression of a coding sequence segment in plant cells. Accordingly, a plant promoter need not be of plant origin, but may originate from viruses or microorganisms. For expression in plants, the nucleic acid molecule to be expressed must, as described herein, be linked operably to or comprise a suitable promoter which expresses the gene at the right point in time and with the required spatial expression pattern.

Functionally equivalents of a promoter have substantially the same strength and expression pattern as the original promoter. For the identification of functionally equivalent promoters, the promoter strength and/or expression pattern of a candidate promoter may be analysed for example by operably linking the promoter to a reporter gene and assaying the expression level and pattern of the reporter gene in various tissues of the plant. Suitable well-known reporter genes include for example beta-glucuronidase or beta-galactosidase. The promoter activity is assayed by measuring the enzymatic activity of the beta-glucuronidase or beta-galactosidase. The promoter strength and/or expression pattern may then be compared to that of a reference promoter (such as the one used in the methods described herein). Alternatively, promoter strength may be assayed by quantifying mRNA levels or by comparing mRNA levels of the nucleic acid used in the methods described herein, with mRNA levels of housekeeping genes such as 18S rRNA, using methods known in the art, such as Northern blotting with densitometric analysis of autoradiograms, quantitative real-time PCR or RT-PCR (Heid et al., 1996 Genome Methods 6: 986-994).

Constitutive Promoter

A “constitutive promoter” refers to a promoter that is transcriptionally active during most, but not necessarily all, phases of growth and development and under most environmental conditions, in at least one cell, tissue or organ.

A “ubiquitous promoter” is active in substantially all tissues or cells of an organism. A “developmentally-regulated promoter” is active during certain developmental stages or in parts of the plant that undergo developmental changes. Inducible promoter

An “inducible promoter” has induced or increased transcription initiation in response to a chemical (for a review see Gatz 1997, Annu. Rev. Plant Physiol. Plant Mol. Biol., 48:89-108), environmental or physical stimulus, or may be “stress-inducible”, i.e. activated when a plant is exposed to various stress conditions, or a “pathogen-inducible” i.e. activated when a plant is exposed to exposure to various pathogens. Organ-specific/Tissue-specific promoter

An “organ-specific” or “tissue-specific promoter” is one that is capable of preferentially initiating transcription in certain organs or tissues, such as the leaves, roots, seed tissue etc. For example, a “root-specific promoter” is a promoter that is transcriptionally active predominantly in plant roots, substantially to the exclusion of any other parts of a plant, whilst still allowing for any leaky expression in these other plant parts. Promoters able to initiate transcription in certain cells only are referred to herein as “cell-specific”. A “seed-specific promoter” is transcriptionally active predominantly in seed tissue, but not necessarily exclusively in seed tissue (in cases of leaky expression). The seed-specific promoter may be active during seed development and/or during germination. The seed specific promoter may be endosperm/aleurone/embryo specific. Examples of seed-specific promoters are given in Qing Qu and Takaiwa (Plant Biotechnol. J. 2, 113-125, 2004). A “green tissue-specific promoter” as defined herein is a promoter that is transcriptionally active predominantly in green tissue, substantially to the exclusion of any other parts of a plant, whilst still allowing for any leaky expression in these other plant parts.

Another example of a tissue-specific promoter is a meristem-specific promoter, which is transcriptionally active predominantly in meristematic tissue, substantially to the exclusion of any other parts of a plant, whilst still allowing for any leaky expression in these other plant parts.

An “intron” is a portion of non-coding DNA within a eukaryotic gene, which is removed from the primary gene transcript during RNA processing that generates mature and functional mRNA or other type of RNA.

Generally, the term “overexpression” as used herein comprises both, overexpression of polynucleotides (e.g., on the transcriptional level) and overexpression of polypeptides (e.g., on the translation level). In this context, the expression level of a polynucleotide can be easily assessed by the skilled person by methods known in the art, e.g., by quantitative RT-PCR (qRT-PCR), Northern Blot (for assessing the amount of expressed mRNA levels), Dot Blot, Microarray or the like (see, e.g., Sambrook, loc cit; Current Protocols in Molecular Biology, Update May 9, 2012, Print ISSN: 1934-3639, Online ISSN: 1934-3647). Preferably, the amount of expressed polynucleotide is measured by qRT-PCR.

An increase of the activity of the polypeptides used in the method of the invention can for example be achieved by overexpression of the corresponding PDCT.

In this context, the expression level of a polypeptide can be easily assessed by the skilled person by methods known in the art, e.g., by Western Blot, ELISA, EIA, RIA, or the like (see, e.g., Sambrook, loc cit; Current Protocols in Molecular Biology, Update May 9, 2012, Print ISSN: 1934-3639, Online ISSN: 1934-3647). Preferably, the amount of expressed polypeptide is measured by Western Blot.

If not stated otherwise herein, abbreviations and nomenclature, where employed, are deemed standard in the field and commonly used in professional journals such as those cited herein.

Accordingly, the present invention relates to the following items:

A method for the production of a plant, a part thereof, a plant cell, plant seed and/or plant seed oil, wherein the total PUFAs level is increased compared to a control, comprising increasing, compared to the control, a plant, a part thereof, a plant cell, and/or plant seed the activity [e.g. via increasing expression] of one or more PDCT wherein the PDCT is selected from the group consisting of:

(a) a PDCT1 having at least 80% sequence identity with SEQ ID NO: 2, 4, 6, 8, 10, 12, 14, 16, 40, 42, 44, and/or 46;

(b) a PDCT1 encoded by a polynucleotide having at least 80% sequence identity with SEQ ID NO: 1, 3, 5, 7, 9, 11, 13, 15, 39, 41, 43, and/or 45;

(c) a PDCT1 encoded by a polynucleotide that hybridizes under high stringency conditions with (i) a polynucleotide that encodes the amino acid sequence of SEQ ID NO: 2, 4, 6, 8, 10, 12, 14, 16, 40, 42, 44, and/or 46, or (ii) the full-length complement of (i);

(d) a variant of the PDCT1 of SEQ ID NO: 2, 4, 6, 8, 10, 12, 14, 16, 40, 42, 44, and/or 46 comprising a substitution, preferably a conservative substitution, deletion, and/or insertion at one or more positions and having PDCT1 activity;

(e) a PDCT1 encoded by a polynucleotide that differs from SEQ ID NO: 1, 3, 5, 7, 9, 11, 13, 15, 39, 41, 43, and/or 45 due to the degeneracy of the genetic code; and

(f) a fragment of the PDCT1 of (a), (b), (c), (d) or (e) having PDCT1 activity;

and, optionally, isolating the seed oil.

According to the method of the invention, the PDCT can for example be expressed as transgene under control of a heterologous promoter.

Further, the method of the invention relates to a method for increasing the level of DPA, DHA and/or EPA in a plant, a part thereof, a plant cell, and/or plant seed, that is capable to produce DPA, DHA and/or EPA and expresses a Delta-6 elongase, comprising providing a plant, a part thereof, a plant cell, and/or plant seed with an increased activity or expression of one or more PDCT selected from the group consisting of:

(a) a PDCT1 having at least 80% sequence identity with SEQ ID NO: 2, 4, 6, 8, 10, 12, 14, 16, 40, 42, 44, and/or 46;

(b) a PDCT1 encoded by a polynucleotide having at least 80% sequence identity with SEQ ID NO: 1, 3, 5, 7, 9, 11, 13, 15, 39, 41, 43, and/or 45;

(c) a PDCT1 encoded by a polynucleotide that hybridizes under high stringency conditions with (i) a polynucleotide that encodes the amino acid sequence of SEQ ID NO: 2, 4, 6, 8, 10, 12, 14, 16, 40, 42, 44, and/or 46, or (ii) the full-length complement of (i);

(d) a variant of the PDCT1 of SEQ ID NO: 2, 4, 6, 8, 10, 12, 14, 16, 40, 42, 44, and/or 46 comprising a substitution, preferably a conservative substitution, deletion, and/or insertion at one or more positions and having PDCT1 activity;

(e) a PDCT1 encoded by a polynucleotide that differs from SEQ ID NO: 1, 3, 5, 7, 9, 11, 13, 15, 39, 41, 43, and/or 45 due to the degeneracy of the genetic code; and

(f) a fragment of the PDCT1 of (a), (b), (c), (d) or (e) having PDCT1 activity;

Further, the present invention relates to a method for increasing the Delta-6 elongase conversion efficiency in a plant, plant cell, plant seed and/or part thereof, that is capable to produce PUFA and expresses a delta-6 Elongase, comprising increasing, compared to a control, in the plant, plant cell, plant seed and/or part thereof the activity [e.g. via increasing expression] of one or more PDCT selected from the group consisting of:

(a) a PDCT1 having at least 80% sequence identity with SEQ ID NO: 2, 4, 6, 8, 10, 12, 14, 16, 40, 42, 44, and/or 46;

(b) a PDCT1 encoded by a polynucleotide having at least 80% sequence identity with SEQ ID NO: 1, 3, 5, 7, 9, 11, 13, 15, 39, 41, 43, and/or 45;

(c) a PDCT1 encoded by a polynucleotide that hybridizes under high stringency conditions with (i) a polynucleotide that encodes the amino acid sequence of SEQ ID NO: 2, 4, 6, 8, 10, 12, 14, 16, 40, 42, 44, and/or 46, or (ii) the full-length complement of (i);

(d) a variant of the PDCT1 of SEQ ID NO: 2, 4, 6, 8, 10, 12, 14, 16, 40, 42, 44, and/or 46 comprising a substitution, preferably a conservative substitution, deletion, and/or insertion at one or more positions and having PDCT1 activity;

(e) a PDCT1 encoded by a polynucleotide that differs from SEQ ID NO: 1, 3, 5, 7, 9, 11, 13, 15, 39, 41, 43, and/or 45 due to the degeneracy of the genetic code; and

(f) a fragment of the PDCT1 of (a), (b), (c), (d) or (e) having PDCT1 activity.

Further, the Delta-6 desataurase used in the method of the invention is for example a Acyl CoA dependent delta-6 Desaturase.

Further, the method of the invention relates to a method for improving the cellular conversion efficiency from oleic acid to C18 to C22 PUFA in a plant, plant seed, plant cell or part thereof, comprising providing a plant, plant cell, plant seed or part thereof, that is capable to produce C18 to C22 PUFA, comprising increasing the activity [or expression] of one or more PDCT selected from the group consisting of:

(a) a PDCT1 having at least 80% sequence identity with SEQ ID NO: 2, 4, 6, 8, 10, 12, 14, 16, 40, 42, 44, and/or 46;

(b) a PDCT1 encoded by a polynucleotide having at least 80% sequence identity with SEQ ID NO: 1, 3, 5, 7, 9, 11, 13, 15, 39, 41, 43, and/or 45;

(c) a PDCT1 encoded by a polynucleotide that hybridizes under high stringency conditions with (i) a polynucleotide that encodes the amino acid sequence of SEQ ID NO: 2, 4, 6, 8, 10, 12, 14, 16, 40, 42, 44, and/or 46, or (ii) the full-length complement of (i);

(d) a variant of the PDCT1 of SEQ ID NO: 2, 4, 6, 8, 10, 12, 14, 16, 40, 42, 44, and/or 46 comprising a substitution, preferably a conservative substitution, deletion, and/or insertion at one or more positions and having PDCT1 activity;

(e) a PDCT1 encoded by a polynucleotide that differs from SEQ ID NO: 1, 3, 5, 7, 9, 11, 13, 15, 39, 41, 43, and/or 45 due to the degeneracy of the genetic code; and

(f) a fragment of the PDCT1 of (a), (b), (c), (d) or (e) having PDCT1 activity.

Further, the method of the invention relates to a method for producing vlcPUFA in an oil crop plant, comprising

providing a first an oil crop plant variety that is cable to produce the desired vIcPUFA,

providing a second an oil crop plant variety that has an increased activity of one or more PDCT selected from the group consisting of:

a) a PDCT1 having at least 80% sequence identity with SEQ ID NO: 2, 4, 6, 8, 10, 12, 14, 16, 40, 42, 44, and/or 46;

(b) a PDCT1 encoded by a polynucleotide having at least 80% sequence identity with SEQ ID NO: 1, 3, 5, 7, 9, 11, 13, 15, 39, 41, 43, and/or 45;

(c) a PDCT1 encoded by a polynucleotide that hybridizes under high stringency conditions with (i) a polynucleotide that encodes the amino acid sequence of SEQ ID NO: 2, 4, 6, 8, 10, 12, 14, 16, 40, 42, 44, and/or 46, or (ii) the full-length complement of (i);

(d) a variant of the PDCT1 of SEQ ID NO: 2, 4, 6, 8, 10, 12, 14, 16, 40, 42, 44, and/or 46 comprising a substitution, preferably a conservative substitution, deletion, and/or insertion at one or more positions and having PDCT1 activity;

(e) a PDCT1 encoded by a polynucleotide that differs from SEQ ID NO: 1, 3, 5, 7, 9, 11, 13, 15, 39, 41, 43, and/or 45 due to the degeneracy of the genetic code; and

(f) a fragment of the PDCT1 of (a), (b), (c), (d) or (e) having PDCT1 activity;

crossing the first and second an oil crop plant variety,

optionally, measuring the PDCT1 expression rate in first or later generation cells, seeds, plants or part thereof derived from the cross,

optionally, measuring the total PUFA level in in first or later generation cells, seeds, plants or part thereof derived from the cross,

optionally, repeating steps 2 to 5,

planting and growing the plants, and;

isolating the vIcPUFA comprising oil from the seed of first or later generation plants derived from the cross.

According to this invention “derived from the cross” means that the generation of plants that is used to produce the oil is not limited in the generation as long as the features that were introduced into the plant, plant cell or plant seed are resulting from the cross of the first and second oil plant variety. For example, any generation of the plant benefits in its PUFA production from the results of this method, e.g. from the increase of the activity of the PDCT1.

For example, in the method of the invention, the plant, plant seed or plant cell expresses at least one phospholipid-dependent desaturase, preferably selected from the group consisting of d4-, d5-, d6-, Omega-3-desaturase and d12desaturase.

For example, in the method of the invention the plant, plant seed or plant cell expresses at least one phospholipid-dependent desaturase and at least one Acetyl-CoA-dependent desaturase, preferably selected from the group consisting of d4-, d5-, d6-, Omega-3-desaturase and d12desaturase.

For example, in the method of the invention the plant, plant seed or plant cell expresses at least one Delta 6 elongase and/or at least one Delta 6-desaturase.

Further, the present invention relates to a method for the production of a composition comprising the fatty acids GLA, HGLA, SDA and/or ETA, preferably GLA, HGLA, SDA and ETA, even more preferred in total PUFA, in a plant, plant cell, or part seed, or part thereof, cable to produce GLA, HGLA, SDA and/or ETA, comprising providing a plant, plant cell or seed with an increased activity or expression of one or more PDCT selected from the group consisting of:

(a) a PDCT1 having at least 80% sequence identity with SEQ ID NO: 2, 4, 6, 8, 10, 12, 14, 16, 40, 42, 44, and/or 46

(b) a PDCT1 encoded by a polynucleotide having at least 80% sequence identity with SEQ ID NO: 1, 3, 5, 7, 9, 11, 13, 15, 39, 41, 43, and/or 45;

(c) a PDCT1 encoded by a polynucleotide that hybridizes under high stringency conditions with (i) a polynucleotide that encodes the amino acid sequence of SEQ ID NO: 2, 4, 6, 8, 10, 12, 14, 16, 40, 42, 44, and/or 46, or (ii) the full-length complement of (i);

(d) a variant of the PDCT1 of SEQ ID NO: 2, 4, 6, 8, 10, 12, 14, 16, 40, 42, 44, and/or 46 comprising a substitution, preferably a conservative substitution, deletion, and/or insertion at one or more positions and having PDCT1 activity;

(e) a PDCT1 encoded by a polynucleotide that differs from SEQ ID NO: 1, 3, 5, 7, 9, 11, 13, 15, 39, 41, 43, and/or 45 due to the degeneracy of the genetic code; and (f) a fragment of the PDCT1 of (a), (b), (c), (d) or (e) having PDCT1 activity;

and, optionally, isolating the composition comprising the desired fatty acids.

For example, the amount of GLA, HGLA, SDA and/or ETA, more preferred in total PUFAs is increased compared to a control that does not have an increased PDCT activity.

Further, the present invention relates to a method for increasing the level of acids GLA, HGLA, SDA and/or ETA, even more preferred in total PUFA, in a plant, plant cell, or part seed, or part thereof, cable to produce GLA, HGLA, SDA and/or ETA, in a plant, plant cell, seed, and/or a part thereof, comprising providing a plant, plant cell, seed, and/or part thereof with an increased activity or expression of one or more PDCT selected from the group consisting of:

(a) a PDCT1 having at least 80% sequence identity with SEQ ID NO: 2, 4, 6, 8, 10, 12, 14, 16, 40, 42, 44, and/or 46;

(b) a PDCT1 encoded by a polynucleotide having at least 80% sequence identity with SEQ ID NO: 1, 3, 5, 7, 9, 11, 13, 15, 39, 41, 43, and/or 45;

(c) a PDCT1 encoded by a polynucleotide that hybridizes under high stringency conditions with (i) a polynucleotide that encodes the amino acid sequence of SEQ ID NO: 2, 4, 6, 8, 10, 12, 14, 16, 40, 42, 44, and/or 46, or (ii) the full-length complement of (i);

(d) a variant of the PDCT1 of SEQ ID NO: 2, 4, 6, 8, 10, 12, 14, 16, 40, 42, 44, and/or 46 comprising a substitution, preferably a conservative substitution, deletion, and/or insertion at one or more positions and having PDCT1 activity;

(e) a PDCT1 encoded by a polynucleotide that differs from SEQ ID NO: 1, 3, 5, 7, 9, 11, 13, 15, 39, 41, 43, and/or 45 due to the degeneracy of the genetic code; and

(f) a fragment of the PDCT1 of (a), (b), (c), (d) or (e) having PDCT1 activity; and, optionally, isolating the composition comprising the desired fatty acids.

whereby the plant, plant seed or plant cell expresses at least one acyl-CoA dependent desaturase, preferably selected from the group consisting of d4-, d5-, d6-, and d12desaturase and/or at least one PC-dependent elongase selected from the group consisting of d5-, d5d6-, and d6elongase

Thus, for example, the total PUFA level is increased compared to a control, e.g. a plant, plant cell or plant seed that does not show the increased activity of the PDGT1.

Further, for example, the PUFA compositions is characterized by a shift to an increase of C20 PUFAs compared to C18 PUFAs, e.g., the level of C20 PUFAs is higher than C18.

Thus, the present invention also relates to a plant raw oil that comprises more C20 fatty acids than C18 fatty acids, as well as to a plant seed that comprises such an oil, e.g. to a oil seed crop seed, and for example an raw oil derived from or obtained in a seed from B. species or Camelina species as described herein.

Further, the raw oil produced according to the method described herein, can for example be an oil composition isolated from the plant the plant or cell is derived from a Camelina so or Brassica sp. expressing a delta 6 desaturase and having an ALA level that is at least 10%, preferably 20, 30, 40, or 50% more reduced compared to a control.

The method of the invention relates to a method for improved production of the fatty acid GLA, preferably to an increase in total PUFA, in a plant, plant cell, or part seed, or part thereof, cable to produce GLA plant, plant cell, seed or a part thereof, which comprises,

providing a plant, seed, or plant cell capable to produce acids comprising

at least one nucleic acid sequence which encodes at least one D12 desaturase

at least one nucleic acid sequence which encodes at least one omega 3 desaturase,

at least one nucleic acid sequence which encodes a delta 6-desaturase activity,

b) at least one nucleic acid sequence which encodes a delta-6 elongase activity,

c) at least one nucleic acid sequence which encodes a delta-5 desaturase activity,

d) at least one nucleic acid sequence which encodes a delta-5 elongase activity, and

e) at least one nucleic acid sequence which encodes a delta-4 desaturase activity, and

whereby the plant has an increased activity of one or more PDCT selected from the group consisting of:

a) a PDCT1 having at least 80% sequence identity with SEQ ID NO: 2, 4, 6, 8, 10, 12, 14, 16, 40, 42, 44, and/or 46;

(b) a PDCT1 encoded by a polynucleotide having at least 80% sequence identity with SEQ ID NO: 1, 3, 5, 7, 9, 11, 13, 15, 39, 41, 43, and/or 45;

(c) a PDCT1 encoded by a polynucleotide that hybridizes under high stringency conditions with (i) a polynucleotide that encodes the amino acid sequence of SEQ ID NO: 2, 4, 6, 8, 10, 12, 14, 16, 40, 42, 44, and/or 46, or (ii) the full-length complement of (i);

(d) a variant of the PDCT1 of SEQ ID NO: 2, 4, 6, 8, 10, 12, 14, 16, 40, 42, 44, and/or 46 comprising a substitution, preferably a conservative substitution, deletion, and/or insertion at one or more positions and having PDCT1 activity;

(e) a PDCT1 encoded by a polynucleotide that differs from SEQ ID NO: 1, 3, 5, 7, 9, 11, 13, 15, 39, 41, 43, and/or 45 due to the degeneracy of the genetic code; and

(f) a fragment of the PDCT1 of (a), (b), (c), (d) or (e) having PDCT1 activity; and,

optionally, isolating the composition comprising the desired fatty acids.

and wherein at least one desaturase is PC dependent,

and; optionally, isolating the fatty composition comprising EPA, DPA and/or DHA.

The plant or plant cell used in the method of the invention preferably is also capable to produce C20 and/or C22 FA, in particular DHA, EPA and DPA.

The present invention also provides a method as described wherein level of 18:1 is reduced by at least 10%, 15%, 20%, 25%, 30%, 40%, 50%, or more compared to the control and/or wherein ALA is reduced by at least 10%, 20%, 30%, 40%, 50%, or more compared to a control.

Further, according to the method of the invention, for example, one of the following PDCT can be expressed: Camelina sativa PDCT C1, and/or Camelina sativa PDCT C19.

For example in the method of the invention the activity of one or more PDCT can be increased, e.g. as selected from the group consisting of:

a) a PDCT1 having at least 80% sequence identity with SEQ ID NO: 2, 4, 6, 8, 10, 12, 14, 16, 40, 42, 44, and/or 46;

(b) a PDCT1 encoded by a polynucleotide having at least 80% sequence identity with SEQ ID NO: 1, 3, 5, 7, 9, 11, 13, 15, 39, 41, 43, and/or 45;

(c) a PDCT1 encoded by a polynucleotide that hybridizes under high stringency conditions with (i) a polynucleotide that encodes the amino acid sequence of SEQ ID NO: 2, 4, 6, 8, 10, 12, 14, 16, 40, 42, 44, and/or 46, or (ii) the full-length complement of (i);

(d) a variant of the PDCT1 of SEQ ID NO: 2, 4, 6, 8, 10, 12, 14, 16, 40, 42, 44, and/or 46 comprising a substitution, preferably a conservative substitution, deletion, and/or insertion at one or more positions and having PDCT1 activity;

(e) a PDCT1 encoded by a polynucleotide that differs from SEQ ID NO: 1, 3, 5, 7, 9, 11, 13, 15, 39, 41, 43, and/or 45 due to the degeneracy of the genetic code; and

(f) a fragment of the PDCT1 of (a), (b), (c), (d) or (e) having PDCT1 activity; and, optionally, isolating the composition comprising the desired fatty acids.

and

one or more PDCT selected from the group consisting of:

(a) a PDCT19 having at least 80% sequence identity with SEQ ID NO: 36, 38, and/or 48;

(b) a PDCT19 encoded by a polynucleotide having at least 80% sequence identity with SEQ ID NO: 35, 37, and/or 47;

(c) a PDCT19 encoded by a polynucleotide that hybridizes under high stringency conditions with (i) a polynucleotide that encodes the amino acid sequence of SEQ ID NO: 36, 38, and/or 48, or (ii) the full-length complement of (i);

(d) a variant of the PDCT19 of SEQ ID NO: 36, 38, and/or 48 comprising a substitution, preferably a conservative substitution, deletion, and/or insertion at one or more positions and having PDCT19 activity;

(e) a PDCT19 encoded by a polynucleotide that differs from SEQ ID NO: 35, 37, and/or 47 due to the degeneracy of the genetic code; and

(f) a fragment of the PDCT19 of (a), (b), (c), (d) or (e) having PDCT19 activity.

Further, in one embodiment, in the method of the invention a PDCT3 and or a PDCT5 as defined herein is reduced. For example, if the plant used in the method of the invention is B. napus activity of at least one of the following PDCT is reduced: Brassica napus PDCT 5A, and/or Brassica napus PDCT 3A.

The method of the invention, also comprises the step of optionally, isolating the fatty acid composition produced as raw oil. Optionally, the raw oil is formulated to as a fatty acid composition to food or feed.

Further, the method of the invention, for example also comprises the expressing in the plant, plant cell or seed of a further PDCT whereby the PDCT is selected from the group of

(a) a PDCT19 having at least 80% sequence identity with SEQ ID NO: 36, 38, and/or 48;

(b) a PDCT19 encoded by a polynucleotide having at least 80% sequence identity with SEQ ID NO: 35, 37, and/or 47;

(c) a PDCT19 encoded by a polynucleotide that hybridizes under high stringency conditions with (i) a polynucleotide that encodes the amino acid sequence of SEQ ID NO: 36, 38, and/or 48, or (ii) the full-length complement of (i);

(d) a variant of the PDCT19 of SEQ ID NO: 36, 38, and/or 48 comprising a substitution, preferably a conservative substitution, deletion, and/or insertion at one or more positions and having PDCT19 activity;

(e) a PDCT19 encoded by a polynucleotide that differs from SEQ ID NO: 35, 37, and/or 47 due to the degeneracy of the genetic code; and

(f) a fragment of the PDCT19 of (a), (b), (c), (d) or (e) having PDCT19 activity.

and whereby said PDCT19 is expressed under the control of a heterologous promoter.

Further, the method of the invention, for example also comprises the plant, plant cell, plant seed or part has a decreased activity of one or more PDCT selected from the group consisting of:

(a) PDCT3 and/or PDCT5 having at least 80% sequence identity with SEQ ID NO: 18, 20, 22, 24, 26, 28, 30, 32, 50, 52, 54, 56, 58, and/or 60;

(b) PDCT3 and/or PDCT5 encoded by a polynucleotide having at least 80% sequence identity with SEQ ID NO: 17, 19, 21, 23, 27, 29, 31, 49, 51, 53, 55, and/or 57;

(c) PDCT3 and/or PDCT5 encoded by one or more polynucleotides that hybridize under high stringency conditions with (i) a polynucleotide that encodes the amino acid sequence of SEQ ID NO: 18, 20, 22, 24, 26, 28, 30, 32, 50, 52, 54, 56, 58, and/or 60, or (ii) the full-length complement of (i);

(d) variants of the PDCT3 and/or PDCT5 of SEQ ID NO: 18, 20, 22, 24, 26, 28, 30, 32, 50, 52, 54, 56, 58, and/or 60, comprising a substitution, preferably a conservative substitution, deletion, and/or insertion at one or more positions and having PDCT3 and/or PDCT5 activity;

(e) PDCT3 and/or PDCT5 encoded by a polynucleotide that differs from SEQ ID NO: 17, 19, 21, 23, 27, 29, 31, 49, 51, 53, 55, and/or 57 due to the degeneracy of the genetic code; and

(f) fragments of the PDCT3 and/or PDCT5 of (a), (b), (c), (d) or (e) having PDCT3 and/or PDCT5 activity.

For example, in the method of the invention, the increased activity of the PDCT1 can be achieved by expressing de novo or overexpressing a PDCT1. Further, for example, the activity of more than one PDCT1 is increased, overexpressing or expressing de novo the PDCT1 shown in FIG. 6B. Further, for example, the activity of more than one PDCT1 is increased, overexpressing or expressing de novo the PDCT1 shown in FIG. 6C. According to the method of the invention, for example, also a PDCT1 as shown in FIG. 6B and one as shown in FIG. 6C can be expressed or overexpressed to achieve the desired effect of the method.

For example, in the method of the invention, the increased activity of the PDCT19 can be achieved by expressing de novo or overexpressing a PDCT19. Further, for example, the activity of more than one PDCT19 is increased, overexpressing or expressing de novo the PDCT1 shown in FIG. 6D. According to the method of the invention, for example, also a PDCT1 as shown in FIG. 6B and one as shown in FIG. 6C can be expressed or overexpressed together with a PDCT shown in FIG. 6D to achieve the desired effect of the method.

Preferably, the gene that corresponds to the target organism, e.g. the organism in which the activity shall be increased, is overexpressed.

For example, a PDCT3 from B. napus as shown in FIG. 6D is reduced in its activity in the method of the present invention in B. napus. For example, a PDCT5 from B. juncea as shown in FIG. 6F is reduced in its activity in the method of the present inventionin B. juncea.

Accordingly, the present invention also relates to an isolated, a synthetic, or a recombinant polynucleotide comprising:

(a) a nucleic acid sequence having at least 80% sequence identity to SEQ ID NO: 1, 3, 5, 7, 9, 11, 13, 15, 39, 41, 43, and/or 45, wherein the nucleic acid encodes a polypeptide having PDCT1 activity;

(b) a nucleic acid sequence encoding a polypeptide having at least 80% sequence identity to SEQ ID NO: 2, 4, 6, 8, 10, 12, 14, 16, 40, 42, 44, and/or 46, wherein the polypeptide has PDCT1 activity;

(c) a fragment of (a) or (b), wherein the fragment encodes a polypeptide having PDCT1 activity; or

(d) a nucleic acid sequence fully complementary to any of (a) to (c).

Further, the present invention relates to an isolated, a synthetic, or a recombinant polynucleotide comprising polynucleotide of the invention and further:

(a) a nucleic acid sequence having at least 80% sequence identity to SEQ ID NO: 35, 37, and/or 47, wherein the nucleic acid encodes a polypeptide having PDCT19 activity;

(b) a nucleic acid sequence encoding a polypeptide having at least 80% sequence identity to SEQ ID NO: 36, 38, and/or 48, wherein the polypeptide has PDCT19 activity;

(c) a fragment of (a) or (b), wherein the fragment encodes a polypeptide having PDCT19 activity; or

(d) a nucleic acid sequence fully complementary to any of (a) to (c).

Further, the present invention also relates to an isolated, synthetic, or recombinant polypeptide comprising an amino acid sequence of a PDCT, wherein the PDCT is selected from the group consisting of:

(a) a PDCT1 having at least 80% sequence identity with SEQ ID NO: 2, 4, 6, 8, 10, 12, 14, 16, 40, 42, 44, and/or 46;

(b) a PDCT1 encoded by a polynucleotide having at least 80% sequence identity with SEQ ID NO: 1, 3, 5, 7, 9, 11, 13, 15, 39, 41, 43, and/or 45;

(c) a PDCT1 encoded by a polynucleotide that hybridizes under high stringency conditions with (i) a polynucleotide that encodes the amino acid sequence of SEQ ID NO: 2, 4, 6, 8, 10, 12, 14, 16, 40, 42, 44, and/or 46, or (ii) the full-length complement of (i);

(d) a variant of the PDCT1 of SEQ ID NO: 2, 4, 6, 8, 10, 12, 14, 16, 40, 42, 44, and/or 46 comprising a substitution, preferably a conservative substitution, deletion, and/or insertion at one or more positions and having PDCT1 activity;

(e) a PDCT1 encoded by a polynucleotide that differs from SEQ ID NO: 1, 3, 5, 7, 9, 11, 13, 15, 39, 41, 43, and/or 45 due to the degeneracy of the genetic code; and

(f) a fragment of the PDCT1 of (a), (b), (c), (d) or (e) having PDCT1 activity.

Further, the nucleic acid construct of the invention can operably be linked to one or more heterologous control sequences that directs the expression of the protein of interest in a cell, preferably in a plant cell.

For example, the present invention also relates to a nucleic acid construct preferably for expression in plant cells, preferably in seed, or comprised in a host cell, preferably in a Agrobacterium, bacterial cell, plant cell, or seed cell, e.g. derived from an oil crop, e.g. Brassica napus, Brassica juncea, Brassica carrinata, or C. sativa,

Accordingly, the present invention relates to a replacement regulatory element increasing the expression of an endogenous PDCT comprising the polypeptide of the present invention when replacing the endogenous regulatory element.

Further, the present invention relates to a vector comprising the polynucleotide of the invention, or the nucleic acid construct of the invention. For example, the vector of the invention is a plasmid, expression vector, a cosmid, a fosmid, or an artificial chromosome. For example, the vector of the invention comprises a selection marker, a polyadenylation signal, a multiple cloning site, an origin of replication, a promoter, and/or a termination signal.

Further, the present invention relates to a host cell comprising a polynucleotide of the invention, a nucleic acid construct of the invention or a vector of the invention. For example, the host cell is transformed with a polynucleotide of the invention, a nucleic acid construct of the invention or a vector of claim of the invention. Futher, the host cell for example be selected from the group consisting of Agrobacterium, yeast, bacterial, algae or plant cell. Further, the host cell for example stably expresses said polynucleotide or vector.

Also, the present invention relates to composition comprising the polynucleotide of invention or a nucleic acid construct of the invention, and a host cell, preferably the host cell of of the invention, e.g. an Agrobacterium, a yeast or a plant seed cell, wherein the nucleic acid construct is comprised within the host cell.

Accordingly, the present invention also relates to a method of producing the polypeptide of the invention, or the polynucleotide of the invention, comprising the steps of

(a) providing a host cell, preferably the host cell of the invention, e.g. an Agrobacterium, a yeast or a plant seed cell, comprising a polynucleotide encoding a polypeptide of of the invention or the polynucleotide of the invention;

(b) cultivating the host cell of step (a) under conditions conductive for the production of the polypeptide of the invention or the polynucleotide of the invention in the host cell; and

(b) optionally, recovering the polypeptide of the invention or the polynucleotide of the invention.

Further, the present invention relates to a method for the production of a transgenic plant, plant cell, plant seed, a part thereof, or an oil thereof, having an increased amount of GLA, HGLA, SDA and/or ETA, preferably an increased the combination of GLA, HGLA, SDA and ETA, even more preferred in total PUFA, in a plant, plant cell, or part seed, or part thereof, cable to produce GLA having an increased the conversion rate of a phospholipid-dependent desaturase increased relative to control plants, said method comprising:

(i) introducing and expressing in a plant, or part thereof, or plant cell, or plant seed a nucleic acid encoding a polypeptide of the invention; and

(ii) cultivating said plant cell or plant under conditions promoting ALA plus LA level that is less than the level of C18, C20 and C22 PUFAs and/or a conversion rate of a d6des increased relative to control plants.

According to the method of the invention the method for example comprises the following steps:

(i) replacing in a plant cell or plant a regulatory element controlling the expression of the polypeptide as defined in claim 29 or of a nucleic acid molecule encoding the polypeptide by a replacement regulatory element that increased the expression of the polypeptide as defined in claim 29 or of a nucleic acid molecule encoding the polypeptide; and

(ii) cultivating said plant cell or plant under conditions promoting an ALA plus LA level that is less than the level of C18, C20 and C22 PUFAs and/or a conversion rate of a d6desaturse that is increased relative to the control.

Accordingly, the present invention also relates to a transgenic plant, or part thereof, or plant cell, or plant seed obtainable by a method of the present invention. For example, the transgenic plant, or part thereof, or plant cell, or plant seed or plant oil has increased amount of GLA, HGLA, SDA and/or ETA, even more preferred of total PUFA, in the plant, plant cell, or part seed, or part thereof, cable to produce GLA, and/or an increased conversion rate of a phospholipid-dependent desaturase relative to control or parent plants, resulting from the increased activity of the PDCT1 as used in the method of the invention, preferably resulting from the increased expression, of a nucleic acid encoding a PDCT of the invention. The transgenic plant, or part thereof, or plant cell, or plant seed of the invention is for example a transgenic plant, or part thereof, or plant cell, or plant seed that comprises the expression construct of the invention and e.g. is oil crop seed plant, for example a Camelina seed or a Brassica sp seed, or as described herein.

A transgenic plant, or part thereof, or plant cell, or plant seed obtainable by a method according to the present invention, wherein said plant, plant part or plant cell comprises a recombinant nucleic acid encoding a PDCT polypeptide as described for the use the method of the present invention, the polynucleiotid or nucleic acid molecule of the present invention, the polypeptide of the present invention, the vector of the present invention, the expression construct of the present invention, or a replacement regulatory element controlling the expression of the polypeptide as for use in themethod of the present invention, e.g. as the polynucleotide of the present invention or of a nucleic acid molecule encoding the polypeptide.

The present invention also relates to a plant, plant cell, plant seed, or part thereof, for example a oil seed corp seed or cell, or a plant oil, for example a raw oil obtained from or comprised inplant, plant seed, plant cell or part thereof, that comprises C18 to C22 fatty acids, wherein the ALA and LA level is less than the level of the C18 to C22 fatty acids.

Thus, the present invention relates to a plant, or part thereof, a plant seed, a plant cell, or plant oil, wherein the ALA and LA level is preferably less than the level of SDA; ETA, GLA, HGLA, EPA, DHA, and DPA.

Further, the invention relates to a plant, plant part or plant cell transformed with a recombinant nucleic acid encoding a PDCT polypeptide of the invention, a polynucleotide of the invention, a nucleic acid construct of the invention or a vector of the invention or a replacement element controlling the expression the polypeptide of the invention or of a nucleic acid molecule encoding the polypeptide of the invention. For example, the transgenic plant of the invention, or a transgenic plant cell derived therefrom, is an oil crop plant, preferably a Brassica napus, Brassica juncea, Brassica carrinata or Camelina sativa plant

Further, the invention relates A harvestable part of a plant of the invention, for example said harvestable parts are seeds.

Further, the present invention relates to a transgenic pollen grain or any other germ cell/haploid derivate of a cell comprising a recombinant nucleic acid encoding a PDCT polypeptide of the invention, a polynucleotide of the invention, a nucleic acid construct of the invention or a vector of the invention.

Also, the present invention relates to a protein preparation comprising the polypeptide of of the invention, wherein the protein preparation comprises a lyophilized composition/formulation and/or additional enzymes or compounds.

Further, the present invention relates to a raw oil from a B. species or C. species that comprises a reduced ALA level.

Further the present invention relates to a raw oil from a B. species or a C. species that has a ALA plus LA level that is less than the level of C18, C20 and C22 PUFAs.

For example, the raw oil is a seed oil. For example, the raw oil is obtained from the seed or plant of the present invention and is not further processed. The minimum steps for obtaining a raw oil include obtaining seeds and crushing, solvent extracting, or using other physical means (e.g. centrifugation) to separate the oil from the remaining solids (i.e. meal).

Further, the present invention relates to an antibody or a fragment of an antibody specifically binding to the polypeptide of of the invention or a fragment thereof having PDCT1 activity.

Further, the present invention relates to a product derived or produced from a harvestable part of a plant, preferably from the seed of the plant, wherein

the plant comprises a recombinant nucleic acid encoding a PDCT polypeptide of the invention, a polynucleotide of the invention, a nucleic acid construct of the invention or a vector of of the invention or the polypeptide of the invention or is produced according to the method of the invention; or

the product of (a), wherein the product is a dry pellet, a pulp pellet, a pressed stem, a meal, a powder, or a fiber, containing a composition produced from the plant; or

the product of (a), wherein the product comprises an oil, a fat, a fatty acid, a carbohydrate, or a starch, a sap, a juice, a molasses, a syrup, a chaff, or a protein produced from the plant.

Further, the present invention relates to a method of expressing a polynucleotide of the invention, comprising:

(a) providing a host cell comprising a heterologous nucleic acid construct of any of the invention by introducing the nucleic acid construct into the host cell;

(b) cultivating the recombinant host cell of step (a) under conditions conductive for the expression of the polynucleotide; and

(c) optionally, recovering a protein of interest encoded by the polynucleotide.

Also, the present invention describes the use of a PDCT polypeptide of the invention, a polynucleotide of the invention, a nucleic acid construct of the invention or a vector of the invention or the polypeptide of the invention or the polypeptide produced the method of the invention or the method of the invention for producing a plant, cell, seed, seed oil or plant oil comprising EPA, DHA and EPA and having an ALA plus LA level that is less than the level of C18, C20 and C22 PUFAs.

Further, the present invention A meal comprising EPA, DHA and EPA and having an ALA plus LA level that is less than the level of C18, C20 and C22 PUFAs

Preferably, the level of ALA+LA is the plant, seed, oil or meal is 10%, 20%, 30%, 40%, or 50% or more less than the level of total PUFA.

Also, the present invention relates to a feed or food product comprising the plant oil of the invention or a meal produced from the seed of the invention.

Further, the present invention relates to a feed or food composition of the present invention or the product of method of the present invention, comprising no oil derived from animals. Preferably, the feed or food composition does not comprise any fish oil or fats.

Thus, the method of the present invention for example a plant, plant seed, plant raw oil, plant seed oil, plant cell, meal, wherein the level DPA, DHA and/or EPA level is increased.

### FIGURES

FIG. 1 Alignment of PDCT protein sequences

Legend: At: Arabidopsis thaliana, Bn: Brassica napus, Bc: Brassica carinata, Cs: Camelina sativa, Gm: Glycine max, Lu: Linum usitatissimum, Rc: Ricinus communis, Ta: Triticum aestivum, Zm: Zea mays.

* activity demonstrated in other studies

** proteins selected based on homology in BLAST searches of NCBI databases, activity not demonstrated

Color setup: Non-similar, weakly similar: dark grey, conserved: light grey, blocks of similar: medium grey, identical: white

FIG. 2 Alignment of N-terminal region of C. sativa sequences. All differences in the C. satvia proteins are within this region

Color setup: Non-similar, weakly similar: dark grey, conserved: light grey, blocks of similar: medium grey, identical: white

FIG. 3 Phylogenetic tree based on PDCT protein sequences.

Legend: At: Arabidopsis thaliana, Bn: Brassica napus, Bc: Brassica carinata, Cs: Camelina sativa, Gm: Glycine max, Lu: Linum usitatissimum, Rc: Ricinus communis, Ta: Triticum aestivum, Zm: Zea mays.

* activity demonstrated in other studies

** proteins selected based on homology in BLAST searches of NCBI databases, activity not demonstrated

FIG. 4. Pathway and genes in fatty acid synthesis pathway in transgenic Arabidopsis plants.

FIG. 5. Action of PDCT (Modified from Lu et al., 2009)

FIG. 6: Phylogenetic tree based on PDCT protein sequences of Table 5

FIG. 7 decribes the formulas to calculate pathway step conversion efficiencies. S: substrate of pathway step.

P: product of pathway step. Product was always the sum of the immediate product of the conversion at this pathway step, and all downstream products that passed this pathway step in order to be formed. E.g. DHA (22:6n-3 does possess a double bond that was a result of the delta-12-desaturation of oleic acid (18:1n-9) to linoleic acid (18:2n-6).

FIG. 8:

Needle Matrix of PCDT sequences of table 5

FIG. 9:

Conversion rate efficiencies of desaturases.

### EXAMPLES

### Example 1

### Materials and Methods

Cloning of Genes:

RNA from young root tissue of B.napus, B. carinata and C. sativa was reversed transcribed using Superscript III. Primers for cloning cDNAs were based on genomic sequence information from NCBI sequence databases (https://www.ncbi.nlm.nih.gov/) and naming of genes followed the information in these databases. The proofreading enzyme Phusion was used to clone cDNAs, which were transformed into pYes 2.1 prior to sequencing. Seven PDCT like genes were cloned from B. napus, originating from chromosome 1A, 1C, 2C, 3A, 3C, 5A and 5C. Seven genes were cloned from B. carinata, originating from chromosomes 1B, 1C, 2B, 3B,3C, 5B and 5C. Three genes were cloned from C. sativa, originating from chromosomes 1, 15 and 19. Sequences of cDNAs and translation products are given in Table 1.

Sequence Analysis:

All clones were sequenced prior to transformation. The protein alignment and phylogenetic tree were constructed using the software program Vector NTI.

Construction of transformation vectors and Arabidopsis transformation:

Because the C genome genes from B. carinata and B. napus were identical or nearly identical, only C subgenome derived PDCT genes from B. carinata were used in further experiments. PDCT genes were cloned into the pUC-19 Napin-B vector to add the Napin promotor and OSC terminator, as described in Wu et al (2005). The genes including promotors and terminators were removed by restriction enzyme digestion and ligated to pUC19-ABC carrying the Thraustocytrium sp. delta 6 elongase (Sequence ID: KH273553.1) and the P. irregulare delta 6 desaturase (Sequence ID: AF419296.1). The three genes were removed from the vector by restriction enzyme digestion and ligated into the plant binary vector pSUN2-ASC. All vectors were analyzed by restriction digestion before transformation. Controls included an empty vector and a vector containing only the P. irregulare D6 desaturase and the PSE (tc) elongase. The Arabidopsis rod1 (At3g15820) mutant line (Lu et al. 2009), kindly provided by Chaofu Lu, was used as the Arabidopsis host plant. This mutant has a G to A mutation resulting in a premature stop codon in the phosphatidylcholine:diacylglycerol cholinephosphotransferase (PDCT) enzyme encoded by the Arabidopsis ROD1 gene (Lu et al. 2009). Four plants were tested by sequencing, which indicated all were homozygous for the relevant mutation, and seed was collected from these plants and used for transformation. Plant binary vectors were transformed into Agrobacterium tumefaciens strain GV3101-pMP90. The host plant was grown until the bolting stage and transformed using the floral dip method (Clough and Bent, 1998). Essentially, Agrobacterium tumefaciens carrying each vector was grown to mid-log stage, spun down and suspended to an OD600 of 0.8 in 5% sucrose solution containing 0.05% Silwet L-77, and plants were immersed in this solution for 2-3 minutes with gentle agitation. After maturity, seeds were sterilized and germinated on ½X MS selective medium containing 50 mg/L kanamycin for selection of transgenic plants. Positive plants were transplanted into soil and grown to maturity.

GC Analysis:

Twenty T2 seeds from positive T1 plants were used to extract fatty acids. Seeds were placed in a clean glass tube, 2 mL of 3M methanolic HCL was added to each tube, and capped tubes were incubated at 80° C. for 4 hours. After incubation, samples were cooled to room temperature, 1 mL of 0.9% NaCl and 2 mL of hexane was then added to each sample and vortexed. Samples were then centrifuged and the hexane (top) layer was removed and added to clean glass tubes. Samples were evaporated under nitrogen until dry. 80 μL of hexane was added to the tubes and vortexed briefly to resuspend the fatty acids. The solution was then moved to a collection vial containing a GC insert, and GC analysis was performed (Table 2).

The segregation of the transgene was tested by germinating 50-100 seeds on selective media, and testing the fit to a 3:1 hypothesis (Table 3). Seedling progeny of transgenic plants that segregated with a 3:1 ratio (consistent with expression of construct at a single locus) were used for further analysis. GC analysis of 20 seeds from 3-5 lines for each gene was conducted as described above, and fatty acid distribution was determined (Table 4).

### Example 2

### Results

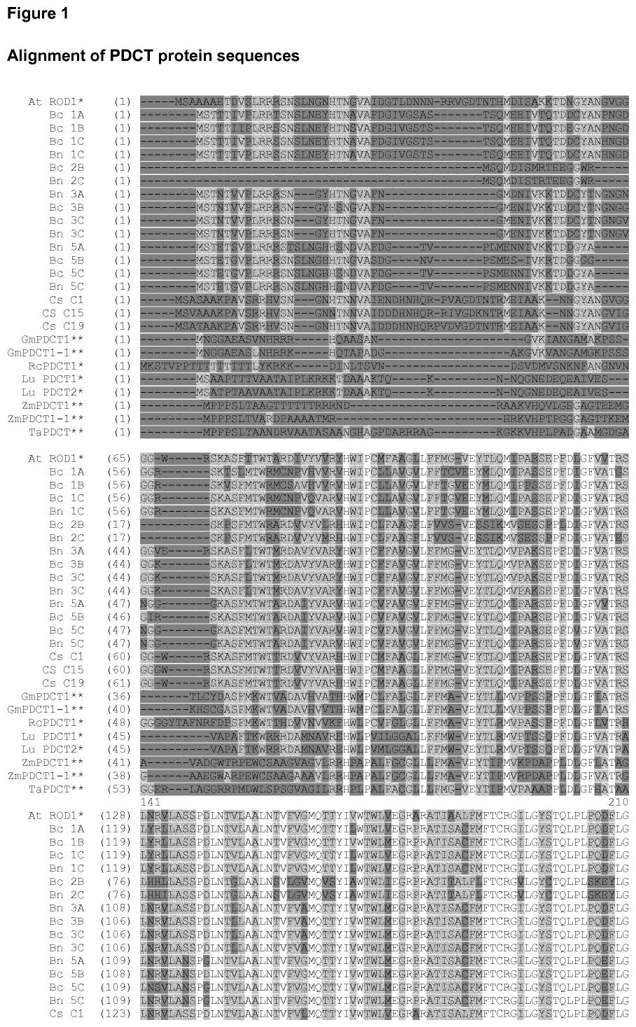

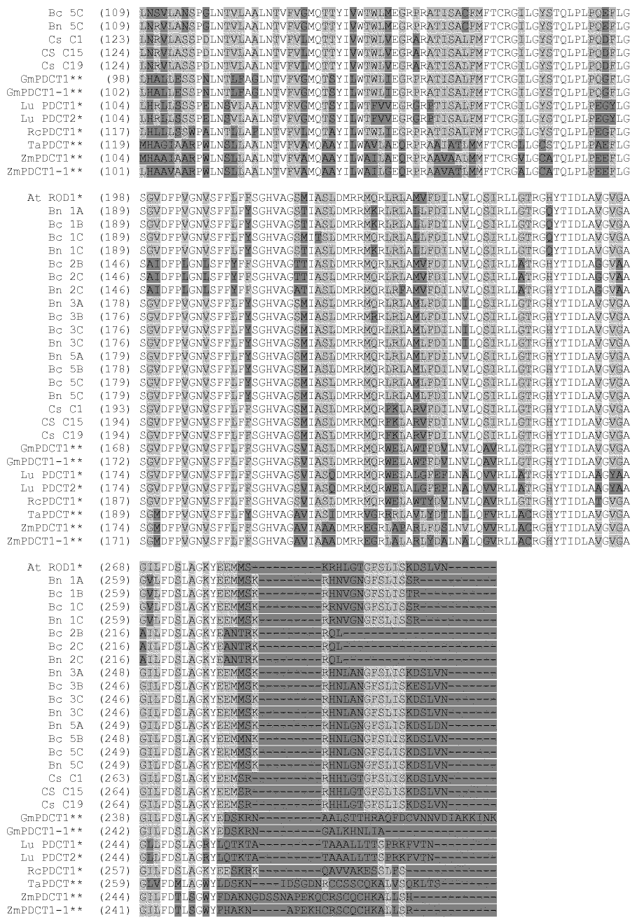

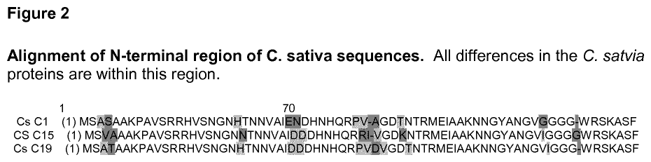

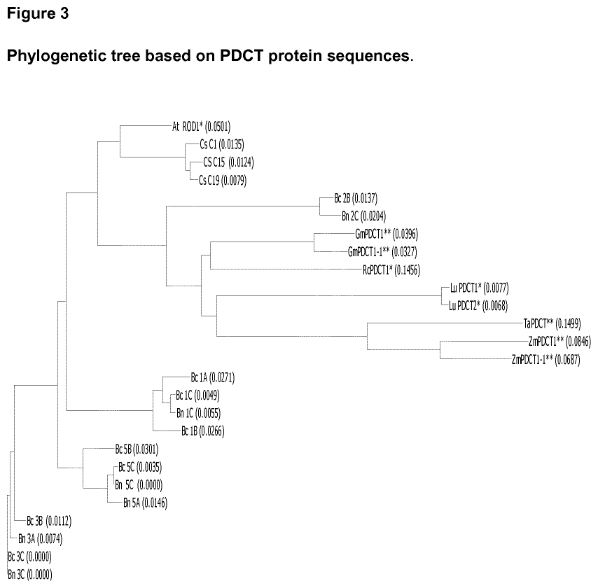

The amino acid sequences of the 19 PDCT genes cloned in this study fell in 5 distinct groups (FIGS. 1, 2 and 3). These groups consisted of the chromosome 1-derived sequences of B. napus and B. carinata, the chromosome 2 sequences of B. napus and B. carinata, the chromosome 3 sequences of B. carinata and B. napus, the chromosome 5 genes of B. napus and B. carinata and the three C. sativa sequences (FIG. 2). The amino acid translations of the C-subgenome derived genes of B. carinata and B. napus were identical or nearly identical, although there were differences in the cDNA sequences (FIG. 1, Table 1). Most of the differences in amino acid sequences occurred in the N-terminal region of the translation products, while blocks of conserved amino acids were found throughout the middle and C-terminal regions (FIG. 1). The Group 1 sequences were about 42 amino acids shorter than the other sequences in this area. The differences among the three C. sativa sequences occurred within the first 60 amino acids (FIG. 1, FIG. 2).

The four subgenome A PDCT genes from Brassica napus, the four subgenome B and four subgenome C genes from Brassica carinata, and all three PDCT genes from Camelina sativa were co-expressed in the Arabidopsis rod1 mutant with the Δ6-desaturase from Pythium irregulare and the Δ6-elonagase from Thraustochytrium. The Arabidopsis rod1 mutant and a wild-type Arabidopsis line (with an active endogenous PDCT gene) were also transformed with the Δ9-desaturase from Pythium irregulare and the Δ6-elonagase from Thraustochytrium, and untransformed wild-type and ROD mutant lines were used for comparison.

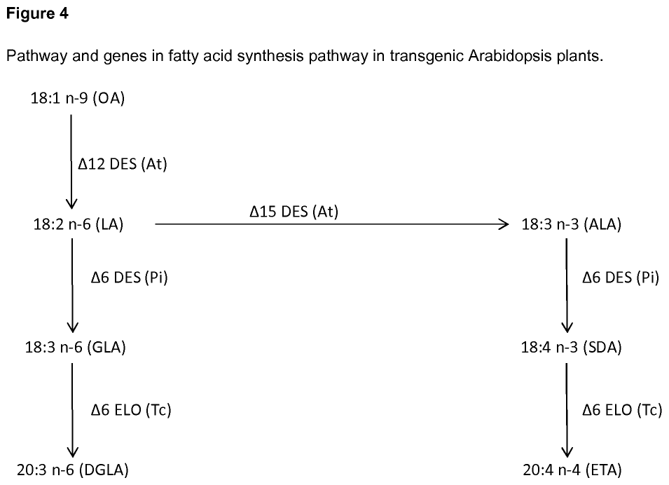

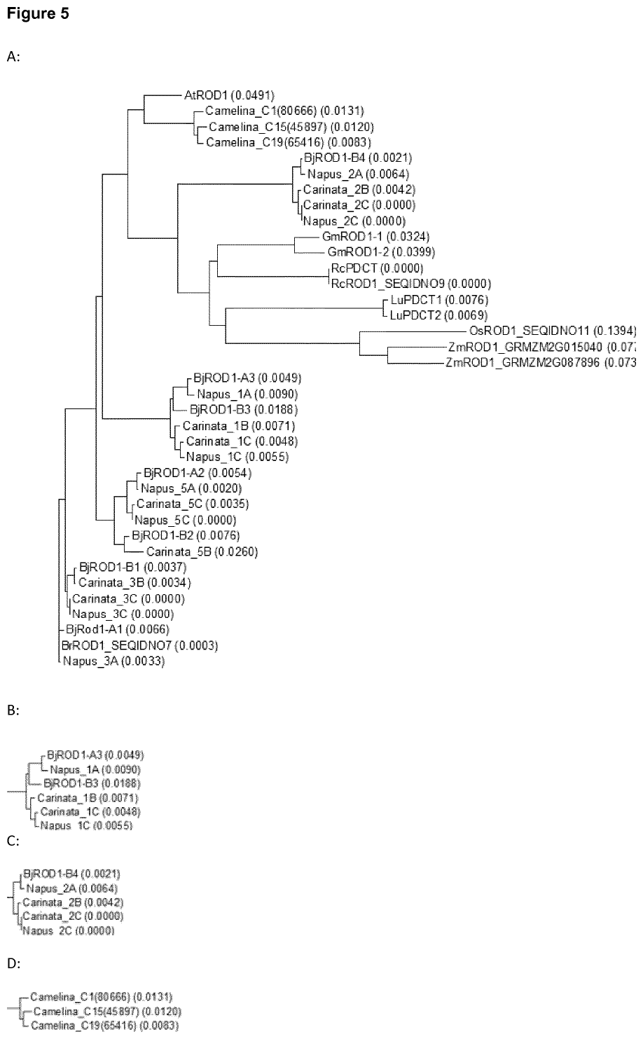

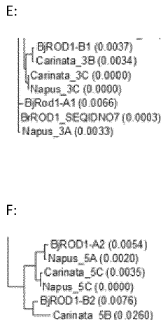

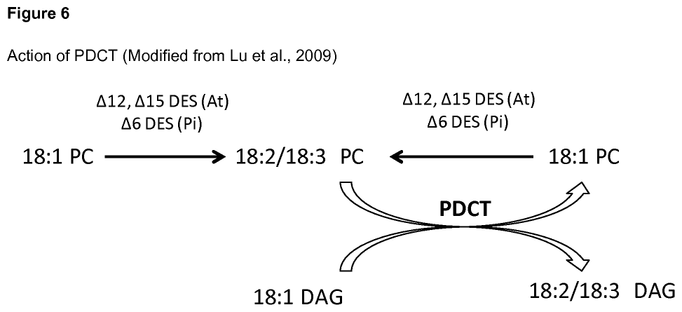

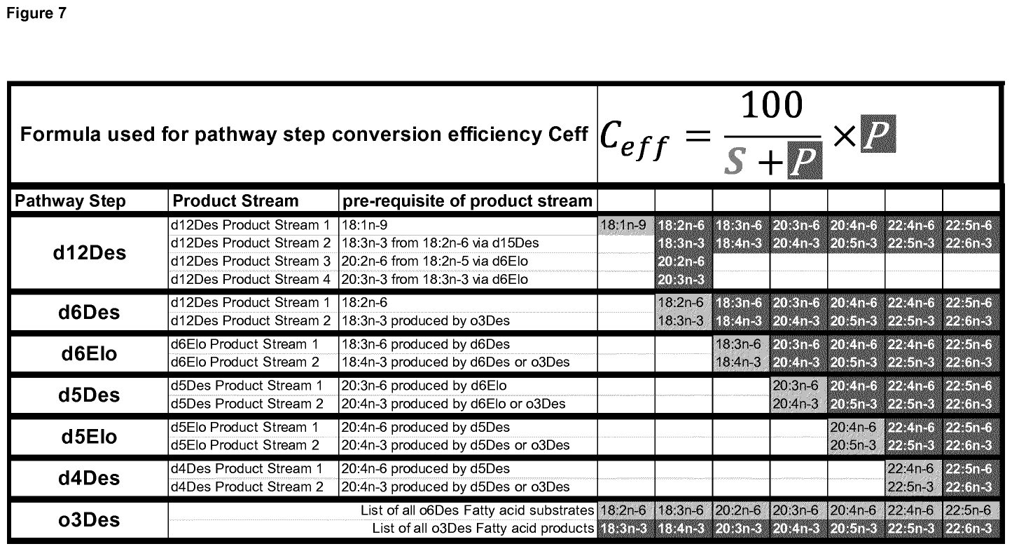

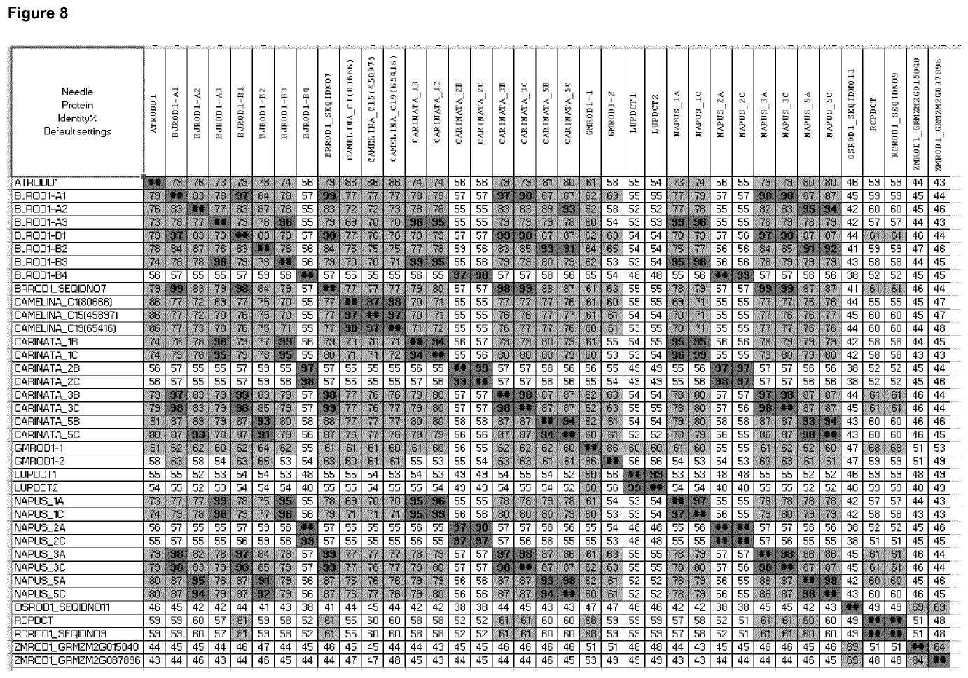

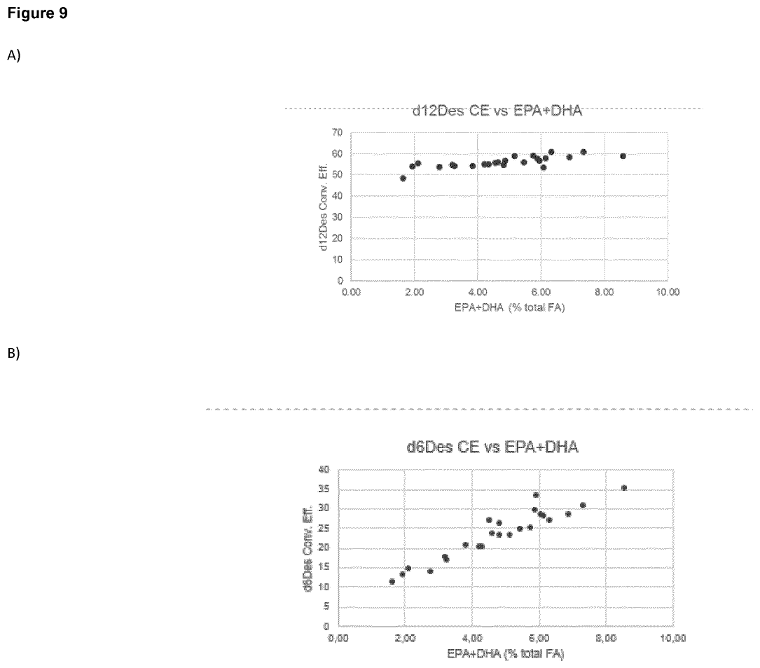

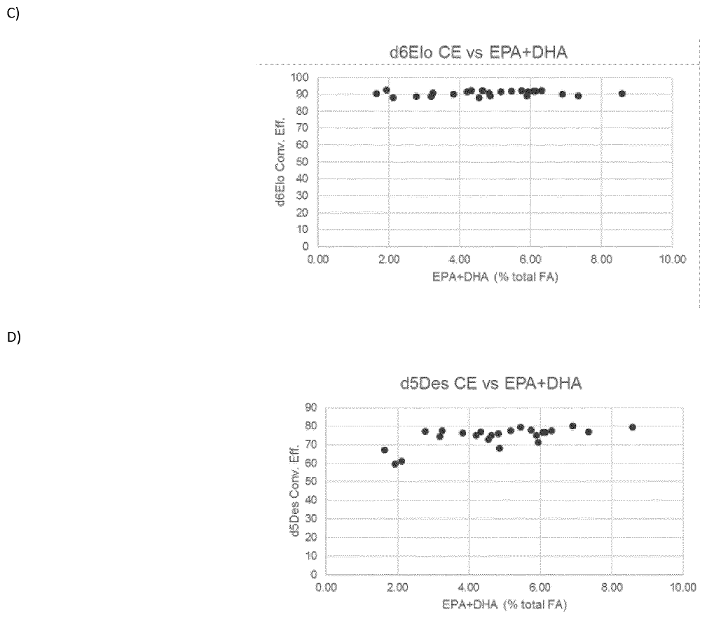

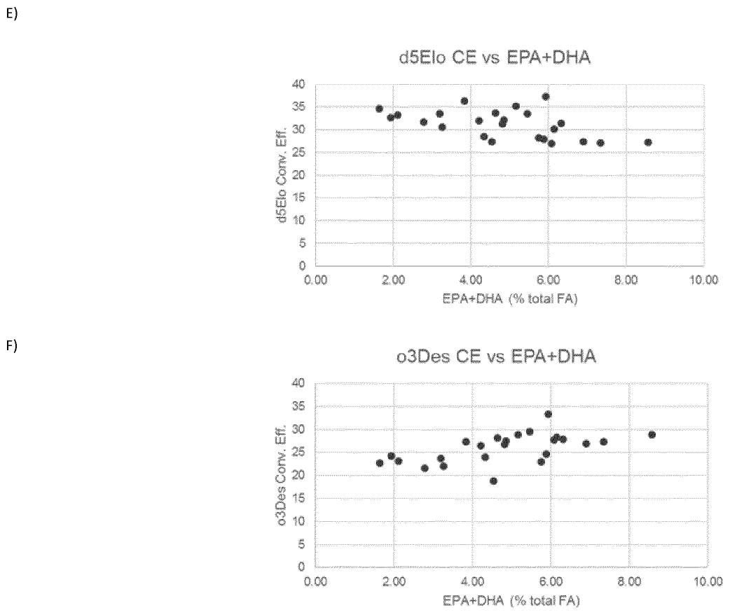

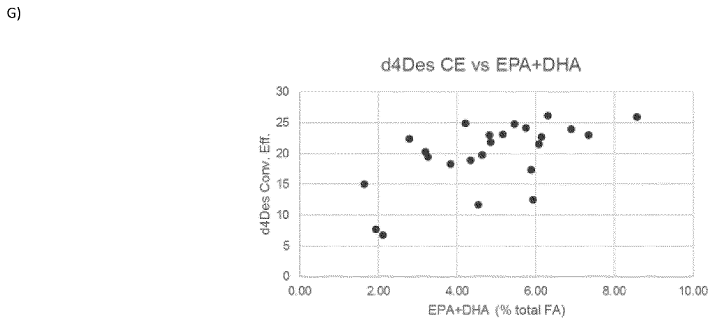

Expression of the Δ6-desaturase and Δ6-elonagase will result in the production of the heterologous fatty acids γ-linolenic acid (GLA ; 18:2 Δ11, 14), stearidonic acid (SDA ; 18:3 Δ6, 9, 12, 15), di-homo γ-linolenic acid (DGLA; 20:3 Δ8, 11, 14) and eicosatetraenoic acid (ETA; 20:4 Δ8.11, 14.17) in Arabidopsis seeds, as shown in FIG. 4. An active PDCT gene will lead to a decrease in the level of OA (18:1 Δ9) and an increase in the level(s) of LA (18:2Δ6, 9), ALA (18:3Δ6, 9, 15) and/or GLA, as shown in FIG. 5.

The presence of a mutation in the ROD1 gene of Arabidopsis has been shown to increase the percent of 18:1 in seed oil (Lu et al., 2009). The percentage of 18:1 in the untransformed rod1 mutant used in this study averaged 30.42%, while seed oil of the untransformed wild-type line contained 15.334% 18:1. Seed oil from Arabidopsis lines carrying group 1 and group 2 chromosome-derived PDCT genes had average 18:1 levels ranging from 25.72-31.12% (Table 2). This was comparable to the level in the ROD mutant lines transformed with only the Δ6-desaturase and Δ6-elonagase (average 30.732%). However, the levels in seeds carrying the subgenome 3A, 3B and 3C derived genes ranged from 14.959-15.871%. Levels in seeds carrying chromosome 5 derived PDCT genes ranged from 11.994-16.696%, and those in seeds carrying the C. sativa genes ranged from 13.288-14.050%. Thus, while the Brassica napus chromosome 3 and chromosome 5 derived genes, and the three C. sativa genes are able to compensate for the mutation in the Arabidopsis PDCT gene, the chromosome 1 and 2 derived genes appear to have little or no effect on 18:1 levels. This suggests that the chromosome 1 and 2 derived genes may have a different function and/or act on different substrates than the Arabidopsis PDCT gene.

Alignment of PDCT-like translation products from a range of species including Triticum aestivum, Arabidopsis thaliana, Zea mays, Ricinus communis, Glycine max, and Linum usitatissimum indicated that substitutions of highly conserved amino acids occurred throughout the B. napus chromosome 1 and chromosome 2 derived proteins. Using numbering based on the Arabidpsis ROD1 sequence as shown in the alignment in FIG. 1, Brassica napus chromosome-1 derived enzymes showed the following changes in conserved regions: position 102: M to T, between 104-105: insertion of E, and 225: H to Q. In addition to these changes in conserved regions, various differences occurred in the less conserved N-terminal region of the protein.

In the case of chromosome 2B and 2C derived proteins from Brassica carinata and Brassica napus respectively, a larger number of substitutions in conserved regions were detected. Using amino acid residue numbering based on the Arabidopsis ROD1 sequence, the following substitutions were detected 98: V/L to F, 101 F to V, 102 M to V, 106: Y to S, 141: L/V to G, 149-150: FV to LG, 158: L/V to A, 176: M to V, 186: S/A to C, 192: P to S, 211: L to Y, and 230: M/V to T. Notably, this threonine substitution at position 230 also occurred in most of the chromosome 1 group proteins, as did the M to T substitution at position 106.

In the untransformed Arabidopsis wild-type lines the decrease in 18:1 is compensated for by an increase in 18:2 compared to rod1 mutant plants (27. 545% in wild-type versus 14.323% in ROD mutant; Table 2) although a slight increase in ALA also occurs (16.066 versus 14.323%). Transgenic lines carrying the elongase and desaturase genes plus chromosome—1 or 2 PDCT genes had LA levels of 8.314-12.165%, while lines carrying chromosome 3 and 5 derived PDCT genes had levels of 18.149-20.142%. The lines carrying the C. sativa genes had 18:2 levels of 11.324% (Chromosome 1 derived PDCT), 19.912% (C15) and 8.635% (C19). ALA levels were also comparatively low in lines carrying the genes had the highest average ALA content (14.826%). However, in lines carrying the Δ6-desaturase and the Δ6-elonagase along with the PDCT gene, the additional 18:2 produced in the presence of the PDCT gene may be used not only to produce ALA, but may also be used in the synthesis of GLA, DGLA, SDA and ETA (FIG. 4). The total levels of these fatty acids were highest in lines carrying the C1 (25.225%) and C19 (24.379%) PDCT genes, and these two lines also had the highest levels of GLA plus HGLA (22.183% and 21.094% respectively). The fatty acid profile of lines carrying the C. sativa C15 gene bore more of a resemblance to the group 5 and group 3 chromosomes, in that the total ALA plus SDA plus ETA (16%) was considerably higher than the total GLA plus HGLA (8.767%). Only in the C1 and C19 lines were total levels of GLA plus HGLA higher than total levels of ALA plus SDA plus ETA (Table 2). Thus, not only do the various PDCTs show differences in overall efficiency, but there also appears to be different substrate preferences among the genes. The Camelina sativa C1 and C19 proteins differed from the C15 protein in only a limited number of amino acids in the N-terminal region of the protein (FIG. 2). Position 3 was valine in C15 and alanine in C1 and C19. Position 4 was alanine in C15, whereas the similar amino acid residues serine and threonine were at position 4 in C1 and C19 respectively. A conserved histidine at position 20 in C1 and C19 was replaced by asparagine in C15, proline-valine residues at positions 35 to 36 in C1 and C19 were replaced with arginine-isoleucine in C15, and a threonine at position 41 was replaced with lysine in C15. Finally, C15 had an insertion of an amino acid (glycine) at position 63. These differences indicated the importance of the N-terminal region of the PDCT enzyme in determining enzyme activity.

Potentially, inactivation of one or more Camelina sativa PDCT enzyme may modulate PDCT activity levels, and might also be beneficial in increasing the levels of specific fatty acids, or in pushing fatty acids towards the ω3 or ω6 pathway. Since B. napus and B. carinata each have four active PDCT genes, it should be possible to achieve a range in PDCT activity levels by combining active and inactive genes. Avoiding rapid transfer onto DAG may allow more efficient transfer to the acyl-CoA pool by the reverse reaction of plant LPCAT enzymes. The reverse reaction of LPCAT has been shown to play an important role in editing PC in plants, and plant LPCATs also show fatty acid selectivity (Lager et al., 2013) This may be of particular interest for the production of VLC-PUFAs, where rapid movement of fatty acids to the DAG pool and subsequently to TAG may not be desirable.

To ensure the differences in activities among the transgenic lines did not reflect differences in copy numbers of PDCT genes, the segregation ratio of T2 plants was checked (Table 3), and T3 seed from lines that fit a 3:1 segregation ratio was used for GC analysis. Results closely resembled those from the T2 generation (Table 4). 18:1 levels in lines carrying chromosome group 1 or 2 derived PDCT genes ranged from 31.26-31.41%, while levels in group 3 and 5 lines ranged from 12.17-14.59%. Levels in lines carrying the C. sativa genes ranged from 12.89 to 14.60%. LA levels in lines carrying group 1 and 2 chromosome genes ranged from 6.58-10.06%, while levels in the group of lines carrying chromosome 3 or 5 derived genes ranged from 15.58-23.54%. Levels in lines carrying C1, C15 and C19 PDCT genes were 11.53, 21.49 and 7.50%, respectively. Again, the low level of LA in C1 and C19 lines was due to the very high levels of GLA plus DGLA in these lines (20.85% in C1 and 23.11% in C19).

### Example 3

### Average Fatty Acid Composition (%) in Different Lipid Classes from Immature Seeds

Thin-layer chromatography (TLC) analysis was performed on immature siliques (from plants homozygous for the desaturase and elongase transgenes) to measure the fatty acid profile in different lipid pools, namely, phosphatyidylcholine (PC), diacylglycerol (DAG), and triacylglycerol (TAG). Briefly, total lipids were extracted from immature siliques by rapid freezing and grinding of green siliques, followed by transferring approximately 500 mg of ground sample into a centrifuge tube with 3 ml of chloroform: methanol: formic acid (10:10:1, v/v/v) and storing overnight at −20° C. After centrifugation, the supernatant was collected, and the pellet was re-extracted with 1.1 ml chloroform: methanol: water (5:5:1, v/v/v). The extractions were combined and washed with 1.5 ml mL 0.2M H3PO4/1M KCl. Lipids in the chloroform phase were dried down, and re-dissolved in 0.2 ml of chloroform. After pre-running and drying the TLC plate, samples were run in hexane/diethyl ether/acetic acid (70:30:1). TAG and DAG were isolated and directly methylated with 3M methanolic HCL. Polar lipids were collected from the plate, extracted and resuspended in chloroform, then re-run in chloroform/methanol/acetic acid/water (60:30:3:1) to separate PC. Bands were visualized by spraying with primulin solution and exposing to UV light. The appropriate silica bands were scraped from the TLC plate, and treated with 2 mL 3M methanolic HCL at 80° C., then analyzed by GC. All fatty acid data are presented as % relative and are shown in Table 7.

The data in Table 7 can be used to understand how the PDCT genes influence the trafficking of fatty acids between different lipid pools. Table 6 shows the average fatty acid composition (%) in different lipid classes from immature seeds of Arabidopsis transformed with D6(Pi) desaturase+ Tc D6Elongase.

The Arabidopsis rod1 mutant (CK mutant) and a wild-type Arabidopsis line (CK WT) (with an active endogenous PDCT gene) were also transformed with the Δ6-desaturase from Pythium irregulare and the Δ6-elonagase from Thraustochytrium, and untransformed wild-type (WT) and ROD mutant lines (Rod mut) were used for comparison.

TABLE 1
Sequenzes:

| | |
|---|---|
| PDCT1 Polypeptide: | SEQ ID No.: 2, 4, 6, 8, 10, 12, 14, 16, 40, 42, 44, and/or 46 |
| PDCT1 Polynucleotide: | SEQ ID No.: 1, 3, 5, 7, 9, 11, 13, 15, 39, 41, 43, and/or 45 |
| PDCT19 Polypeptide: | SEQ ID No.: 36, 38, and/or 48 |
| PDCT19 Polynucleotide: | SEQ ID No.: 35, 37, and/or 47 |
| PDCT3/5 Polypeptide: | SEQ ID No.: 18, 20, 22, 24, 26, 28, 30, 32, 50, 52, 54, 56, 58, |
| | and/or 60 |
| PDCT19 Polynucleotide: | SEQ ID No.: 17, 19, 21, 23, 27, 29, 31, 49, 51, 53, 55, and/or 57 |

Candiates of the PDCT1 that shall have the same activity as PDCT1:

| | | | |
|---|---|---|---|
| GmROD1-1 | 63 | 64 | PDCT1 candiate |
| GmROD1-2 | 65 | 66 | PDCT1 candiate |
| RcPDCT | 67 | 68 | PDCT1 candiate |
| RcROD1_SEQIDNO9 | 69 | 70 | PDCT1 candiate |
| LuPDCT1 | 71 | 72 | PDCT1 candiate |
| LuPDCT2 | 73 | 74 | PDCT1 candiate |

TABLE 2
Average fatty acid composition (%) in seeds of PDCT + D6(Pi) desaturase + Tc
D6Elongase transgenic T2 Arabidopsis.

| | | | | | | | | | | | Total | | |
|---|---|---|---|---|---|---|---|---|---|---|---|---|---|
| | | | | | | | | | | | GLA | Total | |
| | | | | | | | | | | | HGLA | ALA | Total |
| | | | | | | | | | | | SDA | SDA | GLA |
| | 16:0 | 18:0 | 18:1 | 18:2 | GLA | 18:3 | 20:1 | HGLA | SDA | ETA | ETA | ETA | HGLA |
| Napus | 9.063 | 3.290 | 26.438 | 12.165 | 2.205 | 9.916 | 15.452 | 5.634 | 0.231 | 0.995 | 9.065 | 11.142 | 7.893 |
| 1A | | | | | | | | | | | | | |
| Carinata | 8.254 | 3.398 | 29.693 | 11.760 | 2.402 | 10.656 | 17.882 | 1.950 | 0.689 | 0.369 | 5.410 | 11.714 | 4.352 |
| 1C | | | | | | | | | | | | | |
| Carinata | 7.950 | 3.203 | 30.947 | 11.418 | 3.083 | 11.154 | 18.672 | 1.639 | 0.742 | 0.253 | 5.717 | 12.149 | 4.722 |
| 2B | | | | | | | | | | | | | |
| Napus | 8.045 | 3.239 | 29.547 | 11.834 | 3.903 | 10.586 | 17.472 | 1.703 | 0.898 | 0.237 | 6.741 | 11.721 | 5.606 |
| 2C | | | | | | | | | | | | | |
| Napus | 7.915 | 3.004 | 15.871 | 18.613 | 7.984 | 12.877 | 17.168 | 1.827 | 1.226 | 0.090 | 11.127 | 14.193 | 9.811 |
| 3A | | | | | | | | | | | | | |
| Carinata | 7.756 | 3.027 | 15.287 | 18.974 | 7.977 | 13.180 | 17.220 | 1.777 | 1.282 | 0.053 | 11.089 | 14.515 | 9.755 |
| 3B | | | | | | | | | | | | | |
| Carinata | 7.846 | 3.495 | 14.959 | 17.639 | 8.662 | 13.744 | 18.638 | 2.096 | 1.374 | 0.215 | 12.347 | 15.333 | 10.758 |
| 3C | | | | | | | | | | | | | |
| Napus | 7.606 | 3.286 | 16.696 | 18.657 | 6.467 | 14.627 | 18.890 | 1.192 | 1.092 | 0.065 | 8.816 | 15.784 | 7.659 |
| 5A | | | | | | | | | | | | | |
| Carinata | 8.031 | 3.244 | 15.025 | 18.149 | 9.193 | 12.762 | 16.790 | 2.481 | 1.493 | 0.155 | 13.322 | 14.410 | 11.674 |
| 5B | | | | | | | | | | | | | |
| Carinata | 8.429 | 2.905 | 11.994 | 20.812 | 9.901 | 11.717 | 15.036 | 2.453 | 1.653 | 0.102 | 13.821 | 13.184 | 12.354 |
| 5C | | | | | | | | | | | | | |
| C1 | 9.126 | 3.440 | 13.288 | 11.324 | 14.380 | 7.771 | 15.141 | 7.803 | 2.063 | 0.980 | 25.225 | 10.813 | 22.183 |
| (80666) | | | | | | | | | | | | | |
| C15 | 8.196 | 3.489 | 14.367 | 19.912 | 7.366 | 14.826 | 18.397 | 1.401 | 1.158 | 0.016 | 9.941 | 16.000 | 8.767 |
| (45897) | | | | | | | | | | | | | |
| C19 | 7.830 | 3.454 | 14.050 | 8.635 | 14.658 | 7.656 | 15.746 | 6.436 | 2.440 | 0.844 | 24.379 | 10.940 | 21.094 |
| (65416) | | | | | | | | | | | | | |
| CK | 8.936 | 3.290 | 30.732 | 11.866 | 3.172 | 11.105 | 17.449 | 1.905 | 0.602 | 0.198 | 5.877 | 11.905 | 5.077 |
| mutant | | | | | | | | | | | | | |
| CK WT | 7.684 | 3.345 | 12.754 | 22.068 | 7.527 | 13.765 | 18.000 | 1.906 | 0.968 | 0.143 | 10.544 | 14.876 | 9.433 |
| WT | 7.335 | 3.284 | 15.334 | 27.545 | 0.000 | 16.066 | 18.071 | 0.000 | 0.000 | 0.000 | 0.000 | 16.066 | 0.000 |
| ROD | 7.619 | 3.123 | 30.420 | 14.332 | 0.000 | 15.158 | 19.276 | 0.000 | 0.000 | 0.000 | 0.000 | 15.158 | 0.000 |
| mut | | | | | | | | | | | | | |

CK mutant: PDCT mutant with D6(Pi) desaturase + Tc D6Elongase
CK WT: WT Arabidopsis with D6(Pi) desaturase + Tc D6Elongase
WT: Untransformed wild-type Arabidopsis
ROD mut: Untransformed Arabidopsis ROD mutant
Complete data in Appendix 1.

TABLE 3
Segregation ratios of T2 generation to test goodness of fit to 3:1 ratio

| | Plant | Resistant | Susceptible | Hypothesis | | Accept |
|---|---|---|---|---|---|---|
| Group | # | plant | plant | Ratio | p value | hypothesis |
| B. napus 1A | 2 | 50 | 0 | 63:1 | 0.312 | Accept |
| | 4 | 12 | 9 | 3:1 | 0.04 | No |
| | 5 | 71 | 19 | 3:1 | 0.46 | Accept |
| | 6 | 61 | 11 | 3:1 | 0.06 | Accept |
| | 7 | 20 | 45 | 3:1 | 0.249 | Accept |
| | 8 | 40 | 13 | 3:1 | 1 | Accept |
| | 9 | 41 | 26 | 3:1 | 0.012 | No |
| | 10 | 38 | 16 | 3:1 | 0.34 | Accept |
| | 11 | 40 | 16 | 3:1 | 0.537 | Accept |
| | 12 | 65 | 20 | 3:1 | 0.801 | Accept |
| | 13 | 67 | 18 | 3:1 | 0.451 | Accept |
| | 14 | 32 | 15 | 3:1 | 0.316 | Accept |
| | 15 | 50 | 9 | 3:1 | 0.073 | Accept |
| | 16 | 103 | 23 | 3:1 | 0.1 | Accept |
| | 17 | 54 | 19 | 3:1 | 0.786 | Accept |
| | 18 | 35 | 64 | 1:3 | 0.021 | No |
| | 19 | 54 | 18 | 3:1 | 1 | Accept |
| | 20 | 74 | 14 | 3:1 | 0.049 | No |
| | 21 | 22 | 8 | 3:1 | 1 | Accept |
| | 22 | 83 | 23 | 3:1 | 0.498 | Accept |
| | 23 | 52 | 17 | 3:1 | 1 | Accept |
| | 24 | 73 | 16 | 3:1 | 0.14 | Accept |
| | | | | | | Accept |
| | Plant | Resistant | Susceptible | Hypothesis | | hypothesis |
| Group | # | plant | plant | Ratio | p value | or not |
| B. carinata 2B | 1 | 72 | 20 | 3:1 | 0.47 | Accept |
| | 2 | 59 | 19 | 3:1 | 1 | Accept |
| | 3 | 73 | 23 | 3:1 | 0.814 | Accept |
| | 4 | 45 | 15 | 3:1 | 1 | Accept |
| | 5 | 99 | 5 | 15:1 | 0.674 | Accept |
| | 6 | 75 | 9 | 15:1 | 0.065 | Accept |
| | 7 | 103 | 11 | 15:1 | 0.119 | Accept |
| | 8 | 107 | 16 | 3:1 | 0.001 | No |
| | 9 | 98 | 12 | 15:1 | 0.051 | Accept |
| | 10 | 119 | 5 | 15:1 | 0.273 | Accept |
| | 12 | 50 | 0 | 63:1 | 0.312 | Accept |
| | 13 | 136 | 16 | 15:1 | 0.016 | No |
| | 14 | 113 | 19 | 3:1 | 0.005 | No |
| | 15 | 142 | 11 | 15:1 | 0.744 | Accept |
| | 16 | 50 | 5 | 15:1 | 0.235 | Accept |
| | 17 | 84 | 11 | 15:1 | 0.035 | No |
| | 18 | 88 | 29 | 3:1 | 1 | Accept |
| | 19 | 107 | 9 | 15:1 | 0.435 | Accept |
| | 20 | 105 | 10 | 15:1 | 0.242 | Accept |
| | 21 | 101 | 25 | 3:1 | 0.215 | Accept |
| | 22 | 76 | 3 | 15:1 | 0.355 | Accept |
| | 23 | 65 | 16 | 3:1 | 0.302 | Accept |
| | 24 | 51 | 21 | 3:1 | 0.414 | Accept |
| | 25 | 51 | 16 | 3:1 | 0.779 | Accept |
| B. napus 2C | 1 | 95 | 20 | 3:1 | 0.053 | Accept |
| | 2 | 55 | 17 | 3:1 | 0.785 | Accept |
| | 3 | 50 | 2 | 15:1 | 0.552 | Accept |
| | 4 | 120 | 12 | 15:1 | 0.145 | Accept |
| | 5 | 130 | 16 | 15:1 | 0.016 | No |
| | 6 | 160 | 14 | 15:1 | 0.35 | Accept |
| | 7 | 103 | 10 | 15:1 | 0.242 | Accept |
| | 8 | 60 | 19 | 3:1 | 0.796 | Accept |
| | 9 | 74 | 26 | 3:1 | 0.817 | Accept |
| | 10 | 58 | 25 | 3:1 | 0.313 | Accept |
| | 11 | 118 | 12 | 15:1 | 0.144 | Accept |
| | 12 | 98 | 2 | 63:1 | 1 | Accept |
| | 13 | 42 | 24 | 3:1 | 0.027 | No |
| | 14 | 71 | 1 | 63:1 | 1 | Accept |
| | 15 | 75 | 25 | 3:1 | 1 | Accept |
| | 16 | 38 | 29 | 3:1 | 0.001 | No |
| | 17 | 125 | 21 | 3:1 | 0.004 | No |
| | 18 | 143 | 35 | 3:1 | 0.118 | Accept |
| | 19 | 107 | 2 | 63:1 | 1 | Accept |
| | 20 | 81 | 23 | 3:1 | 0.497 | Accept |
| | 21 | 60 | 1 | 63:1 | 1 | Accept |
| | 22 | 92 | 1 | 63:1 | 1 | Accept |
| | Plant | Resistant | Susceptible | Hypothesis | | Accept |
| Group | # | plant | plant | Ratio | p value | hypothesis |
| B. napus 3A | 1 | 67 | 12 | 3:1 | 0.038 | No |
| | 2 | 125 | 39 | 3:1 | 0.718 | Accept |
| | 3 | 29 | 26 | 3:1 | 0 | No |
| | 4 | 92 | 21 | 3:1 | 0.127 | Accept |
| | 5 | 67 | 20 | 3:1 | 0.622 | Accept |
| | 6 | 43 | 19 | 3:1 | 0.342 | Accept |
| | 7 | 55 | 26 | 3:1 | 0.236 | Accept |
| | 8 | 70 | 9 | 15:1 | 0.065 | Accept |
| | 9 | 60 | 7 | 15:1 | 0.122 | Accept |
| | 10 | 63 | 5 | 15:1 | 0.606 | Accept |
| | 11 | 60 | 18 | 3:1 | 0.792 | Accept |
| | 12 | 68 | 22 | 3:1 | 1 | Accept |
| | 13 | 56 | 29 | 3:1 | 0.044 | No |
| | 14 | 69 | 22 | 3:1 | 0.809 | Accept |
| | 15 | 70 | 2 | 63:1 | 0.314 | Accept |
| | 16 | 41 | 13 | 3:1 | 1 | Accept |
| | 17 | 53 | 3 | 15:1 | 1 | Accept |
| | 18 | 47 | 16 | 3:1 | 1 | Accept |
| | 19 | 77 | 13 | 3:1 | 0.027 | No |
| | 20 | 78 | 6 | 15:1 | 0.645 | Accept |
| | 21 | 90 | 15 | 3:1 | 0.013 | No |
| | 22 | 47 | 11 | 3:1 | 0.357 | Accept |
| | 23 | 35 | 11 | 3:1 | 1 | Accept |
| | 24 | 61 | 20 | 3:1 | 1 | Accept |
| B. carinata 3B | 3B-1 | 76 | 7 | 15:1 | 0.3562 | Accept |
| | 3B-2 | 56 | 23 | 3:1 | 0.4376 | Accept |
| | 3B-3 | 24 | 28 | 3:1 | <0.0001 | Reject |
| | 3B-4 | 58 | 15 | 3:1 | 0.415 | Accept |
| | 3B-5 | 27 | 45 | 3:1 | <0.0001 | Reject |
| | 3B-6 | 142 | 37 | 3:1 | 0.168 | Accept |
| | 3B-7 | 85 | 31 | 3:1 | 0.668 | Accept |
| | 3B-8 | 87 | 21 | 3:1 | 0.182 | Accept |
| | 3B-9 | 75 | 24 | 3:1 | 0.817 | Accept |
| | 3B-10 | 97 | 11 | 15:1 | 0.118 | Accept |
| | 3B-11 | 52 | 13 | 3:1 | 0.388 | Accept |
| | 3B-12 | 43 | 18 | 3:1 | 0.372 | Accept |
| | 3B-13 | 75 | 29 | 3:1 | 0.497 | Accept |
| | 3B-14 | 63 | 3 | 15:1 | 0.606 | Accept |
| | 3B-15 | 42 | 16 | 3:1 | 0.539 | Accept |
| | 3B-16 | 70 | 4 | 15:1 | 0.643 | Accept |
| | 3B-17 | 68 | 2 | 63:1 | 0.314 | Accept |
| | 3B-18 | 56 | 23 | 3:1 | 0.438 | Accept |
| | 3B-19 | 59 | 5 | 15:1 | 0.606 | Accept |
| | 3B-20 | 71 | 2 | 63:1 | 0.314 | Accept |
| | 3B-21 | 56 | 2 | 63:1 | 0.313 | Accept |
| | 3B-22 | 58 | 22 | 3:1 | 0.606 | Accept |
| | 3B-23 | 59 | 19 | 3:1 | 1 | Accept |
| | 3B-24 | 65 | 30 | 3:1 | 0.157 | Accept |
| B. carinata 3C | 1 | 128 | 2 | 63:1 | 1 | Accept |
| | 2 | 96 | 17 | 3:1 | 0.017 | No |
| | 5 | 78 | 24 | 3:1 | 0.818 | Accept |
| | 6 | 76 | 8 | 15:1 | 0.167 | Accept |
| | 7 | 50 | 5 | 15:1 | 0.235 | Accept |
| | 8 | 91 | 15 | 3:1 | 0.013 | No |
| | 9 | 75 | 10 | 15:1 | 0.021 | No |
| | 10 | 95 | 17 | 3:1 | 0.016 | No |
| | 11 | 97 | 13 | 15:1 | 0.019 | No |
| | 12 | 38 | 12 | 3:1 | 1 | Accept |
| | 13 | 80 | 10 | 15:1 | 0.091 | Accept |
| | 14 | 42 | 6 | 15:1 | 0.074 | Accept |
| | 15 | 77 | 17 | 3:1 | 0.15 | Accept |
| | 16 | 70 | 5 | 15:1 | 1 | Accept |
| | 17 | 120 | 1 | 63:1 | 0.476 | Accept |
| | 18 | 79 | 24 | 3:1 | 0.65 | Accept |
| | 19 | 61 | 15 | 3:1 | 0.289 | Accept |
| | 20 | 94 | 20 | 3:1 | 0.082 | Accept |
| | 21 | 59 | 13 | 3:1 | 0.174 | Accept |
| | 22 | 109 | 15 | 15:1 | 0.011 | No |
| | 23 | 49 | 19 | 3:1 | 0.575 | Accept |
| | 25 | 53 | 17 | 3:1 | 1 | Accept |
| | 34 | 65 | 22 | 3:1 | 1 | Accept |
| | 39 | 77 | 23 | 3:1 | 0.644 | Accept |
| | 24 | 58 | 1 | 63:1 | 1 | Accept |
| B. napus 5A | 5A-1 | 32 | 17 | 3:1 | 0.097 | Accept |
| | 5A-3 | 53 | 17 | 3:1 | 1 | Accept |
| | 5A-4 | 49 | 14 | 3:1 | 0.563 | Accept |
| | 5A-5 | 50 | 21 | 3:1 | 0.413 | Accept |
| | 5A-6 | 36 | 13 | 3:1 | 0.74 | Accept |
| | 5A-8 | 70 | 6 | 15:1 | 0.644 | Accept |
| | 5A-9 | 35 | 11 | 3:1 | 1 | Accept |
| | 5A--10 | 32 | 15 | 3:1 | 0.316 | Accept |
| | 5A-11 | 47 | 7 | 15:1 | 0.017 | Reject |
| | 5A-12 | 71 | 1 | 63:1 | 1 | Accept |
| | 5A-13 | 52 | 15 | 3:1 | 0.574 | Accept |
| | 5A-14 | 45 | 17 | 3:1 | 0.553 | Accept |
| | 5A-15 | 61 | 28 | 3:1 | 0.14 | Accept |
| | 5A-16 | 61 | 24 | 3:1 | 0.451 | Accept |
| | 5A-17 | 78 | 25 | 3:1 | 0.821 | Accept |
| | 5A-18 | 56 | 24 | 3:1 | 0.302 | Accept |
| | 5A-19 | 46 | 14 | 3:1 | 0.766 | Accept |
| | 5A-20 | 60 | 19 | 3:1 | 0.796 | Accept |
| | 5A-21 | 86 | 14 | 3:1 | 0.011 | Reject |
| | 5A-23 | 54 | 9 | 15:1 | 0.01 | Reject |
| | 5A-25 | 48 | 17 | 3:1 | 0.773 | Accept |
| | 5A-26 | 53 | 18 | 3:1 | 1 | Accept |
| | 5A-1 | 32 | 17 | 3:1 | 0.097 | Accept |
| | 5A-3 | 53 | 17 | 3:1 | 1 | Accept |
| | 5A-4 | 49 | 14 | 3:1 | 0.563 | Accept |
| | 5A-5 | 50 | 21 | 3:1 | 0.413 | Accept |
| B. carinata 5B | 5B-1 | 54 | 3 | 15:1 | 0.6041 | Accept |
| | 5B-2 | 49 | 15 | 3:1 | 0.7728 | Accept |
| | 5B-3 | 50 | 12 | 3:1 | 0.3737 | Accept |
| | 5B-4 | 59 | 20 | 3:1 | 1 | Accept |
| | 5B-5 | 76 | 29 | 3:1 | 0.4976 | Accept |
| | 5B-6 | 58 | 12 | 3:1 | 0.1634 | Accept |
| | 5B-7 | 68 | 21 | 3:1 | 0.806 | Accept |
| | 5B-8 | 67 | 22 | 3:1 | 1 | Accept |
| | 5B-9 | 74 | 18 | 3:1 | 0.229 | Accept |
| | 5B-10 | 112 | 26 | 3:1 | 0.114 | Accept |
| | 5B-11 | 48 | 20 | 3:1 | 0.401 | Accept |
| | 5B-12 | 53 | 21 | 3:1 | 0.416 | Accept |
| | 5B-13 | 57 | 24 | 3:1 | 0.303 | Accept |
| | 5B-14 | 63 | 16 | 3:1 | 0.301 | Accept |
| | 5B-15 | 107 | 9 | 15:1 | 0.435 | Accept |
| | 5B-16 | 99 | 32 | 3:1 | 0.84 | Accept |
| | 5B-17 | 56 | 14 | 3:1 | 0.403 | Accept |
| | 5B-18 | 56 | 19 | 3:1 | 1 | Accept |
| | 5B-19 | 42 | 23 | 3:1 | 0.044 | Reject |
| | 5B-20 | 125 | 7 | 15:1 | 0.715 | Accept |
| | 5B-21 | 26 | 29 | 3:1 | <0.0001 | Reject |
| | 5B-22 | 33 | 11 | 3:1 | 1 | Accept |
| | 5B-23 | 51 | 19 | 3:1 | 0.784 | Accept |
| B. carinata 5C | 5C-18 | 118 | 42 | 3:1 | 0.715 | Yes |
| | 5C-11 | 76 | 26 | 3:1 | 0.8179 | Yes |
| | 5C-15 | 114 | 101 | 3:1 | 0.0001 | No |
| | 5C-12 | 70 | 16 | 3:1 | 0.2095 | Yes |
| | 5C-2 | 52 | 17 | 3:1 | 1 | Yes |
| | 5C-10 | 79 | 3 | 15:1 | 0.356 | Yes |
| | 5C-26 | 88 | 13 | 3:1 | 0.0057 | No |
| | 5C-20 | 59 | 23 | 3:1 | 0.4404 | Yes |
| | 5C-25 | 60 | 16 | 3:1 | 0.4268 | Yes |
| | 5C-5 | 66 | 14 | 3:1 | 0.1213 | Yes |
| | 5C-19 | 45 | 6 | 3:1 | 0.0245 | No |
| | 5C-6 | 95 | 3 | 15:1 | 0.2062 | Yes |
| | 5C-16 | 93 | 94 | 3:1 | 0.0001 | No |
| | 5C-9 | 112 | 7 | 15:1 | 1 | Yes |
| | 5C-17 | 116 | 37 | 3:1 | 0.8516 | Yes |
| | 5C-8 | 156 | 58 | 3:1 | 0.529 | Yes |
| | 5C-13 | 72 | 43 | 3:1 | 0.0026 | No |
| | 5C-1 | 72 | 27 | 3:1 | 0.4817 | Yes |
| | 5C-7 | 140 | 124 | 3:1 | 0.0001 | No |
| | 5C-14 | 41 | 24 | 3:1 | 0.0213 | No |
| | 5C-3 | 64 | 33 | 3:1 | 0.0342 | No |
| | Plant | | | Hypothesis | | Accept |
| Group | # | Resistant | Susceptible | Ratio | p value | hypothesis |
| C. sativa C1 | 80666-15 | 54 | 8 | 3:1 | 0.0684 | Yes |
| | 80666-20 | 50 | 12 | 3:1 | 0.3737 | Yes |
| | 80666-17 | 52 | 20 | 3:1 | 0.5862 | Yes |
| | 80666-13 | 48 | 18 | 3:1 | 0.5657 | Yes |
| | 80666-1 | 24 | 29 | 3:1 | 0.0001 | No |
| | 80666-3 | 39 | 32 | 3:1 | 0.0001 | No |
| | 80666-16 | 45 | 17 | 3:1 | 0.5531 | Yes |
| | 80666-19 | 55 | 18 | 3:1 | 1 | Yes |
| C. sativa C15 | 45897-16 | 68 | 17 | 3:1 | 0.3144 | Yes |
| | 45897-5 | 60 | 20 | 3:1 | 1 | Yes |
| | 45897-18 | 63 | 18 | 3:1 | 0.6063 | Yes |
| | 45897-8 | 82 | 6 | 15:1 | 0.6452 | Yes |
| | 45897-14 | 51 | 16 | 3:1 | 0.7789 | Yes |
| | 45897-1 | 53 | 8 | 3:1 | 0.0374 | No |
| | 45897-15 | 66 | 4 | 15:1 | 1 | Yes |
| | 45897-9 | 55 | 17 | 3:1 | 0.7855 | Yes |
| | 45897-13 | 81 | 19 | 3:1 | 0.1659 | Yes |
| | 45897-12 | 58 | 20 | 3:1 | 0.7919 | Yes |
| | 45897-11 | 58 | 30 | 3:1 | 0.0489 | No |
| | 45897-10 | 59 | 15 | 3:1 | 0.4163 | Yes |
| | 45897-6 | 58 | 17 | 3:1 | 0.5954 | Yes |
| | 45897-7 | 63 | 21 | 3:1 | 1 | Yes |
| | 45897-17 | 53 | 16 | 3:1 | 0.78 | Yes |
| | 45897-19 | 57 | 17 | 3:1 | 0.7864 | Yes |
| | 45897-2 | 56 | 11 | 3:1 | 0.0921 | Yes |
| | 45897-20 | 65 | 19 | 3:1 | 0.6143 | Yes |
| | 45897-3 | 64 | 1 | 63:1 | 1 | Yes |
| | 45897-4 | 63 | 15 | 3:1 | 0.2914 | Yes |
| C. sativa C19 | 65416-1 | 97 | 34 | 3:1 | 0.84 | Accept |
| | 65416-2 | 59 | 22 | 3:1 | 0.61 | Accept |
| | 65416-3 | 81 | 37 | 3:1 | 0.09 | Accept |
| | 65416-4 | 69 | 45 | 3:1 | 0.0002 | No |
| | 65416-5 | 174 | 47 | 3:1 | 0.213 | Accept |
| | 65416-6 | 176 | 37 | 3:1 | 0.01 | No |
| | 65416-7 | 99 | 19 | 3:1 | 0.03 | No |
| | 65416-8 | 123 | 26 | 3:1 | 0.04 | No |
| | 65416-9 | 110 | 18 | 15:1 | 0.0002 | No |
| | 65416-10 | 153 | 14 | 15:1 | 0.192 | Accept |
| | 65416-11 | 97 | 35 | 3:1 | 0.688 | Accept |
| | 65416-12 | 102 | 7 | 15:1 | 1 | Accept |
| | 65416-13 | 92 | 33 | 3:1 | 0.679 | Accept |
| | 65416-14 | 113 | 71 | 3:1 | 0 | No |
| | 65416-15 | 120 | 48 | 3:1 | 0.285 | Accept |
| | 65416-16 | 106 | 60 | 3:1 | 0.0006 | No |
| | 65416-17 | 203 | 63 | 3:1 | 0.67 | Accept |
| | 65416-19 | 165 | 52 | 3:1 | 0.75 | Accept |
| | 65416-20 | 40 | 11 | 3:1 | 0.52 | Accept |
| | 65416-21 | 261 | 78 | 3:1 | 0.38 | Accept |

TABLE 4
Average fatty acid composition (%) in transgenic T3 plants. Complete data in Appendix 2.

| LINE | 16:0 | 18:0 | 18:1 | 18:2 | GLA | 18:3 | 20:1 |
|---|---|---|---|---|---|---|---|
| 1A | 8.96 ± 0.52 | 3.69 ± 0.15 | 31.6 ± 1.49 | 6.58 ± 0.99 | 3.40 ± 0.25 | 6.33 ± 0.54 | 17.0 ± 0.87 |
| 1C | 8.38 ± 0.18 | 3.65 ± 0.09 | 32.3 ± 0.59 | 11.9 ± 3.22 | 3.11 ± 0.69 | 9.50 ± 1.22 | 18.6 ± 0.44 |
| 2B | 8.44 ± 0.61 | 3.54 ± 0.15 | 30.3 ± 2.20 | 10.0 ± 2.27 | 4.07 ± 0.81 | 8.44 ± 2.00 | 17.7 ± 1.04 |
| 2C | 8.36 ± 0.41 | 3.58 ± 0.22 | 31.1 ± 1.70 | 8.79 ± 2.63 | 3.80 ± 0.62 | 8.22 ± 2.67 | 18.1 ± 0.92 |
| 3A | 8.25 ± 0.85 | 3.59 ± 0.54 | 13.8 ± 3.03 | 19.5 ± 1.92 | 8.00 ± 1.21 | 12.1 ± 1.50 | 17.8 ± 1.32 |
| 3B | 8.69 ± 0.32 | 3.25 ± 0.07 | 12.5 ± 1.82 | 18.4 ± 3.35 | 11.5 ± 2.99 | 12.2 ± 2.02 | 16.4 ± 0.82 |
| 3C | 8.13 ± 0.27 | 3.59 ± 0.07 | 14.3 ± 1.09 | 15.5 ± 4.40 | 11.5 ± 3.97 | 10.1 ± 2.36 | 17.7 ± 0.82 |
| 5A | 8.33 ± 0.12 | 3.10 ± 0.12 | 12.1 ± 0.79 | 23.5 ± 6.02 | 8.90 ± 1.49 | 13.6 ± 0.59 | 16.4 ± 0.27 |
| 5B | 8.86 ± 0.36 | 3.28 ± 0.17 | 14.6 ± 8.96 | 15.6 ± 4.73 | 12.7 ± 1.84 | 10.6 ± 2.29 | 16.0 ± 1.13 |
| 5C | 7.73 ± 0.24 | 2.95 ± 0.07 | 14.5 ± 1.10 | 22.0 ± 0.99 | 6.90 ± 1.20 | 13.8 ± 0.87 | 18.0 ± 0.43 |
| C1 | 7.82 ± 0.13 | 3.27 ± 0.15 | 12.8 ± 2.12 | 11.5 ± 7.13 | 15.0 ± 7.82 | 9.42 ± 3.98 | 17.2 ± 0.42 |
| C15 | 7.96 ± 0.06 | 2.96 ± 0.06 | 13.3 ± 0.73 | 21.4 ± 0.44 | 8.29 ± 0.74 | 12.9 ± 0.43 | 17.4 ± 0.09 |
| C19 | 7.70 ± 0.24 | 3.71 ± 0.17 | 14.6 ± 1.62 | 7.50 ± 0.89 | 16.9 ± 2.36 | 7.81 ± 0.74 | 16.9 ± 0.44 |
| | | | | | GLA | | |
| | | | | | DGLA | ALA | |
| | | | | | SDA | SDA | GLA |
| | LINE | DGLA | SDA | ETA | ETA | ETA | DGLA |
| | 1A | 7.49 ± 0.85 | 0.55 ± 0.23 | 1.40 ± 0.22 | 12.83 | 8.27 | 10.89 |
| | 1C | 2.03 ± 0.92 | 0.91 ± 0.22 | 0.38 ± 0.26 | 6.43 | 10.78 | 5.15 |
| | 2B | 3.32 ± 2.22 | 0.79 ± 0.15 | 0.43 ± 0.37 | 8.61 | 9.66 | 7.39 |
| | 2C | 3.69 ± 2.99 | 0.80 ± 0.13 | 0.66 ± 0.56 | 8.96 | 9.69 | 7.50 |
| | 3A | 1.56 ± 0.63 | 1.09 ± 0.20 | 0.00 | 10.64 | 13.27 | 9.55 |
| | 3B | 1.25 ± 0.74 | 1.77 ± 0.92 | 0.00 | 14.60 | 14.05 | 12.83 |
| | 3C | 3.33 ± 3.08 | 1.63 ± 0.67 | 0.28 ± 0.48 | 16.77 | 12.02 | 14.86 |
| | 5A | 1.68 ± 0.69 | 1.18 ± 0.2 | 0.00 | 11.77 | 14.83 | 10.58 |
| | 5B | 1.71 ± 1.53 | 1.85 ± 0.31 | 0.01 ± 0.01 | 16.26 | 12.53 | 14.41 |
| | 5C | 0.87 ± 0.49 | 0.94 ± 0.18 | 0.00 | 8.70 | 14.79 | 7.76 |
| | C1 | 5.79 ± 3.13 | 2.40 ± 1.26 | 0.76 ± 0.51 | 24.02 | 12.58 | 20.85 |
| | C15 | 1.49 ± 0.39 | 1.11 ± 0.13 | 0.02 ± 0.04 | 10.91 | 14.07 | 9.78 |
| | C19 | 6.12 ± 0.67 | 2.57 ± 0.46 | 0.87 ± 0.14 | 26.55 | 11.25 | 23.11 |

TABLE 5

| | SEQ | SEQ | | |
|---|---|---|---|---|
| | ID | ID | | |
| PDCT Name | NA | AA | Activity | Organism |
| Napus_1A | 1 | 2 | PDCT1 | Brasssica napus |
| Napus_2A | 3 | 4 | PDCT1 | Brasssica napus |
| Carinata_1B | 5 | 6 | PDCT1 | Brassica carinata |
| Carinata_1C | 7 | 8 | PDCT1 | Brassica carinata |
| Carinata_2B | 9 | 10 | PDCT1 | Brassica carinata |
| Carinata_2C | 11 | 12 | PDCT1 | Brassica carinata |
| BjROD1-B4 | 13 | 14 | PDCT1 | Brassica juncea |
| BjROD1-A3 | 15 | 16 | PDCT1 | Brassica juncea |
| BjROD1-B3 | 39 | 40 | PDCT1 | Brassica juncea |
| Napus_1C | 41 | 42 | PDCT1 | Brasssica napus |
| Napus_2C | 43 | 44 | PDCT1 | Brasssica napus |
| Consensus PDCT1 | 45 | 46 | PDCT1 | Artificial |
| Napus_3A | 17 | 18 | PDCT3/5 | Brasssica napus |
| Napus_5A | 19 | 20 | PDCT3/5 | Brasssica napus |
| Carinata_3B | 21 | 22 | PDCT3/5 | Brassica carinata |
| Carinata_3C | 23 | 24 | PDCT3/5 | Brassica carinata |
| Carinata_5B | 25 | 26 | PDCT3/5 | Brassica carinata |
| Carinata_5C | 27 | 28 | PDCT3/5 | Brassica carinata |
| BjROD1-A2 | 29 | 30 | PDCT3/5 | Brassica juncea |
| BjROD1-B2 | 31 | 32 | PDCT3/5 | Brassica juncea |
| BjROD1-B1 | 49 | 50 | PDCT3/5 | Brassica juncea |
| BjROD1-A1 | 51 | 52 | PDCT3/5 | Brassica juncea |
| BrROD1_SEQIDNO7 | 53 | 54 | PDCT3/5 | Brassica rapa |
| Napus_5C | 55 | 56 | PDCT3/5 | Brasssica napus |
| Napus_3C | 57 | 58 | PDCT3/5 | Brasssica napus |
| Consensus PDCT3/5 | 59 | 60 | PDCT3/5 | Artificial |
| Camelina_C15(45897) | 33 | 34 | PDCT15 | Camelina sativa |
| Camelina_C19(65416) | 35 | 36 | PDCT19 | Camelina sativa |
| Camelina_C1(80666) | 37 | 38 | PDCT19 | Camelina sativa |
| Consensus PDCT19 | 47 | 48 | PCDT19 | Artificial |
| AtRodD1 | 61 | 62 | | Arabidopsis |
| | | | | thaliana |
| GmROD1-1 | 63 | 64 | PDCT1 | Glycine max |
| | | | candiate | |
| GmROD1-2 | 65 | 66 | PDCT1 | Glycine max |
| | | | candiate | |
| RcPDCT | 67 | 68 | PDCT1 | Ricinis |
| | | | candiate | communis |
| RcROD1_SEQIDNO9 | 69 | 70 | PDCT1 | Ricinis |
| | | | candiate | communis |
| LuPDCT1 | 71 | 72 | PDCT1 | Linum |
| | | | candiate | usitatissimum |
| LuPDCT2 | 73 | 74 | PDCT1 | Linum |
| | | | candiate | usitatissimum |
| OsROD1_SEQIDNO11 | 75 | 76 | / | Oryza sativa |
| ZmROD1_GRMZM2G015040 | 77 | 78 | / | Zea mays |

TABLE 6

| | | Needle |
|---|---|---|
| | | Protein |
| | | Identity % |
| | | Default |
| Seq_1 | Seq_2 | settings |
| ATRODD1 | ATRODD1 | 100 |
| ATRODD1 | BJROD1-A1 | 78.8 |
| ATRODD1 | BJROD1-A2 | 76.1 |
| ATRODD1 | BJROD1-A3 | 72.7 |
| ATRODD1 | BJROD1-B1 | 78.5 |
| ATRODD1 | BJROD1-B2 | 78.1 |
| ATRODD1 | BJROD1-B3 | 73.7 |
| ATRODD1 | BJROD1-B4 | 55.5 |
| ATRODD1 | BRROD1_SEQIDNO7 | 78.8 |
| ATRODD1 | CAMELINA_C1(80666) | 86.1 |
| ATRODD1 | CAMELINA_C15(45897) | 85.8 |
| ATRODD1 | CAMELINA_C19(65416) | 86.2 |
| ATRODD1 | CARINATA_1B | 73.7 |
| ATRODD1 | CARINATA_1C | 74 |
| ATRODD1 | CARINATA_2B | 55.5 |
| ATRODD1 | CARINATA_2C | 55.5 |
| ATRODD1 | CARINATA_3B | 78.5 |
| ATRODD1 | CARINATA_3C | 78.8 |
| ATRODD1 | CARINATA_5B | 80.5 |
| ATRODD1 | CARINATA_5C | 79.8 |
| ATRODD1 | GMROD1-1 | 60.7 |
| ATRODD1 | GMROD1-2 | 58.1 |
| ATRODD1 | LUPDCT1 | 54.6 |
| ATRODD1 | LUPDCT2 | 54.2 |
| ATRODD1 | NAPUS_1A | 72.7 |
| ATRODD1 | NAPUS_1C | 73.7 |
| ATRODD1 | NAPUS_2A | 55.5 |
| ATRODD1 | NAPUS_2C | 55.1 |
| ATRODD1 | NAPUS_3A | 79.2 |
| ATRODD1 | NAPUS_3C | 78.8 |
| ATRODD1 | NAPUS_5A | 79.7 |
| ATRODD1 | NAPUS_5C | 80.1 |
| ATRODD1 | OSROD1_SEQIDNO11 | 45.5 |
| ATRODD1 | RCPDCT | 58.7 |
| ATRODD1 | RCROD1_SEQIDNO9 | 58.7 |
| ATRODD1 | ZMROD1_GRMZM2G015040 | 44.4 |
| ATRODD1 | ZMROD1_GRMZM2G087896 | 42.9 |
| BJROD1-A1 | ATRODD1 | 78.8 |
| BJROD1-A1 | BJROD1-A1 | 100 |
| BJROD1-A1 | BJROD1-A2 | 82.6 |
| BJROD1-A1 | BJROD1-A3 | 77.8 |
| BJROD1-A1 | BJROD1-B1 | 96.8 |
| BJROD1-A1 | BJROD1-B2 | 83.7 |
| BJROD1-A1 | BJROD1-B3 | 77.8 |
| BJROD1-A1 | BJROD1-B4 | 57.1 |
| BJROD1-A1 | BRROD1_SEQIDNO7 | 99.3 |
| BJROD1-A1 | CAMELINA_C1(80666) | 76.8 |
| BJROD1-A1 | CAMELINA_C15(45897) | 76.5 |
| BJROD1-A1 | CAMELINA_C19(65416) | 76.5 |
| BJROD1-A1 | CARINATA_1B | 77.8 |
| BJROD1-A1 | CARINATA_1C | 78.8 |
| BJROD1-A1 | CARINATA_2B | 57.1 |
| BJROD1-A1 | CARINATA_2C | 57.1 |
| BJROD1-A1 | CARINATA_3B | 96.8 |
| BJROD1-A1 | CARINATA_3C | 97.9 |
| BJROD1-A1 | CARINATA_5B | 87.1 |
| BJROD1-A1 | CARINATA_5C | 86.5 |
| BJROD1-A1 | GMROD1-1 | 62.4 |
| BJROD1-A1 | GMROD1-2 | 62.8 |
| BJROD1-A1 | LUPDCT1 | 54.5 |
| BJROD1-A1 | LUPDCT2 | 54.5 |
| BJROD1-A1 | NAPUS_1A | 77.1 |
| BJROD1-A1 | NAPUS_1C | 78.5 |
| BJROD1-A1 | NAPUS_2A | 57.1 |
| BJROD1-A1 | NAPUS_2C | 56.8 |
| BJROD1-A1 | NAPUS_3A | 98.2 |
| BJROD1-A1 | NAPUS_3C | 97.9 |
| BJROD1-A1 | NAPUS_5A | 86.5 |
| BJROD1-A1 | NAPUS_5C | 86.8 |
| BJROD1-A1 | OSROD1_SEQIDNO11 | 45.3 |
| BJROD1-A1 | RCPDCT | 58.6 |
| BJROD1-A1 | RCROD1_SEQIDNO9 | 58.6 |
| BJROD1-A1 | ZMROD1_GRMZM2G015040 | 45.3 |
| BJROD1-A1 | ZMROD1_GRMZM2G087896 | 44.1 |
| BJROD1-A2 | ATRODD1 | 76.1 |
| BJROD1-A2 | BJROD1-A1 | 82.6 |
| BJROD1-A2 | BJROD1-A2 | 100 |
| BJROD1-A2 | BJROD1-A3 | 77.2 |
| BJROD1-A2 | BJROD1-B1 | 83.3 |
| BJROD1-A2 | BJROD1-B2 | 87 |
| BJROD1-A2 | BJROD1-B3 | 77.9 |
| BJROD1-A2 | BJROD1-B4 | 55.4 |
| BJROD1-A2 | BRROD1_SEQIDNO7 | 83 |
| BJROD1-A2 | CAMELINA_C1(80666) | 71.7 |
| BJROD1-A2 | CAMELINA_C15(45897) | 71.9 |
| BJROD1-A2 | CAMELINA_C19(65416) | 72.5 |
| BJROD1-A2 | CARINATA_1B | 77.9 |
| BJROD1-A2 | CARINATA_1C | 78.3 |
| BJROD1-A2 | CARINATA_2B | 55.4 |
| BJROD1-A2 | CARINATA_2C | 55.4 |
| BJROD1-A2 | CARINATA_3B | 83.3 |
| BJROD1-A2 | CARINATA_3C | 83 |
| BJROD1-A2 | CARINATA_5B | 88.8 |
| BJROD1-A2 | CARINATA_5C | 93.3 |
| BJROD1-A2 | GMROD1-1 | 62.1 |
| BJROD1-A2 | GMROD1-2 | 57.5 |
| BJROD1-A2 | LUPDCT1 | 51.5 |
| BJROD1-A2 | LUPDCT2 | 51.5 |
| BJROD1-A2 | NAPUS_1A | 76.6 |
| BJROD1-A2 | NAPUS_1C | 77.9 |
| BJROD1-A2 | NAPUS_2A | 55.4 |
| BJROD1-A2 | NAPUS_2C | 55.1 |
| BJROD1-A2 | NAPUS_3A | 81.7 |
| BJROD1-A2 | NAPUS_3C | 83 |
| BJROD1-A2 | NAPUS_5A | 95.4 |
| BJROD1-A2 | NAPUS_5C | 93.6 |
| BJROD1-A2 | OSROD1_SEQIDNO11 | 42.2 |
| BJROD1-A2 | RCPDCT | 59.7 |
| BJROD1-A2 | RCROD1_SEQIDNO9 | 59.7 |
| BJROD1-A2 | ZMROD1_GRMZM2G015040 | 45.1 |
| BJROD1-A2 | ZMROD1_GRMZM2G087896 | 45.6 |
| BJROD1-A3 | ATRODD1 | 72.7 |
| BJROD1-A3 | BJROD1-A1 | 77.8 |
| BJROD1-A3 | BJROD1-A2 | 77.2 |
| BJROD1-A3 | BJROD1-A3 | 100 |
| BJROD1-A3 | BJROD1-B1 | 78.5 |
| BJROD1-A3 | BJROD1-B2 | 76.1 |
| BJROD1-A3 | BJROD1-B3 | 95.8 |
| BJROD1-A3 | BJROD1-B4 | 55.4 |
| BJROD1-A3 | BRROD1_SEQIDNO7 | 78.5 |
| BJROD1-A3 | CAMELINA_C1(80666) | 69.1 |
| BJROD1-A3 | CAMELINA_C15(45897) | 69.5 |
| BJROD1-A3 | CAMELINA_C19(65416) | 69.5 |
| BJROD1-A3 | CARINATA_1B | 95.8 |
| BJROD1-A3 | CARINATA_1C | 94.5 |
| BJROD1-A3 | CARINATA_2B | 55 |
| BJROD1-A3 | CARINATA_2C | 55.4 |
| BJROD1-A3 | CARINATA_3B | 78.8 |
| BJROD1-A3 | CARINATA_3C | 78.8 |
| BJROD1-A3 | CARINATA_5B | 79.3 |
| BJROD1-A3 | CARINATA_5C | 78.2 |
| BJROD1-A3 | GMROD1-1 | 60.2 |
| BJROD1-A3 | GMROD1-2 | 54.4 |
| BJROD1-A3 | LUPDCT1 | 52.5 |
| BJROD1-A3 | LUPDCT2 | 53.4 |
| BJROD1-A3 | NAPUS_1A | 98.6 |
| BJROD1-A3 | NAPUS_1C | 95.5 |
| BJROD1-A3 | NAPUS_2A | 55.4 |
| BJROD1-A3 | NAPUS_2C | 55 |
| BJROD1-A3 | NAPUS_3A | 78.3 |
| BJROD1-A3 | NAPUS_3C | 78.8 |
| BJROD1-A3 | NAPUS_5A | 78.2 |
| BJROD1-A3 | NAPUS_5C | 78.5 |
| BJROD1-A3 | OSROD1_SEQIDNO11 | 41.8 |
| BJROD1-A3 | RCPDCT | 57 |
| BJROD1-A3 | RCROD1_SEQIDNO9 | 57 |
| BJROD1-A3 | ZMROD1_GRMZM2G015040 | 43.7 |
| BJROD1-A3 | ZMROD1_GRMZM2G087896 | 42.7 |
| BJROD1-B1 | ATRODD1 | 78.5 |
| BJROD1-B1 | BJROD1-A1 | 96.8 |
| BJROD1-B1 | BJROD1-A2 | 83.3 |
| BJROD1-B1 | BJROD1-A3 | 78.5 |
| BJROD1-B1 | BJROD1-B1 | 100 |
| BJROD1-B1 | BJROD1-B2 | 83.3 |
| BJROD1-B1 | BJROD1-B3 | 78.5 |
| BJROD1-B1 | BJROD1-B4 | 56.8 |
| BJROD1-B1 | BRROD1_SEQIDNO7 | 97.5 |
| BJROD1-B1 | CAMELINA_C1(80666) | 76.5 |
| BJROD1-B1 | CAMELINA_C15(45897) | 75.8 |
| BJROD1-B1 | CAMELINA_C19(65416) | 75.8 |
| BJROD1-B1 | CARINATA_1B | 78.5 |
| BJROD1-B1 | CARINATA_1C | 79.2 |
| BJROD1-B1 | CARINATA_2B | 56.8 |
| BJROD1-B1 | CARINATA_2C | 56.8 |
| BJROD1-B1 | CARINATA_3B | 99.3 |
| BJROD1-B1 | CARINATA_3C | 98.2 |
| BJROD1-B1 | CARINATA_5B | 86.8 |
| BJROD1-B1 | CARINATA_5C | 86.8 |
| BJROD1-B1 | GMROD1-1 | 61.5 |
| BJROD1-B1 | GMROD1-2 | 62.8 |
| BJROD1-B1 | LUPDCT1 | 53.9 |
| BJROD1-B1 | LUPDCT2 | 53.9 |
| BJROD1-B1 | NAPUS_1A | 77.8 |
| BJROD1-B1 | NAPUS_1C | 79.2 |
| BJROD1-B1 | NAPUS_2A | 56.8 |
| BJROD1-B1 | NAPUS_2C | 56.4 |
| BJROD1-B1 | NAPUS_3A | 96.8 |
| BJROD1-B1 | NAPUS_3C | 98.2 |
| BJROD1-B1 | NAPUS_5A | 86.8 |
| BJROD1-B1 | NAPUS_5C | 87.2 |
| BJROD1-B1 | OSROD1_SEQIDNO11 | 43.8 |
| BJROD1-B1 | RCPDCT | 60.8 |
| BJROD1-B1 | RCROD1_SEQIDNO9 | 60.8 |
| BJROD1-B1 | ZMROD1_GRMZM2G015040 | 45.8 |
| BJROD1-B1 | ZMROD1_GRMZM2G087896 | 44.1 |
| BJROD1-B2 | ATRODD1 | 78.1 |
| BJROD1-B2 | BJROD1-A1 | 83.7 |
| BJROD1-B2 | BJROD1-A2 | 87 |
| BJROD1-B2 | BJROD1-A3 | 76.1 |
| BJROD1-B2 | BJROD1-B1 | 83.3 |
| BJROD1-B2 | BJROD1-B2 | 100 |
| BJROD1-B2 | BJROD1-B3 | 77.5 |
| BJROD1-B2 | BJROD1-B4 | 56.1 |
| BJROD1-B2 | BRROD1_SEQIDNO7 | 84 |
| BJROD1-B2 | CAMELINA_C1(80666) | 75.1 |
| BJROD1-B2 | CAMELINA_C15(45897) | 75.2 |
| BJROD1-B2 | CAMELINA_C19(65416) | 75.2 |
| BJROD1-B2 | CARINATA_1B | 77.1 |
| BJROD1-B2 | CARINATA_1C | 77.5 |
| BJROD1-B2 | CARINATA_2B | 58.9 |
| BJROD1-B2 | CARINATA_2C | 56.1 |
| BJROD1-B2 | CARINATA_3B | 83.3 |
| BJROD1-B2 | CARINATA_3C | 85.2 |
| BJROD1-B2 | CARINATA_5B | 93.3 |
| BJROD1-B2 | CARINATA_5C | 91.2 |
| BJROD1-B2 | GMROD1-1 | 64.1 |
| BJROD1-B2 | GMROD1-2 | 65 |
| BJROD1-B2 | LUPDCT1 | 53.8 |
| BJROD1-B2 | LUPDCT2 | 53.8 |
| BJROD1-B2 | NAPUS_1A | 75.4 |
| BJROD1-B2 | NAPUS_1C | 77.1 |
| BJROD1-B2 | NAPUS_2A | 56.1 |
| BJROD1-B2 | NAPUS_2C | 55.7 |
| BJROD1-B2 | NAPUS_3A | 84.2 |
| BJROD1-B2 | NAPUS_3C | 85.2 |
| BJROD1-B2 | NAPUS_5A | 90.8 |
| BJROD1-B2 | NAPUS_5C | 91.5 |
| BJROD1-B2 | OSROD1_SEQIDNO11 | 41.3 |
| BJROD1-B2 | RCPDCT | 59.1 |
| BJROD1-B2 | RCROD1_SEQIDNO9 | 59.1 |
| BJROD1-B2 | ZMROD1_GRMZM2G015040 | 47.1 |
| BJROD1-B2 | ZMROD1_GRMZM2G087896 | 45.6 |
| BJROD1-B3 | ATRODD1 | 73.7 |
| BJROD1-B3 | BJROD1-A1 | 77.8 |
| BJROD1-B3 | BJROD1-A2 | 77.9 |
| BJROD1-B3 | BJROD1-A3 | 95.8 |
| BJROD1-B3 | BJROD1-B1 | 78.5 |
| BJROD1-B3 | BJROD1-B2 | 77.5 |
| BJROD1-B3 | BJROD1-B3 | 100 |
| BJROD1-B3 | BJROD1-B4 | 56.1 |
| BJROD1-B3 | BRROD1_SEQIDNO7 | 78.5 |
| BJROD1-B3 | CAMELINA_C1(80666) | 70.4 |
| BJROD1-B3 | CAMELINA_C15(45897) | 70.2 |
| BJROD1-B3 | CAMELINA_C19(65416) | 70.9 |
| BJROD1-B3 | CARINATA_1B | 98.6 |
| BJROD1-B3 | CARINATA_1C | 94.5 |
| BJROD1-B3 | CARINATA_2B | 55.4 |
| BJROD1-B3 | CARINATA_2C | 56.4 |
| BJROD1-B3 | CARINATA_3B | 78.8 |
| BJROD1-B3 | CARINATA_3C | 78.8 |
| BJROD1-B3 | CARINATA_5B | 80.2 |
| BJROD1-B3 | CARINATA_5C | 78.8 |
| BJROD1-B3 | GMROD1-1 | 61.5 |
| BJROD1-B3 | GMROD1-2 | 52.7 |
| BJROD1-B3 | LUPDCT1 | 53.3 |
| BJROD1-B3 | LUPDCT2 | 53.6 |
| BJROD1-B3 | NAPUS_1A | 95.2 |
| BJROD1-B3 | NAPUS_1C | 95.5 |
| BJROD1-B3 | NAPUS_2A | 56.1 |
| BJROD1-B3 | NAPUS_2C | 55.7 |
| BJROD1-B3 | NAPUS_3A | 78.3 |
| BJROD1-B3 | NAPUS_3C | 78.8 |
| BJROD1-B3 | NAPUS_5A | 78.9 |
| BJROD1-B3 | NAPUS_5C | 79.2 |
| BJROD1-B3 | OSROD1_SEQIDNO11 | 43.2 |
| BJROD1-B3 | RCPDCT | 57.6 |
| BJROD1-B3 | RCROD1_SEQIDNO9 | 57.6 |
| BJROD1-B3 | ZMROD1_GRMZM2G015040 | 44 |
| BJROD1-B3 | ZMROD1_GRMZM2G087896 | 44.7 |
| BJROD1-B4 | ATRODD1 | 55.5 |
| BJROD1-B4 | BJROD1-A1 | 57.1 |
| BJROD1-B4 | BJROD1-A2 | 55.4 |
| BJROD1-B4 | BJROD1-A3 | 55.4 |
| BJROD1-B4 | BJROD1-B1 | 56.8 |
| BJROD1-B4 | BJROD1-B2 | 59 |
| BJROD1-B4 | BJROD1-B3 | 56.1 |
| BJROD1-B4 | BJROD1-B4 | 100 |
| BJROD1-B4 | BRROD1_SEQIDNO7 | 57.1 |
| BJROD1-B4 | CAMELINA_C1(80666) | 55.4 |
| BJROD1-B4 | CAMELINA_C15(45897) | 55.2 |
| BJROD1-B4 | CAMELINA_C19(65416) | 55.2 |
| BJROD1-B4 | CARINATA_1B | 56.4 |
| BJROD1-B4 | CARINATA_1C | 55.4 |
| BJROD1-B4 | CARINATA_2B | 97.4 |
| BJROD1-B4 | CARINATA_2C | 98.3 |
| BJROD1-B4 | CARINATA_3B | 57.1 |
| BJROD1-B4 | CARINATA_3C | 56.8 |
| BJROD1-B4 | CARINATA_5B | 58.2 |
| BJROD1-B4 | CARINATA_5C | 55.5 |
| BJROD1-B4 | GMROD1-1 | 54.9 |
| BJROD1-B4 | GMROD1-2 | 53.5 |
| BJROD1-B4 | LUPDCT1 | 48.4 |
| BJROD1-B4 | LUPDCT2 | 48.4 |
| BJROD1-B4 | NAPUS_1A | 55.4 |
| BJROD1-B4 | NAPUS_1C | 55.7 |
| BJROD1-B4 | NAPUS_2A | 99.6 |
| BJROD1-B4 | NAPUS_2C | 99.1 |
| BJROD1-B4 | NAPUS_3A | 57.4 |
| BJROD1-B4 | NAPUS_3C | 56.8 |
| BJROD1-B4 | NAPUS_5A | 55.5 |
| BJROD1-B4 | NAPUS_5C | 55.5 |
| BJROD1-B4 | OSROD1_SEQIDNO11 | 37.7 |
| BJROD1-B4 | RCPDCT | 51.6 |
| BJROD1-B4 | RCROD1_SEQIDNO9 | 51.6 |
| BJROD1-B4 | ZMROD1_GRMZM2G015040 | 44.6 |
| BJROD1-B4 | ZMROD1_GRMZM2G087896 | 45.1 |
| BRROD1_SEQIDNO7 | ATRODD1 | 78.8 |
| BRROD1_SEQIDNO7 | BJROD1-A1 | 99.3 |
| BRROD1_SEQIDNO7 | BJROD1-A2 | 83 |
| BRROD1_SEQIDNO7 | BJROD1-A3 | 78.5 |
| BRROD1_SEQIDNO7 | BJROD1-B1 | 97.5 |
| BRROD1_SEQIDNO7 | BJROD1-B2 | 84 |
| BRROD1_SEQIDNO7 | BJROD1-B3 | 78.5 |
| BRROD1_SEQIDNO7 | BJROD1-B4 | 57.1 |
| BRROD1_SEQIDNO7 | BRROD1_SEQIDNO7 | 100 |
| BRROD1_SEQIDNO7 | CAMELINA_C1(80666) | 76.8 |
| BRROD1_SEQIDNO7 | CAMELINA_C15(45897) | 76.5 |
| BRROD1_SEQIDNO7 | CAMELINA_C19(65416) | 76.5 |
| BRROD1_SEQIDNO7 | CARINATA_1B | 78.5 |
| BRROD1_SEQIDNO7 | CARINATA_1C | 79.5 |
| BRROD1_SEQIDNO7 | CARINATA_2B | 57.1 |
| BRROD1_SEQIDNO7 | CARINATA_2C | 57.1 |
| BRROD1_SEQIDNO7 | CARINATA_3B | 97.5 |
| BRROD1_SEQIDNO7 | CARINATA_3C | 98.6 |
| BRROD1_SEQIDNO7 | CARINATA_5B | 87.5 |
| BRROD1_SEQIDNO7 | CARINATA_5C | 86.8 |
| BRROD1_SEQIDNO7 | GMROD1-1 | 61.2 |
| BRROD1_SEQIDNO7 | GMROD1-2 | 63.1 |
| BRROD1_SEQIDNO7 | LUPDCT1 | 54.5 |
| BRROD1_SEQIDNO7 | LUPDCT2 | 54.5 |
| BRROD1_SEQIDNO7 | NAPUS_1A | 77.8 |
| BRROD1_SEQIDNO7 | NAPUS_1C | 79.2 |
| BRROD1_SEQIDNO7 | NAPUS_2A | 57.1 |
| BRROD1_SEQIDNO7 | NAPUS_2C | 56.8 |
| BRROD1_SEQIDNO7 | NAPUS_3A | 98.9 |
| BRROD1_SEQIDNO7 | NAPUS_3C | 98.6 |
| BRROD1_SEQIDNO7 | NAPUS_5A | 86.8 |
| BRROD1_SEQIDNO7 | NAPUS_5C | 87.2 |
| BRROD1_SEQIDNO7 | OSROD1_SEQIDNO11 | 41.4 |
| BRROD1_SEQIDNO7 | RCPDCT | 60.8 |
| BRROD1_SEQIDNO7 | RCROD1_SEQIDNO9 | 60.8 |
| BRROD1_SEQIDNO7 | ZMROD1_GRMZM2G015040 | 46.2 |
| BRROD1_SEQIDNO7 | ZMROD1_GRMZM2G087896 | 44.1 |
| CAMELINA_C1(80666) | ATRODD1 | 86.1 |
| CAMELINA_C1(80666) | BJROD1-A1 | 76.8 |
| CAMELINA_C1(80666) | BJROD1-A2 | 71.7 |
| CAMELINA_C1(80666) | BJROD1-A3 | 69.1 |
| CAMELINA_C1(80666) | BJROD1-B1 | 76.5 |
| CAMELINA_C1(80666) | BJROD1-B2 | 75.1 |
| CAMELINA_C1(80666) | BJROD1-B3 | 70.4 |
| CAMELINA_C1(80666) | BJROD1-B4 | 55.4 |
| CAMELINA_C1(80666) | BRROD1_SEQIDNO7 | 76.8 |
| CAMELINA_C1(80666) | CAMELINA_C1(80666) | 100 |
| CAMELINA_C1(80666) | CAMELINA_C15(45897) | 96.6 |
| CAMELINA_C1(80666) | CAMELINA_C19(65416) | 98 |
| CAMELINA_C1(80666) | CARINATA_1B | 70.4 |
| CAMELINA_C1(80666) | CARINATA_1C | 71.1 |
| CAMELINA_C1(80666) | CARINATA_2B | 55.4 |
| CAMELINA_C1(80666) | CARINATA_2C | 55.4 |
| CAMELINA_C1(80666) | CARINATA_3B | 76.5 |
| CAMELINA_C1(80666) | CARINATA_3C | 76.8 |
| CAMELINA_C1(80666) | CARINATA_5B | 77.1 |
| CAMELINA_C1(80666) | CARINATA_5C | 75.7 |
| CAMELINA_C1(80666) | GMROD1-1 | 60.8 |
| CAMELINA_C1(80666) | GMROD1-2 | 60.1 |
| CAMELINA_C1(80666) | LUPDCT1 | 55 |
| CAMELINA_C1(80666) | LUPDCT2 | 55.3 |
| CAMELINA_C1(80666) | NAPUS_1A | 69.1 |
| CAMELINA_C1(80666) | NAPUS_1C | 70.8 |
| CAMELINA_C1(80666) | NAPUS_2A | 55.4 |
| CAMELINA_C1(80666) | NAPUS_2C | 55.1 |
| CAMELINA_C1(80666) | NAPUS_3A | 76.9 |
| CAMELINA_C1(80666) | NAPUS_3C | 76.8 |
| CAMELINA_C1(80666) | NAPUS_5A | 75.3 |
| CAMELINA_C1(80666) | NAPUS_5C | 76 |
| CAMELINA_C1(80666) | OSROD1_SEQIDNO11 | 43.8 |
| CAMELINA_C1(80666) | RCPDCT | 55.4 |
| CAMELINA_C1(80666) | RCROD1_SEQIDNO9 | 55.4 |
| CAMELINA_C1(80666) | ZMROD1_GRMZM2G015040 | 451 |
| CAMELINA_C1(80666) | ZMROD1_GRMZM2G087896 | 47 |
| CAMELINA_C15(45897) | ATRODD1 | 85.8 |
| CAMELINA_C15(45897) | BJROD1-A1 | 76.5 |
| CAMELINA_C15(45897) | BJROD1-A2 | 71.9 |
| CAMELINA_C15(45897) | BJROD1-A3 | 69.5 |
| CAMELINA_C15(45897) | BJROD1-B1 | 75.8 |
| CAMELINA_C15(45897) | BJROD1-B2 | 75.2 |
| CAMELINA_C15(45897) | BJROD1-B3 | 70.2 |
| CAMELINA_C15(45897) | BJROD1-B4 | 55.2 |
| CAMELINA_C15(45897) | BRROD1_SEQIDNO7 | 76.5 |
| CAMELINA_C15(45897) | CAMELINA_C1(80666) | 96.6 |
| CAMELINA_C15(45897) | CAMELINA_C15(45897) | 100 |
| CAMELINA_C15(45897) | CAMELINA_C19(65416) | 97.3 |
| CAMELINA_C15(45897) | CARINATA_1B | 70.2 |
| CAMELINA_C15(45897) | CARINATA_1C | 70.9 |
| CAMELINA_C15(45897) | CARINATA_2B | 55.2 |
| CAMELINA_C15(45897) | CARINATA_2C | 55.2 |
| CAMELINA_C15(45897) | CARINATA_3B | 75.8 |
| CAMELINA_C15(45897) | CARINATA_3C | 76.2 |
| CAMELINA_C15(45897) | CARINATA_5B | 76.8 |
| CAMELINA_C15(45897) | CARINATA_5C | 76.6 |
| CAMELINA_C15(45897) | GMROD1-1 | 61 |
| CAMELINA_C15(45897) | GMROD1-2 | 60.7 |
| CAMELINA_C15(45897) | LUPDCT1 | 53.9 |
| CAMELINA_C15(45897) | LUPDCT2 | 54.2 |
| CAMELINA_C15(45897) | NAPUS_1A | 69.5 |
| CAMELINA_C15(45897) | NAPUS_1C | 70.5 |
| CAMELINA_C15(45897) | NAPUS_2A | 55.2 |
| CAMELINA_C15(45897) | NAPUS_2C | 54.9 |
| CAMELINA_C15(45897) | NAPUS_3A | 76.5 |
| CAMELINA_C15(45897) | NAPUS_3C | 76.2 |
| CAMELINA_C15(45897) | NAPUS_5A | 75.6 |
| CAMELINA_C15(45897) | NAPUS_5C | 76.9 |
| CAMELINA_C15(45897) | OSROD1_SEQIDNO11 | 45.4 |
| CAMELINA_C15(45897) | RCPDCT | 59.8 |
| CAMELINA_C15(45897) | RCROD1_SEQIDNO9 | 59.8 |
| CAMELINA_C15(45897) | ZMROD1_GRMZM2G015040 | 45 |
| CAMELINA_C15(45897) | ZMROD1_GRMZM2G087896 | 46.5 |
| CAMELINA_C19(65416) | ATRODD1 | 86.2 |
| CAMELINA_C19(65416) | BJROD1-A1 | 76.5 |
| CAMELINA_C19(65416) | BJROD1-A2 | 72.5 |
| CAMELINA_C19(65416) | BJROD1-A3 | 69.5 |
| CAMELINA_C19(65416) | BJROD1-B1 | 75.8 |
| CAMELINA_C19(65416) | BJROD1-B2 | 75.2 |
| CAMELINA_C19(65416) | BJROD1-B3 | 70.9 |
| CAMELINA_C19(65416) | BJROD1-B4 | 55.2 |
| CAMELINA_C19(65416) | BRROD1_SEQIDNO7 | 76.5 |
| CAMELINA_C19(65416) | CAMELINA_C1(80666) | 98 |
| CAMELINA_C19(65416) | CAMELINA_C15(45897) | 97.3 |
| CAMELINA_C19(65416) | CAMELINA_C19(65416) | 100 |
| CAMELINA_C19(65416) | CARINATA_1B | 70.9 |
| CAMELINA_C19(65416) | CARINATA_1C | 71.5 |
| CAMELINA_C19(65416) | CARINATA_2B | 55.2 |
| CAMELINA_C19(65416) | CARINATA_2C | 55.2 |
| CAMELINA_C19(65416) | CARINATA_3B | 75.8 |
| CAMELINA_C19(65416) | CARINATA_3C | 76.5 |
| CAMELINA_C19(65416) | CARINATA_5B | 76.8 |
| CAMELINA_C19(65416) | CARINATA_5C | 76.1 |
| CAMELINA_C19(65416) | GMROD1-1 | 60.3 |
| CAMELINA_C19(65416) | GMROD1-2 | 60.5 |
| CAMELINA_C19(65416) | LUPDCT1 | 52.6 |
| CAMELINA_C19(65416) | LUPDCT2 | 55.3 |
| CAMELINA_C19(65416) | NAPUS_1A | 69.5 |
| CAMELINA_C19(65416) | NAPUS_1C | 71.2 |
| CAMELINA_C19(65416) | NAPUS_2A | 55.2 |
| CAMELINA_C19(65416) | NAPUS_2C | 54.9 |
| CAMELINA_C19(65416) | NAPUS_3A | 77.2 |
| CAMELINA_C19(65416) | NAPUS_3C | 76.5 |
| CAMELINA_C19(65416) | NAPUS_5A | 76.2 |
| CAMELINA_C19(65416) | NAPUS_5C | 76.4 |
| CAMELINA_C19(65416) | OSROD1_SEQIDNO11 | 43.9 |
| CAMELINA_C19(65416) | RCPDCT | 59.9 |
| CAMELINA_C19(65416) | RCROD1_SEQIDNO9 | 59.9 |
| CAMELINA_C19(65416) | ZMROD1_GRMZM2G015040 | 438 |
| CAMELINA_C19(65416) | ZMROD1_GRMZM2G087896 | 47.7 |
| CARINATA_1B | ATRODD1 | 73.7 |
| CARINATA_1B | BJROD1-A1 | 77.8 |
| CARINATA_1B | BJROD1-A2 | 77.9 |
| CARINATA_1B | BJROD1-A3 | 95.8 |
| CARINATA_1B | BJROD1-B1 | 78.5 |
| CARINATA_1B | BJROD1-B2 | 77.1 |
| CARINATA_1B | BJROD1-B3 | 98.6 |
| CARINATA_1B | BJROD1-B4 | 56.4 |
| CARINATA_1B | BRROD1_SEQIDNO7 | 78.5 |
| CARINATA_1B | CAMELINA_C1(80666) | 70.4 |
| CARINATA_1B | CAMELINA_C15(45897) | 70.2 |
| CARINATA_1B | CAMELINA_C19(65416) | 70.9 |
| CARINATA_1B | CARINATA_1B | 100 |
| CARINATA_1B | CARINATA_1C | 93.8 |
| CARINATA_1B | CARINATA_2B | 56.4 |
| CARINATA_1B | CARINATA_2C | 56.7 |
| CARINATA_1B | CARINATA_3B | 78.8 |
| CARINATA_1B | CARINATA_3C | 78.8 |
| CARINATA_1B | CARINATA_5B | 80.2 |
| CARINATA_1B | CARINATA_5C | 78.8 |
| CARINATA_1B | GMROD1-1 | 61.1 |
| CARINATA_1B | GMROD1-2 | 55.3 |
| CARINATA_1B | LUPDCT1 | 54.1 |
| CARINATA_1B | LUPDCT2 | 54.5 |
| CARINATA_1B | NAPUS_1A | 94.5 |
| CARINATA_1B | NAPUS_1C | 94.8 |
| CARINATA_1B | NAPUS_2A | 56.4 |
| CARINATA_1B | NAPUS_2C | 56.1 |
| CARINATA_1B | NAPUS_3A | 78.3 |
| CARINATA_1B | NAPUS_3C | 78.8 |
| CARINATA_1B | NAPUS_5A | 78.9 |
| CARINATA_1B | NAPUS_5C | 79.2 |
| CARINATA_1B | OSROD1_SEQIDNO11 | 42.3 |
| CARINATA_1B | RCPDCT | 57.6 |
| CARINATA_1B | RCROD1_SEQIDNO9 | 57.6 |
| CARINATA_1B | ZMROD1_GRMZM2G015040 | 44 |
| CARINATA_1B | ZMROD1_GRMZM2G087896 | 44.7 |
| CARINATA_1C | ATRODD1 | 74 |
| CARINATA_1C | BJROD1-A1 | 78.8 |
| CARINATA_1C | BJROD1-A2 | 78.3 |
| CARINATA_1C | BJROD1-A3 | 94.5 |
| CARINATA_1C | BJROD1-B1 | 79.2 |
| CARINATA_1C | BJROD1-B2 | 77.5 |
| CARINATA_1C | BJROD1-B3 | 94.5 |
| CARINATA_1C | BJROD1-B4 | 55.4 |
| CARINATA_1C | BRROD1_SEQIDNO7 | 79.5 |
| CARINATA_1C | CAMELINA_C1(80666) | 71.1 |
| CARINATA_1C | CAMELINA_C15(45897) | 70.9 |
| CARINATA_1C | CAMELINA_C19(65416) | 71.5 |
| CARINATA_1C | CARINATA_1B | 93.8 |
| CARINATA_1C | CARINATA_1C | 100 |
| CARINATA_1C | CARINATA_2B | 55.4 |
| CARINATA_1C | CARINATA_2C | 55.7 |
| CARINATA_1C | CARINATA_3B | 79.5 |
| CARINATA_1C | CARINATA_3C | 79.9 |
| CARINATA_1C | CARINATA_5B | 80.3 |
| CARINATA_1C | CARINATA_5C | 79.2 |
| CARINATA_1C | GMROD1-1 | 60.3 |
| CARINATA_1C | GMROD1-2 | 52.9 |
| CARINATA_1C | LUPDCT1 | 53.3 |
| CARINATA_1C | LUPDCT2 | 53.6 |
| CARINATA_1C | NAPUS_1A | 95.5 |
| CARINATA_1C | NAPUS_1C | 99 |
| CARINATA_1C | NAPUS_2A | 55.4 |
| CARINATA_1C | NAPUS_2C | 55 |
| CARINATA_1C | NAPUS_3A | 79.3 |
| CARINATA_1C | NAPUS_3C | 79.9 |
| CARINATA_1C | NAPUS_5A | 79.3 |
| CARINATA_1C | NAPUS_5C | 79.5 |
| CARINATA_1C | OSROD1_SEQIDNO11 | 41.7 |
| CARINATA_1C | RCPDCT | 57.9 |
| CARINATA_1C | RCROD1_SEQIDNO9 | 57.9 |
| CARINATA_1C | ZMROD1_GRMZM2G015040 | 42.9 |
| CARINATA_1C | ZMROD1_GRMZM2G087896 | 42.7 |
| CARINATA_2B | ATRODD1 | 55.5 |
| CARINATA_2B | BJROD1-A1 | 57.1 |
| CARINATA_2B | BJROD1-A2 | 55.4 |
| CARINATA_2B | BJROD1-A3 | 55 |
| CARINATA_2B | BJROD1-B1 | 56.8 |
| CARINATA_2B | BJROD1-B2 | 58.9 |
| CARINATA_2B | BJROD1-B3 | 55.4 |
| CARINATA_2B | BJROD1-B4 | 97.4 |
| CARINATA_2B | BRROD1_SEQIDNO7 | 57.1 |
| CARINATA_2B | CAMELINA_C1(80666) | 55.4 |
| CARINATA_2B | CAMELINA_C15(45897) | 55.2 |
| CARINATA_2B | CAMELINA_C19(65416) | 55.2 |
| CARINATA_2B | CARINATA_1B | 56.4 |
| CARINATA_2B | CARINATA_1C | 55.4 |
| CARINATA_2B | CARINATA_2B | 100 |
| CARINATA_2B | CARINATA_2C | 99.1 |
| CARINATA_2B | CARINATA_3B | 57.1 |
| CARINATA_2B | CARINATA_3C | 56.8 |
| CARINATA_2B | CARINATA_5B | 58.2 |
| CARINATA_2B | CARINATA_5C | 55.5 |
| CARINATA_2B | GMROD1-1 | 55.7 |
| CARINATA_2B | GMROD1-2 | 54.6 |
| CARINATA_2B | LUPDCT1 | 49.3 |
| CARINATA_2B | LUPDCT2 | 49.3 |
| CARINATA_2B | NAPUS_1A | 55 |
| CARINATA_2B | NAPUS_1C | 55.7 |
| CARINATA_2B | NAPUS_2A | 97 |
| CARINATA_2B | NAPUS_2C | 96.6 |
| CARINATA_2B | NAPUS_3A | 57.4 |
| CARINATA_2B | NAPUS_3C | 56.8 |
| CARINATA_2B | NAPUS_5A | 55.5 |
| CARINATA_2B | NAPUS_5C | 55.5 |
| CARINATA_2B | OSROD1_SEQIDNO11 | 38.1 |
| CARINATA_2B | RCPDCT | 52.3 |
| CARINATA_2B | RCROD1_SEQIDNO9 | 52.3 |
| CARINATA_2B | ZMROD1_GRMZM2G015040 | 44.9 |
| CARINATA_2B | ZMROD1_GRMZM2G087896 | 44.2 |
| CARINATA_2C | ATRODD1 | 55.5 |
| CARINATA_2C | BJROD1-A1 | 57.1 |
| CARINATA_2C | BJROD1-A2 | 55.4 |
| CARINATA_2C | BJROD1-A3 | 55.4 |
| CARINATA_2C | BJROD1-B1 | 56.8 |
| CARINATA_2C | BJROD1-B2 | 59 |
| CARINATA_2C | BJROD1-B3 | 56.4 |
| CARINATA_2C | BJROD1-B4 | 98.3 |
| CARINATA_2C | BRROD1_SEQIDNO7 | 57.1 |
| CARINATA_2C | CAMELINA_C1(80666) | 55.4 |
| CARINATA_2C | CAMELINA_C15(45897) | 55.2 |
| CARINATA_2C | CAMELINA_C19(65416) | 55.2 |
| CARINATA_2C | CARINATA_1B | 56.7 |
| CARINATA_2C | CARINATA_1C | 55.7 |
| CARINATA_2C | CARINATA_2B | 99.1 |
| CARINATA_2C | CARINATA_2C | 100 |
| CARINATA_2C | CARINATA_3B | 57.1 |
| CARINATA_2C | CARINATA_3C | 56.8 |
| CARINATA_2C | CARINATA_5B | 58.2 |
| CARINATA_2C | CARINATA_5C | 55.5 |
| CARINATA_2C | GMROD1-1 | 55.3 |
| CARINATA_2C | GMROD1-2 | 53.9 |
| CARINATA_2C | LUPDCT1 | 48.7 |
| CARINATA_2C | LUPDCT2 | 48.7 |
| CARINATA_2C | NAPUS_1A | 55.4 |
| CARINATA_2C | NAPUS_1C | 56.1 |
| CARINATA_2C | NAPUS_2A | 97.9 |
| CARINATA_2C | NAPUS_2C | 97.4 |
| CARINATA_2C | NAPUS_3A | 57.4 |
| CARINATA_2C | NAPUS_3C | 56.8 |
| CARINATA_2C | NAPUS_5A | 55.5 |
| CARINATA_2C | NAPUS_5C | 55.5 |
| CARINATA_2C | OSROD1_SEQIDNO11 | 38.1 |
| CARINATA_2C | RCPDCT | 51.9 |
| CARINATA_2C | RCROD1_SEQIDNO9 | 51.9 |
| CARINATA_2C | ZMROD1_GRMZM2G015040 | 45.3 |
| CARINATA_2C | ZMROD1_GRMZM2G087896 | 46.2 |
| CARINATA_3B | ATRODD1 | 78.5 |
| CARINATA_3B | BJROD1-A1 | 96.8 |
| CARINATA_3B | BJROD1-A2 | 83.3 |
| CARINATA_3B | BJROD1-A3 | 78.8 |
| CARINATA_3B | BJROD1-B1 | 99.3 |
| CARINATA_3B | BJROD1-B2 | 83.3 |
| CARINATA_3B | BJROD1-B3 | 78.8 |
| CARINATA_3B | BJROD1-B4 | 57.1 |
| CARINATA_3B | BRROD1_SEQIDNO7 | 97.5 |
| CARINATA_3B | CAMELINA_C1(80666) | 76.5 |
| CARINATA_3B | CAMELINA_C15(45897) | 75.8 |
| CARINATA_3B | CAMELINA_C19(65416) | 75.8 |
| CARINATA_3B | CARINATA_1B | 78.8 |
| CARINATA_3B | CARINATA_1C | 79.5 |
| CARINATA_3B | CARINATA_2B | 57.1 |
| CARINATA_3B | CARINATA_2C | 57.1 |
| CARINATA_3B | CARINATA_3B | 100 |
| CARINATA_3B | CARINATA_3C | 98.2 |
| CARINATA_3B | CARINATA_5B | 86.8 |
| CARINATA_3B | CARINATA_5C | 86.8 |
| CARINATA_3B | GMROD1-1 | 61.9 |
| CARINATA_3B | GMROD1-2 | 63.1 |
| CARINATA_3B | LUPDCT1 | 54.2 |
| CARINATA_3B | LUPDCT2 | 54.2 |
| CARINATA_3B | NAPUS_1A | 78.2 |
| CARINATA_3B | NAPUS_1C | 79.5 |
| CARINATA_3B | NAPUS_2A | 57.1 |
| CARINATA_3B | NAPUS_2C | 56.8 |
| CARINATA_3B | NAPUS_3A | 96.8 |
| CARINATA_3B | NAPUS_3C | 98.2 |
| CARINATA_3B | NAPUS_5A | 86.8 |
| CARINATA_3B | NAPUS_5C | 87.2 |
| CARINATA_3B | OSROD1_SEQIDNO11 | 44.1 |
| CARINATA_3B | RCPDCT | 61.1 |
| CARINATA_3B | RCROD1_SEQIDNO9 | 61.1 |
| CARINATA_3B | ZMROD1_GRMZM2G015040 | 46.4 |
| CARINATA_3B | ZMROD1_GRMZM2G087896 | 44.4 |
| CARINATA_3C | ATRODD1 | 78.8 |
| CARINATA_3C | BJROD1-A1 | 97.9 |
| CARINATA_3C | BJROD1-A2 | 83 |
| CARINATA_3C | BJROD1-A3 | 78.8 |
| CARINATA_3C | BJROD1-B1 | 98.2 |
| CARINATA_3C | BJROD1-B2 | 85.2 |
| CARINATA_3C | BJROD1-B3 | 78.8 |
| CARINATA_3C | BJROD1-B4 | 56.8 |
| CARINATA_3C | BRROD1_SEQIDNO7 | 98.6 |
| CARINATA_3C | CAMELINA_C1(80666) | 76.8 |
| CARINATA_3C | CAMELINA_C15(45897) | 76.2 |
| CARINATA_3C | CAMELINA_C19(65416) | 76.5 |
| CARINATA_3C | CARINATA_1B | 78.8 |
| CARINATA_3C | CARINATA_1C | 79.9 |
| CARINATA_3C | CARINATA_2B | 56.8 |
| CARINATA_3C | CARINATA_2C | 56.8 |
| CARINATA_3C | CARINATA_3B | 98.2 |
| CARINATA_3C | CARINATA_3C | 100 |
| CARINATA_3C | CARINATA_5B | 87.1 |
| CARINATA_3C | CARINATA_5C | 86.8 |
| CARINATA_3C | GMROD1-1 | 61.9 |
| CARINATA_3C | GMROD1-2 | 63.1 |
| CARINATA_3C | LUPDCT1 | 54.5 |
| CARINATA_3C | LUPDCT2 | 54.5 |
| CARINATA_3C | NAPUS_1A | 78.2 |
| CARINATA_3C | NAPUS_1C | 79.5 |
| CARINATA_3C | NAPUS_2A | 56.8 |
| CARINATA_3C | NAPUS_2C | 56.4 |
| CARINATA_3C | NAPUS_3A | 98.2 |
| CARINATA_3C | NAPUS_3C | 100 |
| CARINATA_3C | NAPUS_5A | 86.8 |
| CARINATA_3C | NAPUS_5C | 87.2 |
| CARINATA_3C | OSROD1_SEQIDNO11 | 44.9 |
| CARINATA_3C | RCPDCT | 60.8 |
| CARINATA_3C | RCROD1_SEQIDNO9 | 60.8 |
| CARINATA_3C | ZMROD1_GRMZM2G015040 | 45.8 |
| CARINATA_3C | ZMROD1_GRMZM2G087896 | 44.4 |
| CARINATA_5B | ATRODD1 | 80.5 |
| CARINATA_5B | BJROD1-A1 | 87.1 |
| CARINATA_5B | BJROD1-A2 | 88.8 |
| CARINATA_5B | BJROD1-A3 | 79.3 |
| CARINATA_5B | BJROD1-B1 | 86.8 |
| CARINATA_5B | BJROD1-B2 | 93.3 |
| CARINATA_5B | BJROD1-B3 | 80.2 |
| CARINATA_5B | BJROD1-B4 | 58.2 |
| CARINATA_5B | BRROD1_SEQIDNO7 | 87.5 |
| CARINATA_5B | CAMELINA_C1(80666) | 77.1 |
| CARINATA_5B | CAMELINA_C15(45897) | 76.8 |
| CARINATA_5B | CAMELINA_C19(65416) | 76.8 |
| CARINATA_5B | CARINATA_1B | 80.2 |
| CARINATA_5B | CARINATA_1C | 80.3 |
| CARINATA_5B | CARINATA_2B | 58.2 |
| CARINATA_5B | CARINATA_2C | 58.2 |
| CARINATA_5B | CARINATA_3B | 86.8 |
| CARINATA_5B | CARINATA_3C | 87.1 |
| CARINATA_5B | CARINATA_5B | 100 |
| CARINATA_5B | CARINATA_5C | 93.7 |
| CARINATA_5B | GMROD1-1 | 61.7 |
| CARINATA_5B | GMROD1-2 | 61.2 |
| CARINATA_5B | LUPDCT1 | 54.1 |
| CARINATA_5B | LUPDCT2 | 54.1 |
| CARINATA_5B | NAPUS_1A | 78.6 |
| CARINATA_5B | NAPUS_1C | 79.9 |
| CARINATA_5B | NAPUS_2A | 58.2 |
| CARINATA_5B | NAPUS_2C | 57.8 |
| CARINATA_5B | NAPUS_3A | 86.5 |
| CARINATA_5B | NAPUS_3C | 87.1 |
| CARINATA_5B | NAPUS_5A | 92.7 |
| CARINATA_5B | NAPUS_5C | 94.1 |
| CARINATA_5B | OSROD1_SEQIDNO11 | 42.6 |
| CARINATA_5B | RCPDCT | 59.9 |
| CARINATA_5B | RCROD1_SEQIDNO9 | 59.9 |
| CARINATA_5B | ZMROD1_GRMZM2G015040 | 46.4 |
| CARINATA_5B | ZMROD1_GRMZM2G087896 | 45.8 |
| CARINATA_5C | ATRODD1 | 79.8 |
| CARINATA_5C | BJROD1-A1 | 86.5 |
| CARINATA_5C | BJROD1-A2 | 93.3 |
| CARINATA_5C | BJROD1-A3 | 78.2 |
| CARINATA_5C | BJROD1-B1 | 86.8 |
| CARINATA_5C | BJROD1-B2 | 91.2 |
| CARINATA_5C | BJROD1-B3 | 78.8 |
| CARINATA_5C | BJROD1-B4 | 55.5 |
| CARINATA_5C | BRROD1_SEQIDNO7 | 86.8 |
| CARINATA_5C | CAMELINA_C1(80666) | 75.7 |
| CARINATA_5C | CAMELINA_C15(45897) | 76.6 |
| CARINATA_5C | CAMELINA_C19(65416) | 76.1 |
| CARINATA_5C | CARINATA_1B | 78.8 |
| CARINATA_5C | CARINATA_1C | 79.2 |
| CARINATA_5C | CARINATA_2B | 55.5 |
| CARINATA_5C | CARINATA_2C | 55.5 |
| CARINATA_5C | CARINATA_3B | 86.8 |
| CARINATA_5C | CARINATA_3C | 86.8 |
| CARINATA_5C | CARINATA_5B | 93.7 |
| CARINATA_5C | CARINATA_5C | 100 |
| CARINATA_5C | GMROD1-1 | 60.3 |
| CARINATA_5C | GMROD1-2 | 61.1 |
| CARINATA_5C | LUPDCT1 | 51.7 |
| CARINATA_5C | LUPDCT2 | 51.7 |
| CARINATA_5C | NAPUS_1A | 77.5 |
| CARINATA_5C | NAPUS_1C | 78.8 |
| CARINATA_5C | NAPUS_2A | 55.5 |
| CARINATA_5C | NAPUS_2C | 55.1 |
| CARINATA_5C | NAPUS_3A | 85.5 |
| CARINATA_5C | NAPUS_3C | 86.8 |
| CARINATA_5C | NAPUS_5A | 97.5 |
| CARINATA_5C | NAPUS_5C | 99.6 |
| CARINATA_5C | OSROD1_SEQIDNO11 | 42.5 |
| CARINATA_5C | RCPDCT | 59.9 |
| CARINATA_5C | RCROD1_SEQIDNO9 | 59.9 |
| CARINATA_5C | ZMROD1_GRMZM2G015040 | 46.4 |
| CARINATA_5C | ZMROD1_GRMZM2G087896 | 44.8 |
| GMROD1-1 | ATRODD1 | 60.7 |
| GMROD1-1 | BJROD1-A1 | 62.4 |
| GMROD1-1 | BJROD1-A2 | 62.1 |
| GMROD1-1 | BJROD1-A3 | 60.2 |
| GMROD1-1 | BJROD1-B1 | 61.5 |
| GMROD1-1 | BJROD1-B2 | 64.1 |
| GMROD1-1 | BJROD1-B3 | 61.5 |
| GMROD1-1 | BJROD1-B4 | 54.9 |
| GMROD1-1 | BRROD1_SEQIDNO7 | 61.2 |
| GMROD1-1 | CAMELINA_C1(80666) | 60.8 |
| GMROD1-1 | CAMELINA_C15(45897) | 61 |
| GMROD1-1 | CAMELINA_C19(65416) | 60.3 |
| GMROD1-1 | CARINATA_1B | 61.1 |
| GMROD1-1 | CARINATA_1C | 60.3 |
| GMROD1-1 | CARINATA_2B | 55.7 |
| GMROD1-1 | CARINATA_2C | 55.3 |
| GMROD1-1 | CARINATA_3B | 61.9 |
| GMROD1-1 | CARINATA_3C | 61.9 |
| GMROD1-1 | CARINATA_5B | 61.7 |
| GMROD1-1 | CARINATA_5C | 60.3 |
| GMROD1-1 | GMROD1-1 | 100 |
| GMROD1-1 | GMROD1-2 | 86.3 |
| GMROD1-1 | LUPDCT1 | 60.1 |
| GMROD1-1 | LUPDCT2 | 60.1 |
| GMROD1-1 | NAPUS_1A | 60.5 |
| GMROD1-1 | NAPUS_1C | 60.3 |
| GMROD1-1 | NAPUS_2A | 54.9 |
| GMROD1-1 | NAPUS_2C | 54.6 |
| GMROD1-1 | NAPUS_3A | 61.2 |
| GMROD1-1 | NAPUS_3C | 61.9 |
| GMROD1-1 | NAPUS_5A | 62.3 |
| GMROD1-1 | NAPUS_5C | 60.3 |
| GMROD1-1 | OSROD1_SEQIDNO11 | 47.1 |
| GMROD1-1 | RCPDCT | 68.2 |
| GMROD1-1 | RCROD1_SEQIDNO9 | 68.2 |
| GMROD1-1 | ZMROD1_GRMZM2G015040 | 51.4 |
| GMROD1-1 | ZMROD1_GRMZM2G087896 | 53.1 |
| GMROD1-2 | ATRODD1 | 58.1 |
| GMROD1-2 | BJROD1-A1 | 62.8 |
| GMROD1-2 | BJROD1-A2 | 57.5 |
| GMROD1-2 | BJROD1-A3 | 54.4 |
| GMROD1-2 | BJROD1-B1 | 62.8 |
| GMROD1-2 | BJROD1-B2 | 65 |
| GMROD1-2 | BJROD1-B3 | 52.7 |
| GMROD1-2 | BJROD1-B4 | 53.5 |
| GMROD1-2 | BRROD1_SEQIDNO7 | 63.1 |
| GMROD1-2 | CAMELINA_C1(80666) | 60.1 |
| GMROD1-2 | CAMELINA_C15(45897) | 60.7 |
| GMROD1-2 | CAMELINA_C19(65416) | 60.5 |
| GMROD1-2 | CARINATA_1B | 55.3 |
| GMROD1-2 | CARINATA_1C | 52.9 |
| GMROD1-2 | CARINATA_2B | 54.6 |
| GMROD1-2 | CARINATA_2C | 53.9 |
| GMROD1-2 | CARINATA_3B | 63.1 |
| GMROD1-2 | CARINATA_3C | 63.1 |
| GMROD1-2 | CARINATA_5B | 61.2 |
| GMROD1-2 | CARINATA_5C | 61.1 |
| GMROD1-2 | GMROD1-1 | 86.3 |
| GMROD1-2 | GMROD1-2 | 100 |
| GMROD1-2 | LUPDCT1 | 56.1 |
| GMROD1-2 | LUPDCT2 | 56.1 |
| GMROD1-2 | NAPUS_1A | 54.4 |
| GMROD1-2 | NAPUS_1C | 52.9 |
| GMROD1-2 | NAPUS_2A | 53.5 |
| GMROD1-2 | NAPUS_2C | 53.2 |
| GMROD1-2 | NAPUS_3A | 62.7 |
| GMROD1-2 | NAPUS_3C | 63.1 |
| GMROD1-2 | NAPUS_5A | 61 |
| GMROD1-2 | NAPUS_5C | 61.1 |
| GMROD1-2 | OSROD1_SEQIDNO11 | 46.5 |
| GMROD1-2 | RCPDCT | 59.3 |
| GMROD1-2 | RCROD1_SEQIDNO9 | 59.3 |
| GMROD1-2 | ZMROD1_GRMZM2G015040 | 50.9 |
| GMROD1-2 | ZMROD1_GRMZM2G087896 | 49 |
| LUPDCT1 | ATRODD1 | 54.6 |
| LUPDCT1 | BJROD1-A1 | 54.5 |
| LUPDCT1 | BJROD1-A2 | 51.5 |
| LUPDCT1 | BJROD1-A3 | 52.5 |
| LUPDCT1 | BJROD1-B1 | 53.9 |
| LUPDCT1 | BJROD1-B2 | 53.8 |
| LUPDCT1 | BJROD1-B3 | 53.3 |
| LUPDCT1 | BJROD1-B4 | 48.4 |
| LUPDCT1 | BRROD1_SEQIDNO7 | 54.5 |
| LUPDCT1 | CAMELINA_C1(80666) | 55 |
| LUPDCT1 | CAMELINA_C15(45897) | 53.9 |
| LUPDCT1 | CAMELINA_C19(65416) | 52.6 |
| LUPDCT1 | CARINATA_1B | 54.1 |
| LUPDCT1 | CARINATA_1C | 53.3 |
| LUPDCT1 | CARINATA_2B | 49.3 |
| LUPDCT1 | CARINATA_2C | 48.7 |
| LUPDCT1 | CARINATA_3B | 54.2 |
| LUPDCT1 | CARINATA_3C | 54.5 |
| LUPDCT1 | CARINATA_5B | 54.1 |
| LUPDCT1 | CARINATA_5C | 51.7 |
| LUPDCT1 | GMROD1-1 | 60.1 |
| LUPDCT1 | GMROD1-2 | 56.1 |
| LUPDCT1 | LUPDCT1 | 100 |
| LUPDCT1 | LUPDCT2 | 98.6 |
| LUPDCT1 | NAPUS_1A | 52.9 |
| LUPDCT1 | NAPUS_1C | 53.3 |
| LUPDCT1 | NAPUS_2A | 48.4 |
| LUPDCT1 | NAPUS_2C | 48 |
| LUPDCT1 | NAPUS_3A | 54.9 |
| LUPDCT1 | NAPUS_3C | 54.5 |
| LUPDCT1 | NAPUS_5A | 52 |
| LUPDCT1 | NAPUS_5C | 52 |
| LUPDCT1 | OSROD1_SEQIDNO11 | 45.9 |
| LUPDCT1 | RCPDCT | 59.2 |
| LUPDCT1 | RCROD1_SEQIDNO9 | 59.2 |
| LUPDCT1 | ZMROD1_GRMZM2G015040 | 48.1 |
| LUPDCT1 | ZMROD1_GRMZM2G087896 | 49 |
| LUPDCT2 | ATRODD1 | 54.2 |
| LUPDCT2 | BJROD1-A1 | 54.5 |
| LUPDCT2 | BJROD1-A2 | 51.5 |
| LUPDCT2 | BJROD1-A3 | 53.4 |
| LUPDCT2 | BJROD1-B1 | 53.9 |
| LUPDCT2 | BJROD1-B2 | 53.8 |
| LUPDCT2 | BJROD1-B3 | 53.6 |
| LUPDCT2 | BJROD1-B4 | 48.4 |
| LUPDCT2 | BRROD1_SEQIDNO7 | 54.5 |
| LUPDCT2 | CAMELINA_C1(80666) | 55.3 |
| LUPDCT2 | CAMELINA_C15(45897) | 54.2 |
| LUPDCT2 | CAMELINA_C19(65416) | 55.3 |
| LUPDCT2 | CARINATA_1B | 54.5 |
| LUPDCT2 | CARINATA_1C | 53.6 |
| LUPDCT2 | CARINATA_2B | 49.3 |
| LUPDCT2 | CARINATA_2C | 48.7 |
| LUPDCT2 | CARINATA_3B | 54.2 |
| LUPDCT2 | CARINATA_3C | 54.5 |
| LUPDCT2 | CARINATA_5B | 54.1 |
| LUPDCT2 | CARINATA_5C | 51.7 |
| LUPDCT2 | GMROD1-1 | 60.1 |
| LUPDCT2 | GMROD1-2 | 56.1 |
| LUPDCT2 | LUPDCT1 | 98.6 |
| LUPDCT2 | LUPDCT2 | 100 |
| LUPDCT2 | NAPUS_1A | 53.8 |
| LUPDCT2 | NAPUS_1C | 53.6 |
| LUPDCT2 | NAPUS_2A | 48.4 |
| LUPDCT2 | NAPUS_2C | 48 |
| LUPDCT2 | NAPUS_3A | 54.9 |
| LUPDCT2 | NAPUS_3C | 54.5 |
| LUPDCT2 | NAPUS_5A | 52 |
| LUPDCT2 | NAPUS_5C | 52 |
| LUPDCT2 | OSROD1_SEQIDNO11 | 46.3 |
| LUPDCT2 | RCPDCT | 59.2 |
| LUPDCT2 | RCROD1_SEQIDNO9 | 59.2 |
| LUPDCT2 | ZMROD1_GRMZM2G015040 | 47.8 |
| LUPDCT2 | ZMROD1_GRMZM2G087896 | 48.6 |
| NAPUS_1A | ATRODD1 | 72.7 |
| NAPUS_1A | BJROD1-A1 | 77.1 |
| NAPUS_1A | BJROD1-A2 | 76.6 |
| NAPUS_1A | BJROD1-A3 | 98.6 |
| NAPUS_1A | BJROD1-B1 | 77.8 |
| NAPUS_1A | BJROD1-B2 | 75.4 |
| NAPUS_1A | BJROD1-B3 | 95.2 |
| NAPUS_1A | BJROD1-B4 | 55.4 |
| NAPUS_1A | BRROD1_SEQIDNO7 | 77.8 |
| NAPUS_1A | CAMELINA_C1(80666) | 69.1 |
| NAPUS_1A | CAMELINA_C15(45897) | 69.5 |
| NAPUS_1A | CAMELINA_C19(65416) | 69.5 |
| NAPUS_1A | CARINATA_1B | 94.5 |
| NAPUS_1A | CARINATA_1C | 95.5 |
| NAPUS_1A | CARINATA_2B | 55 |
| NAPUS_1A | CARINATA_2C | 55.4 |
| NAPUS_1A | CARINATA_3B | 78.2 |
| NAPUS_1A | CARINATA_3C | 78.2 |
| NAPUS_1A | CARINATA_5B | 78.6 |
| NAPUS_1A | CARINATA_5C | 77.5 |
| NAPUS_1A | GMROD1-1 | 60.5 |
| NAPUS_1A | GMROD1-2 | 54.4 |
| NAPUS_1A | LUPDCT1 | 52.9 |
| NAPUS_1A | LUPDCT2 | 53.8 |
| NAPUS_1A | NAPUS_1A | 100 |
| NAPUS_1A | NAPUS_1C | 96.5 |
| NAPUS_1A | NAPUS_2A | 55.4 |
| NAPUS_1A | NAPUS_2C | 55 |
| NAPUS_1A | NAPUS_3A | 77.6 |
| NAPUS_1A | NAPUS_3C | 78.2 |
| NAPUS_1A | NAPUS_5A | 77.6 |
| NAPUS_1A | NAPUS_5C | 77.8 |
| NAPUS_1A | OSROD1_SEQIDNO11 | 42.4 |
| NAPUS_1A | RCPDCT | 57.3 |
| NAPUS_1A | RCROD1_SEQIDNO9 | 57.3 |
| NAPUS_1A | ZMROD1_GRMZM2G015040 | 44 |
| NAPUS_1A | ZMROD1_GRMZM2G087896 | 43 |
| NAPUS_1C | ATRODD1 | 73.7 |
| NAPUS_1C | BJROD1-A1 | 78.5 |
| NAPUS_1C | BJROD1-A2 | 77.9 |
| NAPUS_1C | BJROD1-A3 | 95.5 |
| NAPUS_1C | BJROD1-B1 | 79.2 |
| NAPUS_1C | BJROD1-B2 | 77.1 |
| NAPUS_1C | BJROD1-B3 | 95.5 |
| NAPUS_1C | BJROD1-B4 | 55.7 |
| NAPUS_1C | BRROD1_SEQIDNO7 | 79.2 |
| NAPUS_1C | CAMELINA_C1(80666) | 70.8 |
| NAPUS_1C | CAMELINA_C15(45897) | 70.5 |
| NAPUS_1C | CAMELINA_C19(65416) | 71.2 |
| NAPUS_1C | CARINATA_1B | 94.8 |
| NAPUS_1C | CARINATA_1C | 99 |
| NAPUS_1C | CARINATA_2B | 55.7 |
| NAPUS_1C | CARINATA_2C | 56.1 |
| NAPUS_1C | CARINATA_3B | 79.5 |
| NAPUS_1C | CARINATA_3C | 79.5 |
| NAPUS_1C | CARINATA_5B | 79.9 |
| NAPUS_1C | CARINATA_5C | 78.8 |
| NAPUS_1C | GMROD1-1 | 60.3 |
| NAPUS_1C | GMROD1-2 | 52.9 |
| NAPUS_1C | LUPDCT1 | 53.3 |
| NAPUS_1C | LUPDCT2 | 53.6 |
| NAPUS_1C | NAPUS_1A | 96.5 |
| NAPUS_1C | NAPUS_1C | 100 |
| NAPUS_1C | NAPUS_2A | 55.7 |
| NAPUS_1C | NAPUS_2C | 55.4 |
| NAPUS_1C | NAPUS_3A | 79 |
| NAPUS_1C | NAPUS_3C | 79.5 |
| NAPUS_1C | NAPUS_5A | 78.9 |
| NAPUS_1C | NAPUS_5C | 79.2 |
| NAPUS_1C | OSROD1_SEQIDNO11 | 42 |
| NAPUS_1C | RCPDCT | 57.9 |
| NAPUS_1C | RCROD1_SEQIDNO9 | 57.9 |
| NAPUS_1C | ZMROD1_GRMZM2G015040 | 43.3 |
| NAPUS_1C | ZMROD1_GRMZM2G087896 | 43 |
| NAPUS_2A | ATRODD1 | 55.5 |
| NAPUS_2A | BJROD1-A1 | 57.1 |
| NAPUS_2A | BJROD1-A2 | 55.4 |
| NAPUS_2A | BJROD1-A3 | 55.4 |
| NAPUS_2A | BJROD1-B1 | 56.8 |
| NAPUS_2A | BJROD1-B2 | 59 |
| NAPUS_2A | BJROD1-B3 | 56.1 |
| NAPUS_2A | BJROD1-B4 | 99.6 |
| NAPUS_2A | BRROD1_SEQIDNO7 | 57.1 |
| NAPUS_2A | CAMELINA_C1(80666) | 55.4 |
| NAPUS_2A | CAMELINA_C15(45897) | 55.2 |
| NAPUS_2A | CAMELINA_C19(65416) | 55.2 |
| NAPUS_2A | CARINATA_1B | 56.4 |
| NAPUS_2A | CARINATA_1C | 55.4 |
| NAPUS_2A | CARINATA_2B | 97 |
| NAPUS_2A | CARINATA_2C | 97.9 |
| NAPUS_2A | CARINATA_3B | 57.1 |
| NAPUS_2A | CARINATA_3C | 56.8 |
| NAPUS_2A | CARINATA_5B | 58.2 |
| NAPUS_2A | CARINATA_5C | 55.5 |
| NAPUS_2A | GMROD1-1 | 54.9 |
| NAPUS_2A | GMROD1-2 | 53.5 |
| NAPUS_2A | LUPDCT1 | 48.4 |
| NAPUS_2A | LUPDCT2 | 48.4 |
| NAPUS_2A | NAPUS_1A | 55.4 |
| NAPUS_2A | NAPUS_1C | 55.7 |
| NAPUS_2A | NAPUS_2A | 100 |
| NAPUS_2A | NAPUS_2C | 99.6 |
| NAPUS_2A | NAPUS_3A | 57.4 |
| NAPUS_2A | NAPUS_3C | 56.8 |
| NAPUS_2A | NAPUS_5A | 55.5 |
| NAPUS_2A | NAPUS_5C | 55.5 |
| NAPUS_2A | OSROD1_SEQIDNO11 | 38.1 |
| NAPUS_2A | RCPDCT | 51.6 |
| NAPUS_2A | RCROD1_SEQIDNO9 | 51.6 |
| NAPUS_2A | ZMROD1_GRMZM2G015040 | 44.9 |
| NAPUS_2A | ZMROD1_GRMZM2G087896 | 45.5 |
| NAPUS_2C | ATRODD1 | 55.1 |
| NAPUS_2C | BJROD1-A1 | 56.8 |
| NAPUS_2C | BJROD1-A2 | 55.1 |
| NAPUS_2C | BJROD1-A3 | 55 |
| NAPUS_2C | BJROD1-B1 | 56.4 |
| NAPUS_2C | BJROD1-B2 | 58.6 |
| NAPUS_2C | BJROD1-B3 | 55.7 |
| NAPUS_2C | BJROD1-B4 | 99.1 |
| NAPUS_2C | BRROD1_SEQIDNO7 | 56.8 |
| NAPUS_2C | CAMELINA_C1(80666) | 55.1 |
| NAPUS_2C | CAMELINA_C15(45897) | 54.9 |
| NAPUS_2C | CAMELINA_C19(65416) | 54.9 |
| NAPUS_2C | CARINATA_1B | 56.1 |
| NAPUS_2C | CARINATA_1C | 55 |
| NAPUS_2C | CARINATA_2B | 96.6 |
| NAPUS_2C | CARINATA_2C | 97.4 |
| NAPUS_2C | CARINATA_3B | 56.8 |
| NAPUS_2C | CARINATA_3C | 56.4 |
| NAPUS_2C | CARINATA_5B | 57.8 |
| NAPUS_2C | CARINATA_5C | 55.1 |
| NAPUS_2C | GMROD1-1 | 54.6 |
| NAPUS_2C | GMROD1-2 | 53.2 |
| NAPUS_2C | LUPDCT1 | 48 |
| NAPUS_2C | LUPDCT2 | 48 |
| NAPUS_2C | NAPUS_1A | 55 |
| NAPUS_2C | NAPUS_1C | 55.4 |
| NAPUS_2C | NAPUS_2A | 99.6 |
| NAPUS_2C | NAPUS_2C | 100 |
| NAPUS_2C | NAPUS_3A | 57.1 |
| NAPUS_2C | NAPUS_3C | 56.4 |
| NAPUS_2C | NAPUS_5A | 55.1 |
| NAPUS_2C | NAPUS_5C | 55.1 |
| NAPUS_2C | OSROD1_SEQIDNO11 | 38.1 |
| NAPUS_2C | RCPDCT | 51.2 |
| NAPUS_2C | RCROD1_SEQIDNO9 | 51.2 |
| NAPUS_2C | ZMROD1_GRMZM2G015040 | 44.6 |
| NAPUS_2C | ZMROD1_GRMZM2G087896 | 45.1 |
| NAPUS_3A | ATRODD1 | 79.2 |
| NAPUS_3A | BJROD1-A1 | 98.2 |
| NAPUS_3A | BJROD1-A2 | 81.7 |
| NAPUS_3A | BJROD1-A3 | 78.3 |
| NAPUS_3A | BJROD1-B1 | 96.8 |
| NAPUS_3A | BJROD1-B2 | 84.2 |
| NAPUS_3A | BJROD1-B3 | 78.3 |
| NAPUS_3A | BJROD1-B4 | 57.4 |
| NAPUS_3A | BRROD1_SEQIDNO7 | 98.9 |
| NAPUS_3A | CAMELINA_C1(80666) | 76.9 |
| NAPUS_3A | CAMELINA_C15(45897) | 76.5 |
| NAPUS_3A | CAMELINA_C19(65416) | 77.2 |
| NAPUS_3A | CARINATA_1B | 78.3 |
| NAPUS_3A | CARINATA_1C | 79.3 |
| NAPUS_3A | CARINATA_2B | 57.4 |
| NAPUS_3A | CARINATA_2C | 57.4 |
| NAPUS_3A | CARINATA_3B | 96.8 |
| NAPUS_3A | CARINATA_3C | 98.2 |
| NAPUS_3A | CARINATA_5B | 86.5 |
| NAPUS_3A | CARINATA_5C | 85.5 |
| NAPUS_3A | GMROD1-1 | 61.2 |
| NAPUS_3A | GMROD1-2 | 62.7 |
| NAPUS_3A | LUPDCT1 | 54.9 |
| NAPUS_3A | LUPDCT2 | 54.9 |
| NAPUS_3A | NAPUS_1A | 77.6 |
| NAPUS_3A | NAPUS_1C | 79 |
| NAPUS_3A | NAPUS_2A | 57.4 |
| NAPUS_3A | NAPUS_2C | 57.1 |
| NAPUS_3A | NAPUS_3A | 100 |
| NAPUS_3A | NAPUS_3C | 98.2 |
| NAPUS_3A | NAPUS_5A | 85.5 |
| NAPUS_3A | NAPUS_5C | 85.9 |
| NAPUS_3A | OSROD1_SEQIDNO11 | 44.6 |
| NAPUS_3A | RCPDCT | 61 |
| NAPUS_3A | RCROD1_SEQIDNO9 | 61 |
| NAPUS_3A | ZMROD1_GRMZM2G015040 | 45.9 |
| NAPUS_3A | ZMROD1_GRMZM2G087896 | 43.8 |
| NAPUS_3C | ATRODD1 | 78.8 |
| NAPUS_3C | BJROD1-A1 | 97.9 |
| NAPUS_3C | BJROD1-A2 | 83 |
| NAPUS_3C | BJROD1-A3 | 78.8 |
| NAPUS_3C | BJROD1-B1 | 98.2 |
| NAPUS_3C | BJROD1-B2 | 85.2 |
| NAPUS_3C | BJROD1-B3 | 78.8 |
| NAPUS_3C | BJROD1-B4 | 56.8 |
| NAPUS_3C | BRROD1_SEQIDNO7 | 98.6 |
| NAPUS_3C | CAMELINA_C1(80666) | 76.8 |
| NAPUS_3C | CAMELINA_C15(45897) | 76.2 |
| NAPUS_3C | CAMELINA_C19(65416) | 76.5 |
| NAPUS_3C | CARINATA_1B | 78.8 |
| NAPUS_3C | CARINATA_1C | 79.9 |
| NAPUS_3C | CARINATA_2B | 56.8 |
| NAPUS_3C | CARINATA_2C | 56.8 |
| NAPUS_3C | CARINATA_3B | 98.2 |
| NAPUS_3C | CARINATA_3C | 100 |
| NAPUS_3C | CARINATA_5B | 87.1 |
| NAPUS_3C | CARINATA_5C | 86.8 |
| NAPUS_3C | GMROD1-1 | 61.9 |
| NAPUS_3C | GMROD1-2 | 63.1 |
| NAPUS_3C | LUPDCT1 | 54.5 |
| NAPUS_3C | LUPDCT2 | 54.5 |
| NAPUS_3C | NAPUS_1A | 78.2 |
| NAPUS_3C | NAPUS_1C | 79.5 |
| NAPUS_3C | NAPUS_2A | 56.8 |
| NAPUS_3C | NAPUS_2C | 56.4 |
| NAPUS_3C | NAPUS_3A | 98.2 |
| NAPUS_3C | NAPUS_3C | 100 |
| NAPUS_3C | NAPUS_5A | 86.8 |
| NAPUS_3C | NAPUS_5C | 87.2 |
| NAPUS_3C | OSROD1_SEQIDNO11 | 44.9 |
| NAPUS_3C | RCPDCT | 60.8 |
| NAPUS_3C | RCROD1_SEQIDNO9 | 60.8 |
| NAPUS_3C | ZMROD1_GRMZM2G015040 | 45.8 |
| NAPUS_3C | ZMROD1_GRMZM2G087896 | 44.4 |
| NAPUS_5A | ATRODD1 | 79.7 |
| NAPUS_5A | BJROD1-A1 | 86.5 |
| NAPUS_5A | BJROD1-A2 | 95.4 |
| NAPUS_5A | BJROD1-A3 | 78.2 |
| NAPUS_5A | BJROD1-B1 | 86.8 |
| NAPUS_5A | BJROD1-B2 | 90.8 |
| NAPUS_5A | BJROD1-B3 | 78.9 |
| NAPUS_5A | BJROD1-B4 | 55.5 |
| NAPUS_5A | BRROD1_SEQIDNO7 | 86.8 |
| NAPUS_5A | CAMELINA_C1(80666) | 75.3 |
| NAPUS_5A | CAMELINA_C15(45897) | 75.6 |
| NAPUS_5A | CAMELINA_C19(65416) | 76.2 |
| NAPUS_5A | CARINATA_1B | 78.9 |
| NAPUS_5A | CARINATA_1C | 79.3 |
| NAPUS_5A | CARINATA_2B | 55.5 |
| NAPUS_5A | CARINATA_2C | 55.5 |
| NAPUS_5A | CARINATA_3B | 86.8 |
| NAPUS_5A | CARINATA_3C | 86.8 |
| NAPUS_5A | CARINATA_5B | 92.7 |
| NAPUS_5A | CARINATA_5C | 97.5 |
| NAPUS_5A | GMROD1-1 | 62.3 |
| NAPUS_5A | GMROD1-2 | 61 |
| NAPUS_5A | LUPDCT1 | 52 |
| NAPUS_5A | LUPDCT2 | 52 |
| NAPUS_5A | NAPUS_1A | 77.6 |
| NAPUS_5A | NAPUS_1C | 78.9 |
| NAPUS_5A | NAPUS_2A | 55.5 |
| NAPUS_5A | NAPUS_2C | 55.1 |
| NAPUS_5A | NAPUS_3A | 85.5 |
| NAPUS_5A | NAPUS_3C | 86.8 |
| NAPUS_5A | NAPUS_5A | 100 |
| NAPUS_5A | NAPUS_5C | 97.9 |
| NAPUS_5A | OSROD1_SEQIDNO11 | 42.2 |
| NAPUS_5A | RCPDCT | 60.2 |
| NAPUS_5A | RCROD1_SEQIDNO9 | 60.2 |
| NAPUS_5A | ZMROD1_GRMZM2G015040 | 45.2 |
| NAPUS_5A | ZMROD1_GRMZM2G087896 | 45.6 |
| NAPUS_5C | ATRODD1 | 80.1 |
| NAPUS_5C | BJROD1-A1 | 86.8 |
| NAPUS_5C | BJROD1-A2 | 93.6 |
| NAPUS_5C | BJROD1-A3 | 78.5 |
| NAPUS_5C | BJROD1-B1 | 87.2 |
| NAPUS_5C | BJROD1-B2 | 91.5 |
| NAPUS_5C | BJROD1-B3 | 79.2 |
| NAPUS_5C | BJROD1-B4 | 55.5 |
| NAPUS_5C | BRROD1_SEQIDNO7 | 87.2 |
| NAPUS_5C | CAMELINA_C1(80666) | 76 |
| NAPUS_5C | CAMELINA_C15(45897) | 76.9 |
| NAPUS_5C | CAMELINA_C19(65416) | 76.4 |
| NAPUS_5C | CARINATA_1B | 79.2 |
| NAPUS_5C | CARINATA_1C | 79.5 |
| NAPUS_5C | CARINATA_2B | 55.5 |
| NAPUS_5C | CARINATA_2C | 55.5 |
| NAPUS_5C | CARINATA_3B | 87.2 |
| NAPUS_5C | CARINATA_3C | 87.2 |
| NAPUS_5C | CARINATA_5B | 94.1 |
| NAPUS_5C | CARINATA_5C | 99.6 |
| NAPUS_5C | GMROD1-1 | 60.3 |
| NAPUS_5C | GMROD1-2 | 61.1 |
| NAPUS_5C | LUPDCT1 | 52 |
| NAPUS_5C | LUPDCT2 | 52 |
| NAPUS_5C | NAPUS_1A | 77.8 |
| NAPUS_5C | NAPUS_1C | 79.2 |
| NAPUS_5C | NAPUS_2A | 55.5 |
| NAPUS_5C | NAPUS_2C | 55.1 |
| NAPUS_5C | NAPUS_3A | 85.9 |
| NAPUS_5C | NAPUS_3C | 87.2 |
| NAPUS_5C | NAPUS_5A | 97.9 |
| NAPUS_5C | NAPUS_5C | 100 |
| NAPUS_5C | OSROD1_SEQIDNO11 | 42.5 |
| NAPUS_5C | RCPDCT | 59.9 |
| NAPUS_5C | RCROD1_SEQIDNO9 | 59.9 |
| NAPUS_5C | ZMROD1_GRMZM2G015040 | 46.4 |
| NAPUS_5C | ZMROD1_GRMZM2G087896 | 44.8 |
| OSROD1_SEQIDNO11 | ATRODD1 | 45.5 |
| OSROD1_SEQIDNO11 | BJROD1-A1 | 45.3 |
| OSROD1_SEQIDNO11 | BJROD1-A2 | 42.2 |
| OSROD1_SEQIDNO11 | BJROD1-A3 | 41.8 |
| OSROD1_SEQIDNO11 | BJROD1-B1 | 43.8 |
| OSROD1_SEQIDNO11 | BJROD1-B2 | 41.3 |
| OSROD1_SEQIDNO11 | BJROD1-B3 | 43.2 |
| OSROD1_SEQIDNO11 | BJROD1-B4 | 37.7 |
| OSROD1_SEQIDNO11 | BRROD1_SEQIDNO7 | 41.4 |
| OSROD1_SEQIDNO11 | CAMELINA_C1(80666) | 43.8 |
| OSROD1_SEQIDNO11 | CAMELINA_C15(45897) | 45.4 |
| OSROD1_SEQIDNO11 | CAMELINA_C19(65416) | 43.9 |
| OSROD1_SEQIDNO11 | CARINATA_1B | 42.3 |
| OSROD1_SEQIDNO11 | CARINATA_1C | 41.7 |
| OSROD1_SEQIDNO11 | CARINATA_2B | 38.1 |
| OSROD1_SEQIDNO11 | CARINATA_2C | 38.1 |
| OSROD1_SEQIDNO11 | CARINATA_3B | 44.1 |
| OSROD1_SEQIDNO11 | CARINATA_3C | 44.9 |
| OSROD1_SEQIDNO11 | CARINATA_5B | 42.6 |
| OSROD1_SEQIDNO11 | CARINATA_5C | 42.5 |
| OSROD1_SEQIDNO11 | GMROD1-1 | 47.1 |
| OSROD1_SEQIDNO11 | GMROD1-2 | 46.5 |
| OSROD1_SEQIDNO11 | LUPDCT1 | 45.9 |
| OSROD1_SEQIDNO11 | LUPDCT2 | 46.3 |
| OSROD1_SEQIDNO11 | NAPUS_1A | 42.4 |
| OSROD1_SEQIDNO11 | NAPUS_1C | 42 |
| OSROD1_SEQIDNO11 | NAPUS_2A | 38.1 |
| OSROD1_SEQIDNO11 | NAPUS_2C | 38.1 |
| OSROD1_SEQIDNO11 | NAPUS_3A | 44.6 |
| OSROD1_SEQIDNO11 | NAPUS_3C | 44.9 |
| OSROD1_SEQIDNO11 | NAPUS_5A | 42.2 |
| OSROD1_SEQIDNO11 | NAPUS_5C | 42.5 |
| OSROD1_SEQIDNO11 | OSROD1_SEQIDNO11 | 100 |
| OSROD1_SEQIDNO11 | RCPDCT | 48.9 |
| OSROD1_SEQIDNO11 | RCROD1_SEQIDNO9 | 48.9 |
| OSROD1_SEQIDNO11 | ZMROD1_GRMZM2G015040 | 69.1 |
| OSROD1_SEQIDNO11 | ZMROD1_GRMZM2G087896 | 68.9 |
| RCPDCT | ATRODD1 | 58.7 |
| RCPDCT | BJROD1-A1 | 58.6 |
| RCPDCT | BJROD1-A2 | 59.7 |
| RCPDCT | BJROD1-A3 | 57 |
| RCPDCT | BJROD1-B1 | 60.8 |
| RCPDCT | BJROD1-B2 | 59.1 |
| RCPDCT | BJROD1-B3 | 57.6 |
| RCPDCT | BJROD1-B4 | 51.6 |
| RCPDCT | BRROD1_SEQIDNO7 | 60.8 |
| RCPDCT | CAMELINA_C1(80666) | 55.4 |
| RCPDCT | CAMELINA_C15(45897) | 59.8 |
| RCPDCT | CAMELINA_C19(65416) | 59.9 |
| RCPDCT | CARINATA_1B | 57.6 |
| RCPDCT | CARINATA_1C | 57.9 |
| RCPDCT | CARINATA_2B | 52.3 |
| RCPDCT | CARINATA_2C | 51.9 |
| RCPDCT | CARINATA_3B | 61.1 |
| RCPDCT | CARINATA_3C | 60.8 |
| RCPDCT | CARINATA_5B | 59.9 |
| RCPDCT | CARINATA_5C | 59.9 |
| RCPDCT | GMROD1-1 | 68.2 |
| RCPDCT | GMROD1-2 | 59.3 |
| RCPDCT | LUPDCT1 | 59.2 |
| RCPDCT | LUPDCT2 | 59.2 |
| RCPDCT | NAPUS_1A | 57.3 |
| RCPDCT | NAPUS_1C | 57.9 |
| RCPDCT | NAPUS_2A | 51.6 |
| RCPDCT | NAPUS_2C | 51.2 |
| RCPDCT | NAPUS_3A | 61 |
| RCPDCT | NAPUS_3C | 60.8 |
| RCPDCT | NAPUS_5A | 60.2 |
| RCPDCT | NAPUS_5C | 59.9 |
| RCPDCT | OSROD1_SEQIDNO11 | 48.9 |
| RCPDCT | RCPDCT | 100 |
| RCPDCT | RCROD1_SEQIDNO9 | 100 |
| RCPDCT | ZMROD1_GRMZM2G015040 | 51.3 |
| RCPDCT | ZMROD1_GRMZM2G087896 | 48.2 |
| RCROD1_SEQIDNO9 | ATRODD1 | 58.7 |
| RCROD1_SEQIDNO9 | BJROD1-A1 | 58.6 |
| RCROD1_SEQIDNO9 | BJROD1-A2 | 59.7 |
| RCROD1_SEQIDNO9 | BJROD1-A3 | 57 |
| RCROD1_SEQIDNO9 | BJROD1-B1 | 60.8 |
| RCROD1_SEQIDNO9 | BJROD1-B2 | 59.1 |
| RCROD1_SEQIDNO9 | BJROD1-B3 | 57.6 |
| RCROD1_SEQIDNO9 | BJROD1-B4 | 51.6 |
| RCROD1_SEQIDNO9 | BRROD1_SEQIDNO7 | 60.8 |
| RCROD1_SEQIDNO9 | CAMELINA_C1(80666) | 55.4 |
| RCROD1_SEQIDNO9 | CAMELINA_C15(45897) | 59.8 |
| RCROD1_SEQIDNO9 | CAMELINA_C19(65416) | 59.9 |
| RCROD1_SEQIDNO9 | CARINATA_1B | 57.6 |
| RCROD1_SEQIDNO9 | CARINATA_1C | 57.9 |
| RCROD1_SEQIDNO9 | CARINATA_2B | 52.3 |
| RCROD1_SEQIDNO9 | CARINATA_2C | 51.9 |
| RCROD1_SEQIDNO9 | CARINATA_3B | 61.1 |
| RCROD1_SEQIDNO9 | CARINATA_3C | 60.8 |
| RCROD1_SEQIDNO9 | CARINATA_5B | 59.9 |
| RCROD1_SEQIDNO9 | CARINATA_5C | 59.9 |
| RCROD1_SEQIDNO9 | GMROD1-1 | 68.2 |
| RCROD1_SEQIDNO9 | GMROD1-2 | 59.3 |
| RCROD1_SEQIDNO9 | LUPDCT1 | 59.2 |
| RCROD1_SEQIDNO9 | LUPDCT2 | 59.2 |
| RCROD1_SEQIDNO9 | NAPUS_1A | 57.3 |
| RCROD1_SEQIDNO9 | NAPUS_1C | 57.9 |
| RCROD1_SEQIDNO9 | NAPUS_2A | 51.6 |
| RCROD1_SEQIDNO9 | NAPUS_2C | 51.2 |
| RCROD1_SEQIDNO9 | NAPUS_3A | 61 |
| RCROD1_SEQIDNO9 | NAPUS_3C | 60.8 |
| RCROD1_SEQIDNO9 | NAPUS_5A | 60.2 |
| RCROD1_SEQIDNO9 | NAPUS_5C | 59.9 |
| RCROD1_SEQIDNO9 | OSROD1_SEQIDNO11 | 48.9 |
| RCROD1_SEQIDNO9 | RCPDCT | 100 |
| RCROD1_SEQIDNO9 | RCROD1_SEQIDNO9 | 100 |
| RCROD1_SEQIDNO9 | ZMROD1_GRMZM2G015040 | 51.3 |
| RCROD1_SEQIDNO9 | ZMROD1_GRMZM2G087896 | 48.2 |
| ZMROD1_GRMZM2G015040 | ATRODD1 | 44.4 |
| ZMROD1_GRMZM2G015040 | BJROD1-A1 | 45.3 |
| ZMROD1_GRMZM2G015040 | BJROD1-A2 | 45.1 |
| ZMROD1_GRMZM2G015040 | BJROD1-A3 | 43.7 |
| ZMROD1_GRMZM2G015040 | BJROD1-B1 | 45.8 |
| ZMROD1_GRMZM2G015040 | BJROD1-B2 | 47.1 |
| ZMROD1_GRMZM2G015040 | BJROD1-B3 | 44 |
| ZMROD1_GRMZM2G015040 | BJROD1-B4 | 44.6 |
| ZMROD1_GRMZM2G015040 | BRROD1_SEQIDNO7 | 46.2 |
| ZMROD1_GRMZM2G015040 | CAMELINA_C1(80666) | 45.1 |
| ZMROD1_GRMZM2G015040 | CAMELINA_C15(45897) | 45 |
| ZMROD1_GRMZM2G015040 | CAMELINA_C19(65416) | 43.8 |
| ZMROD1_GRMZM2G015040 | CARINATA_1B | 44 |
| ZMROD1_GRMZM2G015040 | CARINATA_1C | 42.9 |
| ZMROD1_GRMZM2G015040 | CARINATA_2B | 44.9 |
| ZMROD1_GRMZM2G015040 | CARINATA_2C | 45.3 |
| ZMROD1_GRMZM2G015040 | CARINATA_3B | 46.4 |
| ZMROD1_GRMZM2G015040 | CARINATA_3C | 45.8 |
| ZMROD1_GRMZM2G015040 | CARINATA_5B | 46.4 |
| ZMROD1_GRMZM2G015040 | CARINATA_5C | 46.4 |
| ZMROD1_GRMZM2G015040 | GMROD1-1 | 51.4 |
| ZMROD1_GRMZM2G015040 | GMROD1-2 | 50.9 |
| ZMROD1_GRMZM2G015040 | LUPDCT1 | 48.1 |
| ZMROD1_GRMZM2G015040 | LUPDCT2 | 47.8 |
| ZMROD1_GRMZM2G015040 | NAPUS_1A | 44 |
| ZMROD1_GRMZM2G015040 | NAPUS_1C | 43.3 |
| ZMROD1_GRMZM2G015040 | NAPUS_2A | 44.9 |
| ZMROD1_GRMZM2G015040 | NAPUS_2C | 44.6 |
| ZMROD1_GRMZM2G015040 | NAPUS_3A | 45.9 |
| ZMROD1_GRMZM2G015040 | NAPUS_3C | 45.8 |
| ZMROD1_GRMZM2G015040 | NAPUS_5A | 45.2 |
| ZMROD1_GRMZM2G015040 | NAPUS_5C | 46.4 |
| ZMROD1_GRMZM2G015040 | OSROD1_SEQIDNO11 | 69.1 |
| ZMROD1_GRMZM2G015040 | RCPDCT | 51.3 |
| ZMROD1_GRMZM2G015040 | RCROD1_SEQIDNO9 | 51.3 |
| ZMROD1_GRMZM2G015040 | ZMROD1_GRMZM2G015040 | 100 |
| ZMROD1_GRMZM2G015040 | ZMROD1_GRMZM2G087896 | 83.9 |
| ZMROD1_GRMZM2G087896 | ATRODD1 | 42.9 |
| ZMROD1_GRMZM2G087896 | BJROD1-A1 | 44.1 |
| ZMROD1_GRMZM2G087896 | BJROD1-A2 | 45.6 |
| ZMROD1_GRMZM2G087896 | BJROD1-A3 | 42.7 |
| ZMROD1_GRMZM2G087896 | BJROD1-B1 | 44.1 |
| ZMROD1_GRMZM2G087896 | BJROD1-B2 | 45.6 |
| ZMROD1_GRMZM2G087896 | BJROD1-B3 | 44.7 |
| ZMROD1_GRMZM2G087896 | BJROD1-B4 | 43.6 |
| ZMROD1_GRMZM2G087896 | BRROD1_SEQIDNO7 | 44.1 |
| ZMROD1_GRMZM2G087896 | CAMELINA_C1(80666) | 47 |
| ZMROD1_GRMZM2G087896 | CAMELINA_C15(45897) | 46.5 |
| ZMROD1_GRMZM2G087896 | CAMELINA_C19(65416) | 47.7 |
| ZMROD1_GRMZM2G087896 | CARINATA_1B | 44.7 |
| ZMROD1_GRMZM2G087896 | CARINATA_1C | 42.7 |
| ZMROD1_GRMZM2G087896 | CARINATA_2B | 44.2 |
| ZMROD1_GRMZM2G087896 | CARINATA_2C | 44.7 |
| ZMROD1_GRMZM2G087896 | CARINATA_3B | 44.4 |
| ZMROD1_GRMZM2G087896 | CARINATA_3C | 44.4 |
| ZMROD1_GRMZM2G087896 | CARINATA_5B | 45.8 |
| ZMROD1_GRMZM2G087896 | CARINATA_5C | 44.8 |
| ZMROD1_GRMZM2G087896 | GMROD1-1 | 53.1 |
| ZMROD1_GRMZM2G087896 | GMROD1-2 | 49 |
| ZMROD1_GRMZM2G087896 | LUPDCT1 | 49 |
| ZMROD1_GRMZM2G087896 | LUPDCT2 | 48.6 |
| ZMROD1_GRMZM2G087896 | NAPUS_1A | 43 |
| ZMROD1_GRMZM2G087896 | NAPUS_1C | 43 |
| ZMROD1_GRMZM2G087896 | NAPUS_2A | 44 |
| ZMROD1_GRMZM2G087896 | NAPUS_2C | 43.6 |
| ZMROD1_GRMZM2G087896 | NAPUS_3A | 43.8 |
| ZMROD1_GRMZM2G087896 | NAPUS_3C | 44.4 |
| ZMROD1_GRMZM2G087896 | NAPUS_5A | 45.6 |
| ZMROD1_GRMZM2G087896 | NAPUS_5C | 44.8 |
| ZMROD1_GRMZM2G087896 | OSROD1_SEQIDNO11 | 68.9 |
| ZMROD1_GRMZM2G087896 | RCPDCT | 48.2 |
| ZMROD1_GRMZM2G087896 | RCROD1_SEQIDNO9 | 48.2 |
| ZMROD1_GRMZM2G087896 | ZMROD1_GRMZM2G015040 | 83.9 |
| ZMROD1_GRMZM2G087896 | ZMROD1_GRMZM2G087896 | 100 |

TABLE 7
Average fatty acid composition (%) in different lipid classes from immature seeds

| | 16:0 | 18:0 | 18:1 | 18:2 | GLA | 18:3 | SDA | 20:0 | 20:1 | 20:2 | DGLA | 22:1 |
|---|---|---|---|---|---|---|---|---|---|---|---|---|
| TAG | | | | | | | | | | | | |
| 1C | 10.3 | 5.4 | 37.0 | 13.6 | 4.9 | 6.4 | 1.0 | 2.1 | 15.3 | 0.4 | 1.7 | 1.6 |
| 2C | 9.9 | 5.8 | 40.3 | 10.8 | 5.7 | 5.3 | 0.7 | 1.9 | 15.0 | 0.3 | 2.4 | 1.4 |
| CK | 10.6 | 4.9 | 36.8 | 17.6 | 1.7 | 6.7 | 0.1 | 2.0 | 15.0 | 0.5 | 1.8 | 1.5 |
| mutant | | | | | | | | | | | | |
| CK WT | 9.4 | 4.7 | 19.5 | 22.0 | 11.6 | 7.9 | 1.6 | 2.2 | 16.6 | 1.2 | 1.2 | 1.4 |
| WT | 8.8 | 4.4 | 22.2 | 31.2 | 0.0 | 11.3 | 0.0 | 2.2 | 16.6 | 1.5 | 0.0 | 1.6 |
| Rod mut | 10.2 | 4.5 | 31.4 | 20.9 | 0.0 | 10.2 | 0.0 | 2.6 | 16.7 | 0.7 | 0.0 | 2.2 |
| PC | | | | | | | | | | | | |
| 1C | 22.9 | 2.4 | 3.8 | 40.3 | 5.4 | 21.7 | 1.2 | 0.0 | 0.2 | 0.8 | 0.7 | 0.1 |
| 2C | 20.8 | 2.2 | 3.6 | 40.8 | 7.9 | 21.6 | 1.1 | 0.0 | 0.3 | 0.9 | 0.4 | 0.2 |
| CK | 18.1 | 1.8 | 3.4 | 46.7 | 2.7 | 25.3 | 0.3 | 0.0 | 0.2 | 0.9 | 0.1 | 0.2 |
| mutant | | | | | | | | | | | | |
| CK WT | 27.3 | 3.6 | 4.0 | 40.0 | 3.9 | 18.3 | 0.5 | 0.0 | 0.7 | 0.9 | 0.2 | 0.0 |
| WT | 22.6 | 2.6 | 5.8 | 45.9 | 0.0 | 20.6 | 0.0 | 0.0 | 1.1 | 0.9 | 0.0 | 0.0 |
| Rod mut | 18.2 | 2.0 | 2.8 | 48.2 | 0.0 | 27.5 | 0.0 | 0.0 | 0.0 | 0.9 | 0.0 | 0.0 |
| DAG | | | | | | | | | | | | |
| 1C | 16.5 | 8.7 | 28.4 | 17.8 | 3.7 | 7.0 | 0.0 | 4.4 | 7.6 | 0.0 | 1.1 | 4.8 |
| 2C | 15.5 | 8.2 | 32.5 | 11.5 | 6.0 | 6.1 | 0.0 | 3.9 | 10.0 | 0.0 | 2.2 | 4.0 |
| CK | 19.9 | 10.3 | 32.9 | 16.2 | 1.1 | 5.0 | 0.0 | 3.2 | 10.4 | 0.0 | 0.0 | 0.8 |
| mutant | | | | | | | | | | | | |
| CK WT | 17.9 | 5.2 | 13.6 | 35.6 | 7.8 | 10.9 | 0.0 | 1.7 | 5.1 | 0.0 | 0.8 | 1.4 |
| WT | 17.1 | 7.2 | 24.4 | 34.1 | 0.0 | 5.9 | 0.0 | 2.4 | 6.7 | 0.0 | 0.0 | 2.2 |
| Rod mut | 18.2 | 9.7 | 25.1 | 19.2 | 0.0 | 7.3 | 0.0 | 5.2 | 9.9 | 0.0 | 0.0 | 5.4 |

### REFERENCES

Philip D. Bates, Abdelhak Fatihi, Anna R. Snapp, Anders S. Carlsson, John Browse, Chaofu Lu (2012) Acyl Editing and Headgroup Exchange Are the Major Mechanisms That Direct Polyunsaturated Fatty Acid Flux into Triacylglycerols. Plant Physiology 160(3), 1530-1539

Clough S J, Bent A F. Floral dip: a simplified method forAgrobacterium-mediated transformation of Arabidopsis thaliana. The plant journal. 1998 Dec. 1; 16(6):735-43.

Genet T, Labuschagne M T, Hugo A. Capillary gas chromatography analysis of Ethiopian mustard to determine variability of fatty acid composition. Journal of the Science of Food and Agriculture. 2004 Oct. 1; 84(13):1663-70.

Iskandarov U, Kim H J, Cahoon E B. Camelina: an emerging oilseed platform for advanced biofuels and bio-based materials. InPlants and BioEnergy 2014 (pp. 131-140). Springer New York

Lager I, Yilmaz J L, Zhou X R, Jasieniecka K, Kazachkov M, Wang P, Zou J, Weselake R, Smith M A, Bayon S, Dyer J M. Plant acyl-CoA: lysophosphatidylcholine acyltransferases (LPCATs) have different specificities in their forward and reverse reactions. Journal of Biological Chemistry. 2013 Dec. 27; 288(52):36902-14.

Lu C, Xin Z, Ren Z, Miguel M. An enzyme regulating triacylglycerol composition is encoded by the ROD1 gene of Arabidopsis. Proceedings of the National Academy of Sciences. 2009 Nov. 3; 106(44):18837-42.

Liu J W, Huang Y S, DeMichele S, Bergana M, E Jr Bobik, Hastilow C, Chuang L T, Mukerji P, Knutzon D (2001) Evaluation of the seed oils from a canola plant genetically transformed to produce high levels of gamma-linolenic acid. In: Huang Y S, Ziboh V A (eds) Gamma-linolenic acid: recent advances in biotechnology and clinical applications. AOCS Press, Champaign, Ill., pp 61-71

Petrie, J. R., Shrestha, P., Zhou, X. R., Mansour, M. P., Li, Q., Belide, S., Nichols, P. D. and Singh, S. P. (2012) Metabolic engineering of seeds with fish oil-like levels of DHA. PLoS ONE, 7, e49165.

Ruiz-Lopez N, Haslam R P, Napier J A, Sayanova O. Successful high-level accumulation of fish oil omega-3 long-chain polyunsaturated fatty acids in a transgenic oilseed crop. The Plant Journal. 2014 Jan. 1; 77(2):198-208.

Singer S D, Weselake R J, Rahman H. Development and characterization of low α-linolenic acid Brassica oleracea lines bearing a novel mutation in a ‘class a’ FATTY ACID DESATURASE 3 gene. BMC genetics. 2014 Aug. 29; 15(1):94.

Ursin V M. Modification of plant lipids for human health: development of functional land-based omega-3 fatty acids. The Journal of nutrition. 2003 Dec. 1; 133(12):4271-4.

Venegas-Caleron, Monica; Sayanova, Olga; Napier, Johnathan A. (2010) An alternative to fish oils: Metabolic engineering of oil-seed crops to produce omega-3 long chain polyunsaturated fatty acids. Progress In Lipid Research 49(2), 108-119.

Aruna D Wickramarathna, odrigo M P Siloto, Elzbieta Mietkiewska, Stacy D Singer, Xue Pan, Randall J Weselake (2015) Heterologous expression of flax PHOSPHOLIPID:DIACYLGLYCEROL CHOLINEPHOSPHOTRANSFERASE (PDCT) increases polyuns;;aturated fatty acid content in yeast and Arabidopsis seeds. BMC Biotechnology 15 article 63

Wu G, Truksa M, Datla N, Vrinten P, Bauer J, Zank T, Cirpus P, Heinz E, Qiu X. Stepwise engineering to produce high yields of very long-chain polyunsaturated fatty acids in plants. Nature biotechnology. 2005 August; 23(8):1013

1. A plant raw oil comprising PUFAs, e.g. vIcPUFAs, as for example, EPA, DHA, and/or DPA that further comprises
i. a level of the 18:2 fatty acid in % (w/w) in the triacylglycerol (TAG) fraction that is between 80 and 120% of the level as the 18:2 fatty acid level in % (w/w) in the diacylglyerol (DAG) fraction,
ii. a level of the 22:1 fatty acid in % (w/w) in the triacylglycerol fraction that is higher than the 22:1 fatty acid level in % (w/w) in the diacylglyerol fraction, and/or
iii. a level of SDA in % (w/w) in the phosphatyidylcholine (PC) fraction is higher than the SDA level in % (w/w) in the triacylglycerol fraction.

2. A method for the production of a composition comprising the fatty acid GLA in a plant, or part thereof, like a plant cell, and/or part seed, or part thereof,
wherein the level of the 18:2 fatty acid in % (w/w) in the triacylglycerol (TAG) fraction is around the same level as the 18:2 fatty acid level in % (w/w) in the diacylglyerol (DAG) fraction, comprising,
providing a plant cable to produce GLA and having an increased activity or expression of one or more PDCT compared to the wild type, the PDCT selected from the group consisting of:
(a) a PDCT1 having at least 80% sequence identity with SEQ ID NO: 2, 4, 6, 8, 10, 12, 14, 16, 40, 42, 44, and/or 46;
(b) a PDCT1 encoded by a polynucleotide having at least 80% sequence identity with SEQ ID NO: 1, 3, 5, 7, 9, 11, 13, 15, 39, 41, 43, and/or 45;
(c) a PDCT1 encoded by a polynucleotide that hybridizes under high stringency conditions with (i) a polynucleotide that encodes the amino acid sequence of SEQ ID NO: 2, 4, 6, 8, 10, 12, 14, 16, 40, 42, 44, and/or 46, or (ii) the full-length complement of (i);
(d) a variant of the PDCT1 of SEQ ID NO: 2, 4, 6, 8, 10, 12, 14, 16, 40, 42, 44, and/or 46 comprising a substitution, deletion, and/or insertion at one or more positions and having PDCT1 activity;
(e) a PDCT1 encoded by a polynucleotide that differs from SEQ ID NO: 1, 3, 5, 7, 9, 11, 13, 15, 39, 41, 43, and/or 45 due to the degeneracy of the genetic code; and
(f) a fragment of the PDCT1 of (a), (b), (c), (d) or (e) having PDCT1 activity;
and, optionally, the seed oil is isolated.

3. Method for increasing the Delta-6 elongase conversion efficiency in a plant, plant cell, plant seed and/or part thereof, that is capable to produce PUFA and expresses a Delta-6 elongase, comprising increasing, compared to a control, in the plant, plant cell, plant seed and/or part thereof the activity or expression rate of one or more PDCT selected from the group consisting of:
(a) a PDCT1 having at least 80% sequence identity with SEQ ID NO: 2, 4, 6, 8, 10, 12, 14, 16, 40, 42, 44, and/or 46;
(b) a PDCT1 encoded by a polynucleotide having at least 80% sequence identity with SEQ ID NO: 1, 3, 5, 7, 9, 11, 13, 15, 39, 41, 43, and/or 45;
(c) a PDCT1 encoded by a polynucleotide that hybridizes under high stringency conditions with (i) a polynucleotide that encodes the amino acid sequence of SEQ ID NO: 2, 4, 6, 8, 10, 12, 14, 16, 40, 42, 44, and/or 46, or (ii) the full-length complement of (i);
(d) a variant of the PDCT1 of SEQ ID NO: 2, 4, 6, 8, 10, 12, 14, 16, 40, 42, 44, and/or 46 comprising a substitution, deletion, and/or insertion at one or more positions and having PDCT1 activity;
(e) a PDCT1 encoded by a polynucleotide that differs from SEQ ID NO: 1, 3, 5, 7, 9, 11, 13, 15, 39, 41, 43, and/or 45 due to the degeneracy of the genetic code; and
(f) a fragment of the PDCT1 of (a), (b), (c), (d) or (e) having PDCT1 activity.

4. Method for improving the cellular conversion efficiency from oleic acid to C18 to C22 PUFA in a plant, plant seed, plant cell or part thereof, comprising providing a plant, plant cell, plant seed or part thereof, that is capable to produce C18 to C22 PUFA, comprising increasing the activity of one or more PDCT selected from the group consisting of:
(a) a PDCT1 having at least 80% sequence identity with SEQ ID NO: 2, 4, 6, 8, 10, 12, 14, 16, 40, 42, 44, and/or 46;
(b) a PDCT1 encoded by a polynucleotide having at least 80% sequence identity with SEQ ID NO: 1, 3, 5, 7, 9, 11, 13, 15, 39, 41, 43, and/or 45;
(c) a PDCT1 encoded by a polynucleotide that hybridizes under high stringency conditions with (i) a polynucleotide that encodes the amino acid sequence of SEQ ID NO: 2, 4, 6, 8, 10, 12, 14, 16, 40, 42, 44, and/or 46, or (ii) the full-length complement of (i),
(d) a variant of the PDCT1 of SEQ ID NO: 2, 4, 6, 8, 10, 12, 14, 16, 40, 42, 44, and/or 46 comprising a substitution, preferably a conservative substitution, deletion, and/or insertion at one or more positions and having PDCT1 activity;
(e) a PDCT1 encoded by a polynucleotide that differs from SEQ ID NO: 1, 3, 5, 7, 9, 11, 13, 15, 39, 41, 43, and/or 45 due to the degeneracy of the genetic code; and
(f) a fragment of the PDCT1 of (a), (b), (c), (d) or (e) having PDCT1 activity.
and expressing a phospholipid-dependent desaturase and, optionally, at least one Acetyl-CoA-dependent desaturase

5. A method for producing vIcPUFA in an oil crop plant, comprising
providing a first an oil crop plant variety that is cable to produce the desired vlcPUFA,
providing a second an oil crop plant variety that has an increased activity of one or more PDCT selected from the group consisting of:
(a) a PDCT1 having at least 80% sequence identity with SEQ ID NO: 2, 4, 6, 8, 10, 12, 14, 16, 40, 42, 44, and/or 46;
(b) a PDCT1 encoded by a polynucleotide having at least 80% sequence identity with SEQ ID NO: 1, 3, 5, 7, 9, 11, 13, 15, 39, 41, 43, and/or 45;
(c) a PDCT1 encoded by a polynucleotide that hybridizes under high stringency conditions with (i) a polynucleotide that encodes the amino acid sequence of SEQ ID NO: 2, 4, 6, 8, 10, 12, 14, 16, 40, 42, 44, and/or 46, or (ii) the full-length complement of (i);
(d) a variant of the PDCT1 of SEQ ID NO: 2, 4, 6, 8, 10, 12, 14, 16, 40, 42, 44, and/or 46 comprising a substitution, deletion, and/or insertion at one or more positions and having PDCT1 activity;
(e) a PDCT1 encoded by a polynucleotide that differs from SEQ ID NO: 1, 3, 5, 7, 9, 11, 13, 15, 39, 41, 43, and/or 45 due to the degeneracy of the genetic code; and
(f) a fragment of the PDCT1 of (a), (b), (c), (d) or (e) having PDCT1 activity;
crossing the first and second an oil crop plant variety,
optionally, measuring the PDCT1 expression rate in first or later generation cells, seeds, plants or part thereof derived from the cross,
optionally, measuring the total PUFA level in in first or later generation cells, seeds, plants or part thereof derived from the cross,
optionally, repeating steps 2 to 5,
planting and growing the plant, and
isolating the vIcPUFA comprising oil from the seed of first or later generation plants derived from the cross.

6. Method for the production of a composition comprising the fatty acids GLA in a plant, or part thereof, plant cell, and/or part seed, or part thereof, cable to produce GLA comprising providing a plant, plant cell or seed with an increased activity or expression of one or more PDCT selected from the group consisting of:
(a) a PDCT1 having at least 80% sequence identity with SEQ ID NO: 2, 4, 6, 8, 10, 12, 14, 16, 40, 42, 44, and/or 46;
(b) a PDCT1 encoded by a polynucleotide having at least 80% sequence identity with SEQ ID NO: 1, 3, 5, 7, 9, 11, 13, 15, 39, 41, 43, and/or 45;
(c) a PDCT1 encoded by a polynucleotide that hybridizes under high stringency conditions with (i) a polynucleotide that encodes the amino acid sequence of SEQ ID NO: 2, 4, 6, 8, 10, 12, 14, 16, 40, 42, 44, and/or 46, or (ii) the full-length complement of (i);
(d) a variant of the PDCT1 of SEQ ID NO: 2, 4, 6, 8, 10, 12, 14, 16, 40, 42, 44, and/or 46 comprising a substitution, deletion, and/or insertion at one or more positions and having PDCT1 activity;
(e) a PDCT1 encoded by a polynucleotide that differs from SEQ ID NO: 1, 3, 5, 7, 9, 11, 13, 15, 39, 41, 43, and/or 45 due to the degeneracy of the genetic code; and
(f) a fragment of the PDCT1 of (a), (b), (c), (d) or (e) having PDCT1 activity;
and, optionally, isolating the composition comprising the desired fatty acids.

7. The method of claim 6, whereby the plant, plant seed or plant cell expresses at least one phospholipid-dependent desaturased.

8. The method or oil of claim 1, whereby the plant, plant seed or plant cell expresses at least one phospholipid-dependent desaturase and at least one Acetyl-CoA-dependent desaturase.

9. The method or oil of claim 1, whereby the plant, plant seed or plant cell expresses at least one acyl-CoA dependent desaturase and/or at least one PC-dependent elongase selected from the group consisting of d5-, d5d6-, and d6elongase.

10. The method or oil of claim 1, whereby the plant, plant seed or plant cell expresses at least one Delta-6 elongase and/or at least one Delta-6-desaturase.

11. The method or oil of claim 1, wherein the total PUFA level in the plant, plant seed, plant cell or an oil derived therefrom is increased compared to a control.

12. The method or oil of claim 1, wherein the ratio of C20 fatty acids to C18 is increased.

13. The method or oil of claim 1, wherein a composition isolated from the plant the plant or cell is derived that is a raw oil.

14. A method or oil of claim 1 comprising, providing a plant, seed, or plant cell capable to produce acids EPA, DPA and/or DHA, the plant, seed, or plant cell comprising:
at least one nucleic acid sequence which encodes at least one Delta-12 desaturase
at least one nucleic acid sequence which encodes at least one Omega 3 desaturase,
at least one nucleic acid sequence which encodes a Delta 6-desaturase activity,
b) at least one nucleic acid sequence which encodes a Delta-6 elongase activity,
c) at least one nucleic acid sequence which encodes a Delta-5 desaturase activity,
d) at least one nucleic acid sequence which encodes a Delta-5 elongase activity, and
e) at least one nucleic acid sequence which encodes a Delta-4 desaturase activity

15. The method or oil of claim 1, wherein an oil is produced with a ratio of 18:1 fatty acids to total fatty acid content (w/w) is reduced by at least 10% compared to the control and/or wherein ratio ALA to total fatty acid content is reduced by at least 20%.

16. The method or oil of claim 1, wherein at least one of the following PDCT has an increased activity or is overexpressed:
napus_1A, napus_2A, napus_1C, and/or napus_2C PDCT when the plant, plant cell or plant seed is a Brassica sp., or a B. napus plant, plant cell or plant seed
carinata_1B, carinata_1C, carinata_2B, and/or carinata_2C PDCT when the plant, plant cell or plant seed is a Brassica sp., or a B. carinata plant, plant cell or plant seed, or
BjROD1-B4, BjROD1-A3, and/or BjROD1-B3 PDCT, when the plant, plant cell or plant seed is a Brassica sp., or a B. juncea plant, plant cell or plant seed.

17. The method or oil of claim 1, comprising, compared to a control, in the plant or part thereof, or seed, or plant cell the increasing the activity or expressing a PDCT
of one or more PDCT selected from the group consisting of:
a) a PDCT1 having at least 80% sequence identity with SEQ ID NO: 2, 4, 6, 8, 10, 12, 14, 16, 40, 42, 44, and/or 46;
(b) a PDCT1 encoded by a polynucleotide having at least 80% sequence identity with SEQ ID NO: 1, 3, 5, 7, 9, 11, 13, 15, 39, 41, 43, and/or 45;
(c) a PDCT1 encoded by a polynucleotide that hybridizes under high stringency conditions with (i) a polynucleotide that encodes the amino acid sequence of SEQ ID NO: 2, 4, 6, 8, 10, 12, 14, 16, 40, 42, 44, and/or 46, or (ii) the full-length complement of (i),
(d) a variant of the PDCT1 of SEQ ID NO: 2, 4, 6, 8, 10, 12, 14, 16, 40, 42, 44, and/or 46 comprising a substitution, deletion, and/or insertion at one or more positions and having PDCT1 activity;
(e) a PDCT1 encoded by a polynucleotide that differs from SEQ ID NO: 1, 3, 5, 7, 9, 11, 13, 15, 39, 41, 43, and/or 45 due to the degeneracy of the genetic code; and
(f) a fragment of the PDCT1 of (a), (b), (c), (d) or (e) having PDCT1 activity; and, optionally, isolating the composition comprising the desired fatty acids.
and
one or more PDCT selected from the group consisting of:
(a) a PDCT1 having at least 80% sequence identity with SEQ ID NO: 2, 4, 6, 8, 10, 12, 14, 16, 40, 42, 44, and/or 46;
(b) a PDCT1 encoded by a polynucleotide having at least 80% sequence identity with SEQ ID NO: 1, 3, 5, 7, 9, 11, 13, 15, 39, 41, 43, and/or 45;
(c) a PDCT1 encoded by a polynucleotide that hybridizes under high stringency conditions with (i) a polynucleotide that encodes the amino acid sequence of SEQ ID NO: 2, 4, 6, 8, 10, 12, 14, 16, 40, 42, 44, and/or 46, or (ii) the full-length complement of (i);
(d) a variant of the PDCT1 of SEQ ID NO: 2, 4, 6, 8, 10, 12, 14, 16, 40, 42, 44, and/or 46 comprising a substitution, deletion, and/or insertion at one or more positions and having PDCT activity;
(e) a PDCT1 encoded by a polynucleotide that differs from SEQ ID NO: 1, 3, 5, 7, 9, 11, 13, 15, 39, 41, 43, and/or 45 due to the degeneracy of the genetic code; and
(f) a fragment of the PDCT of (a), (b), (c), (d) or (e) having PDCT1 activity

18. The method or oil of claim 1, wherein the delta 6 elongase conversion efficiency is increased

19. The method or oil of any one claim 1, wherein the expression or activity of at least one PDCT3 and/or a PDCT5 is reduced, the PDCT3 and/or a PDCT5 selected from
(a) PDCT3 and/or PDCT5 having at least 80% sequence identity with SEQ ID NO: 18, 20, 22, 24, 26, 28, 30, 32, 50, 52, 54, 56, 58, and/or 60;
(b) PDCT3 and/or PDCT5 encoded by a polynucleotide having at least 80% sequence identity with SEQ ID NO: 17, 19, 21, 23, 27, 29, 31, 49, 51, 53, 55, and/or 57;
(c) PDCT3 and/or PDCT5 encoded by one or more polynucleotides that hybridize under high stringency conditions with (i) a polynucleotide that encodes the amino acid sequence of SEQ ID NO: 18, 20, 22, 24, 26, 28, 30, 32, 50, 52, 54, 56, 58, and/or 60, or (ii) the full-length complement of (i);
(d) variants of the PDCT3 and/or PDCT5 of SEQ ID NO: 18, 20, 22, 24, 26, 28, 30, 32, 50, 52, 54, 56, 58, and/or 60, comprising a substitution, preferably a conservative substitution, deletion, and/or insertion at one or more positions and having PDCT3 and/or PDCT5 activity;
(e) PDCT3 and/or PDCT5 encoded by a polynucleotide that differs from SEQ ID NO: 17, 19, 21, 23, 27, 29, 31, 49, 51, 53, 55, and/or 57 due to the degeneracy of the genetic code; and
(f) fragments of the PDCT3 and/or PDCT5 of (a), (b), (c), (d) or (e) having PDCT3 and/or PDCT5 activity.

20. The method or oil of claim 1, whereby a heterologous PDCT1 or PDCT19 is expressed de novo.

21. A method for the production of food or feed comprising EPA, DHA and/or DPA, isolating the fatty acid composition and formulating the fatty acid composition to food or feed.

22. An isolated, a synthetic, or a recombinant polynucleotide comprising:
(a) a nucleic acid sequence having at least 80% sequence identity to SEQ ID NO: 1, 3, 5, 7, 9, 11, 13, 15, 39, 41, 43, and/or 45, wherein the nucleic acid encodes a polypeptide having PDCT1 activity;
(b) a nucleic acid sequence encoding a polypeptide having at least 80% sequence identity to SEQ ID NO: 2, 4, 6, 8, 10, 12, 14, 16, 40, 42, 44, and/or 46, wherein the polypeptide has PDCT1 activity;
(c) a fragment of (a) or (b), wherein the fragment encodes a polypeptide having PDCT1 activity; or
(d) a nucleic acid sequence fully complementary to any of (a) to (c).

23. An isolated, a synthetic, or a recombinant polynucleotide comprising polynucleotide of claim 22 and:
(a) a nucleic acid sequence having at least 80% sequence identity to SEQ ID NO: 35, 37, or 47, wherein the nucleic acid encodes a polypeptide having PDCT19 activity;
(b) a nucleic acid sequence encoding a polypeptide having at least 80% sequence identity to SEQ ID NO: 36, 38, or 48, wherein the polypeptide has PDCT19 activity;
(c) a fragment of (a) or (b), wherein the fragment encodes a polypeptide having PDCT19 activity; or
(d) a nucleic acid sequence fully complementary to any of (a) to (c).

24. A nucleic acid construct comprising a polynucleotide of claim 22, operably linked to one or more heterologous control sequences that directs the expression of the protein of interest in a cell, preferably in a plant cell.

25. A vector comprising the polynucleotide of claim 22 or a nucleic acid construct.

26. A host cell comprising a polynucleotide of claim 22, a nucleic acid construct or a vector.

27. The host cell of claim 26, wherein said host cell is selected from the group consisting of Agrobacterium, yeast, bacterial, algae or plant cell.

28. A method of producing the polypeptide of selected from the groups consisting of:
(a) a PDCT1 having at least 80% sequence identity with SEQ ID NO: 2, 4, 6, 8, 10, 12, 14, 16, 40, 42, 44, and/or 46;
(b) a PDCT1 encoded by a polynucleotide having at least 80% sequence identity with SEQ ID NO: 1, 3, 5, 7, 9, 11, 13, 15, 39, 41, 43, and/or 45;
(c) a PDCT1 encoded by a polynucleotide that hybridizes under high stringency conditions with (i) a polynucleotide that encodes the amino acid sequence of SEQ ID NO: 2, 4, 6, 8, 10, 12, 14, 16, 40, 42, 44, and/or 46, or (ii) the full-length complement of (i),
(d) a variant of the PDCT1 of SEQ ID NO: 2, 4, 6, 8, 10, 12, 14, 16, 40, 42, 44, and/or 46 comprising a substitution, deletion, and/or insertion at one or more positions and having PDCT1 activity;
(e) a PDCT1 encoded by a polynucleotide that differs from SEQ ID NO: 1, 3, 5, 7, 9, 11, 13, 15, 39, 41, 43, and/or 45 due to the degeneracy of the genetic code; and
(f) a fragment of the PDCT1 of (a), (b), (c), (d) or (e) having PDCT1 activity
or the polynucleotide of claim 22, comprising the steps of
(i) providing a host cell,
(ii) cultivating the host cell of step (a) under conditions conductive for the production of the polypeptide or the polynucleotide in the host cell; and
(iii) optionally, recovering the polypeptide or or the polynucleotide.

29. A method for the production of a transgenic plant, plant cell, plant seed, a part thereof, or an oil thereof, having an increased amount of GLA and/or an increase in total PUFA and/or an increased ratio of C20 fatty acids to C18, and/or an increased the conversion rate of a Delta-6 elongase relative to control plants, said method comprising:
(i) introducing and expressing in a plant, or part thereof, or plant cell, or plant seed a nucleic acid encoding a polypeptide as defined in claim 28; and
(ii) cultivating said plant cell or plant under conditions an increased amount of GLA and/or an increase in total PUFA and/or an increased ratio of C20 fatty acids to C18, and/or an increased the conversion rate of a Delta-6 elongase relative to control plants.

30. A method for the production of a transgenic plant, plant cell, plant seed, a part thereof, or an oil thereof, having an increased amount of GLA and/or an increase in total PUFA and/or an increased ratio of C20 fatty acids to C18, and/or an increased the conversion rate of a Delta-6 elongase relative to control plants, said method comprising
(i) replacing in a plant cell or plant a regulatory element controlling the endogenous expression of the polypeptide as defined in claim 28 or of an endogenous nucleic acid molecule encoding the polypeptide by a replacement regulatory element that increases the expression of the endogenous polypeptide or of the endogenous nucleic acid molecule encoding the polypeptide; and
(ii) cultivating said plant cell or plant under conditions promoting an increased amount of GLA and/or an increase in total PUFA and/or an increased ratio of C20 fatty acids to C18, and/or an increased the conversion rate of a Delta-6 elongase relative to control plants.

31. A transgenic plant, or part thereof, or plant cell, or plant seed obtainable by a method according to claim 29.

32. A transgenic plant, or part thereof, or plant cell, or plant seed having an increased amount of GLA and/or an increase in total PUFA and/or an increased ratio of C20 fatty acids to C18, and/or an increased the conversion rate of a Delta-6 elongase relative to control plants, resulting from the increased activity or expression rate of the PDCT as defined in claim 29 in a seed cell.

33. A transgenic plant, or part thereof, or plant cell, or plant seed according to claim 31, wherein said transgenic plant, or part thereof, or plant cell, or plant seed is a Camelina seed or a Brassica sp seed.

34. A transgenic plant, or part thereof, or plant cell, or plant seed comprising a nucleic acid construct of claim 24.

35. A transgenic plant according to claim 31, or a transgenic plant cell or a plant seed derived therefrom, wherein said plant, or cell or seed is an oil crop plant.

36. The plant of claim 31, wherein the expression or activity of at least one PDCT3 and/or a PDCT5 is reduced, the PDCT3 and/or a PDCT5 selected from
(a) PDCT3 and/or PDCT5 having at least 80% sequence identity with SEQ ID NO: 18, 20, 22, 24, 26, 28, 30, 32, 50, 52, 54, 56, 58, and/or 60;
(b) PDCT3 and/or PDCT5 encoded by a polynucleotide having at least 80% sequence identity with SEQ ID NO: 17, 19, 21, 23, 27, 29, 31, 49, 51, 53, 55, and/or 57;
(c) PDCT3 and/or PDCT5 encoded by one or more polynucleotides that hybridize under high stringency conditions with (i) a polynucleotide that encodes the amino acid sequence of SEQ ID NO: 18, 20, 22, 24, 26, 28, 30, 32, 50, 52, 54, 56, 58, and/or 60, or (ii) the full-length complement of (i);
(d) variants of the PDCT3 and/or PDCT5 of SEQ ID NO: 18, 20, 22, 24, 26, 28, 30, 32, 50, 52, 54, 56, 58, and/or 60, comprising a substitution, deletion, and/or insertion at one or more positions and having PDCT3 and/or PDCT5 activity;
(e) PDCT3 and/or PDCT5 encoded by a polynucleotide that differs from SEQ ID NO: 17, 19, 21, 23, 27, 29, 31, 49, 51, 53, 55, and/or 57 due to the degeneracy of the genetic code; and
(f) fragments of the PDCT3 and/or PDCT5 of (a), (b), (c), (d) or (e) having PDCT3 and/or PDCT5 activity.

37. A protein preparation comprising the polypeptide of claim 29, wherein the protein preparation comprises a lyophilized composition/formulation and/or additional enzymes or compounds.

38. A plant raw oil derived from the plant of claim 31.

39. A plant raw oil having an increased amount of GLA and/or an increase in total PUFA and/or an increased ratio of C20 fatty acids to C18.

40. An antibody or a fragment thereof specifically binding to the polypeptide as defined in claim 28.

41. A product derived or produced from a harvestable part of a plant of claim 31,
wherein the product is a dry pellet, a pulp pellet, a pressed stem, a meal, a powder, or a fibre, containing a composition produced from the plant; or
wherein the product comprises an oil, a fat, a fatty acid, a carbohydrate, or a starch, a sap, a juice, a molasses, a syrup, a chaff, or a protein produced from the plant.

42. (canceled)

43. A feed or food product comprising the plant oil of claim 38.

44. The feed composition of claim 43, comprising no oil derived from animals.

45. A method for the production of a composition comprising the fatty acid 22:1 in a plant, or part thereof, like a plant cell, and/or part seed, or part thereof,
wherein the level of the 22:1 fatty acid in % (w/w) in the triacylglycerol fraction is higher than the 22:1 fatty acid level in % (w/w) in the diacylglyerol fraction, comprising,
providing a plant cable to produce GLA and having an increased activity or expression of one or more PDCT compared to the wild type, the PDCT selected from the group consisting of:
(a) a PDCT1 having at least 80% sequence identity with SEQ ID NO: 2, 4, 6, 8, 10, 12, 14, 16, 40, 42, 44, and/or 46;
(b) a PDCT1 encoded by a polynucleotide having at least 80% sequence identity with SEQ ID NO: 1, 3, 5, 7, 9, 11, 13, 15, 39, 41, 43, and/or 45;
(c) a PDCT1 encoded by a polynucleotide that hybridizes under high stringency conditions with (i) a polynucleotide that encodes the amino acid sequence of SEQ ID NO: 2, 4, 6, 8, 10, 12, 14, 16, 40, 42, 44, and/or 46, or (ii) the full-length complement of (i),
(d) a variant of the PDCT1 of SEQ ID NO: 2, 4, 6, 8, 10, 12, 14, 16, 40, 42, 44, and/or 46 comprising a substitution, deletion, and/or insertion at one or more positions and having PDCT1 activity;
(e) a PDCT1 encoded by a polynucleotide that differs from SEQ ID NO: 1, 3, 5, 7, 9, 11, 13, 15, 39, 41, 43, and/or 45 due to the degeneracy of the genetic code; and
(f) a fragment of the PDCT1 of (a), (b), (c), (d) or (e) having PDCT1 activity;

46. A method for the production of a composition comprising the fatty acid SDA in a plant, or part thereof, like a plant cell, and/or part seed, or part thereof,
wherein the level of SDA in % (w/w) in the phosphatyidylcholine (PC) fraction is higher than the SDA level in % (w/w) in the triacylglycerol fraction, comprising,
providing a plant cable to produce SDA and having an increased activity or expression of one or more PDCT compared to the wild type, the PDCT selected from the group consisting of:
(a) a PDCT1 having at least 80% sequence identity with SEQ ID NO: 2, 4, 6, 8, 10, 12, 14, 16, 40, 42, 44, and/or 46;
(b) a PDCT1 encoded by a polynucleotide having at least 80% sequence identity with SEQ ID NO: 1, 3, 5, 7, 9, 11, 13, 15, 39, 41, 43, and/or 45;
(c) a PDCT1 encoded by a polynucleotide that hybridizes under high stringency conditions with (i) a polynucleotide that encodes the amino acid sequence of SEQ ID NO: 2, 4, 6, 8, 10, 12, 14, 16, 40, 42, 44, and/or 46, or (ii) the full-length complement of (i);
(d) a variant of the PDCT1 of SEQ ID NO: 2, 4, 6, 8, 10, 12, 14, 16, 40, 42, 44, and/or 46 comprising a substitution, preferably a conservative substitution, deletion, and/or insertion at one or more positions and having PDCT1 activity;
(e) a PDCT1 encoded by a polynucleotide that differs from SEQ ID NO: 1, 3, 5, 7, 9, 11, 13, 15, 39, 41, 43, and/or 45 due to the degeneracy of the genetic code; and
(f) a fragment of the PDCT1 of (a), (b), (c), (d) or (e) having PDCT1 activity;

47. Method for increasing the level of DPA, DHA and/or EPA in a plant, a part thereof, a plant cell, and/or plant seed, that is capable to produce DPA, DHA and/or EPA and, comprising providing a plant, a part thereof, a plant cell, and/or plant seed expressing a Delta-6 elongase and having an increasing the endogenous PDCT activity or expressing a heterologous PDCT or of one or more PDCT selected from the group consisting of:
(a) a PDCT1 having at least 80% sequence identity with SEQ ID NO: 2, 4, 6, 8, 10, 12, 14, 16, 40, 42, 44, and/or 46;
(b) a PDCT1 encoded by a polynucleotide having at least 80% sequence identity with SEQ ID NO: 1, 3, 5, 7, 9, 11, 13, 15, 39, 41, 43, and/or 45;
(c) a PDCT1 encoded by a polynucleotide that hybridizes under high stringency conditions with (i) a polynucleotide that encodes the amino acid sequence of SEQ ID NO: 2, 4, 6, 8, 10, 12, 14, 16, 40, 42, 44, and/or 46, or (ii) the full-length complement of (i);
(d) a variant of the PDCT1 of SEQ ID NO: 2, 4, 6, 8, 10, 12, 14, 16, 40, 42, 44, and/or 46 comprising a substitution, deletion, and/or insertion at one or more positions and having PDCT1 activity;
(e) a PDCT1 encoded by a polynucleotide that differs from SEQ ID NO: 1, 3, 5, 7, 9, 11, 13, 15, 39, 41, 43, and/or 45 due to the degeneracy of the genetic code; and
(f) a fragment of the PDCT1 of (a), (b), (c), (d) or (e) having PDCT1 activity;

48. A method to produce a plant or a part thereof, the plant cell, and/or the plant seed that comprises an oil that is characterized by
i. the level of the 18:2 fatty acid in % (w/w) in the triacylglycerol (TAG) fraction that is between 80 and 120% of the level as the 18:2 fatty acid level in % (w/w) in the diacylglyerol (DAG) fraction
ii. the total PUFAs is increased,
iii. a ratio of 18:1 fatty acids to total fatty acid content (w/w) is 10% less compared to the control and/or wherein ratio ALA to total fatty acid content is reduced by at least 20%.
lv: an increased amount of GLA,
v. an increased ratio of C20 fatty acids to C18,
vi. the level of the 22:1 fatty acid in % (w/w) in the triacylglycerol fraction is higher than the 22:1 fatty acid level in % (w/w) in the diacylglyerol fraction,
vii. the level of SDA in % (w/w) in the phosphatyidylcholine (PC) fraction is higher than the SDA level in % (w/w) in the triacylglycerol fraction

49. Method for the production of a plant, a part thereof, a plant cell, plant seed and/or plant seed oil, wherein the total PUFAs level is increased compared to a control, comprising increasing, in a plant, a part thereof, a plant cell, and/or plant seed the activity or expression rate of one or more PDCT compared to the control, wherein the PDCT is selected from the group consisting of:
(a) a PDCT1 having at least 80% sequence identity with SEQ ID NO: 2, 4, 6, 8, 10, 12, 14, 16, 40, 42, 44, and/or 46;
(b) a PDCT1 encoded by a polynucleotide having at least 80% sequence identity with SEQ ID NO: 1, 3, 5, 7, 9, 11, 13, 15, 39, 41, 43, and/or 45;
(c) a PDCT1 encoded by a polynucleotide that hybridizes under high stringency conditions with (i) a polynucleotide that encodes the amino acid sequence of SEQ ID NO: 2, 4, 6, 8, 10, 12, 14, 16, 40, 42, 44, and/or 46, or (ii) the full-length complement of (i);
(d) a variant of the PDCT1 of SEQ ID NO: 2, 4, 6, 8, 10, 12, 14, 16, 40, 42, 44, and/or 46 comprising a substitution, deletion, and/or insertion at one or more positions and having PDCT1 activity;
(e) a PDCT1 encoded by a polynucleotide that differs from SEQ ID NO: 1, 3, 5, 7, 9, 11, 13, 15, 39, 41, 43, and /or 45 due to the degeneracy of the genetic code; and
(f) a fragment of the PDCT1 of (a), (b), (c), (d) or (e) having PDCT1 activity;
and isolating the seed oil.
